(19) United States
(12) Patent Application Publication
(10) Pub. No.: US 2022/0204476 A1
(43) Pub. Date: Jun. 30, 2022

(54) RNA VIRUS INHIBITOR COMPOUNDS AND USES THEREOF

(71) Applicant: The Governors of the University of Alberta, Edmonton (CA)

(72) Inventors: James A. Nieman, Sherwood Park (CA); M. Joanne Lemieux, Edmonton (CA); D. Lorne Tyrrell, Edmonton (CA); Bing Bai, Edmonton (CA); Alexandr Belovodskiy, Edmonton (CA); Mostofa Hena, Edmonton (CA); Appan Srinivas Kandadai, Edmonton (CA); Michael A. Joyce, Edmonton (CA)

(21) Appl. No.: 17/556,700

(22) Filed: Dec. 20, 2021

Related U.S. Application Data
(60) Provisional application No. 63/128,622, filed on Dec. 21, 2020.

(51) Int. Cl. C07D 401/14; C07D 401/12; C07D 409/12; C07D 409/14; C07D 471/04; C07D 405/12; C07D 405/14; C07D 211/76; C07D 513/04; C07D 413/12; A61P 31/14

(52) U.S. Cl. CPC C07D 401/14; C07D 401/12; C07D 409/12; C07D 409/14; C07D 471/04; C07B 2200/05; C07D 405/14; C07D 211/76; C07D 513/04; C07D 413/12; A61P 31/14; C07D 405/12

(57) ABSTRACT

The present disclosure provides compounds and methods for inhibiting a virus infection, such as a Baltimore Group IV RNA virus infection, such as rhinovirus, coxsackievirus, norovirus and coronavirus. Aspects of the present disclosure also include methods of treating a virus infection in a subject.

### CROSS-REFERENCE TO RELATED APPLICATIONS

This application claims the benefit of U.S. Provisional Application No. 63/128,622, filed Dec. 21, 2020, the disclosure of which is incorporated herein by reference.

### INTRODUCTION

Ribonucleic acid (RNA) viruses have genomes made of RNA. RNA viruses may be categorized based on their genetic material by the Baltimore classification strategy. The groups include, for example, double-stranded RNA (dsRNA) viruses (Group III), positive sense single-stranded RNA viruses (+ssRNA) viruses (Group IV), and negative sense single-stranded RNA (−ssRNA) viruses (Group V). Single-stranded RNA (ssRNA) viruses cause many diseases in wildlife, domestic animals and humans. These viruses are genetically and antigenically diverse, exhibiting broad tissue tropisms and a wide pathogenic potential. The incubation periods of some of the most pathogenic viruses, e.g. the caliciviruses, are very short. Viral replication and expression of virulence factors may overwhelm early defense mechanisms (Xu 1991) and cause acute and severe symptoms.

Group IV RNA viruses contain a single strand of viral mRNA (also known as a positive/plus strand of genomic RNA). Positive sense RNA can be translated directly into protein, without a DNA intermediate and without creating a complementary RNA strand. The positive strand RNA genome is independently infectious, for most Group IV viruses. This means that in the absence of a capsid, envelope, or enclosed proteins, the RNA molecule, when inserted into a cell, is capable of using host cell machinery to construct additional viruses. Six subclasses of the Group IV single-stranded positive-sense RNA viruses include: Picornaviridae, Togaviridae, Coronaviridae, Hepeviridae, Caliciviridae, Flaviviridae, and Astroviridae (Berman (2012) Taxonomic Guide to Infectious Diseases. 237-246.).

Coronaviruses are a group of enveloped positive-sense single-stranded RNA viruses that are members of the Coronaviridae family, which are members of Group IV viruses. Since the turn of the millennium, three closely related coronaviruses have infected humans and spread internationally: the 2003 epidemic of Severe Acute Respirator Syndrome (SARS), 2012 Middle East respiratory syndrome (MERS) outbreak and the current Coronavirus Disease 2019 (COVID-19) pandemic. In each instance, these coronaviruses are thought to have originated from an animal reservoir and then ‘jumped’ to humans either directly or through an intermediate species. COVID-19 is caused by the Severe Acute Respiratory Syndrome Coronavirus 2 (SARS-CoV-2 or SARS2).

Noroviruses are a group of non-enveloped, positive-sense single-stranded RNA viruses that are members of the Caliciviridae family, which are members of Group IV viruses. Noroviruses is the most common cause of gastroenteritis and cases result in approximately 200,000 deaths globally per year.

Rhinoviruses have single-stranded positive sense RNA genomes and are not enveloped. They are members of the Picornaviridae family, which are members of Group IV viruses. Rhinoviruses are a predominant cause of the common cold. Rhinoviruses belong to the genus Enterovirus.

Coxsackieviruses are non-enveloped, positive-sense single-stranded RNA viruses that are members of the Picornaviridae family, which are members of Group IV viruses. Coxsackieviruses cause a variety of infections and are among the leading cause of aseptic meningitis. Coxsackieviruses belong to the genus Enterovirus.

### SUMMARY

The present disclosure provides compounds and methods for inhibiting a virus infection, such as a Baltimore Group IV RNA virus infection. Aspects of the present disclosure also include methods of treating a virus infection in a subject.

Aspects of the present disclosure include a compound of formula (I):

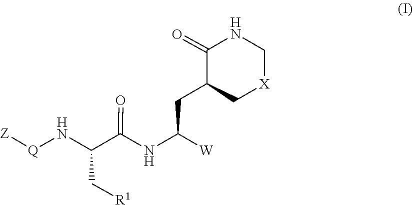

wherein:

W is selected from —CN and —C(═O)CH₂O—R²;

R¹ is selected from C₁₋₄ alkyl, substituted C₁₋₄ alkyl, and C₃₋₆ cycloalkyl;

R² is selected from aryl, substituted aryl, heterocycle, substituted heterocycle, heteroaryl, substituted heteroaryl and —C(═O)R³;

R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl;

X is selected from —CH₂— or is absent;

Q is selected from —CH₂—, —SO₂— and —C(═O)—; and

Z is selected from C₃₋₆ cycloalkyl, substituted C₃₋₆ cycloalkyl, heterocycle, arylalkoxy, substituted arylalkoxy, substituted heterocycle, heteroaryl, and substituted heteroaryl;

or a pharmaceutically acceptable salt, solvate, hydrate, or isotopic variant thereof;

with the proviso that if X is absent R³ is not 2,6-dichlorophenyl, 2,6-difluorophenyl, 2,6-dimethylphenyl, 2-cyanophenyl, 4-cyano-2-fluorophenyl, 4-chloro-2-hydroxyphenyl, 2,6-dimethoxyphenyl, 4-chlorophenyl, 4-methoxyphenyl, 4-cyanophenyl, 4-fluorophenyl, 4-methylphenyl, phenyl, cyclopropyl and t-butyl.

In some embodiments, W is CN. In some embodiments, W is —C(═O)CH₂O—R².

In some embodiments, R¹ is selected from —CH₂CH₃, —CH(CH₃)₂, —C(CH₃)₃, cyclopropyl, cyclobutyl, cyclopentyl and cyclohexyl. In some embodiments, R¹ is —CH(CH₃)₂. In some embodiments, R¹ is —C(CH₃)₃.

In some embodiments, X is absent. In some embodiments, X is —CH₂—.

In some embodiments, R² is selected from aryl, substituted aryl, heteroaryl, substituted heteroaryl and —C(═O)R³. In some embodiments, R² is selected from phenyl, 4-substituted aryl and 2-pyridyl. In some embodiments, R² is —C(═O)R³.

In some embodiments, R³ is selected from substituted C₄₋₆ alkyl, substituted C₃₋₆ cycloalkyl, phenyl, substituted aryl, heterocycle, substituted heterocycle, and substituted heteroaryl. In some embodiments, R³ is selected from —C(CH₃)₃, —C(CH₃)₂CF₃, and —C(CH₃)₂CN.

In some embodiments, R³ is selected from:

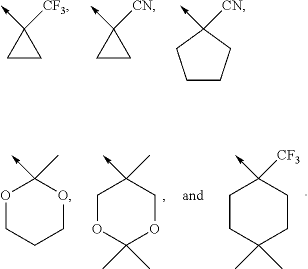

In some embodiments, R³ is selected from phenyl, substituted phenyl, substituted thiazole, substituted pyrazole and substituted pyridyl.

In some embodiments, R³ is selected from:

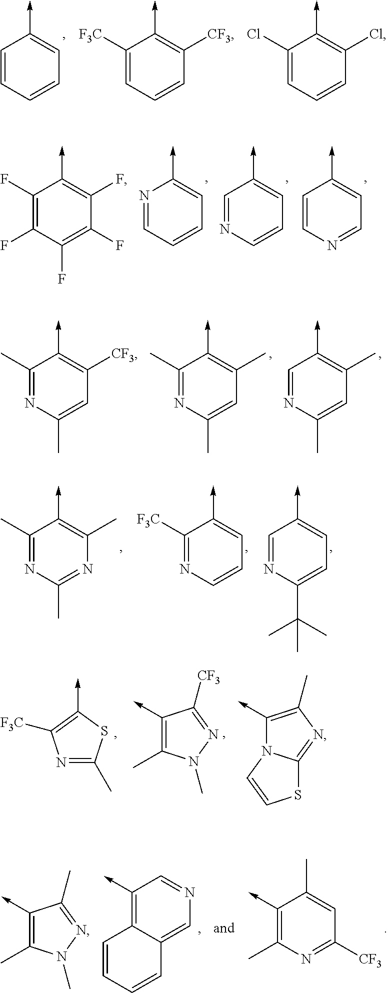

In some embodiments, Q is —C(═O)—.

In some embodiments, Z is selected from cyclopropyl, substituted cyclopropyl, cyclopentyl, substituted cyclopentyl, cyclohexyl, arylalkoxy, substituted arylalkoxy, tetrahydrofuran, substituted benzothiazole, benzofuran, substituted benzofuran, indoline, substituted indoline, indole, substituted indole, imidazole, substituted imidazole, pyridinyl, substituted pyridinyl, benzodioxine, substituted benzodioxine, piperidinyl, substituted piperidinyl, pyrrolidinyl, substituted pyrrolidinyl, oxazolyl, and substituted oxazolyl.

In some embodiments, Z is selected from phenylmethoxy and (3-chlorophenyl)methoxy. In some embodiments, Z is 4-methoxyindole.

In some embodiments, the compound is of formula (Ia):

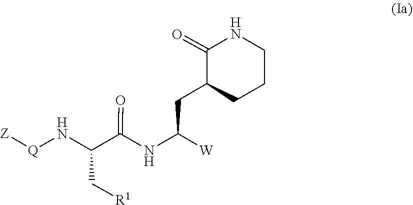

wherein:

W is selected from —CN and —C(═O)CH₂O—R²;

R¹ is selected from C₁₋₄ alkyl, substituted C₁₋₄ alkyl, and C₃₋₆ cycloalkyl;

R² is selected from aryl, substituted aryl, heterocycle, substituted heterocycle, heteroaryl, substituted heteroaryl and —C(═O)R³;

R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl;

Q is selected from —CH₂—, —SO₂— and —C(═O)—; and

Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl;

or a pharmaceutically acceptable salt, solvate, hydrate, or isotopic variant thereof.

In some embodiments, the compound is of formula (Ib):

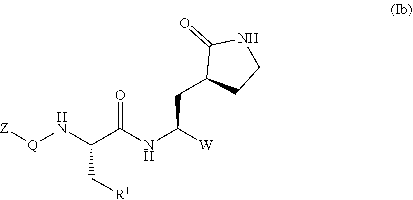

wherein:

W is selected from —CN and —C(═O)CH₂O—R²;

R¹ is selected from C₁₋₄ alkyl, substituted C₁₋₄ alkyl, and C₃₋₆ cycloalkyl;

R² is selected from aryl, substituted aryl, heterocycle, substituted heterocycle, heteroaryl, substituted heteroaryl and —C(═O)R³;

R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl;

Q is selected from —CH₂—, —SO₂— and —C(═O)—; and

Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl;

or a pharmaceutically acceptable salt, solvate, hydrate, or isotopic variant thereof;

with the proviso that R³ is not 2,6-dichlorophenyl, 2,6-difluorophenyl, 2,6-dimethylphenyl, 2-cyanophenyl, 4-cyano-2-fluorophenyl, 4-chloro-2-hydroxyphenyl, 2,6-dimethoxyphenyl, 4-chlorophenyl, 4-methoxyphenyl, 4-cyanophenyl, 4-fluorophenyl, 4-methylphenyl, phenyl, cyclopropyl and t-butyl.

In some embodiments, the compound is selected from the following structures:

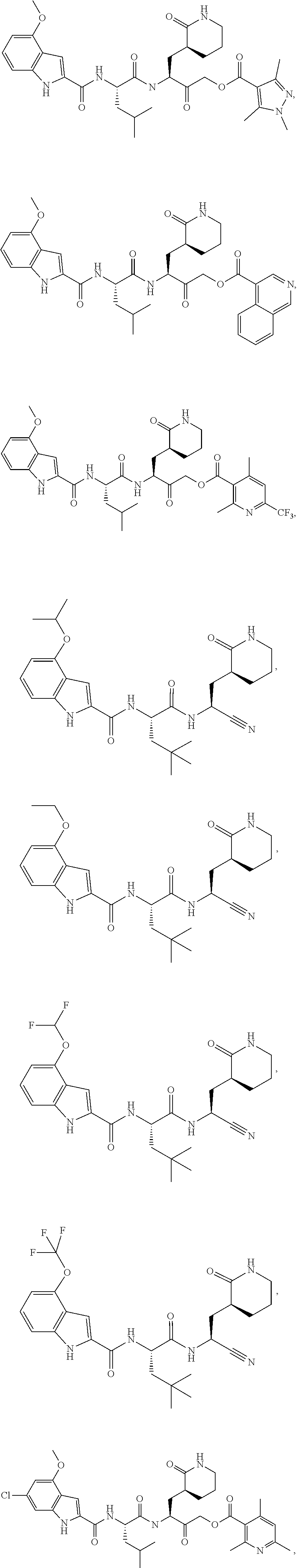

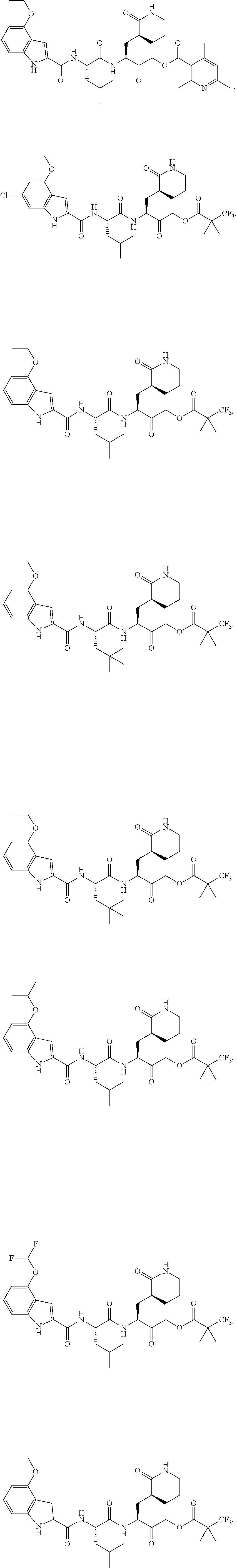

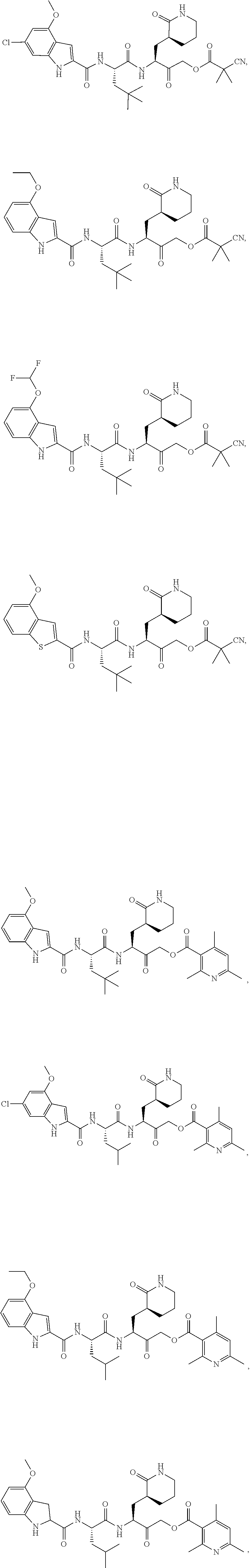

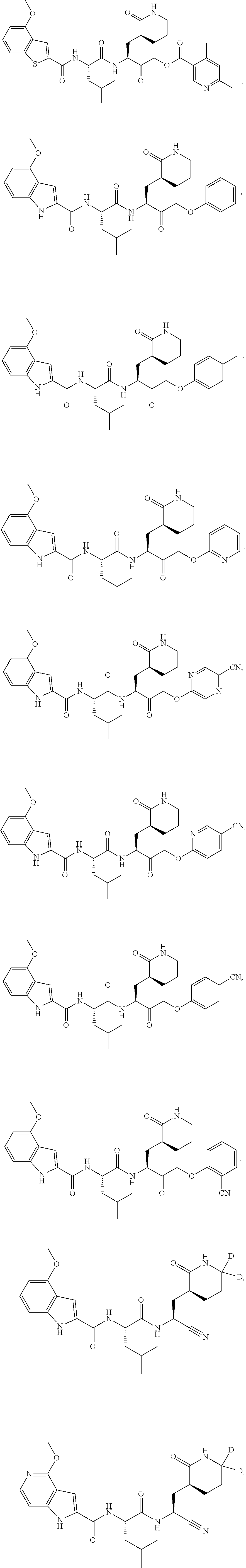

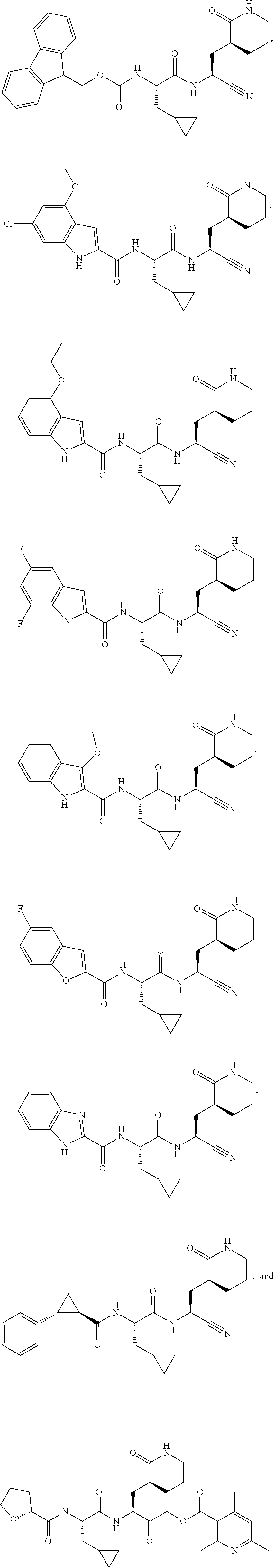

In some embodiments, the compound is selected from the following structures:

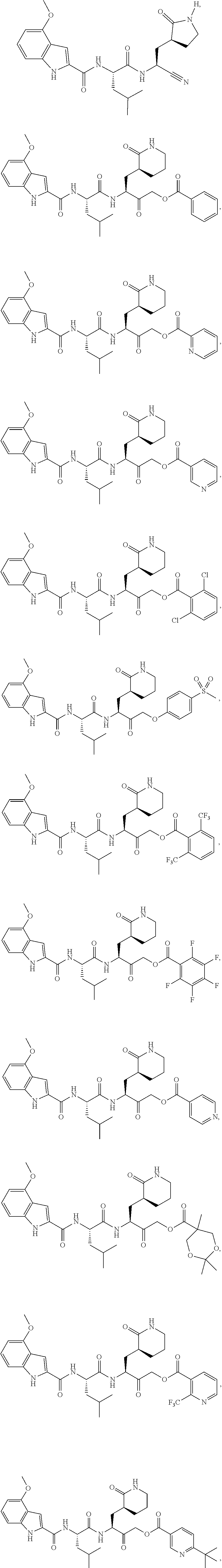

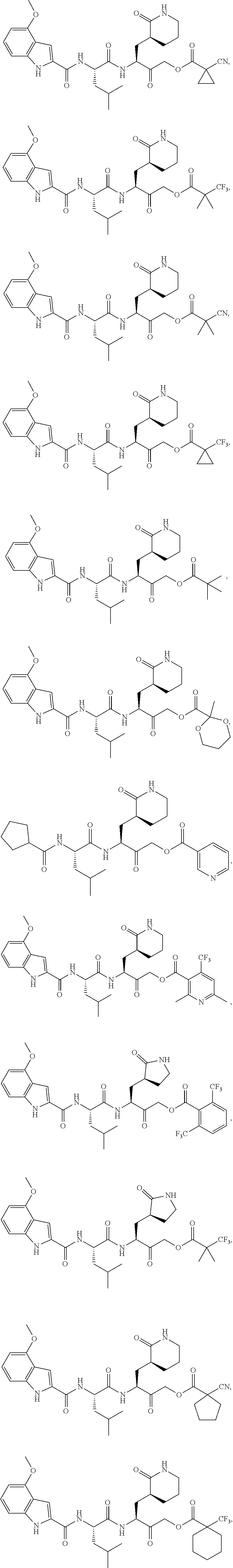

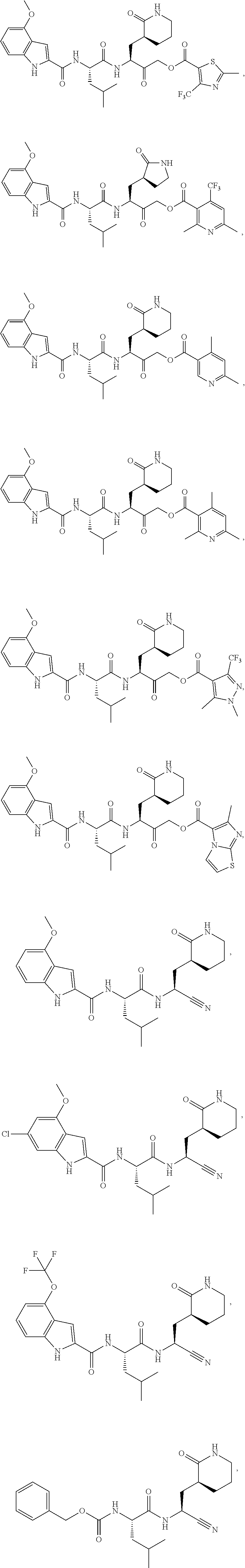

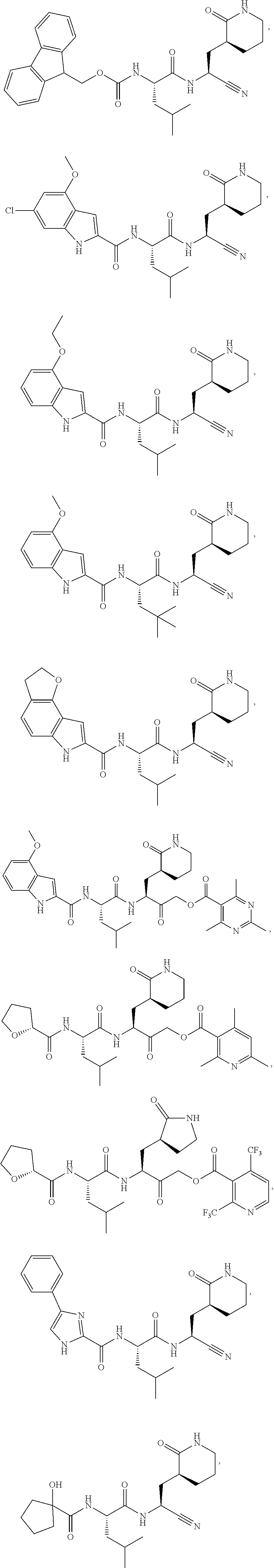

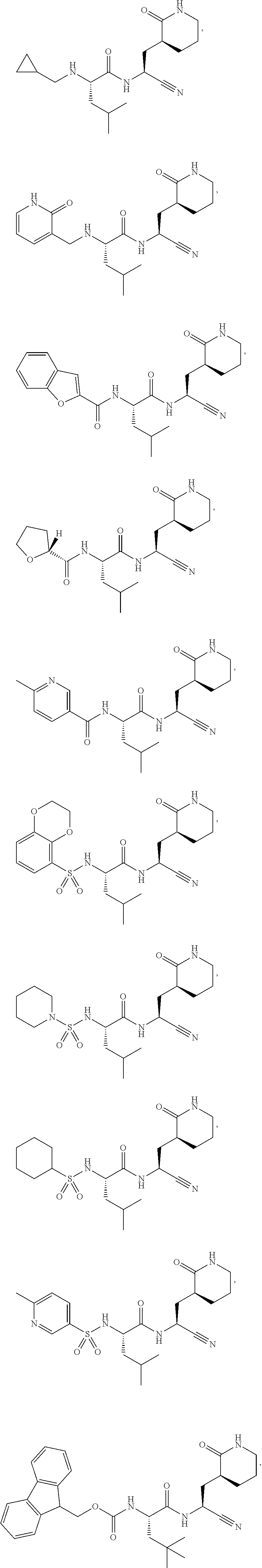

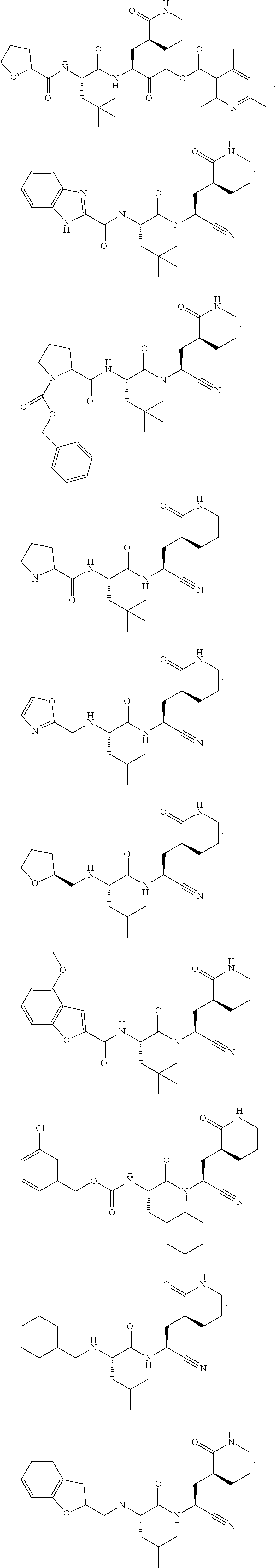

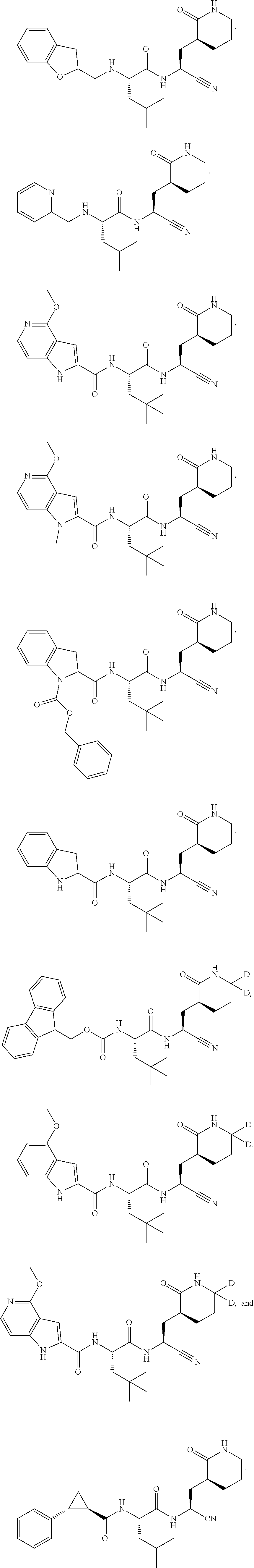

Aspects of the present disclosure include a method of inhibiting a Baltimore Group IV RNA virus in a cell infected with a Baltimore Group IV RNA virus, the method comprising contacting the cell with a compound according to the present disclosure.

In some embodiments, the Baltimore Group IV RNA virus is selected from the family of Picornaviridae, Calciviridae and Coronaviridae.

In some embodiments, the Baltimore Group IV RNA virus is selected from rhinovirus, coxsackievirus, norovirus and coronavirus.

In some embodiments, the Baltimore Group IV RNA virus is coronavirus.

In some embodiments, the coronavirus is one that causes disease in mammals. In some embodiments, the coronavirus causes disease in companion animals or livestock. In some embodiments, the coronavirus is a feline coronavirus. In some embodiments, the coronavirus is feline infectious peritonitis. In some embodiments, the coronavirus is a human coronavirus.

In some embodiments, the coronavirus is selected from Severe Acute Respiratory Syndrome coronavirus 2 (SARS-CoV-2), Severe Acute Respiratory syndrome coronavirus 1 (SARS-CoV-1) and Middle Eastern Respiratory syndrome-related coronavirus (MERS-CoV).

Aspects of the present disclosure include a method of treating a Baltimore Group IV RNA virus infection in a mammal, the method comprising administering to the mammal an effective amount of a compound according to the present disclosure.

In some embodiments, the mammal is selected from a companion animal and livestock. In some embodiments, the mammal is a feline. In some embodiments, the mammal is a human.

In some embodiments, the Baltimore Group IV RNA virus is selected from rhinovirus, coxsackie virus, norovirus and coronavirus.

In some embodiments, the Baltimore Group IV RNA virus is selected from norovirus, and coronavirus.

In some embodiments, the Baltimore Group IV RNA virus is human norovirus.

In some embodiments, the Baltimore Group IV RNA virus is a coronavirus that causes disease in mammals.

In some embodiments, the coronavirus is a feline coronavirus. In some embodiments, the feline coronavirus is feline infectious peritonitis.

In some embodiments, the coronavirus is a human coronavirus. In some embodiments, the human coronavirus is selected from Severe Acute Respiratory Syndrome coronavirus 2 (SARS-CoV-2), Severe Acute Respiratory syndrome coronavirus 1 (SARS-CoV-1) and Middle Eastern Respiratory syndrome-related coronavirus (MERS-CoV).

### BRIEF DESCRIPTION OF THE DRAWINGS

FIG. 1 shows exemplary concentration response curves for compounds screened for SARS-CoV-2 3CLP inhibition using a Fluorescence resonance energy transfer (FRET) assay.

FIG. 2 shows exemplary concentration response curves for compounds screened for SARS-CoV-2 3CLP inhibition using a Fluorescence resonance energy transfer (FRET) assay.

FIG. 3 shows exemplary concentration response curves for compounds screened for SARS-CoV-2 3CLP inhibition using a Fluorescence resonance energy transfer (FRET) assay.

FIG. 4 shows exemplary concentration response curves for compounds screened for inhibition of SARS-CoV-2 viral replication in an in vitro plaque reduction assay.

FIG. 5 shows exemplary concentration response curves for compounds for cytotoxicity using a cell viability assay.

### DEFINITIONS

The following terms have the following meanings unless otherwise indicated. Any undefined terms have their art recognized meanings.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain (except the C₁ carbon atom) have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-aryl, —SO-heteroaryl, —SO₂-alkyl, —SO₂-aryl, —SO₂-heteroaryl, and —NR^{a}R^{b}, wherein R^{a} and R^{b} may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰— and the like, where R¹⁰ is chosen from chosen from hydrogen, optionally substituted alkyl, cycloalkyl, alkenyl, cycloalkenyl, alkynyl, aryl, heteroaryl and heterocyclic. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group.

Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO— substituted alkyl, —SO-aryl, —SO-heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, NR²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “alkoxycarbonylamino” refers to the group —NR^{d}C(O)OR^{d} where each R^{d} is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Sulfonylamino” refers to the group —NR²¹SO₂R²², wherein R²¹ and R²² independently are selected from hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

“Amino” refers to the group —NH₂.

The term “substituted amino” refers to the group —NR^{m}R^{m} where each R^{m} is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O— substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Carbocycle” refers to non-aromatic or aromatic cyclic groups, such as cycloalkyl, cycloalkenyl, cycloalkynyl, and aryl groups as defined herein. A carbocycle group may be unsubstituted or substituted as defined herein.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic. To satisfy valence requirements, any heteroatoms in such heteroaryl rings may or may not be bonded to H or a substituent group, e.g., an alkyl group or other substituent as described herein. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from nitrogen, sulfur, or oxygen, where, in fused ring systems, one or more of the rings can be cycloalkyl, heterocyclyl, aryl, or heteroaryl, provided that the point of attachment is through the non-aromatic ring. Fused ring systems include compounds where two rings share two adjacent atoms. In fused heterocycle systems one or both of the two fused rings can be heterocyclyl. In certain embodiments, the nitrogen and/or sulfur atom(s) of the heterocyclic group are optionally oxidized to provide for the N-oxide, —S(O)—, or —SO₂— moieties. To satisfy valence requirements, any heteroatoms in such heterocyclic rings may or may not be bonded to one or more H or one or more substituent group(s), e.g., an alkyl group or other substituent as described herein.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, 1,2,3,4-tetrahydroquinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, 3,4-dihydro-1,4-benzoxazine, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂— substituted cycloalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —SO₂R⁷⁰, —SO₂O⁻M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻ M⁺, —P(O)(OR⁷⁰)₂, —C(O)R⁷⁰, —C(S)R⁷⁰, —C(NR⁷⁰)R⁷⁰, —C(O)O⁻ M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of 0, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —SO₂R⁷⁰, —SO₃⁻M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

“Pharmaceutically effective amount” and “therapeutically effective amount” refer to an amount of a compound sufficient to treat a specified disorder or disease or one or more of its symptoms and/or to prevent the occurrence of the disease or disorder. In reference to tumorigenic proliferative disorders, a pharmaceutically or therapeutically effective amount comprises an amount sufficient to, among other things, cause the tumor to shrink or decrease the growth rate of the tumor.

By “treating” or “treatment” is meant that at least an amelioration of the symptoms associated with the condition afflicting the subject is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the subject no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state or prophylactic treatment of a subject; (ii) inhibition, that is, arresting the development or further development of clinical symptoms, e.g., mitigating or completely inhibiting an active disease; and/or (iii) relief, that is, causing the regression of clinical symptoms or alleviating one or more symptoms of the disease or medical condition in the subject.

The terms “polypeptide,” “peptide,” and “protein” are used interchangeably herein to refer to a polymeric form of amino acids of any length. Unless specifically indicated otherwise, “polypeptide,” “peptide,” and “protein” can include genetically coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, proteins which contain at least one N-terminal methionine residue (e.g., to facilitate production in a recombinant host cell); immunologically tagged proteins; and the like.

“Native amino acid sequence” or “parent amino acid sequence” are used interchangeably herein to refer to the amino acid sequence of a polypeptide prior to modification to include a modified amino acid residue.

The terms “amino acid analog,” “unnatural amino acid,” and the like may be used interchangeably, and include amino acid-like compounds that are similar in structure and/or overall shape to one or more amino acids commonly found in naturally occurring proteins (e.g., Ala or A, Cys or C, Asp or D, Glu or E, Phe or F, Gly or G, His or H, Ile or I, Lys or K, Leu or L, Met or M, Asn or N, Pro or P, Gln or Q, Arg or R, Ser or S, Thr or T, Val or V, Trp or W, Tyr or Y). Amino acid analogs also include natural amino acids with modified side chains or backbones. Amino acid analogs also include amino acid analogs with the same stereochemistry as in the naturally occurring D-form, as well as the L-form of amino acid analogs. In some instances, the amino acid analogs share backbone structures, and/or the side chain structures of one or more natural amino acids, with difference(s) being one or more modified groups in the molecule. Such modification may include, but is not limited to, substitution of an atom (such as N) for a related atom (such as S), addition of a group (such as methyl, or hydroxyl, etc.) or an atom (such as Cl or Br, etc.), deletion of a group, substitution of a covalent bond (single bond for double bond, etc.), or combinations thereof. For example, amino acid analogs may include α-hydroxy acids, and α-amino acids, and the like.

The terms “amino acid side chain” or “side chain of an amino acid” and the like may be used to refer to the substituent attached to the α-carbon of an amino acid residue, including natural amino acids, unnatural amino acids, and amino acid analogs. An amino acid side chain can also include an amino acid side chain as described in the context of the modified amino acids and/or conjugates described herein.

As used herein the term “isolated” is meant to describe a compound of interest that is in an environment different from that in which the compound naturally occurs. “Isolated” is meant to include compounds that are within samples that are substantially enriched for the compound of interest and/or in which the compound of interest is partially or substantially purified.

As used herein, the term “substantially purified” refers to a compound that is removed from its natural environment and is at least 60% free, at least 75% free, at least 80% free, at least 85% free, at least 90% free, at least 95% free, at least 98% free, or more than 98% free, from other components with which it is naturally associated.

The term “physiological conditions” is meant to encompass those conditions compatible with living cells, e.g., predominantly aqueous conditions of a temperature, pH, salinity, etc. that are compatible with living cells.

As used herein, the term “chronic administration” refers to repeated administration of a compound to a subject. In such treatment, the compound can be administered at least once a week, such as at least once a day, or at least twice or three times a day for a period of at least one month, such as for example five months or more.

As used herein, the term “cysteine protease” refers to a protease having a nucleophilic thiol group in the active site. Cysteine proteases from different organisms can have significantly different cleavage sites. In many RNA class IV viruses, such as coronaviruses, rhinovirus, coxackieviruses and noroviruses, a well-conserved consensus sequence for the 3-chymotrypsin protease (3CP) and 3-chymotrypsin-like protease (3CLP) are observed. For these viruses, this is the main protease (also known as Mpro) responsible for cleaving the polyprotein generated from translation of the viral genome, which liberates the active viral proteins that are critical for viral replication. As this is not a host protease responsible for other critical functions, producing drugs that are highly selective for this viral protease will allow viral replication to be stopped and minimize toxicity for the host. To obtain sufficient inhibition of the protease activity and selectivity over other protease classes, the catalytic mechanism must also be considered in inhibitor design. For cysteine proteases, forming a covalent bond to the catalytic sulfur will ablate activity as it is vital to the cleavage mechanism; however, in some instances, excessive reactivity of the electrophile will also react with serine proteases, other cysteine proteases and other thiols resulting in toxicity. A moiety that forms the covalent bond to the sulfur in the inhibitor is termed the warhead.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present invention and are disclosed herein just as if each and every such sub-combination was individually and explicitly disclosed herein.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DETAILED DESCRIPTION

The present disclosure provides compounds and methods for inhibiting a virus infection, such as a Baltimore Group IV RNA virus infection. Aspects of the present disclosure also include methods of treating a virus infection in a subject.

### Compounds

Formula (I)

In certain embodiments, compounds of the present disclosure include a compound of formula (I):

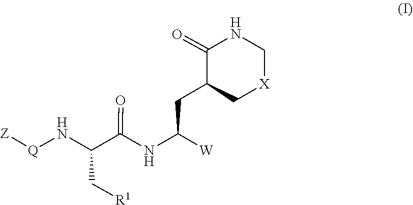

wherein:

W is selected from —CN and —C(═O)CH₂O—R²;

R¹ is selected from C₁₋₄ alkyl, substituted C₁₋₄ alkyl, and C₃₋₆ cycloalkyl;

R² is selected from aryl, substituted aryl, heterocycle, substituted heterocycle, heteroaryl, substituted heteroaryl and —C(═O)R³;

R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl;

X is selected from —CH₂— or is absent;

Q is selected from —CH₂—, —SO₂— and —C(═O)—; and

Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl;

or a pharmaceutically acceptable salt, solvate, hydrate, or isotopic variant thereof;

with the proviso that if X is absent R³ is not 2,6-dichlorophenyl, 2,6-difluorophenyl, 2,6-dimethylphenyl, 2-cyanophenyl, 4-cyano-2-fluorophenyl, 4-chloro-2-hydroxyphenyl, 2,6-dimethoxyphenyl, 4-chlorophenyl, 4-methoxyphenyl, 4-cyanophenyl, 4-fluorophenyl, 4-methylphenyl, phenyl, cyclopropyl and t-butyl.

In certain embodiments, W is selected from —CN and —C(═O)CH₂O—R². In some instances, W is CN. In some instances, W is —C(═O)CH₂O—R².

In certain embodiments, R¹ is selected from C₁₋₄ alkyl, substituted C₁₋₄ alkyl, and C₃₋₆ cycloalkyl. In some instances, R¹ is C₁₋₄ alkyl (e.g., methyl, ethyl, propyl, or butyl). In some instances, R¹ is substituted C₁₋₄ alkyl (e.g., substituted methyl, substituted ethyl, substituted propyl, or substituted butyl). In some instances, R¹ is C₃₋₆ cycloalkyl (e.g., cyclopropyl, cyclobutyl, cyclopentyl or cyclohexyl). For example, R¹ can be selected from —CH₂CH₃, —CH(CH₃)₂, —C(CH₃)₃, cyclopropyl, cyclobutyl, cyclopentyl and cyclohexyl. In some instances, R¹ is —CH₂CH₃. In some instances, R¹ is —CH(CH₃)₂. In some instances, R¹ is —C(CH₃)₃. In some instances, R¹ is cyclopropyl. In some instances, R¹ is cyclobutyl. In some instances, R¹ is cyclopentyl. In some instances, R¹ is cyclohexyl.

In certain embodiments, R² is selected from aryl, substituted aryl, heterocycle, substituted heterocycle, heteroaryl, substituted heteroaryl and —C(═O)R³. In certain embodiments, R² is selected from aryl, substituted aryl, heteroaryl, substituted heteroaryl and —C(═O)R³.

In some instances, R² is aryl (e.g., phenyl). In some instances, R² is substituted aryl (e.g., substituted phenyl). In some instances, R² is heterocycle. In some instances, R² is substituted heterocycle. In some instances, R² is heteroaryl. In some instances, R² is substituted heteroaryl. In some instances, R² is —C(═O)R³. For example, R² can be phenyl. In some cases, R² is 2-substituted aryl, such as, but not limited to 2-substituted phenyl. In some cases, R² is 3-substituted aryl, such as, but not limited to 3-substituted phenyl. In some cases, R² is 4-substituted aryl, such as, but not limited to 4-substituted phenyl. In some cases, R² is 2-pyridyl. In some cases, R² is 3-pyridyl. In some cases, R² is 4-pyridyl. In some cases, R² is substituted 2-pyridyl. In some cases, R² is substituted 3-pyridyl. In some cases, R² is substituted 4-pyridyl. In some cases, R² is pyrazinyl. In some cases, R² is substituted pyrazinyl. For example, R² can be selected from, but is not limited to, the following groups:

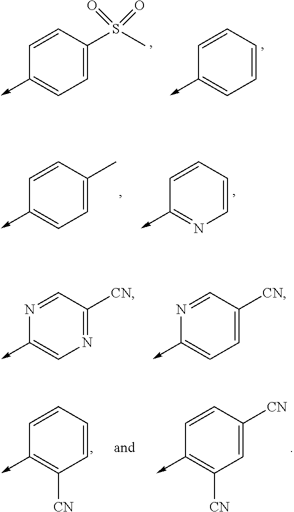

In certain embodiments, R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl. In certain embodiments, R³ is selected from substituted C₄₋₆ alkyl, substituted C₃₋₆ cycloalkyl, phenyl, substituted aryl, heterocycle, substituted heterocycle, and substituted heteroaryl.

In some instances, R³ is C₄₋₆ alkyl (e.g., butyl, pentyl, or hexyl). In some instances, R³ is substituted C₄₋₆ alkyl (e.g., substituted butyl, substituted pentyl, or substituted hexyl). In some instances, R³ is substituted C₄₋₆ alkyl, such as halo-C₄₋₆ alkyl. For example, R³ can be —C(CH₃)₃, —C(CH₃)₂CF₃, and —C(CH₃)₂CN. In some cases, R³ is —C(CH₃)₃. In some cases, R³ is —C(CH₃)₂CF₃. In some cases, R³ is —C(CH₃)₂CN.

In some instances, R³ is C₃₋₆ cycloalkyl (e.g., cyclopropyl, cyclobutyl, cyclopentyl, or cyclohexyl). In some instances, R³ is substituted C₃₋₆ cycloalkyl (e.g., substituted cyclopropyl, substituted cyclobutyl, substituted cyclopentyl, or substituted cyclohexyl). In some instances, R³ is heterocycle (e.g., 1,3-dioxane). In some instances, R³ is substituted heterocycle (e.g., substituted 1,3-dioxane). For example, R³ can be selected from, but is not limited to, the following groups:

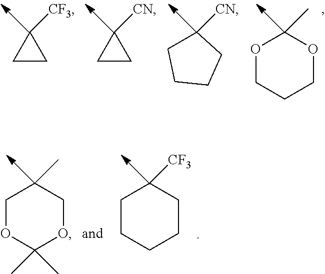

As used throughout the present disclosure, the arrow-bonds indicate the point of attachment of a group or substituent to a compound.

In some instances, R³ is aryl (e.g., phenyl). In some instances, R³ is substituted aryl (e.g., substituted phenyl). In some instances, R³ is heteroaryl. In some instances, R³ is substituted heteroaryl. For example, R³ can be selected from phenyl, substituted phenyl, substituted thiazole, substituted pyrazole, and substituted pyridyl. In some cases, R³ is 2-pyridyl. In some cases, R³ is 3-pyridyl. In some cases, R³ is 4-pyridyl. In some cases, R³ is substituted 2-pyridyl. In some cases, R³ is substituted 3-pyridyl. In some cases, R³ is substituted 4-pyridyl. In some cases, R³ is thiazole. In some cases, R³ is substituted thiazole. In some cases, R³ is pyrazole. In some cases, R³ is substituted pyrazole. In some cases, R³ is imidazothiazole. In some cases, R³ is substituted imidazothiazole. In some cases, R³ is isoquinoline. In some cases, R³ is substituted isoquinoline.

In some cases, R³ can be selected from, but is not limited to, the following groups:

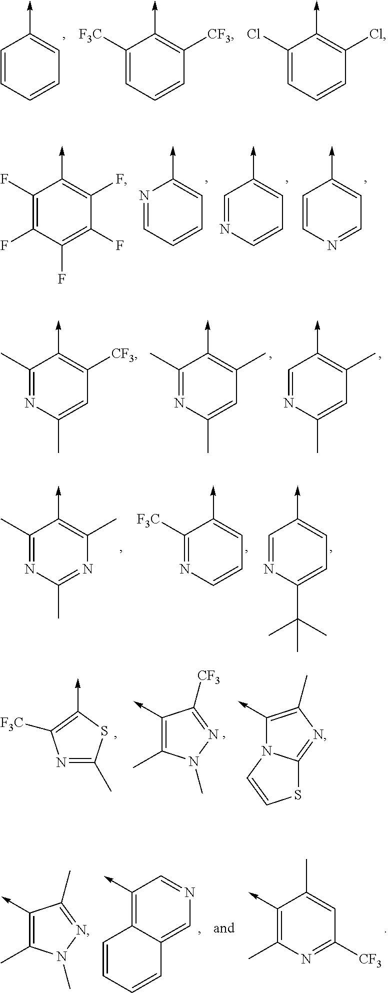

In certain embodiments, X is selected from —CH₂— or is absent. In some instances, X is —CH₂—. In some instances, X is absent.

In some embodiments of formula (I) where X is absent, the compounds of formula (I) do not include compounds where R³ is selected from 2,6-dichlorophenyl, 2,6-difluorophenyl, 2,6-dimethylphenyl, 2-cyanophenyl, 4-cyano-2-fluorophenyl, 4-chloro-2-hydroxyphenyl, 2,6-dimethoxyphenyl, 4-chlorophenyl, 4-methoxyphenyl, 4-cyanophenyl, 4-fluorophenyl, 4-methylphenyl, phenyl, cyclopropyl and t-butyl.

In certain embodiments, Q is selected from —CH₂—, —SO₂— and —C(═O)—. In some instances, Q is —CH₂—. In some instances, Q is —SO₂—. In some instances, Q is —C(═O)—.

In certain embodiments, Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl. In some instances, Z is C₃₋₆ cycloalkyl (e.g., cyclopropyl, cyclobutyl, cyclopentyl, or cyclohexyl). In some instances, Z is substituted C₃₋₆ cycloalkyl (e.g., substituted cyclopropyl, substituted cyclobutyl, substituted cyclopentyl, or substituted cyclohexyl). In some instances, Z is arylmethoxy or (substituted aryl)methoxy, such as, but not limited to phenylmethoxy, (3-chlorophenyl)methoxy, or 9H-fluoren-9-yl)methoxy. In some instances, Z is heterocycle. In some instances, Z is substituted heterocycle. In some instances, Z is heteroaryl. In some instances, Z is substituted heteroaryl. In some cases, Z is cyclopropyl. In some cases, Z is substituted cyclopropyl (e.g., phenylcyclopropyl, such as 2-phenylcyclopropyl). In some cases, Z can be cyclopentyl. In some cases, Z is substituted cyclopentyl (e.g., hydroxycyclopentyl). In some cases, Z is cyclohexyl. In some cases, Z is substituted cyclohexyl. In some cases, Z is tetrahydrofuran. In some cases, Z is substituted tetrahydrofuran. In some cases, Z is benzothiazole. In some cases, Z is substituted benzothiazole. In some cases, Z is benzofuran. In some cases, Z is substituted benzofuran. For example, Z can be 4-methoxy-1-benzofuran or 5-fluoro-1-benzofuran. In some cases, Z is indoline. In some cases, Z is substituted indoline. In some cases, Z is indole. In some cases, Z is substituted indole. For instance, Z can be 3-methoxy-1H-indole, 4-methoxy-1H-indole, 6-chloro-4-methoxy-1H-indole, 4-[(propan-2-yl)oxy]-1H-indole, 4-ethoxy-1H-indole, 4-(difluoromethoxy)-1H-indole, 4-(trifluoromethoxy)-1H-indole, 3,6-dihydro-2H-furo[2,3-e]indole, benzyl indoline-1-carboxylate, or 5,7-difluoro-1H-indole. In some cases, Z is imidazole. In some cases, Z is substituted imidazole. For instance, Z can be 4-phenyl-1H-imidazole or 1H-benzimidazole. In some cases, Z is pyridinyl. In some cases, Z is substituted pyridinyl. For example, Z can be 6-methylpyridine, 2-oxo-1,2-dihydropyridinyl, 4-methoxy-1H-pyrrolo[3,2-c]pyridine, or 4-methoxy-1-methyl-1H-pyrrolo[3,2-c]pyridine. In some cases, Z is benzodioxine. In some cases, Z is substituted benzodioxine. For example, Z can be 2,3-dihydro-1,4-benzodioxine. In some cases, Z is piperidinyl. In some cases, Z is substituted piperidinyl. In some cases, Z is pyrrolidinyl. In some cases, Z is substituted pyrrolidinyl. For example, Z can be benzyloxy)carbonyl]prolyl. In some cases, Z is oxazolyl. In some cases, Z is substituted oxazolyl.

Formula (Ia)

In certain embodiments of the compound of formula (I), X is —CH₂—. Accordingly, in certain embodiments, compounds of the present disclosure include a compound of formula (Ia):

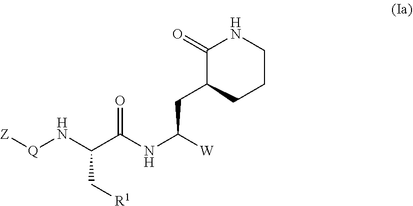

wherein:

W is selected from —CN and —C(═O)CH₂O—R²;

R¹ is selected from C₁₋₄ alkyl, substituted C₁₋₄ alkyl, and C₃₋₆ cycloalkyl;

R² is selected from aryl, substituted aryl, heterocycle, substituted heterocycle, heteroaryl, substituted heteroaryl and —C(═O)R³;

R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl;

Q is selected from —CH₂—, —SO₂— and —C(═O)—; and

Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl;

or a pharmaceutically acceptable salt, solvate, hydrate, or isotopic variant thereof.

In certain embodiments, W is selected from —CN and —C(═O)CH₂O—R². In some instances, W is CN. In some instances, W is —C(═O)CH₂O—R².

In certain embodiments, R¹ is selected from C₁₋₄ alkyl, substituted C₁₋₄ alkyl, and C₃₋₆ cycloalkyl. In some instances, R¹ is C₁₋₄ alkyl (e.g., methyl, ethyl, propyl, or butyl). In some instances, R¹ is substituted C₁₋₄ alkyl (e.g., substituted methyl, substituted ethyl, substituted propyl, or substituted butyl). In some instances, R¹ is C₃₋₆ cycloalkyl (e.g., cyclopropyl, cyclobutyl, cyclopentyl or cyclohexyl). For example, R¹ can be selected from —CH₂CH₃, —CH(CH₃)₂, —C(CH₃)₃, cyclopropyl, cyclobutyl, cyclopentyl and cyclohexyl. In some instances, R¹ is —CH₂CH₃. In some instances, R¹ is —CH(CH₃)₂. In some instances, R¹ is —C(CH₃)₃. In some instances, R¹ is cyclopropyl. In some instances, R¹ is cyclobutyl. In some instances, R¹ is cyclopentyl. In some instances, R¹ is cyclohexyl.

In certain embodiments, R² is selected from aryl, substituted aryl, heterocycle, substituted heterocycle, heteroaryl, substituted heteroaryl and —C(═O)R³. In certain embodiments, R² is selected from aryl, substituted aryl, heteroaryl, substituted heteroaryl and —C(═O)R³.

In some instances, R² is aryl (e.g., phenyl). In some instances, R² is substituted aryl (e.g., substituted phenyl). In some instances, R² is heterocycle. In some instances, R² is substituted heterocycle. In some instances, R² is heteroaryl. In some instances, R² is substituted heteroaryl. In some instances, R² is —C(═O)R³. For example, R² can be phenyl. In some cases, R² is 2-substituted aryl, such as, but not limited to 2-substituted phenyl. In some cases, R² is 3-substituted aryl, such as, but not limited to 3-substituted phenyl. In some cases, R² is 4-substituted aryl, such as, but not limited to 4-substituted phenyl. In some cases, R² is 2-pyridyl. In some cases, R² is 3-pyridyl. In some cases, R² is 4-pyridyl. In some cases, R² is substituted 2-pyridyl. In some cases, R² is substituted 3-pyridyl. In some cases, R² is substituted 4-pyridyl. In some cases, R² is pyrazinyl. In some cases, R² is substituted pyrazinyl. For example, R² can be selected from, but is not limited to, the following groups:

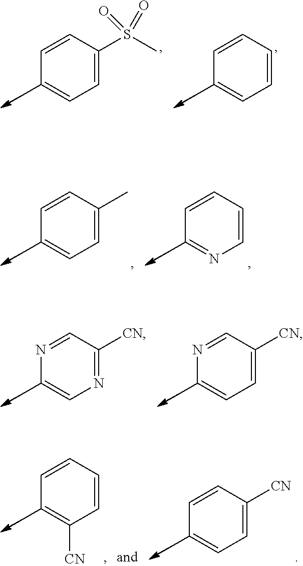

In certain embodiments, R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl. In certain embodiments, R³ is selected from substituted C₄₋₆ alkyl, substituted C₃₋₆ cycloalkyl, phenyl, substituted aryl, heterocycle, substituted heterocycle, and substituted heteroaryl.

In some instances, R³ is C₄₋₆ alkyl (e.g., butyl, pentyl, or hexyl). In some instances, R³ is substituted C₄₋₆ alkyl (e.g., substituted butyl, substituted pentyl, or substituted hexyl). In some instances, R³ is substituted C₄₋₆ alkyl, such as halo-C₄₋₆ alkyl. For example, R³ can be —C(CH₃)₃, —C(CH₃)₂CF₃, and —C(CH₃)₂CN. In some cases, R³ is —C(CH₃)₃. In some cases, R³ is —C(CH₃)₂CF₃. In some cases, R³ is —C(CH₃)₂CN.

In some instances, R³ is C₃₋₆ cycloalkyl (e.g., cyclopropyl, cyclobutyl, cyclopentyl, or cyclohexyl). In some instances, R³ is substituted C₃₋₆ cycloalkyl (e.g., substituted cyclopropyl, substituted cyclobutyl, substituted cyclopentyl, or substituted cyclohexyl). In some instances, R³ is heterocycle (e.g., 1,3-dioxane). In some instances, R³ is substituted heterocycle (e.g., substituted 1,3-dioxane). For example, R³ can be selected from, but is not limited to, the following groups:

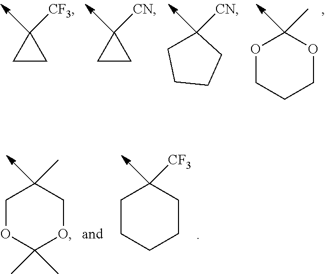

As used throughout the present disclosure, the arrow-bonds indicate the point of attachment of a group or substituent to a compound.

In some instances, R³ is aryl (e.g., phenyl). In some instances, R³ is substituted aryl (e.g., substituted phenyl). In some instances, R³ is heteroaryl. In some instances, R³ is substituted heteroaryl. For example, R³ can be selected from phenyl, substituted phenyl, substituted thiazole, substituted pyrazole, and substituted pyridyl. In some cases, R³ is 2-pyridyl. In some cases, R³ is 3-pyridyl. In some cases, R³ is 4-pyridyl. In some cases, R³ is substituted 2-pyridyl. In some cases, R³ is substituted 3-pyridyl. In some cases, R³ is substituted 4-pyridyl. In some cases, R³ is thiazole. In some cases, R³ is substituted thiazole. In some cases, R³ is pyrazole. In some cases, R³ is substituted pyrazole. In some cases, R³ is imidazothiazole. In some cases, R³ is substituted imidazothiazole. In some cases, R³ is isoquinoline. In some cases, R³ is substituted isoquinoline.

In some cases, R³ can be selected from, but is not limited to, the following groups:

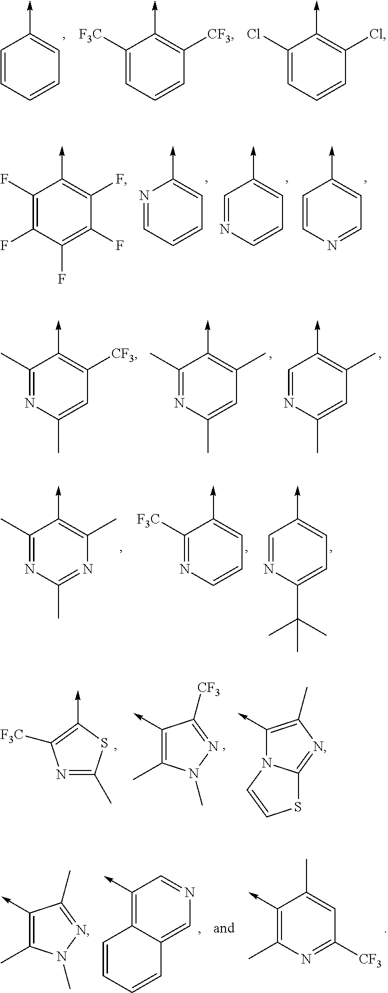

In certain embodiments, Q is selected from —CH₂—, —SO₂— and —C(═O)—. In some instances, Q is —CH₂—. In some instances, Q is —SO₂—. In some instances, Q is —C(═O)—.

In certain embodiments, Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl. In some instances, Z is C₃₋₆ cycloalkyl (e.g., cyclopropyl, cyclobutyl, cyclopentyl, or cyclohexyl). In some instances, Z is substituted C₃₋₆ cycloalkyl (e.g., substituted cyclopropyl, substituted cyclobutyl, substituted cyclopentyl, or substituted cyclohexyl). In some instances, Z is arylmethoxy or (substituted aryl)methoxy, such as, but not limited to phenylmethoxy, (3-chlorophenyl)methoxy, or 9H-fluoren-9-yl)methoxy. In some instances, Z is heterocycle. In some instances, Z is substituted heterocycle. In some instances, Z is heteroaryl. In some instances, Z is substituted heteroaryl. In some cases, Z is cyclopropyl. In some cases, Z is substituted cyclopropyl (e.g., phenylcyclopropyl, such as 2-phenylcyclopropyl). In some cases, Z can be cyclopentyl. In some cases, Z is substituted cyclopentyl (e.g., hydroxycyclopentyl). In some cases, Z is cyclohexyl. In some cases, Z is substituted cyclohexyl. In some cases, Z is tetrahydrofuran. In some cases, Z is substituted tetrahydrofuran. In some cases, Z is benzothiazole. In some cases, Z is substituted benzothiazole. In some cases, Z is benzofuran. In some cases, Z is substituted benzofuran. For example, Z can be 4-methoxy-1-benzofuran or 5-fluoro-1-benzofuran. In some cases, Z is indoline. In some cases, Z is substituted indoline. In some cases, Z is indole. In some cases, Z is substituted indole. For instance, Z can be 3-methoxy-1H-indole, 4-methoxy-1H-indole, 6-chloro-4-methoxy-1H-indole, 4-[(propan-2-yl)oxy]-1H-indole, 4-ethoxy-1H-indole, 4-(difluoromethoxy)-1H-indole, 4-(trifluoromethoxy)-1H-indole, 3,6-dihydro-2H-furo[2,3-e]indole, benzyl indoline-1-carboxylate, or 5,7-difluoro-1H-indole. In some cases, Z is imidazole. In some cases, Z is substituted imidazole. For instance, Z can be 4-phenyl-1H-imidazole or 1H-benzimidazole. In some cases, Z is pyridinyl. In some cases, Z is substituted pyridinyl. For example, Z can be 6-methylpyridine, 2-oxo-1,2-dihydropyridinyl, 4-methoxy-1H-pyrrolo[3,2-c]pyridine, or 4-methoxy-1-methyl-1H-pyrrolo[3,2-c]pyridine. In some cases, Z is benzodioxine. In some cases, Z is substituted benzodioxine. For example, Z can be 2,3-dihydro-1,4-benzodioxine. In some cases, Z is piperidinyl. In some cases, Z is substituted piperidinyl. In some cases, Z is pyrrolidinyl. In some cases, Z is substituted pyrrolidinyl. For example, Z can be benzyloxy)carbonyl]prolyl. In some cases, Z is oxazolyl. In some cases, Z is substituted oxazolyl.

Formula (Ib)

In certain embodiments of the compound of formula (I), X is absent. Accordingly, in certain embodiments, compounds of the present disclosure include a compound of formula (Ib):

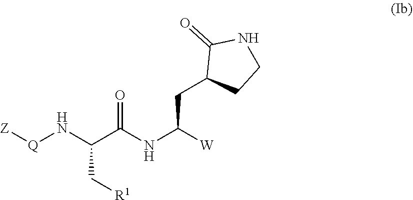

wherein:

W is selected from —CN and —C(═O)CH₂O—R²;

R¹ is selected from C₁₋₄ alkyl, substituted C₁₋₄ alkyl, and C₃₋₆ cycloalkyl;

R² is selected from aryl, substituted aryl, heterocycle, substituted heterocycle, heteroaryl, substituted heteroaryl and —C(═O)R³;

R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl;

Q is selected from —CH₂—, —SO₂— and —C(═O)—; and

Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl;

or a pharmaceutically acceptable salt, solvate, hydrate, or isotopic variant thereof;

with the proviso that R³ is not 2,6-dichlorophenyl, 2,6-difluorophenyl, 2,6-dimethylphenyl, 2-cyanophenyl, 4-cyano-2-fluorophenyl, 4-chloro-2-hydroxyphenyl, 2,6-dimethoxyphenyl, 4-chlorophenyl, 4-methoxyphenyl, 4-cyanophenyl, 4-fluorophenyl, 4-methylphenyl, phenyl, cyclopropyl and t-butyl.

In certain embodiments, W is selected from —CN and —C(═O)CH₂O—R². In some instances, W is CN. In some instances, W is —C(═O)CH₂O—R².

In certain embodiments, R¹ is selected from C₁₋₄ alkyl, substituted C₁₋₄ alkyl, and C₃₋₆ cycloalkyl. In some instances, R¹ is C₁₋₄ alkyl (e.g., methyl, ethyl, propyl, or butyl). In some instances, R¹ is substituted C₁₋₄ alkyl (e.g., substituted methyl, substituted ethyl, substituted propyl, or substituted butyl). In some instances, R¹ is C₃₋₆ cycloalkyl (e.g., cyclopropyl, cyclobutyl, cyclopentyl or cyclohexyl). For example, R¹ can be selected from —CH₂CH₃, —CH(CH₃)₂, —C(CH₃)₃, cyclopropyl, cyclobutyl, cyclopentyl and cyclohexyl. In some instances, R¹ is —CH₂CH₃. In some instances, R¹ is —CH(CH₃)₂. In some instances, R¹ is —C(CH₃)₃. In some instances, R¹ is cyclopropyl. In some instances, R¹ is cyclobutyl. In some instances, R¹ is cyclopentyl. In some instances, R¹ is cyclohexyl.

In certain embodiments, R² is selected from aryl, substituted aryl, heterocycle, substituted heterocycle, heteroaryl, substituted heteroaryl and —C(═O)R³. In certain embodiments, R² is selected from aryl, substituted aryl, heteroaryl, substituted heteroaryl and —C(═O)R³.

In some instances, R² is aryl (e.g., phenyl). In some instances, R² is substituted aryl (e.g., substituted phenyl). In some instances, R² is heterocycle. In some instances, R² is substituted heterocycle. In some instances, R² is heteroaryl. In some instances, R² is substituted heteroaryl. In some instances, R² is —C(═O)R³. For example, R² can be phenyl. In some cases, R² is 2-substituted aryl, such as, but not limited to 2-substituted phenyl. In some cases, R² is 3-substituted aryl, such as, but not limited to 3-substituted phenyl. In some cases, R² is 4-substituted aryl, such as, but not limited to 4-substituted phenyl. In some cases, R² is 2-pyridyl. In some cases, R² is 3-pyridyl. In some cases, R² is 4-pyridyl. In some cases, R² is substituted 2-pyridyl. In some cases, R² is substituted 3-pyridyl. In some cases, R² is substituted 4-pyridyl. In some cases, R² is pyrazinyl. In some cases, R² is substituted pyrazinyl. For example, R² can be selected from, but is not limited to, the following groups:

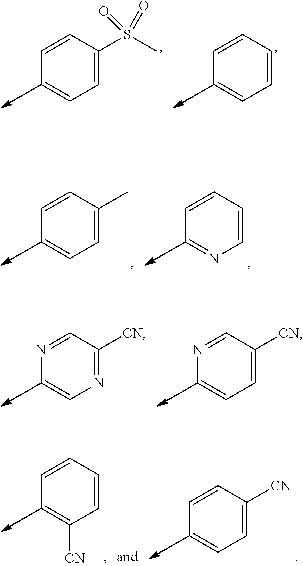

In certain embodiments, R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl. In certain embodiments, R³ is selected from substituted C₄₋₆ alkyl, substituted C₃₋₆ cycloalkyl, phenyl, substituted aryl, heterocycle, substituted heterocycle, and substituted heteroaryl.

In some instances, R³ is C₄₋₆ alkyl (e.g., butyl, pentyl, or hexyl). In some instances, R³ is substituted C₄₋₆ alkyl (e.g., substituted butyl, substituted pentyl, or substituted hexyl). In some instances, R³ is substituted C₄₋₆ alkyl, such as halo-C₄₋₆ alkyl. For example, R³ can be —C(CH₃)₂CF₃, and —C(CH₃)₂CN. In some cases, R³ is —C(CH₃)₂CF₃. In some cases, R³ is —C(CH₃)₂CN.

In some instances, R³ is C₃₋₆ cycloalkyl. In some instances, R³ is C₄₋₆ cycloalkyl (e.g., cyclobutyl, cyclopentyl, or cyclohexyl). In some instances, R³ is substituted C₃₋₆ cycloalkyl (e.g., substituted cyclopropyl, substituted cyclobutyl, substituted cyclopentyl, or substituted cyclohexyl). In some instances, R³ is heterocycle (e.g., 1,3-dioxane). In some instances, R³ is substituted heterocycle (e.g., substituted 1,3-dioxane). For example, R³ can be selected from, but is not limited to, the following groups:

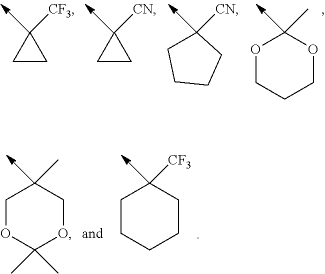

As used throughout the present disclosure, the arrow-bonds indicate the point of attachment of a group or substituent to a compound.

In some instances, R³ is aryl (e.g., naphthyl). In some instances, R³ is substituted aryl (e.g., substituted phenyl). In some instances, R³ is heteroaryl. In some instances, R³ is substituted heteroaryl. For example, R³ can be selected from phenyl, substituted phenyl, substituted thiazole, substituted pyrazole, and substituted pyridyl. In some cases, R³ is 2-pyridyl. In some cases, R³ is 3-pyridyl. In some cases, R³ is 4-pyridyl. In some cases, R³ is substituted 2-pyridyl. In some cases, R³ is substituted 3-pyridyl. In some cases, R³ is substituted 4-pyridyl. In some cases, R³ is thiazole. In some cases, R³ is substituted thiazole. In some cases, R³ is pyrazole. In some cases, R³ is substituted pyrazole. In some cases, R³ is imidazothiazole. In some cases, R³ is substituted imidazothiazole. In some cases, R³ is isoquinoline. In some cases, R³ is substituted isoquinoline.

In some cases, R³ can be selected from, but is not limited to, the following groups:

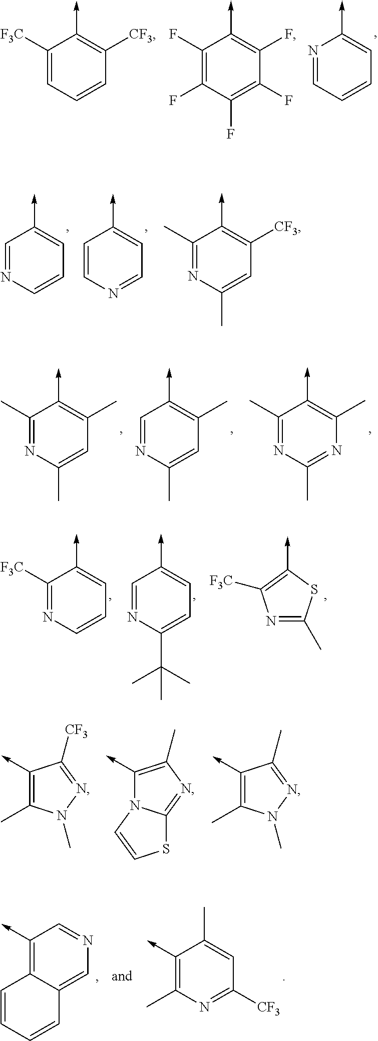

In certain embodiments, R³ is not 2,6-dichlorophenyl, 2,6-difluorophenyl, 2,6-dimethylphenyl, 2-cyanophenyl, 4-cyano-2-fluorophenyl, 4-chloro-2-hydroxyphenyl, 2,6-dimethoxyphenyl, 4-chlorophenyl, 4-methoxyphenyl, 4-cyanophenyl, 4-fluorophenyl, 4-methylphenyl, phenyl, cyclopropyl and t-butyl. In some cases, R³ is not 2,6-dichlorophenyl. In some cases, R³ is not 2,6-difluorophenyl. In some cases, R³ is not 2,6-dimethylphenyl. In some cases, R³ is not 2-cyanophenyl. In some cases, R³ is not 4-cyano-2-fluorophenyl. In some cases, R³ is not 4-chloro-2-hydroxyphenyl. In some cases, R³ is not 2,6-dimethoxyphenyl. In some cases, R³ is not 4-chlorophenyl. In some cases, R³ is not 4-methoxyphenyl. In some cases, R³ is not 4-cyanophenyl. In some cases, R³ is not 4-fluorophenyl. In some cases, R³ is not 4-methylphenyl. In some cases, R³ is not phenyl. In some cases, R³ is not cyclopropyl. In some cases, R³ is not t-butyl.

In certain embodiments, Q is selected from —CH₂—, —SO₂— and —C(═O)—. In some instances, Q is —CH₂—. In some instances, Q is —SO₂—. In some instances, Q is —C(═O)—.

In certain embodiments, Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl. In some instances, Z is C₃₋₆ cycloalkyl (e.g., cyclopropyl, cyclobutyl, cyclopentyl, or cyclohexyl). In some instances, Z is substituted C₃₋₆ cycloalkyl (e.g., substituted cyclopropyl, substituted cyclobutyl, substituted cyclopentyl, or substituted cyclohexyl). In some instances, Z is arylmethoxy or (substituted aryl)methoxy, such as, but not limited to phenylmethoxy, (3-chlorophenyl)methoxy, or 9H-fluoren-9-yl)methoxy. In some instances, Z is heterocycle. In some instances, Z is substituted heterocycle. In some instances, Z is heteroaryl. In some instances, Z is substituted heteroaryl. In some cases, Z is cyclopropyl. In some cases, Z is substituted cyclopropyl (e.g., phenylcyclopropyl, such as 2-phenylcyclopropyl). In some cases, Z can be cyclopentyl. In some cases, Z is substituted cyclopentyl (e.g., hydroxycyclopentyl). In some cases, Z is cyclohexyl. In some cases, Z is substituted cyclohexyl. In some cases, Z is tetrahydrofuran. In some cases, Z is substituted tetrahydrofuran. In some cases, Z is benzothiazole. In some cases, Z is substituted benzothiazole. In some cases, Z is benzofuran. In some cases, Z is substituted benzofuran. For example, Z can be 4-methoxy-1-benzofuran or 5-fluoro-1-benzofuran. In some cases, Z is indoline. In some cases, Z is substituted indoline. In some cases, Z is indole. In some cases, Z is substituted indole. For instance, Z can be 3-methoxy-1H-indole, 4-methoxy-1H-indole, 6-chloro-4-methoxy-1H-indole, 4-[(propan-2-yl)oxy]-1H-indole, 4-ethoxy-1H-indole, 4-(difluoromethoxy)-1H-indole, 4-(trifluoromethoxy)-1H-indole, 3,6-dihydro-2H-furo[2,3-e]indole, benzyl indoline-1-carboxylate, or 5,7-difluoro-1H-indole. In some cases, Z is imidazole. In some cases, Z is substituted imidazole. For instance, Z can be 4-phenyl-1H-imidazole or 1H-benzimidazole. In some cases, Z is pyridinyl. In some cases, Z is substituted pyridinyl. For example, Z can be 6-methylpyridine, 2-oxo-1,2-dihydropyridinyl, 4-methoxy-1H-pyrrolo[3,2-c]pyridine, or 4-methoxy-1-methyl-1H-pyrrolo[3,2-c]pyridine. In some cases, Z is benzodioxine. In some cases, Z is substituted benzodioxine. For example, Z can be 2,3-dihydro-1,4-benzodioxine. In some cases, Z is piperidinyl. In some cases, Z is substituted piperidinyl. In some cases, Z is pyrrolidinyl. In some cases, Z is substituted pyrrolidinyl. For example, Z can be benzyloxy)carbonyl]prolyl. In some cases, Z is oxazolyl. In some cases, Z is substituted oxazolyl.

In some embodiments, the compounds of formula (Ib) do not include compounds where R³ is selected from 2,6-dichlorophenyl, 2,6-difluorophenyl, 2,6-dimethylphenyl, 2-cyanophenyl, 4-cyano-2-fluorophenyl, 4-chloro-2-hydroxyphenyl, 2,6-dimethoxyphenyl, 4-chlorophenyl, 4-methoxyphenyl, 4-cyanophenyl, 4-fluorophenyl, 4-methylphenyl, phenyl, cyclopropyl and t-butyl.

Compounds of the present disclosure (e.g., compounds of formulae (I), (Ia) and (Ib) as described herein) also include an enantiomer, a mixture of enantiomers, a mixture of two or more diastereomers, a tautomer, a mixture of two or more tautomers, or an isotopic variant thereof.

In addition, compounds of the present disclosure (e.g., compounds of formulae (I), (Ia) and (Ib) as described herein) also include a pharmaceutically acceptable salt, solvate, or hydrate thereof.

In certain embodiments, compounds of the present disclosure (e.g., compounds that find use in the methods of the present disclosure) include compounds selected from:

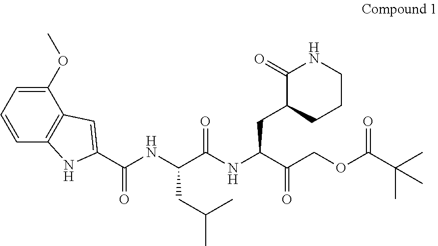 [SMILES: COc1cccc2[nH]c(C(=O)N[C@@H](CC(C)C)C(=O)N[C@@H](C[C@@H]3CCCNC3=O)C(=O)COC(=O)C(C)(C)C)cc12]

(3S)-3-{[N-(4-methoxy-1H-indole-2-carbonyl)-L-leucyl]amino}-2-oxo-4-[(3S)-2-oxopiperidin-3-yl]butyl 2,2-dimethylpropanoate,

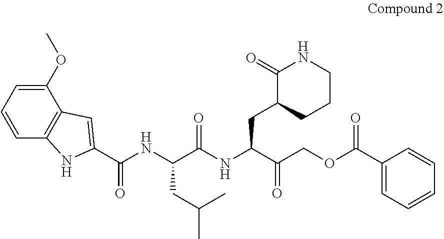 [SMILES: COc1cccc2[nH]c(C(=O)N[C@@H](CC(C)C)C(=O)N[C@@H](C[C@@H]3CCCNC3=O)C(=O)COC(=O)c3ccccc3)cc12]

(3S)-3-{[N-(4-methoxy-1H-indole-2-carbonyl)-L-leucyl]amino}-2-oxo-4-[(3S)-2-oxopiperidin-3-yl]butyl benzoate,

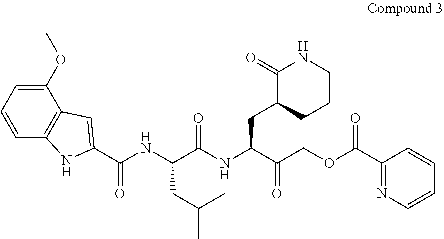 [SMILES: COc1cccc2[nH]c(C(=O)N[C@@H](CC(C)C)C(=O)N[C@@H](C[C@@H]3CCCNC3=O)C(=O)COC(=O)c3ccccn3)cc12]

(3S)-3-{[N-(4-methoxy-1H-indole-2-carbonyl)-L-leucyl]amino}-2-oxo-4-[(3S)-2-oxopiperidin-3-yl]butyl pyridine-2-carboxylate,

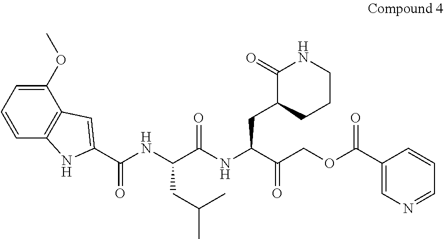 [SMILES: COc1cccc2[nH]c(C(=O)N[C@@H](CC(C)C)C(=O)N[C@@H](C[C@@H]3CCCNC3=O)C(=O)COC(=O)c3cccnc3)cc12]

(3S)-3-{[N-(4-methoxy-1H-indole-2-carbonyl)-L-leucyl]amino}-2-oxo-4-[(3S)-2-oxopiperidin-3-yl]butyl pyridine-3-carboxylate,

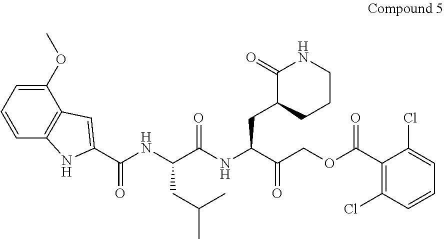 [SMILES: COc1cccc2[nH]c(C(=O)N[C@@H](CC(C)C)C(=O)N[C@@H](C[C@@H]3CCCNC3=O)C(=O)COC(=O)c3c(Cl)cccc3Cl)cc12]

(3S)-3-{[N-(4-methoxy-1H-indole-2-carbonyl)-L-leucyl]amino}-2-oxo-4-[(3S)-2-oxopiperidin-3-yl]butyl 2,6-dichlorobenzoate,

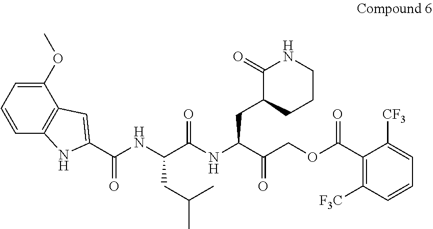 [SMILES: COc1cccc2[nH]c(C(=O)N[C@@H](CC(C)C)C(=O)N[C@@H](C[C@@H]3CCCNC3=O)C(=O)COC(=O)c3c(C(F)(F)F)cccc3C(F)(F)F)cc12]

(3S)-3-{[N-(4-methoxy-1H-indole-2-carbonyl)-L-leucyl]amino}-2-oxo-4-[(3S)-2-oxopiperidin-3-yl]butyl2,6-bis(trifluoromethyl)benzoate,

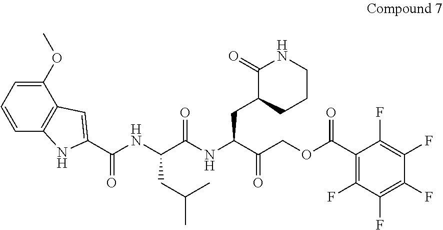 [SMILES: COc1cccc2[nH]c(C(=O)N[C@@H](CC(C)C)C(=O)N[C@@H](C[C@@H]3CCCNC3=O)C(=O)COC(=O)c3c(F)c(F)c(F)c(F)c3F)cc12]

(3S)-3-{[N-(4-methoxy-1H-indole-2-carbonyl)-L-leucyl]amino}-2-oxo-4-[(3S)-2-oxopiperidin-3-yl]butyl pentafluorobenzoate,

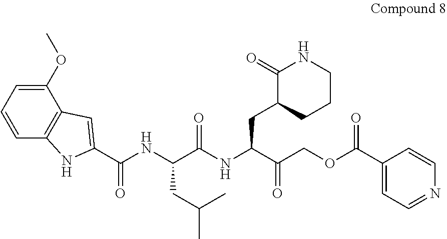 [SMILES: COc1cccc2[nH]c(C(=O)N[C@@H](CC(C)C)C(=O)N[C@@H](C[C@@H]3CCCNC3=O)C(=O)COC(=O)c3ccncc3)cc12]

(3S)-3-{[N-(4-methoxy-1H-indole-2-carbonyl)-L-leucyl]amino}-2-oxo-4-[(3S)-2-oxopiperidin-3-yl]butyl pyridine-4-carboxylate,

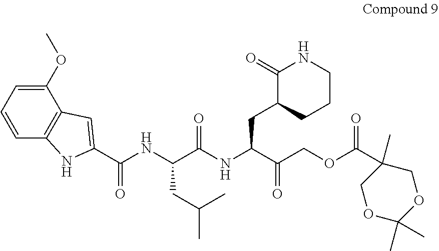 [SMILES: COc1cccc2[nH]c(C(=O)N[C@@H](CC(C)C)C(=O)N[C@@H](C[C@@H]3CCCNC3=O)C(=O)COC(=O)C3(C)COC(C)(C)OC3)cc12]

(3S)-3-{[N-(4-methoxy-1H-indole-2-carbonyl)-L-leucyl]amino}-2-oxo-4-[(3S)-2-oxopiperidin-3-yl]butyl2,2,5-trimethyl-1,3-dioxane-5-carboxylate,

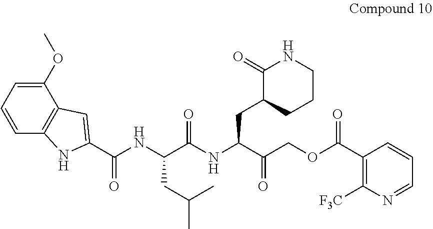 [SMILES: COc1cccc2[nH]c(C(=O)N[C@@H](CC(C)C)C(=O)N[C@@H](C[C@@H]3CCCNC3=O)C(=O)COC(=O)c3cccnc3C(F)(F)F)cc12]

(3S)-3-{[N-(4-methoxy-1H-indole-2-carbonyl)-L-leucyl]amino}-2-oxo-4-[(3S)-2-oxopiperidin-3-yl]butyl 2-(trifluoromethyl)pyridine-3-carboxylate,

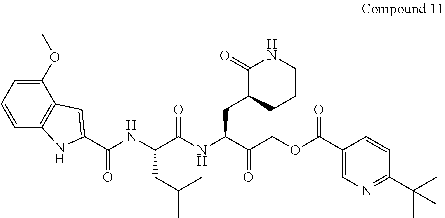 [SMILES: COc1cccc2[nH]c(C(=O)N[C@@H](CC(C)C)C(=O)N[C@@H](C[C@@H]3CCCNC3=O)C(=O)COC(=O)c3ccc(C(C)(C)C)nc3)cc12]

(3S)-3-{[N-(4-methoxy-1H-indole-2-carbonyl)-L-leucyl]amino}-2-oxo-4-[(3S)-2-oxopiperidin-3-yl]butyl 6-tert-butylpyridine-3-carboxylate,

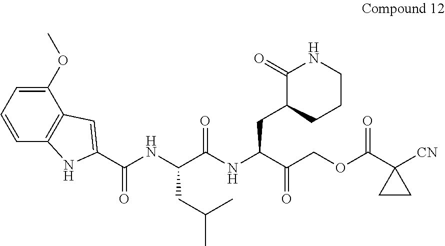 [SMILES: COc1cccc2[nH]c(C(=O)N[C@@H](CC(C)C)C(=O)N[C@@H](C[C@@H]3CCCNC3=O)C(=O)COC(=O)C3(C#N)CC3)cc12]

(3S)-3-{[N-(4-methoxy-1H-indole-2-carbonyl)-L-leucyl]amino}-2-oxo-4-[(3S)-2-oxopiperidin-3-yl]butyl 1-cyanocyclopropane-1-carboxylate,

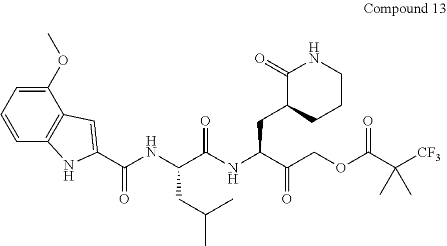 [SMILES: COc1cccc2[nH]c(C(=O)N[C@@H](CC(C)C)C(=O)N[C@@H](C[C@@H]3CCCNC3=O)C(=O)COC(=O)C(C)(C)C(F)(F)F)cc12]

(3S)-3-{[N-(4-methoxy-1H-indole-2-carbonyl)-L-leucyl]amino}-2-oxo-4-[(3S)-2-oxopiperidin-3-yl]butyl 3,3,3-trifluoro-2,2-dimethylpropanoate,

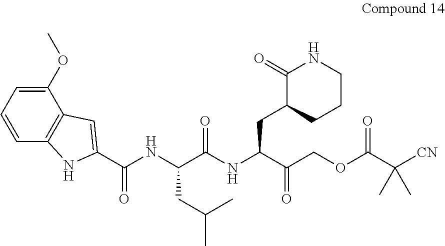 [SMILES: COc1cccc2[nH]c(C(=O)N[C@@H](CC(C)C)C(=O)N[C@@H](C[C@@H]3CCCNC3=O)C(=O)COC(=O)C(C)(C)C#N)cc12]

(3S)-3-{[N-(4-methoxy-1H-indole-2-carbonyl)-L-leucyl]amino}-2-oxo-4-[(3S)-2-oxopiperidin-3-yl]butyl 2-cyano-2-methylpropanoate,

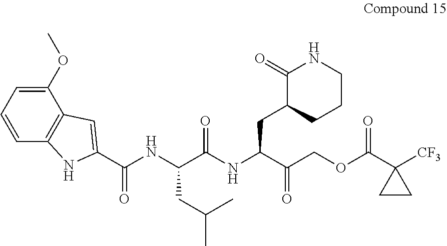 [SMILES: COc1cccc2[nH]c(C(=O)N[C@@H](CC(C)C)C(=O)N[C@@H](C[C@@H]3CCCNC3=O)C(=O)COC(=O)C3(C(F)(F)F)CC3)cc12]

(3S)-3-{[N-(4-methoxy-1H-indole-2-carbonyl)-L-leucyl]amino}-2-oxo-4-[(3S)-2-oxopiperidin-3-yl]butyl 1-(trifluoromethyl)cyclopropane-1-carboxylate,

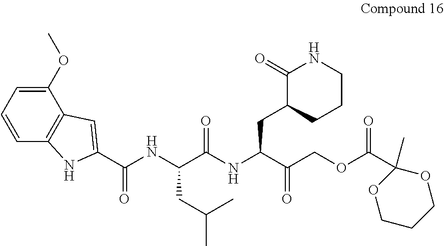 [SMILES: COc1cccc2[nH]c(C(=O)N[C@@H](CC(C)C)C(=O)N[C@@H](C[C@@H]3CCCNC3=O)C(=O)COC(=O)C3(C)OCCCO3)cc12]

(3S)-3-{[N-(4-methoxy-1H-indole-2-carbonyl)-L-leucyl]amino}-2-oxo-4-[(3S)-2-oxopiperidin-3-yl]butyl 2-methyl-1,3-dioxane-2-carboxylate,

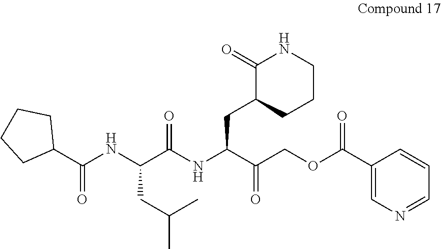 [SMILES: CC(C)C[C@H](NC(=O)C1CCCC1)C(=O)N[C@@H](C[C@@H]1CCCNC1=O)C(=O)COC(=O)c1cccnc1]

(3S)-3-{[N-(cyclopentanecarbonyl)-L-leucyl]amino}-2-oxo-4-[(3S)-2-oxopiperidin-3-yl]butyl pyridine-3-carboxylate,

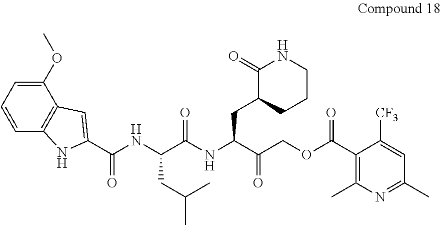 [SMILES: COc1cccc2[nH]c(C(=O)N[C@@H](CC(C)C)C(=O)N[C@@H](C[C@@H]3CCCNC3=O)C(=O)COC(=O)c3c(C(F)(F)F)cc(C)nc3C)cc12]

(3S)-3-{[N-(4-methoxy-1H-indole-2-carbonyl)-L-leucyl]amino}-2-oxo-4-[(3S)-2-oxopiperidin-3-yl]butyl 2,6-dimethyl-4-(trifluoromethyl)pyridine-3-carboxylate,

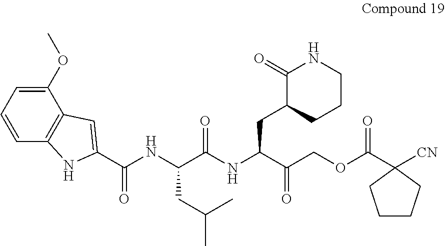 [SMILES: COc1cccc2[nH]c(C(=O)N[C@@H](CC(C)C)C(=O)N[C@@H](C[C@@H]3CCCNC3=O)C(=O)COC(=O)C3(C#N)CCCC3)cc12]

(3S)-3-{[N-(4-methoxy-1H-indole-2-carbonyl)-L-leucyl]amino}-2-oxo-4-[(3S)-2-oxopiperidin-3-yl]butyl 1-cyanocyclopentane-1-carboxylate,

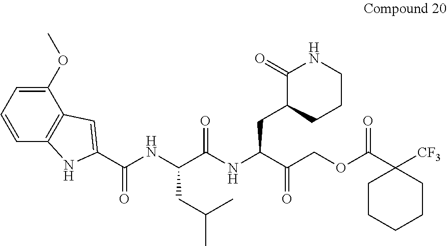 [SMILES: COc1cccc2[nH]c(C(=O)N[C@@H](CC(C)C)C(=O)N[C@@H](C[C@@H]3CCCNC3=O)C(=O)COC(=O)C3(C(F)(F)F)CCCCC3)cc12]

(3S)-3-{[N-(4-methoxy-1H-indole-2-carbonyl)-L-leucyl]amino}-2-oxo-4-[(3S)-2-oxopiperidin-3-yl]butyl 1-(trifluoromethyl)cyclohexane-1-carboxylate,

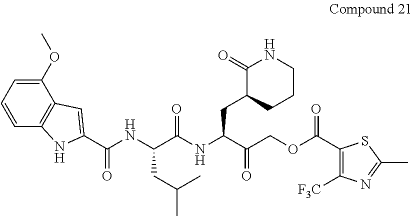 [SMILES: COc1cccc2[nH]c(C(=O)N[C@@H](CC(C)C)C(=O)N[C@@H](C[C@@H]3CCCNC3=O)C(=O)COC(=O)c3sc(C)nc3C(F)(F)F)cc12]

(3S)-3-{[N-(4-methoxy-1H-indole-2-carbonyl)-L-leucyl]amino}-2-oxo-4-[(3S)-2-oxopiperidin-3-yl]butyl 2-methyl-4-(trifluoromethyl)-1,3-thiazole-5-carboxylate,

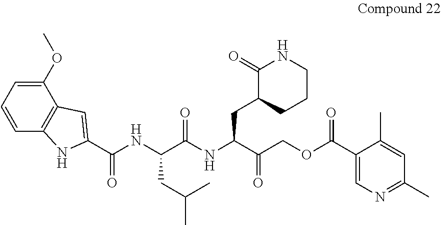 [SMILES: COc1cccc2[nH]c(C(=O)N[C@@H](CC(C)C)C(=O)N[C@@H](C[C@@H]3CCCNC3=O)C(=O)COC(=O)c3cnc(C)cc3C)cc12]

(3S)-3-{[N-(4-methoxy-1H-indole-2-carbonyl)-L-leucyl]amino}-2-oxo-4-[(3S)-2-oxopiperidin-3-yl]butyl 4,6-dimethylpyridine-3-carboxylate,

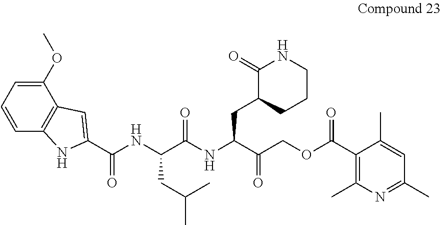 [SMILES: COc1cccc2[nH]c(C(=O)N[C@@H](CC(C)C)C(=O)N[C@@H](C[C@@H]3CCCNC3=O)C(=O)COC(=O)c3c(C)cc(C)nc3C)cc12]

(3S)-3-{[N-(4-methoxy-1H-indole-2-carbonyl)-L-leucyl]amino}-2-oxo-4-[(3S)-2-oxopiperidin-3-yl]butyl 2,4,6-trimethylpyridine-3-carboxylate,

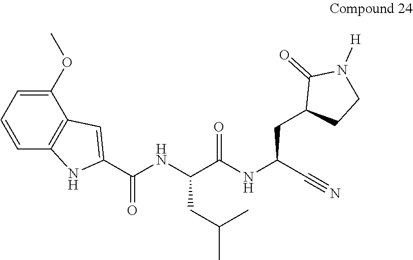 [SMILES: COc1cccc2[nH]c(C(=O)N[C@@H](CC(C)C)C(=O)N[C@H](C#N)C[C@@H]3CCNC3=O)cc12]

N-[(2S)-1-({(1S)-1-cyano-2-[(3S)-2-oxopyrrolidin-3-yl]ethyl}amino)-4-methyl-1-oxopentan-2-yl]-4-methoxy-1H-indole-2-carboxamide,

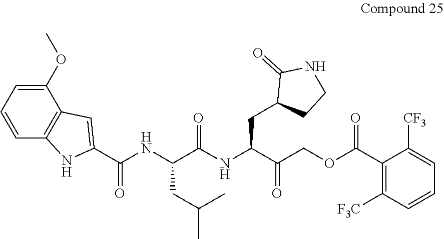 [SMILES: COc1cccc2[nH]c(C(=O)N[C@@H](CC(C)C)C(=O)N[C@@H](C[C@@H]3CCNC3=O)C(=O)COC(=O)c3c(C(F)(F)F)cccc3C(F)(F)F)cc12]

(3S)-3-{[N-(4-methoxy-1H-indole-2-carbonyl)-L-leucyl]amino}-2-oxo-4-[(3S)-2-oxopyrrolidin-3-yl]butyl2,6-bis(trifluoromethyl)benzoate,

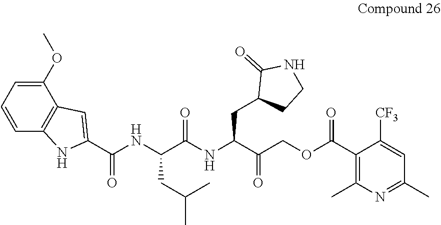 [SMILES: COc1cccc2[nH]c(C(=O)N[C@@H](CC(C)C)C(=O)N[C@@H](C[C@@H]3CCNC3=O)C(=O)COC(=O)c3c(C(F)(F)F)cc(C)nc3C)cc12]

(3S)-3-{[N-(4-methoxy-1H-indole-2-carbonyl)-L-leucyl]amino}-2-oxo-4-[(3S)-2-oxopyrrolidin-3-yl]butyl2,6-dimethyl-4-(trifluoromethyl)pyridine-3-carboxylate,

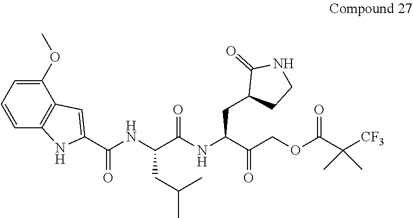 [SMILES: COc1cccc2[nH]c(C(=O)N[C@@H](CC(C)C)C(=O)N[C@@H](C[C@@H]3CCNC3=O)C(=O)COC(=O)C(C)(C)C(F)(F)F)cc12]

(3S)-3-{[N-(4-methoxy-1H-indole-2-carbonyl)-L-leucyl]amino}-2-oxo-4-[(3S)-2-oxopyrrolidin-3-yl]butyl 3,3,3-trifluoro-2,2-dimethylpropanoate,

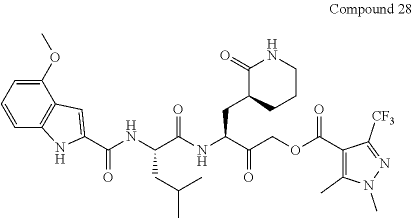 [SMILES: COc1cccc2[nH]c(C(=O)N[C@@H](CC(C)C)C(=O)N[C@@H](C[C@@H]3CCCNC3=O)C(=O)COC(=O)c3c(C(F)(F)F)nn(C)c3C)cc12]

(3S)-3-{[N-(4-methoxy-1H-indole-2-carbonyl)-L-leucyl]amino}-2-oxo-4-[(3S)-2-oxopiperidin-3-yl]butyl 1,5-dimethyl-3-(trifluoromethyl)-1H-pyrazole-4-carboxylate,

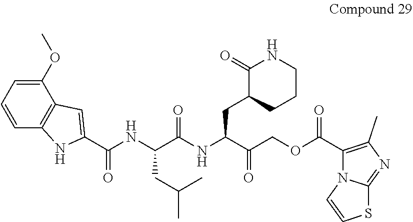 [SMILES: COc1cccc2[nH]c(C(=O)N[C@@H](CC(C)C)C(=O)N[C@@H](C[C@@H]3CCCNC3=O)C(=O)COC(=O)c3c(C)nc4sccn34)cc12]

(3S)-3-{[N-(4-methoxy-1H-indole-2-carbonyl)-L-leucyl]amino}-2-oxo-4-[(3S)-2-oxopiperidin-3-yl]butyl 6-methylimidazo[2,1-b][1,3]thiazole-5-carboxylate, and

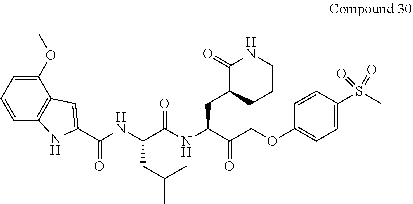 [SMILES: COc1cccc2[nH]c(C(=O)N[C@@H](CC(C)C)C(=O)N[C@@H](C[C@@H]3CCCNC3=O)C(=O)COc3ccc(S(C)(=O)=O)cc3)cc12]

N-[(2S)-1-({(2S)-4-[4-(methanesulfonyl)phenoxy]-3-oxo-1-[(3S)-2-oxopiperidin-3-yl]butan-2-yl}amino)-4-methyl-1-oxopentan-2-yl]-4-methoxy-1H-indole-2-carboxamide.

In certain embodiments, compounds of the present disclosure (e.g., compounds that find use in the methods of the present disclosure) include compounds selected from:

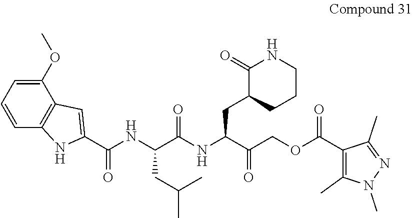 [SMILES: COc1cccc2[nH]c(C(=O)N[C@@H](CC(C)C)C(=O)N[C@@H](C[C@@H]3CCCNC3=O)C(=O)COC(=O)c3c(C)nn(C)c3C)cc12]

(3S)-3-{[N-(4-methoxy-1H-indole-2-carbonyl)-L-leucyl]amino}-2-oxo-4-[(3S)-2-oxopiperidin-3-yl]butyl 1,3,5-trimethyl-1H-pyrazole-4-carboxylate

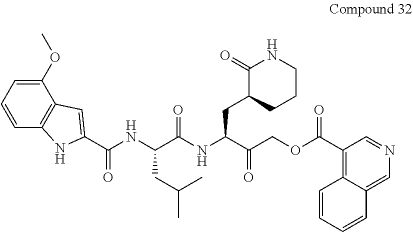 [SMILES: COc1cccc2[nH]c(C(=O)N[C@@H](CC(C)C)C(=O)N[C@@H](C[C@@H]3CCCNC3=O)C(=O)COC(=O)c3cncc4ccccc34)cc12]

(3S)-3-{[N-(4-methoxy-1H-indole-2-carbonyl)-L-leucyl]amino}-2-oxo-4-[(3S)-2-oxopiperidin-3-yl]butyl isoquinoline-4-carboxylate,

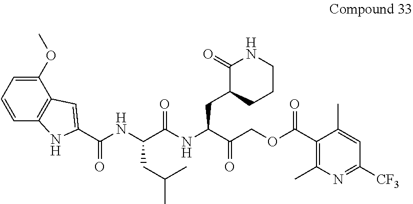 [SMILES: COc1cccc2[nH]c(C(=O)N[C@@H](CC(C)C)C(=O)N[C@@H](C[C@@H]3CCCNC3=O)C(=O)COC(=O)c3c(C)cc(C(F)(F)F)nc3C)cc12]

(3S)-3-{[N-(4-methoxy-1H-indole-2-carbonyl)-L-leucyl]amino}-2-oxo-4-[(3S)-2-oxopiperidin-3-yl]butyl 2,4-dimethyl-6-(trifluoromethyl)pyridine-3-carboxylate,

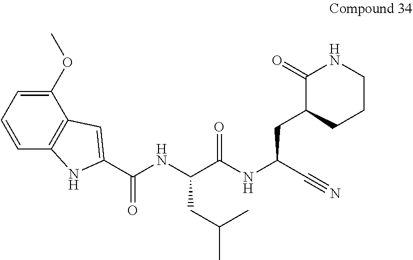 [SMILES: COc1cccc2[nH]c(C(=O)N[C@@H](CC(C)C)C(=O)N[C@H](C#N)C[C@@H]3CCCNC3=O)cc12]

N-[(2S)-1-({(1S)-1-cyano-2-[(3S)-2-oxopiperidin-3-yl]ethyl}amino)-4-methyl-1-oxopentan-2-yl]-4-methoxy-1H-indole-2-carboxamide,

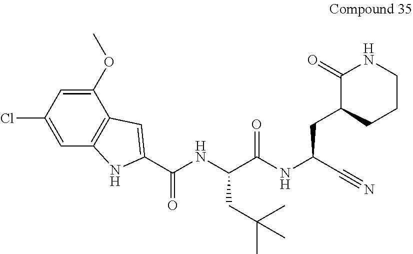 [SMILES: COc1cc(Cl)cc2[nH]c(C(=O)N[C@@H](CC(C)(C)C)C(=O)N[C@H](C#N)C[C@@H]3CCCNC3=O)cc12]

6-chloro-N-[(2S)-1-({(1S)-1-cyano-2-[(3S)-2-oxopiperidin-3-yl]ethyl}amino)-4,4-dimethyl-1-oxopentan-2-yl]-4-methoxy-1H-indole-2-carboxamide,

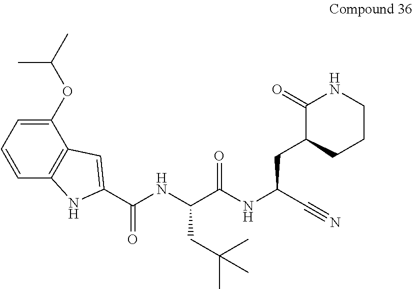 [SMILES: CC(C)Oc1cccc2[nH]c(C(=O)N[C@@H](CC(C)(C)C)C(=O)N[C@H](C#N)C[C@@H]3CCCNC3=O)cc12]

N-[(2S)-1-({(1S)-1-cyano-2-[(3S)-2-oxopiperidin-3-yl]ethyl}amino)-4,4-dimethyl-1-oxopentan-2-yl]-4-[(propan-2-yl)oxy]-1H-indole-2-carboxamide,

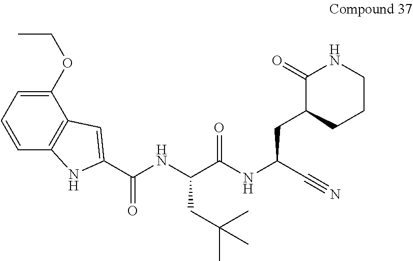 [SMILES: CCOc1cccc2[nH]c(C(=O)N[C@@H](CC(C)(C)C)C(=O)N[C@H](C#N)C[C@@H]3CCCNC3=O)cc12]

N-[(2S)-1-({(1S)-1-cyano-2-[(3S)-2-oxopiperidin-3-yl]ethyl}amino)-4,4-dimethyl-1-oxopentan-2-yl]-4-ethoxy-1H-indole-2-carboxamide,

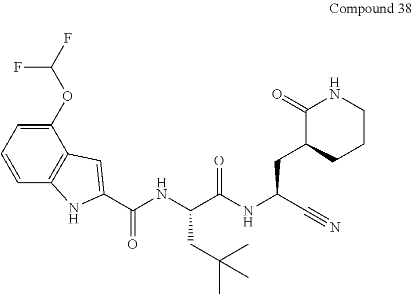 [SMILES: CC(C)(C)C[C@H](NC(=O)c1cc2c(OC(F)F)cccc2[nH]1)C(=O)N[C@H](C#N)C[C@@H]1CCCNC1=O]

N-[(2S)-1-({(1S)-1-cyano-2-[(3S)-2-oxopiperidin-3-yl]ethyl}amino)-4,4-dimethyl-1-oxopentan-2-yl]-4-(difluoromethoxy)-1H-indole-2-carboxamide,

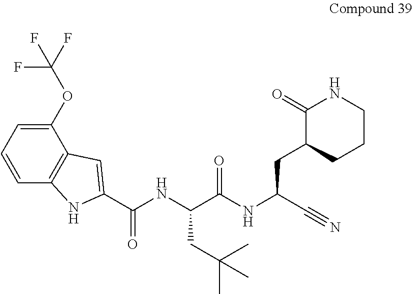 [SMILES: CC(C)(C)C[C@H](NC(=O)c1cc2c(OC(F)(F)F)cccc2[nH]1)C(=O)N[C@H](C#N)C[C@@H]1CCCNC1=O]

N-[(2S)-1-({(1S)-1-cyano-2-[(3S)-2-oxopiperidin-3-yl]ethyl}amino)-4,4-dimethyl-1-oxopentan-2-yl]-4-(trifluoromethoxy)-1H-indole-2-carboxamide,

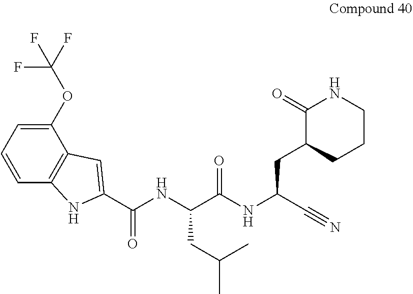 [SMILES: CC(C)C[C@H](NC(=O)c1cc2c(OC(F)(F)F)cccc2[nH]1)C(=O)N[C@H](C#N)C[C@@H]1CCCNC1=O]

N-[(2S)-1-({(1S)-1-cyano-2-[(3S)-2-oxopiperidin-3-yl]ethyl}amino)-4-methyl-1-oxopentan-2-yl]-4-(trifluoromethoxy)-1H-indole-2-carboxamide,

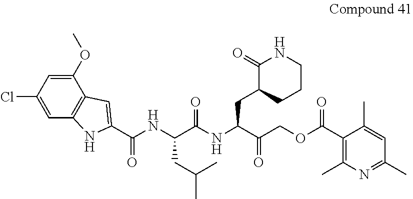 [SMILES: COc1cc(Cl)cc2[nH]c(C(=O)N[C@@H](CC(C)C)C(=O)N[C@@H](C[C@@H]3CCCNC3=O)C(=O)COC(=O)c3c(C)cc(C)nc3C)cc12]

(3S)-3-{[N-(6-chloro-4-methoxy-1H-indole-2-carbonyl)-L-leucyl]amino}-2-oxo-4-[(3S)-2-oxopiperidin-3-yl]butyl 2,4,6-trimethylpyridine-3-carboxylate,

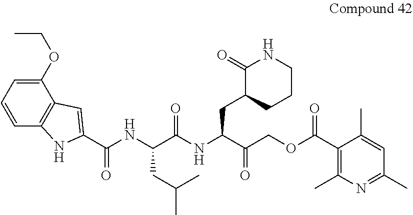 [SMILES: CCOc1cccc2[nH]c(C(=O)N[C@@H](CC(C)C)C(=O)N[C@@H](C[C@@H]3CCCNC3=O)C(=O)COC(=O)c3c(C)cc(C)nc3C)cc12]

(3S)-3-{[N-(4-ethoxy-1H-indole-2-carbonyl)-L-leucyl]amino}-2-oxo-4-[(3S)-2-oxopiperidin-3-yl]butyl2,4,6-trimethylpyridine-3-carboxylate,

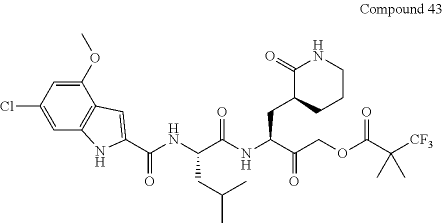 [SMILES: COc1cc(Cl)cc2[nH]c(C(=O)N[C@@H](CC(C)C)C(=O)N[C@@H](C[C@@H]3CCCNC3=O)C(=O)COC(=O)C(C)(C)C(F)(F)F)cc12]

(3S)-3-{[N-(6-chloro-4-methoxy-1H-indole-2-carbonyl)-L-leucyl]amino}-2-oxo-4-[(3S)-2-oxopiperidin-3-yl]butyl3,3,3-trifluoro-2,2-dimethylpropanoate,

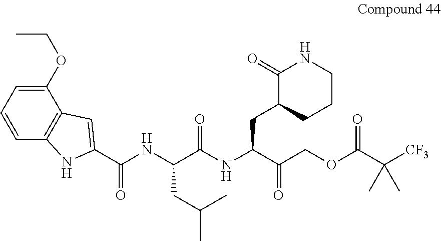 [SMILES: CCOc1cccc2[nH]c(C(=O)N[C@@H](CC(C)C)C(=O)N[C@@H](C[C@@H]3CCCNC3=O)C(=O)COC(=O)C(C)(C)C(F)(F)F)cc12]

(3S)-3-{[N-(4-ethoxy-1H-indole-2-carbonyl)-L-leucyl]amino}-2-oxo-4-[(3S)-2-oxopiperidin-3-yl]butyl3,3,3-trifluoro-2,2-dimethylpropanoate,

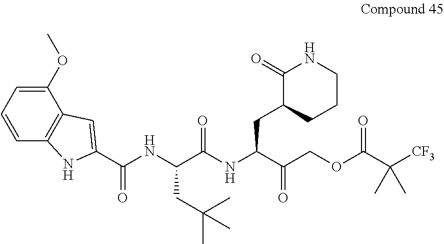 [SMILES: COc1cccc2[nH]c(C(=O)N[C@@H](CC(C)(C)C)C(=O)N[C@@H](C[C@@H]3CCCNC3=O)C(=O)COC(=O)C(C)(C)C(F)(F)F)cc12]

(3S)-3-{[N-(4-methoxy-1H-indole-2-carbonyl)-4-methyl-L-leucyl]amino}-2-oxo-4-[(3S)-2-oxopiperidin-3-yl]butyl 3,3,3-trifluoro-2,2-dimethylpropanoate,

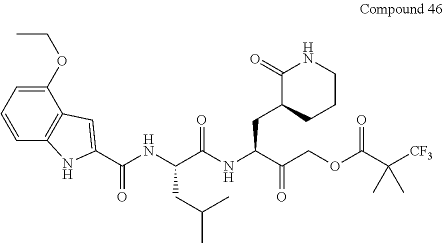 [SMILES: CCOc1cccc2[nH]c(C(=O)N[C@@H](CC(C)C)C(=O)N[C@@H](C[C@@H]3CCCNC3=O)C(=O)COC(=O)C(C)(C)C(F)(F)F)cc12]

(3S)-3-{[N-(4-ethoxy-1H-indole-2-carbonyl)-4-methyl-L-leucyl]amino}-2-oxo-4-[(3S)-2-oxopiperidin-3-yl]butyl3,3,3-trifluoro-2,2-dimethylpropanoate,

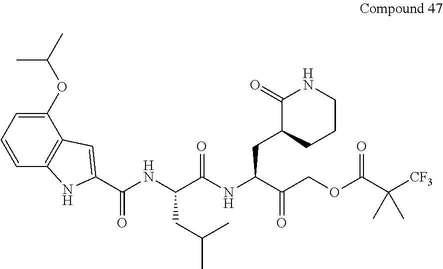 [SMILES: CC(C)C[C@H](NC(=O)c1cc2c(OC(C)C)cccc2[nH]1)C(=O)N[C@@H](C[C@@H]1CCCNC1=O)C(=O)COC(=O)C(C)(C)C(F)(F)F]

(3S)-2-oxo-4-[(3S)-2-oxopiperidin-3-yl]-3-[(N-{4-[(propan-2-yl)oxy]-1H-indole-2-carbonyl}-L-leucyl)amino]butyl3,3,3-trifluoro-2,2-dimethylpropanoate,

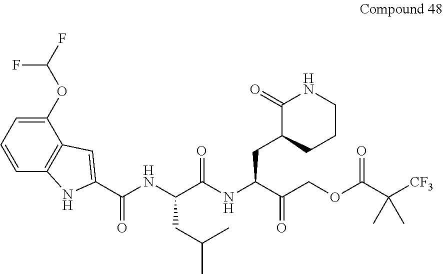 [SMILES: CC(C)C[C@H](NC(=O)c1cc2c(OC(F)F)cccc2[nH]1)C(=O)N[C@@H](C[C@@H]1CCCNC1=O)C(=O)COC(=O)C(C)(C)C(F)(F)F]

(3S)-3-({N-[4-(difluoromethoxy)-1H-indole-2-carbonyl]-L-leucyl}amino)-2-oxo-4-[(3S)-2-oxopiperidin-3-yl]butyl3,3,3-trifluoro-2,2-dimethylpropanoate,

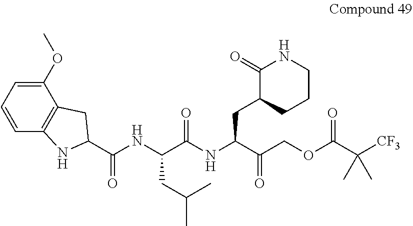 [SMILES: COc1cccc2c1CC(C(=O)N[C@@H](CC(C)C)C(=O)N[C@@H](C[C@@H]1CCCNC1=O)C(=O)COC(=O)C(C)(C)C(F)(F)F)N2]

(3S)-3-{[N-(4-methoxy-2,3-dihydro-1H-indole-2-carbonyl)-L-leucyl]amino}-2-oxo-4-[(3S)-2-oxopiperidin-3-yl]butyl 3,3,3-trifluoro-2,2-dimethylpropanoate,

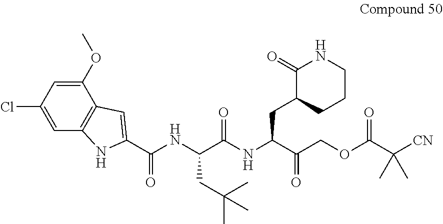 [SMILES: COc1cc(Cl)cc2[nH]c(C(=O)N[C@@H](CC(C)(C)C)C(=O)N[C@@H](C[C@@H]3CCCNC3=O)C(=O)COC(=O)C(C)(C)C#N)cc12]

(3S)-3-{[N-(6-chloro-4-methoxy-1H-indole-2-carbonyl)-4-methyl-L-leucyl]amino}-2-oxo-4-[(3S)-2-oxopiperidin-3-yl]butyl 2-cyano-2-methylpropanoate,

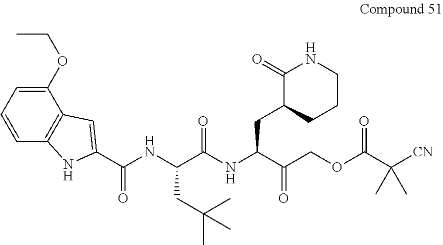 [SMILES: CCOc1cccc2[nH]c(C(=O)N[C@@H](CC(C)(C)C)C(=O)N[C@@H](C[C@@H]3CCCNC3=O)C(=O)COC(=O)C(C)(C)C#N)cc12]

(3S)-3-{[N-(4-ethoxy-1H-indole-2-carbonyl)-4-methyl-L-leucyl]amino}-2-oxo-4-[(3S)-2-oxopiperidin-3-yl]butyl 2-cyano-2-methylpropanoate,

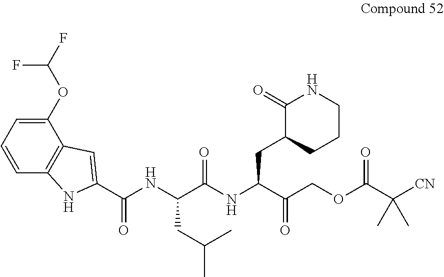 [SMILES: CC(C)C[C@H](NC(=O)c1cc2c(OC(F)F)cccc2[nH]1)C(=O)N[C@@H](C[C@@H]1CCCNC1=O)C(=O)COC(=O)C(C)(C)C#N]

(3S)-3-({N-[4-(difluoromethoxy)-1H-indole-2-carbonyl]-4-methyl-L-leucyl}amino)-2-oxo-4-[(3S)-2-oxopiperidin-3-yl]butyl 2-cyano-2-methylpropanoate,

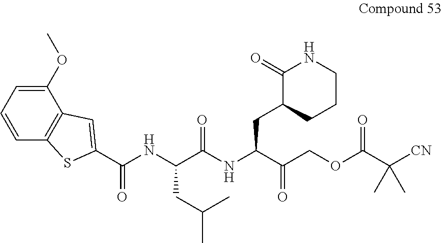 [SMILES: COc1cccc2sc(C(=O)N[C@@H](CC(C)C)C(=O)N[C@@H](C[C@@H]3CCCNC3=O)C(=O)COC(=O)C(C)(C)C#N)cc12]

(3S)-3-{[N-(4-methoxy-1-benzothiophene-2-carbonyl)-L-leucyl]amino}-2-oxo-4-[(3S)-2-oxopiperidin-3-yl]butyl 2-cyano-2-methylpropanoate,

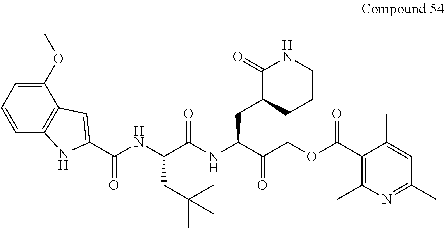 [SMILES: COc1cccc2[nH]c(C(=O)N[C@@H](CC(C)(C)C)C(=O)N[C@@H](C[C@@H]3CCCNC3=O)C(=O)COC(=O)c3c(C)cc(C)nc3C)cc12]

(3S)-3-{[N-(4-methoxy-1H-indole-2-carbonyl)-4-methyl-L-leucyl]amino}-2-oxo-4-[(3S)-2-oxopiperidin-3-yl]butyl 2,4,6-trimethylpyridine-3-carboxylate,

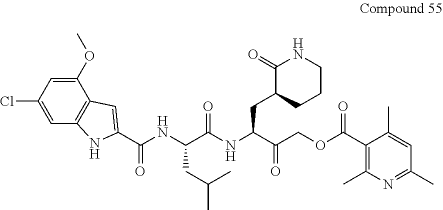 [SMILES: COc1cc(Cl)cc2[nH]c(C(=O)N[C@@H](CC(C)C)C(=O)N[C@@H](C[C@@H]3CCCNC3=O)C(=O)COC(=O)c3c(C)cc(C)nc3C)cc12]

(3S)-3-{[N-(6-chloro-4-methoxy-1H-indole-2-carbonyl)-L-leucyl]amino}-2-oxo-4-[(3S)-2-oxopiperidin-3-yl]butyl 2,4,6-trimethylpyridine-3-carboxylate,

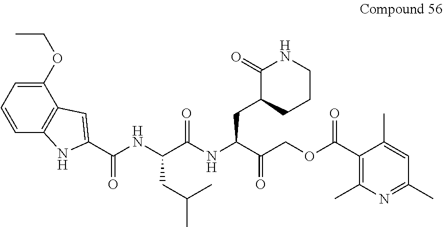 [SMILES: CCOc1cccc2[nH]c(C(=O)N[C@@H](CC(C)C)C(=O)N[C@@H](C[C@@H]3CCCNC3=O)C(=O)COC(=O)c3c(C)cc(C)nc3C)cc12]

(3S)-3-{[N-(4-ethoxy-1H-indole-2-carbonyl)-L-leucyl]amino}-2-oxo-4-[(3S)-2-oxopiperidin-3-yl]butyl 2,4,6-trimethylpyridine-3-carboxylate,

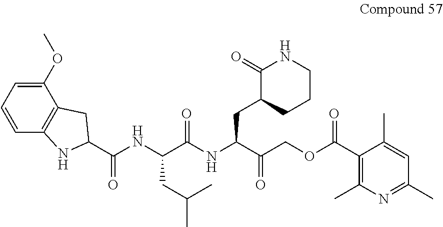 [SMILES: COc1cccc2c1CC(C(=O)N[C@@H](CC(C)C)C(=O)N[C@@H](C[C@@H]1CCCNC1=O)C(=O)COC(=O)c1c(C)cc(C)nc1C)N2]

(3S)-3-{[N-(4-methoxy-2,3-dihydro-1H-indole-2-carbonyl)-L-leucyl]amino}-2-oxo-4-[(3S)-2-oxopiperidin-3-yl]butyl 2,4,6-trimethylpyridine-3-carboxylate,

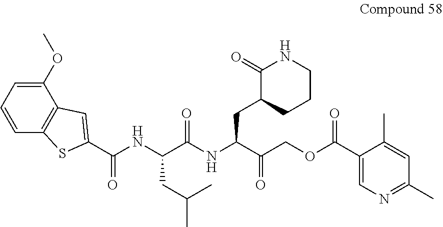 [SMILES: COc1cccc2sc(C(=O)N[C@@H](CC(C)C)C(=O)N[C@@H](C[C@@H]3CCCNC3=O)C(=O)COC(=O)c3cnc(C)cc3C)cc12]

(3S)-3-{[N-(4-methoxy-1-benzothiophene-2-carbonyl)-L-leucyl]amino}-2-oxo-4-[(3S)-2-oxopiperidin-3-yl]butyl 4,6-dimethylpyridine-3-carboxylate,

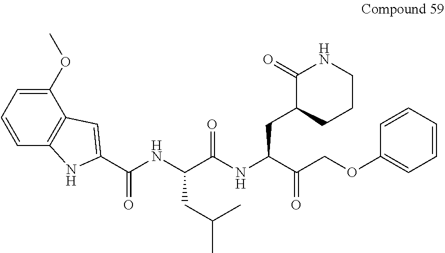 [SMILES: COc1cccc2[nH]c(C(=O)N[C@@H](CC(C)C)C(=O)N[C@@H](C[C@@H]3CCCNC3=O)C(=O)COc3ccccc3)cc12]

4-methoxy-N-[(2S)-4-methyl-1-oxo-1-({(2S)-3-oxo-1-[(3S)-2-oxopiperidin-3-yl]-4-phenoxybutan-2-yl}amino)pentan-2-yl]-1H-indole-2-carboxamide,

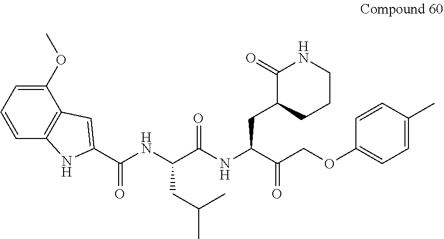 [SMILES: COc1cccc2[nH]c(C(=O)N[C@@H](CC(C)C)C(=O)N[C@@H](C[C@@H]3CCCNC3=O)C(=O)COc3ccc(C)cc3)cc12]

4-methoxy-N-[(2S)-4-methyl-1-({(2S)-4-(4-methylphenoxy)-3-oxo-1-[(3S)-2-oxopiperidin-3-yl]butan-2-yl}amino)-1-oxopentan-2-yl]-1H-indole-2-carboxamide,

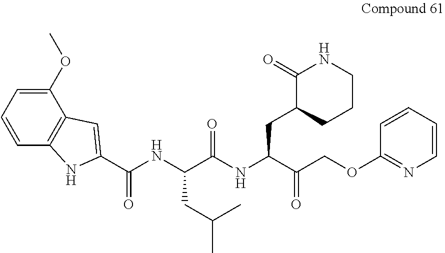 [SMILES: COc1cccc2[nH]c(C(=O)N[C@@H](CC(C)C)C(=O)N[C@@H](C[C@@H]3CCCNC3=O)C(=O)COc3ccccn3)cc12]

4-methoxy-N-[(2S)-4-methyl-1-oxo-1-({(2S)-3-oxo-1-[(3S)-2-oxopiperidin-3-yl]-4-[(pyridin-2-yl)oxy]butan-2-yl}amino)pentan-2-yl]-1H-indole-2-carboxamide,

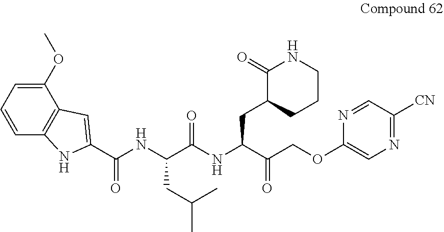 [SMILES: COc1cccc2[nH]c(C(=O)N[C@@H](CC(C)C)C(=O)N[C@@H](C[C@@H]3CCCNC3=O)C(=O)COc3cnc(C#N)cn3)cc12]

N—((S)-1-(((S)-4-((5-cyanopyrazin-2-yl)oxy)-3-oxo-1-((S)-2-oxopiperidin-3-yl)butan-2-yl)amino)-4-methyl-1-oxopentan-2-yl)-4-methoxy-1H-indole-2-carboxamide,

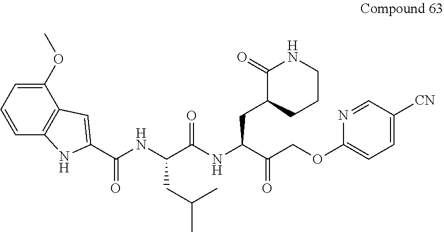 [SMILES: COc1cccc2[nH]c(C(=O)N[C@@H](CC(C)C)C(=O)N[C@@H](C[C@@H]3CCCNC3=O)C(=O)COc3ccc(C#N)cn3)cc12]

N—((S)-1-(((S)-4-((5-cyanopyridin-2-yl)oxy)-3-oxo-1-((S)-2-oxopiperidin-3-yl)butan-2-yl)amino)-4-methyl-1-oxopentan-2-yl)-4-methoxy-1H-indole-2-carboxamide,

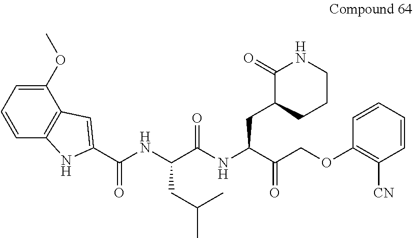 [SMILES: COc1cccc2[nH]c(C(=O)N[C@@H](CC(C)C)C(=O)N[C@@H](C[C@@H]3CCCNC3=O)C(=O)COc3ccccc3C#N)cc12]

N—((S)-1-(((S)-4-(2-cyanophenoxy)-3-oxo-1-((S)-2-oxopiperidin-3-yl)butan-2-yl)amino)-4-methyl-1-oxopentan-2-yl)-4-methoxy-1H-indole-2-carboxamide,

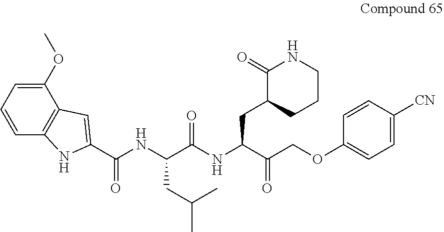 [SMILES: COc1cccc2[nH]c(C(=O)N[C@@H](CC(C)C)C(=O)N[C@@H](C[C@@H]3CCCNC3=O)C(=O)COc3ccc(C#N)cc3)cc12]

N—((S)-1-(((S)-4-(4-cyanophenoxy)-3-oxo-1-((S)-2-oxopiperidin-3-yl)butan-2-yl)amino)-4-methyl-1-oxopentan-2-yl)-4-methoxy-1H-indole-2-carboxamide,

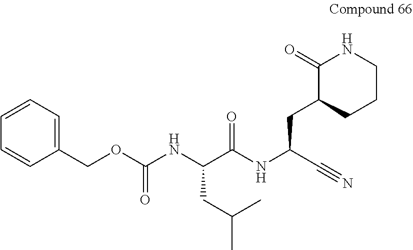 [SMILES: CC(C)C[C@H](NC(=O)OCc1ccccc1)C(=O)N[C@H](C#N)C[C@@H]1CCCNC1=O]

N²-[(benzyloxy)carbonyl]-N-{(1S)-1-cyano-2-[(3S)-2-oxopiperidin-3-yl]ethyl}-L-leucinamide,

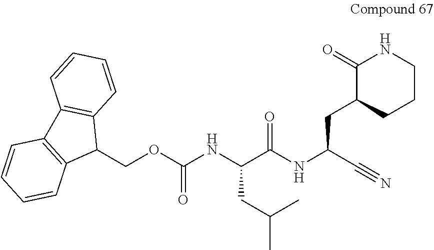 [SMILES: CC(C)C[C@H](NC(=O)OCC1c2ccccc2-c2ccccc21)C(=O)N[C@H](C#N)C[C@@H]1CCCNC1=O]

N-{(1S)-1-cyano-2-[(3S)-2-oxopiperidin-3-yl]ethyl}-N²-{[(9H-fluoren-9-yl)methoxy]carbonyl}-L-leucinamide,

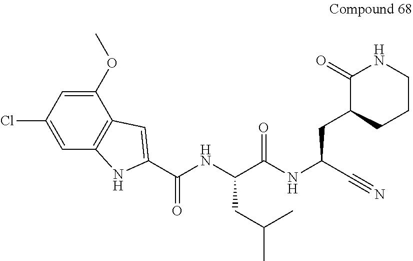 [SMILES: COc1cc(Cl)cc2[nH]c(C(=O)N[C@@H](CC(C)C)C(=O)N[C@H](C#N)C[C@@H]3CCCNC3=O)cc12]

6-chloro-N-[(2S)-1-({(1S)-1-cyano-2-[(3S)-2-oxopiperidin-3-yl]ethyl}amino)-4-methyl-1-oxopentan-2-yl]-4-methoxy-1H-indole-2-carboxamide,

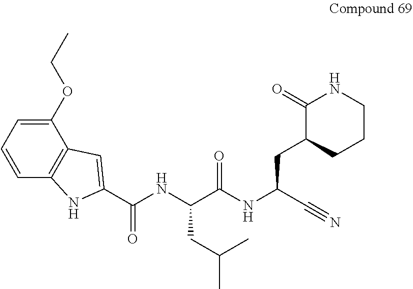 [SMILES: CCOc1cccc2[nH]c(C(=O)N[C@@H](CC(C)C)C(=O)N[C@H](C#N)C[C@@H]3CCCNC3=O)cc12]

N-[(2S)-1-({(1S)-1-cyano-2-[(3S)-2-oxopiperidin-3-yl]ethyl}amino)-4-methyl-1-oxopentan-2-yl]-4-ethoxy-1H-indole-2-carboxamide,

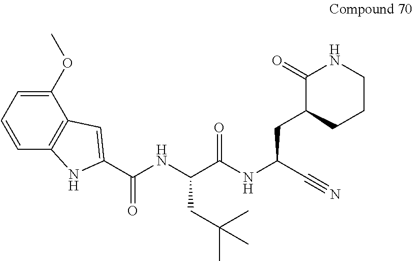 [SMILES: COc1cccc2[nH]c(C(=O)N[C@@H](CC(C)(C)C)C(=O)N[C@H](C#N)C[C@@H]3CCCNC3=O)cc12]

N-[(2S)-1-({(1S)-1-cyano-2-[(3S)-2-oxopiperidin-3-yl]ethyl}amino)-4,4-dimethyl-1-oxopentan-2-yl]-4-methoxy-1H-indole-2-carboxamide,

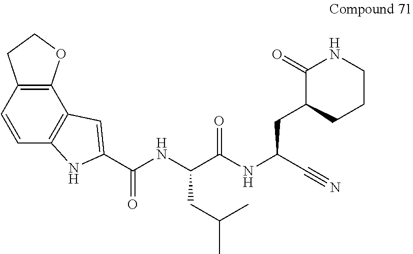 [SMILES: CC(C)C[C@H](NC(=O)c1cc2c3c(ccc2[nH]1)CCO3)C(=O)N[C@H](C#N)C[C@@H]1CCCNC1=O]

N-[(2S)-1-({(1S)-1-cyano-2-[(3S)-2-oxopiperidin-3-yl]ethyl}amino)-4-methyl-1-oxopentan-2-yl]-3,6-dihydro-2H-furo[2,3-e]indole-7-carboxamide,

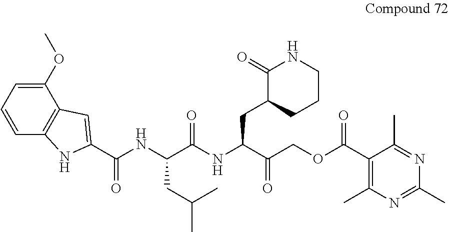 [SMILES: COc1cccc2[nH]c(C(=O)N[C@@H](CC(C)C)C(=O)N[C@@H](C[C@@H]3CCCNC3=O)C(=O)COC(=O)c3c(C)nc(C)nc3C)cc12]

3-{[N-(4-methoxy-1H-indole-2-carbonyl)leucyl]amino}-2-oxo-4-(2-oxopiperidin-3-yl)butyl 2,4,6-trimethylpyrimidine-5-carboxylate,

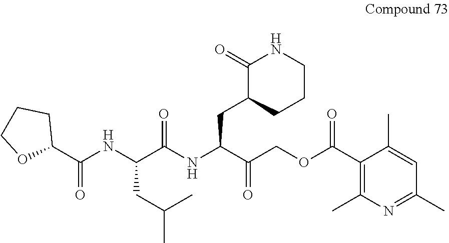 [SMILES: Cc1cc(C)c(C(=O)OCC(=O)[C@H](C[C@@H]2CCCNC2=O)NC(=O)[C@H](CC(C)C)NC(=O)[C@H]2CCCO2)c(C)n1]

(3S)-2-oxo-3-({N-[(2R)-oxolane-2-carbonyl]-L-leucyl}amino)-4-[(3S)-2-oxopiperidin-3-yl]butyl 2,4,6-trimethylpyridine-3-carboxylate,

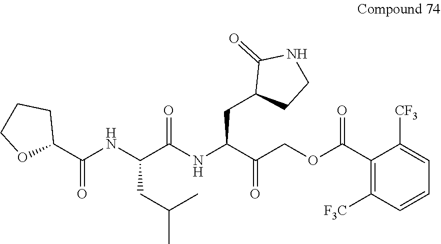 [SMILES: CC(C)C[C@H](NC(=O)[C@H]1CCCO1)C(=O)N[C@@H](C[C@@H]1CCNC1=O)C(=O)COC(=O)c1c(C(F)(F)F)cccc1C(F)(F)F]

(3S)-2-oxo-3-({N-[(2R)-oxolane-2-carbonyl]-L-leucyl}amino)-4-[(3S)-2-oxopyrrolidin-3-yl]butyl 2,6-bis(trifluoromethyl)benzoate,

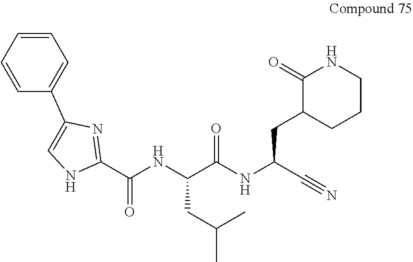 [SMILES: CC(C)C[C@H](NC(=O)c1nc(-c2ccccc2)c[nH]1)C(=O)N[C@H](C#N)CC1CCCNC1=O]

N-{(1S)-1-cyano-2-[(3S)-2-oxopiperidin-3-yl]ethyl}-N²-(4-phenyl-1H-imidazole-2-carbonyl)-L-leucinamide,

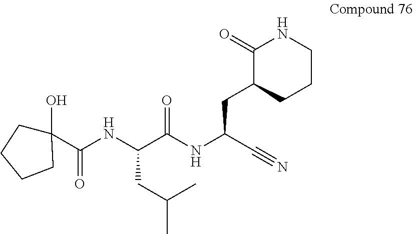 [SMILES: CC(C)C[C@H](NC(=O)C1(O)CCCC1)C(=O)N[C@H](C#N)C[C@@H]1CCCNC1=O]

N-{(1S)-1-cyano-2-[(3S)-2-oxopiperidin-3-yl]ethyl}-N²-(1-hydroxycyclopentane-1-carbonyl)-L-leucinamide,

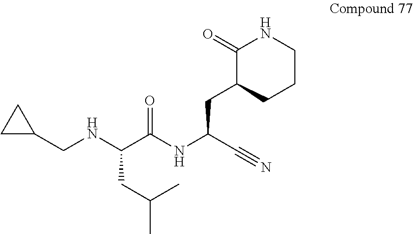 [SMILES: CC(C)C[C@H](NCC1CC1)C(=O)N[C@H](C#N)C[C@@H]1CCCNC1=O]

N-{(1S)-1-cyano-2-[(3S)-2-oxopiperidin-3-yl]ethyl}-N²-(cyclopropylmethyl)-L-leucinamide,

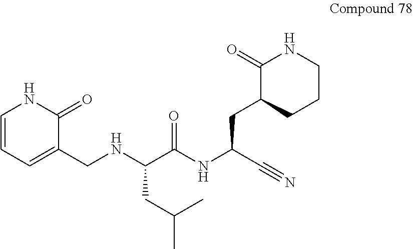 [SMILES: CC(C)C[C@H](NCc1ccc[nH]c1=O)C(=O)N[C@H](C#N)C[C@@H]1CCCNC1=O]

N-{(1S)-1-cyano-2-[(3S)-2-oxopiperidin-3-yl]ethyl}-N²-[(2-oxo-1,2-dihydropyridin-3-yl)methyl]-L-leucinamide,

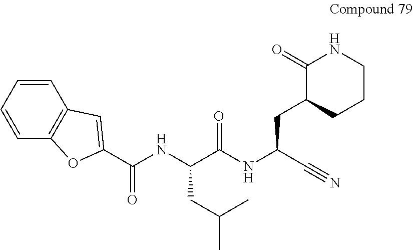 [SMILES: CC(C)C[C@H](NC(=O)c1cc2ccccc2o1)C(=O)N[C@H](C#N)C[C@@H]1CCCNC1=O]

N-[(2S)-1-({(1S)-1-cyano-2-[(3S)-2-oxopiperidin-3-yl]ethyl}amino)-4-methyl-1-oxopentan-2-yl]-1-benzofuran-2-carboxamide,

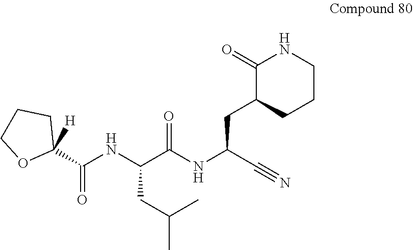

N-{(1S)-1-cyano-2-[(3 S)-2-oxopiperidin-3-yl]ethyl}-N²-[(2R)-oxolane-2-carbonyl]-L-leucinamide,

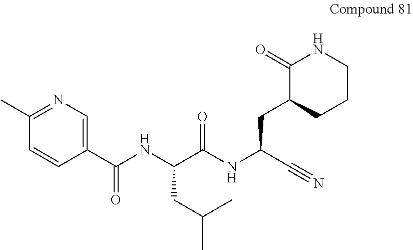 [SMILES: Cc1ccc(C(=O)N[C@@H](CC(C)C)C(=O)N[C@H](C#N)C[C@@H]2CCCNC2=O)cn1]

N-{(1S)-1-cyano-2-[(3S)-2-oxopiperidin-3-yl]ethyl}-N²-(6-methylpyridine-3-carbonyl)-L-leucinamide,

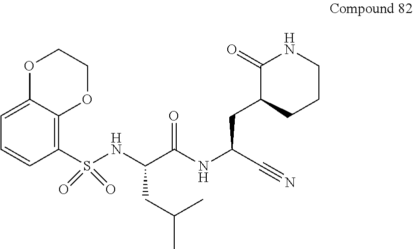 [SMILES: CC(C)C[C@H](NS(=O)(=O)c1cccc2c1OCCO2)C(=O)N[C@H](C#N)C[C@@H]1CCCNC1=O]

N-{(1S)-1-cyano-2-[(3S)-2-oxopiperidin-3-yl]ethyl}-N²-(2,3-dihydro-1,4-benzodioxine-5-sulfonyl)-L-leucinamide,

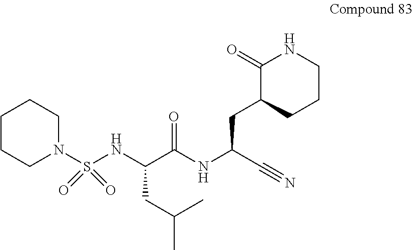 [SMILES: CC(C)C[C@H](NS(=O)(=O)N1CCCCC1)C(=O)N[C@H](C#N)C[C@@H]1CCCNC1=O]

N-{(1S)-1-cyano-2-[(3S)-2-oxopiperidin-3-yl]ethyl}-N²-(piperidine-1-sulfonyl)-L-leucinamide,

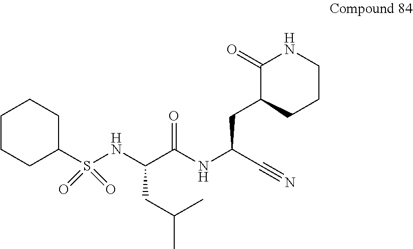 [SMILES: CC(C)C[C@H](NS(=O)(=O)C1CCCCC1)C(=O)N[C@H](C#N)C[C@@H]1CCCNC1=O]

N-{(1S)-1-cyano-2-[(3S)-2-oxopiperidin-3-yl]ethyl}-N²-(cyclohexanesulfonyl)-L-leucinamide,

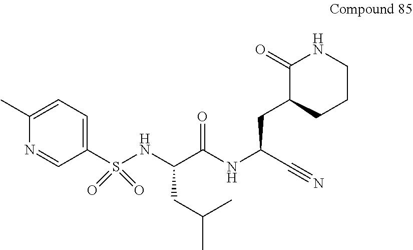 [SMILES: Cc1ccc(S(=O)(=O)N[C@@H](CC(C)C)C(=O)N[C@H](C#N)C[C@@H]2CCCNC2=O)cn1]

N-{(1S)-1-cyano-2-[(3S)-2-oxopiperidin-3-yl]ethyl}-N²-(6-methylpyridine-3-sulfonyl)-L-leucinamide,

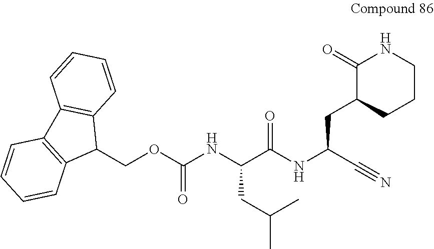 [SMILES: CC(C)C[C@H](NC(=O)OCC1c2ccccc2-c2ccccc21)C(=O)N[C@H](C#N)C[C@@H]1CCCNC1=O]

N-{(1S)-1-cyano-2-[(3S)-2-oxopiperidin-3-yl]ethyl}-N²-{[(9H-fluoren-9-yl)methoxy]carbonyl}-4-methyl-L-leucinamide,

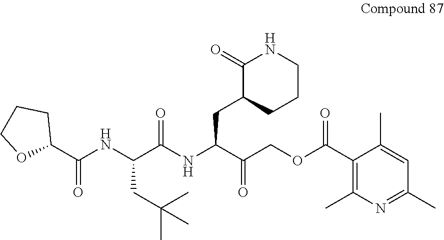 [SMILES: Cc1cc(C)c(C(=O)OCC(=O)[C@H](C[C@@H]2CCCNC2=O)NC(=O)[C@H](CC(C)(C)C)NC(=O)[C@H]2CCCO2)c(C)n1]

(3S)-3-({4-methyl-N-[(2R)-oxolane-2-carbonyl]-L-leucyl}amino)-2-oxo-4-[(3S)-2-oxopiperidin-3-yl]butyl 2,4,6-trimethylpyridine-3-carboxylate,

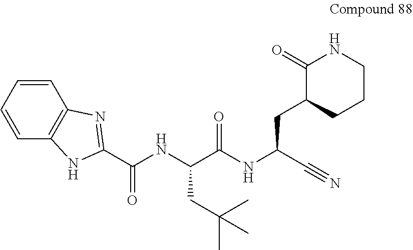 [SMILES: CC(C)(C)C[C@H](NC(=O)c1nc2ccccc2[nH]1)C(=O)N[C@H](C#N)C[C@@H]1CCCNC1=O]

N-[(2S)-1-({(1S)-1-cyano-2-[(3S)-2-oxopiperidin-3-yl]ethyl}amino)-4,4-dimethyl-1-oxopentan-2-yl]-1H-benzimidazole-2-carboxamide,

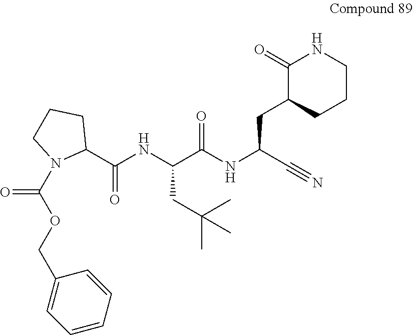 [SMILES: CC(C)(C)C[C@H](NC(=O)C1CCCN1C(=O)OCc1ccccc1)C(=O)N[C@H](C#N)C[C@@H]1CCCNC1=O]

1-[(benzyloxy)carbonyl]prolyl-N-{(1S)-1-cyano-2-[(3S)-2-oxopiperidin-3-yl]ethyl}-4-methyl-L-leucinamide,

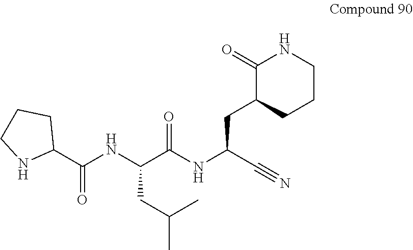 [SMILES: CC(C)C[C@H](NC(=O)C1CCCN1)C(=O)N[C@H](C#N)C[C@@H]1CCCNC1=O]

prolyl-N-{(1S)-1-cyano-2-[(3S)-2-oxopiperidin-3-yl]ethyl}-4-methyl-L-leucinamide,

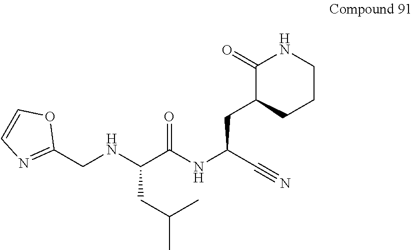 [SMILES: CC(C)C[C@H](NCc1ncco1)C(=O)N[C@H](C#N)C[C@@H]1CCCNC1=O]

N-{(1S)-1-cyano-2-[(3S)-2-oxopiperidin-3-yl]ethyl}-N²-[(1,3-oxazol-2-yl)methyl]-L-leucinamide,

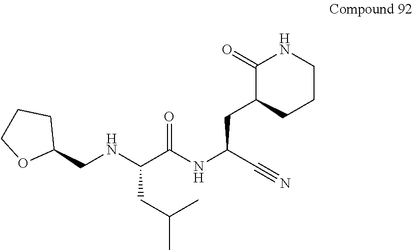 [SMILES: CC(C)C[C@H](NC[C@@H]1CCCO1)C(=O)N[C@H](C#N)C[C@@H]1CCCNC1=O]

N-{(1S)-1-cyano-2-[(3S)-2-oxopiperidin-3-yl]ethyl}-N²-{[(2S)-oxolan-2-yl]methyl}-L-leucinamide,

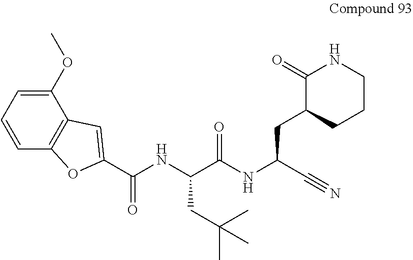 [SMILES: COc1cccc2oc(C(=O)N[C@@H](CC(C)(C)C)C(=O)N[C@H](C#N)C[C@@H]3CCCNC3=O)cc12]

N-[(2S)-1-({(1S)-1-cyano-2-[(3S)-2-oxopiperidin-3-yl]ethyl}amino)-4,4-dimethyl-1-oxopentan-2-yl]-4-methoxy-1-benzofuran-2-carboxamide,

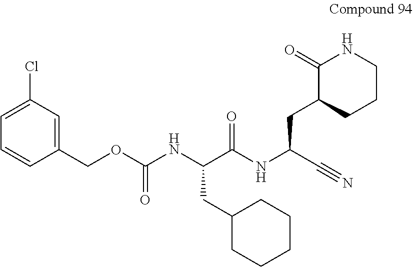 [SMILES: N#C[C@H](C[C@@H]1CCCNC1=O)NC(=O)[C@H](CC1CCCCC1)NC(=O)OCc1cccc(Cl)c1]

(3-chlorophenyl)methyl [(2S)-1-({(1S)-1-cyano-2-[(3S)-2-oxopyrrolidin-3-yl]ethyl}amino)-3-cyclohexyl-1-oxopropan-2-yl]carbamate,

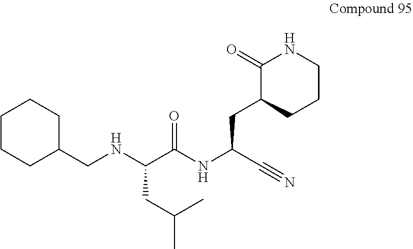 [SMILES: CC(C)C[C@H](NCC1CCCCC1)C(=O)N[C@H](C#N)C[C@@H]1CCCNC1=O]

N-{(1S)-1-cyano-2-[(3S)-2-oxopiperidin-3-yl]ethyl}-N²-(cyclohexylmethyl)-L-leucinamide,

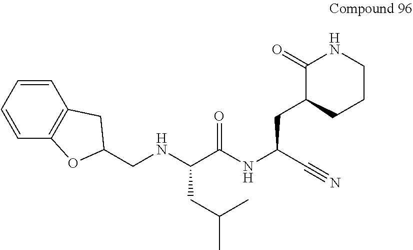 [SMILES: CC(C)C[C@H](NCC1Cc2ccccc2O1)C(=O)N[C@H](C#N)C[C@@H]1CCCNC1=O]

N-{(1S)-1-cyano-2-[(3S)-2-oxopiperidin-3-yl]ethyl}-N²-[(2,3-dihydro-1-benzofuran-2-yl)methyl]-L-leucinamide first diastereomer,

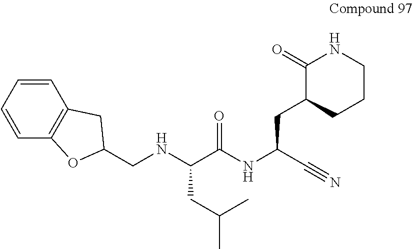 [SMILES: CC(C)C[C@H](NCC1Cc2ccccc2O1)C(=O)N[C@H](C#N)C[C@@H]1CCCNC1=O]

N-{(1S)-1-cyano-2-[(3S)-2-oxopiperidin-3-yl]ethyl}-N²-[(2,3-dihydro-1-benzofuran-2-yl)methyl]-L-leucinamide second diastereomer,

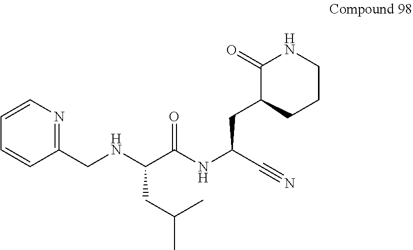 [SMILES: CC(C)C[C@H](NCc1ccccn1)C(=O)N[C@H](C#N)C[C@@H]1CCCNC1=O]

N-{(1S)-1-cyano-2-[(3S)-2-oxopiperidin-3-yl]ethyl}-N²-[(pyridin-2-yl)methyl]-L-leucinamide,

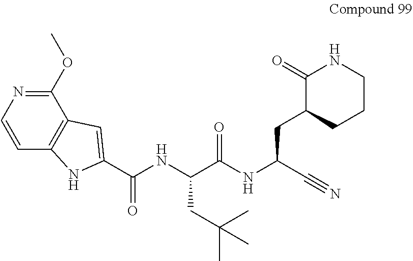 [SMILES: COc1nccc2[nH]c(C(=O)N[C@@H](CC(C)(C)C)C(=O)N[C@H](C#N)C[C@@H]3CCCNC3=O)cc12]

N-[(2S)-1-({(1S)-1-cyano-2-[(3S)-2-oxopiperidin-3-yl]ethyl}amino)-4,4-dimethyl-1-oxopentan-2-yl]-4-methoxy-1H-pyrrolo[3,2-c]pyridine-2-carboxamide,

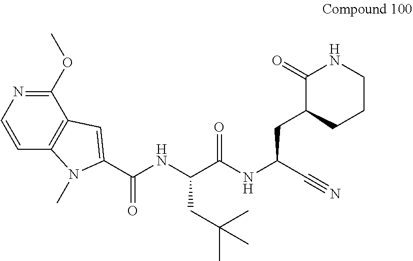 [SMILES: COc1nccc2c1cc(C(=O)N[C@@H](CC(C)(C)C)C(=O)N[C@H](C#N)C[C@@H]1CCCNC1=O)n2C]

N-[(2S)-1-({(1S)-1-cyano-2-[(3S)-2-oxopiperidin-3-yl]ethyl}amino)-4,4-dimethyl-1-oxopentan-2-yl]-4-methoxy-1-methyl-1H-pyrrolo[3,2-c]pyridine-2-carboxamide,

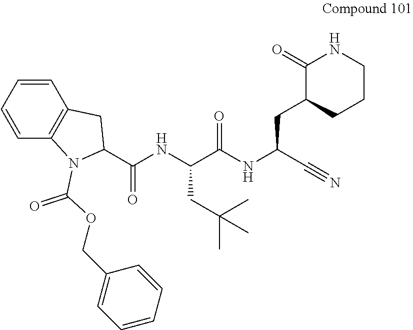 [SMILES: CC(C)(C)C[C@H](NC(=O)C1Cc2ccccc2N1C(=O)OCc1ccccc1)C(=O)N[C@H](C#N)C[C@@H]1CCCNC1=O]

benzyl (2R)-2-{[(2S)-1-({(1S)-1-cyano-2-[(3S)-2-oxopiperidin-3-yl]ethyl}amino)-4,4-dimethyl-1-oxopentan-2-yl]carbamoyl}-2,3-dihydro-1H-indole-1-carboxylate,

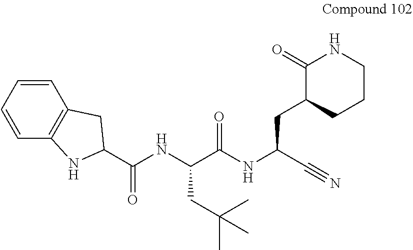 [SMILES: CC(C)(C)C[C@H](NC(=O)C1Cc2ccccc2N1)C(=O)N[C@H](C#N)C[C@@H]1CCCNC1=O]

(2R)—N-[(2S)-1-({(1S)-1-cyano-2-[(3S)-2-oxopiperidin-3-yl]ethyl}amino)-4,4-dimethyl-1-oxopentan-2-yl]-2,3-dihydro-1H-indole-2-carboxamide,

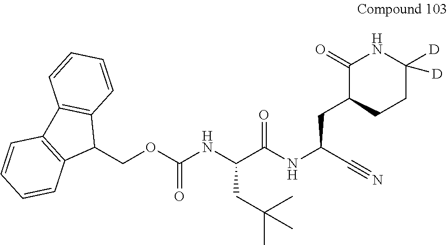 [SMILES: [2H]C1([2H])CC[C@@H](C[C@@H](C#N)NC(=O)[C@H](CC(C)(C)C)NC(=O)OCC2c3ccccc3-c3ccccc32)C(=O)N1]

N-{(1S)-1-cyano-2-[(3S)-2-oxo(6,6-d₂)piperidin-3-yl]ethyl}-N²-{[(9H-fluoren-9-yl)methoxy]carbonyl}-4-methyl-L-leucinamide,

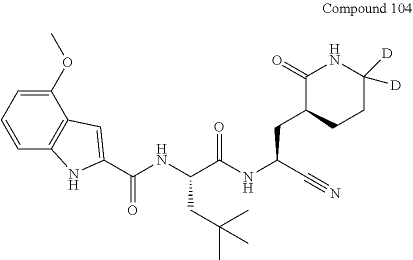 [SMILES: [2H]C1([2H])CC[C@@H](C[C@@H](C#N)NC(=O)[C@H](CC(C)(C)C)NC(=O)c2cc3c(OC)cccc3[nH]2)C(=O)N1]

N-[(2S)-1-({(1S)-1-cyano-2-[(3S)-2-oxo(6,6-d₂)piperidin-3-yl]ethyl}amino)-4,4-dimethyl-1-oxopentan-2-yl]-4-methoxy-1H-indole-2-carboxamide,

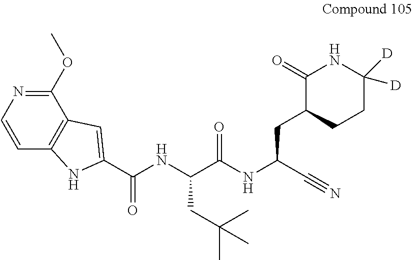 [SMILES: [2H]C1([2H])CC[C@@H](C[C@@H](C#N)NC(=O)[C@H](CC(C)(C)C)NC(=O)c2cc3c(OC)nccc3[nH]2)C(=O)N1]

N-[(2S)-1-({(1S)-1-cyano-2-[(3S)-2-oxo(6,6-d₂)piperidin-3-yl]ethyl}amino)-4,4-dimethyl-1-oxopentan-2-yl]-4-methoxy-1H-pyrrolo[3,2-c]pyridine-2-carboxamide,

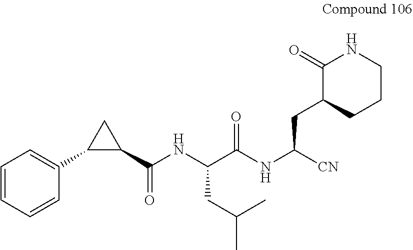 [SMILES: CC(C)C[C@H](NC(=O)[C@@H]1C[C@H]1c1ccccc1)C(=O)N[C@H](C#N)C[C@@H]1CCCNC1=O]

N-{(1S)-1-cyano-2-[(3S)-2-oxopiperidin-3-yl]ethyl}-N²-[(1R,2R)-2-phenylcyclopropane-1-carbonyl]-L-leucinamide,

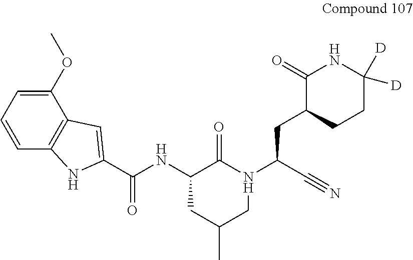

N-[(2S)-1-({(1S)-1-cyano-2-[(3S)-2-oxo(6,6-d₂)piperidin-3-yl]ethyl}amino)-4-methyl-1-oxopentan-2-yl]-4-methoxy-1H-indole-2-carboxamide,

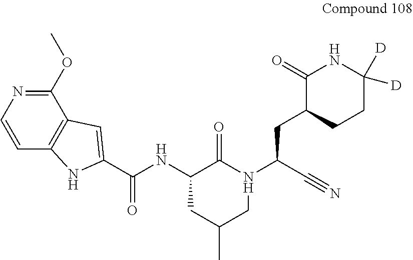

N-[(2S)-1-({(1S)-1-cyano-2-[(3S)-2-oxo(6,6-d₂)piperidin-3-yl]ethyl}amino)-4-methyl-1-oxopentan-2-yl]-4-methoxy-1H-pyrrolo[3,2-c]pyridine-2-carboxamide,

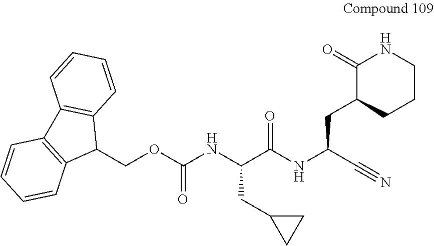 [SMILES: N#C[C@H](C[C@@H]1CCCNC1=O)NC(=O)[C@H](CC1CC1)NC(=O)OCC1c2ccccc2-c2ccccc21]

(9H-fluoren-9-yl)methyl [(2S)-1-({(1S)-1-cyano-2-[(3S)-2-oxopiperidin-3-yl]ethyl}amino)-3-cyclopropyl-1-oxopropan-2-yl]carbamate,

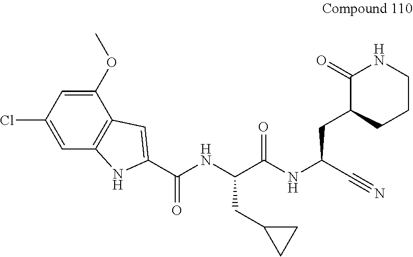 [SMILES: COc1cc(Cl)cc2[nH]c(C(=O)N[C@@H](CC3CC3)C(=O)N[C@H](C#N)C[C@@H]3CCCNC3=O)cc12]

6-chloro-N-[(2S)-1-({(1S)-1-cyano-2-[(3S)-2-oxopiperidin-3-yl]ethyl}amino)-3-cyclopropyl-1-oxopropan-2-yl]-4-methoxy-1H-indole-2-carboxamide,

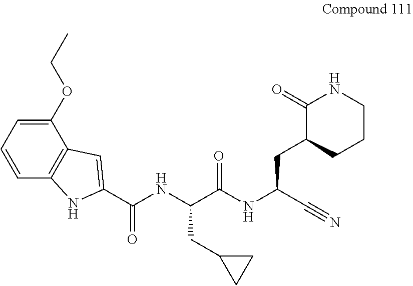 [SMILES: CCOc1cccc2[nH]c(C(=O)N[C@@H](CC3CC3)C(=O)N[C@H](C#N)C[C@@H]3CCCNC3=O)cc12]

N-[(2S)-1-({(1S)-1-cyano-2-[(3S)-2-oxopiperidin-3-yl]ethyl}amino)-3-cyclopropyl-1-oxopropan-2-yl]-4-ethoxy-1H-indole-2-carboxamide,

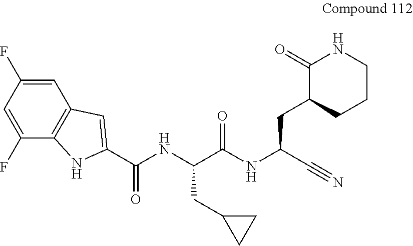 [SMILES: N#C[C@H](C[C@@H]1CCCNC1=O)NC(=O)[C@H](CC1CC1)NC(=O)c1cc2cc(F)cc(F)c2[nH]1]

N-[(2S)-1-({(1S)-1-cyano-2-[(3S)-2-oxopiperidin-3-yl]ethyl}amino)-3-cyclopropyl-1-oxopropan-2-yl]-5,7-difluoro-1H-indole-2-carboxamide,

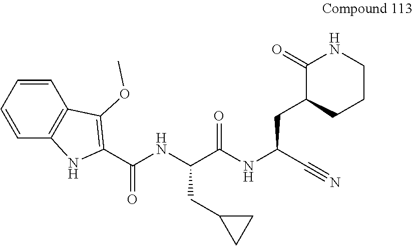 [SMILES: COc1c(C(=O)N[C@@H](CC2CC2)C(=O)N[C@H](C#N)C[C@@H]2CCCNC2=O)[nH]c2ccccc12]

N-[(2S)-1-({(1S)-1-cyano-2-[(3S)-2-oxopiperidin-3-yl]ethyl}amino)-3-cyclopropyl-1-oxopropan-2-yl]-3-methoxy-1H-indole-2-carboxamide,

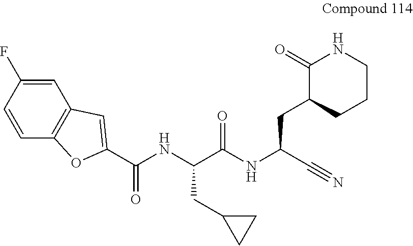 [SMILES: N#C[C@H](C[C@@H]1CCCNC1=O)NC(=O)[C@H](CC1CC1)NC(=O)c1cc2cc(F)ccc2o1]

N-[(2S)-1-({(1S)-1-cyano-2-[(3S)-2-oxopiperidin-3-yl]ethyl}amino)-3-cyclopropyl-1-oxopropan-2-yl]-5-fluoro-1-benzofuran-2-carboxamide,

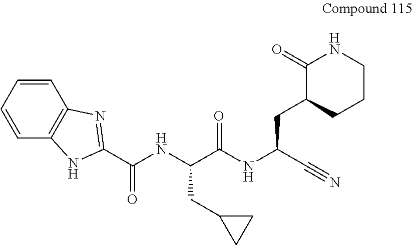 [SMILES: N#C[C@H](C[C@@H]1CCCNC1=O)NC(=O)[C@H](CC1CC1)NC(=O)c1nc2ccccc2[nH]1]

N-[(2S)-1-({(1S)-1-cyano-2-[(3S)-2-oxopiperidin-3-yl]ethyl}amino)-3-cyclopropyl-1-oxopropan-2-yl]-1H-benzimidazole-2-carboxamide,

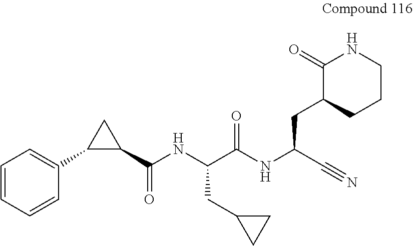 [SMILES: N#C[C@H](C[C@@H]1CCCNC1=O)NC(=O)[C@H](CC1CC1)NC(=O)[C@@H]1C[C@H]1c1ccccc1]

N-{(1S)-1-cyano-2-[(3S)-2-oxopiperidin-3-yl]ethyl}-3-cyclopropyl-N²-[(1R,2R)-2-phenylcyclopropane-1-carbonyl]-L-alaninamide, and

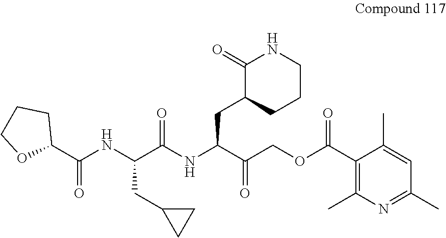 [SMILES: Cc1cc(C)c(C(=O)OCC(=O)[C@H](C[C@@H]2CCCNC2=O)NC(=O)[C@H](CC2CC2)NC(=O)[C@H]2CCCO2)c(C)n1]

(3S)-3-({3-cyclopropyl-N-[(2R)-oxolane-2-carbonyl]-L-alanyl}amino)-2-oxo-4-[(3S)-2-oxopiperidin-3-yl]butyl 2,4,6-trimethylpyridine-3-carboxylate.

The compounds described herein can be isolated by procedures known to those skilled in the art. The compounds described herein may be obtained, for instance, by a resolution technique or by chromatography techniques (e.g., silica gel chromatography, chiral chromatography, etc.). As used herein, the term “isolated” refers to compounds that are non-naturally occurring and can be obtained or purified from synthetic reaction mixtures. Isolated compounds may find use in the pharmaceutical compositions and methods of treatment described herein.

The compounds described also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Thus, the disclosed compounds may be enriched in one or more of these isotopes relative to the natural abundance of such isotope. By way of example, deuterium (²H; D) has a natural abundance of about 0.015%. Accordingly, for approximately every 6,500 hydrogen atoms occurring in nature, there is one deuterium atom. Specifically contemplated herein are compounds enriched in deuterium at one or more positions. Thus, deuterium containing compounds of the disclosure have deuterium at one or more positions (as the case may be) in an abundance of greater than 0.015%. In some embodiments, one or more (e.g., 1, 2, 3, 4, 5, 6, 7 or more) hydrogen atoms of a substituent group (e.g., an R-group) of any one of the subject compounds described herein are substituted with a deuterium.

### METHODS OF USE

The compounds of the present disclosure find use in treatment of a condition or disease in a subject that is amenable to treatment by administration of the compound. Thus, in some embodiments, provided are methods that include administering to a subject a therapeutically effective amount of any of the compounds of the present disclosure. In certain aspects, provided are methods of delivering a compound to a target site in a subject, the method including administering to the subject a pharmaceutical composition including any of the compounds of the present disclosure, where the administering is effective to provide a therapeutically effective amount of the compound at the target site in the subject.

The subject to be treated can be one that is in need of therapy, where the subject to be treated is one amenable to treatment using the compounds disclosed herein. Accordingly, a variety of subjects may be amenable to treatment using the compounds disclosed herein. Generally, such subjects are “mammals”, with humans being of interest. Other subjects can include companion animals or domestic pets (e.g., canine and feline), livestock (e.g., cows, pigs, goats, horses, and the like), rodents (e.g., mice, guinea pigs, and rats, e.g., as in animal models of disease), as well as non-human primates (e.g., chimpanzees, and monkeys). In some instances, the mammal is selected from a companion animal and livestock. In some instances, the mammal is feline. In some instances, the mammal is a human.

The present disclosure provides methods that include delivering a compound of the present disclosure to an individual having a disease, such as methods that include administering to the subject a therapeutically effective amount of a compound of the present disclosure. The methods are useful for treating a wide variety of conditions and/or symptoms associated with a disease. In the context of disease, the term “treating” includes one or more (e.g., each) of: reducing the severity of one or more symptoms, inhibiting the progression, reducing the duration of one or more symptoms, and ameliorating one or more symptoms associated with the disease.

In certain embodiments, methods of the present disclosure include inhibiting a Baltimore Group IV RNA virus in a cell infected with a Baltimore Group IV RNA virus, wherein the method includes contacting the cell with a compound of the present disclosure. In some instances, the contacting includes delivering the compound into the cytosol of the cell by any suitable means. In some instances, the compounds of the present disclosure are effective for inhibiting the viral activity of a Baltimore Group IV RNA virus including any of, e.g., the attachment, penetration, uncoating, replication, assembly, and release of the virus. In some instances, a compound of the present disclosure is effective for treating a Baltimore Group IV RNA virus infection by inhibiting the activity of a protease. The protease may be required for the activity of the virus, e.g., the attachment, penetration, uncoating, replication, assembly, and/or release of the virus. In some instances, compounds of the present disclosure are effective for inhibiting the activity of the protease by inhibiting, e.g., blocking or chemically reacting with, a catalytic domain or catalytic residue(s) of the protease. In some instances, compounds of the present disclosure inhibit the activity of the protease by forming a covalent bond with a catalytic domain or catalytic residue(s). The catalytic domain or catalytic residue(s) may be present in the active site of the protease. In some instances, the protease is a cysteine protease. In some instances, the protease is 3-chymotrypsin protease (3CP). In some instances, the protease is 3-chymotrypsin-like protease (3CLP).

In certain embodiments, methods of the present disclosure include administering a compound of the present disclosure to a subject, where the administering is effective for treating a disease caused by a Baltimore Group IV RNA virus. The methods may include a method of treating a Baltimore Group IV RNA virus infection in a mammal, the method comprising administering to the mammal an effective amount of a compound of the present disclosure. In some embodiments, the methods involve administering an effective amount of a compound according to the present disclosure, a pro-drug thereof or a pharmaceutically acceptable salt thereof to a subject. In certain embodiments, the methods include identifying a subject with a Baltimore Group IV RNA virus infection, e.g., a coronavirus infection, a rhinovirus infection, a coxsackievirus infection, a norovirus infection, and administering a compound of the present disclosure, a pro-drug thereof or a pharmaceutically acceptable salt thereof to the subject. In some instances, the methods include a step (a) of testing a patient for a Baltimore Group IV RNA virus, e.g., before any treatment is administered. The methods may then include step (b) of administering a compound of the present disclosure, a pro-drug thereof or a pharmaceutically acceptable salt thereof to the subject according to any of the embodiments described herein.

A compound of the present disclosure may be administered at any point during a subject's infection with a Baltimore Group IV RNA virus. In certain embodiments, the subject has, has had, is suspected to have, or is suspected to have had a Baltimore Group IV RNA virus infection. A subject with a Baltimore Group IV RNA virus infection may exhibit one or more symptoms including, e.g., a cough, fever or chills, shortness of breath, fatigue, muscle or body aches, new loss of taste or smell, sore throat, headache, congestion, nasal discharge, nausea, vomiting, diarrhea, stomach pain, chest pain or pressure, confusion, inability to wake or stay awake, and bluish lips or face. In some cases, the subject is asymptomatic. In some cases, a subject with a Baltimore Group IV RNA virus infection exhibits one or more syndromes or acute conditions including, e.g., organ failure, acute respiratory distress syndrome, acute kidney injury, and thrombosis. In certain embodiments, the subject has or is expected to develop symptoms associated with a cytokine response, e.g., a cytokine storm caused by the overproduction of inflammatory cytokines. In some cases, the patient may have signs of respiratory distress, e.g., a cough, but does not have acute respiratory distress syndrome. In these embodiments, the patient may not be in intensive care. In any embodiment, the patient may be 60 years old or more, 70 years old or more, or 80 years old or more. In some instances, the patient may be 60 years old or less, such as 50 years old or less, or 40 years old or less, or 30 years old or less, or 20 years old or less. In some instances, the patient may be immunocompromised, such as immunocompromised due to chemotherapy or radiation therapy. The patient may have or may have had one or more other lung diseases in the past. For example, in some cases, the patient has or has a history of having asthma, pneumothorax, atelectasis, bronchitis, chronic obstructive pulmonary disease, lung cancer or pneumonia. In some cases, the infection is a SARS infection. In some cases, the infection is a MERS infection. In some cases, the infection is a COVID-19 infection. In some cases, the infection is Feline Infectious Peritonitis (FIP). In some instances, the subject receives multiple administrations of a compound over a period including, e.g., days, weeks, or months.

The administering can be done any convenient way. Generally, administration is, for example, oral, buccal, parenteral (e.g., intravenous, intraarterial, subcutaneous), intraperitoneal (i.e., into the body cavity), topically, e.g., by inhalation or aeration (i.e., through the mouth or nose), or rectally systemic (i.e., affecting the entire body). For example, the administration may be systemic, e.g., orally (via injection of tablet, pill or liquid) or intravenously (by injection or via a drip, for example). In other embodiments, the administering can be done by pulmonary administration, e.g., using an inhaler or nebulizer. Compounds of the present disclosure or composition comprising the compounds may be administered in dosage unit formulations containing conventional non-toxic pharmaceutically acceptable carriers, adjuvants, and vehicles as desired. The term “topically” may include injection, insertion, implantation, topical application, or parenteral application.

The virus inhibited by the methods may be any of the Baltimore Group IV RNA viruses. In some instances, the Baltimore Group IV RNA virus is selected the family of Picornaviridae, Calciviridae and Coronaviridae. In some instances, the Baltimore Group IV RNA virus is selected from rhinovirus, coxsackievirus, norovirus and coronavirus. In some instances, the Baltimore Group IV RNA virus is selected from norovirus, and coronavirus. In some instances, the Baltimore Group IV RNA virus is human norovirus. In some embodiments, the Baltimore Group IV RNA virus is coronavirus. In certain embodiments, the coronavirus is one that causes disease in mammals. In certain embodiments, the coronavirus causes disease in companion animals or livestock. In certain embodiments, the coronavirus is a feline coronavirus. In certain embodiments, the coronavirus is feline infectious peritonitis. In certain embodiments, the coronavirus is a human coronavirus. In certain embodiments, the coronavirus is selected from Severe Acute Respiratory Syndrome coronavirus 2 (SARS-CoV-2), Severe Acute Respiratory syndrome coronavirus 1 (SARS-CoV-1) and Middle Eastern Respiratory syndrome-related coronavirus (MERS-CoV).

Pharmaceutical Compositions

In certain embodiments, the disclosed compounds are useful for the treatment of a disease or disorder. Accordingly, pharmaceutical compositions comprising at least one disclosed compound are also described herein. For example, the present disclosure provides pharmaceutical compositions that include a therapeutically effective amount of a compound of the present disclosure (or a pharmaceutically acceptable salt or solvate or hydrate or stereoisomer thereof) and a pharmaceutically acceptable excipient.

A pharmaceutical composition that includes a subject compound may be administered to a patient alone, or in combination with other supplementary active agents. For example, one or more compounds according to the present disclosure can be administered to a patient with or without supplementary active agents. The pharmaceutical compositions may be manufactured using any of a variety of processes, including, but not limited to, conventional mixing, dissolving, granulating, dragee-making, levigating, emulsifying, encapsulating, entrapping, lyophilizing, and the like. The pharmaceutical composition can take any of a variety of forms including, but not limited to, a sterile solution, suspension, emulsion, spray dried dispersion, lyophilisate, tablet, microtablets, pill, pellet, capsule, powder, syrup, elixir or any other dosage form suitable for administration.

A compound of the present disclosure may be administered to a subject using any convenient means capable of resulting in the desired reduction in disease condition or symptom. Thus, a compound can be incorporated into a variety of formulations for therapeutic administration. More particularly, a compound can be formulated into pharmaceutical compositions by combination with appropriate pharmaceutically acceptable excipients, carriers or diluents, and may be formulated into preparations in solid, semi-solid, liquid or gaseous forms, such as tablets, capsules, powders, granules, ointments, solutions, suppositories, injections, inhalants, aerosols, and the like.

Formulations for pharmaceutical compositions are described in, for example, Remington's Pharmaceutical Sciences, by E. W. Martin, Mack Publishing Co., Easton, Pa., 19th Edition, 1995, which describes examples of formulations (and components thereof) suitable for pharmaceutical delivery of the disclosed compounds. Pharmaceutical compositions that include at least one of the compounds can be formulated for use in human or veterinary medicine. Particular formulations of a disclosed pharmaceutical composition may depend, for example, on the mode of administration and/or on the location of the subject to be treated. In some embodiments, formulations include a pharmaceutically acceptable excipient in addition to at least one active ingredient, such as a compound of the present disclosure. In other embodiments, other medicinal or pharmaceutical agents, for example, with similar, related or complementary effects on the disease or condition being treated can also be included as active ingredients in a pharmaceutical composition.

Pharmaceutically acceptable carriers useful for the disclosed methods and compositions may depend on the particular mode of administration being employed. In addition to biologically neutral carriers, pharmaceutical compositions to be administered can optionally contain non-toxic auxiliary substances (e.g., excipients), such as wetting or emulsifying agents, preservatives, and pH buffering agents, and the like. The disclosed pharmaceutical compositions may be formulated as a pharmaceutically acceptable salt of a disclosed compound.

The term “unit dosage form,” as used herein, refers to physically discrete units suitable as unitary dosages for human and animal subjects, each unit containing a predetermined quantity of a compound calculated in an amount sufficient to produce the desired effect in association with a pharmaceutically acceptable diluent, excipient, carrier or vehicle. The specifications for a compound depend on the particular compound employed and the effect to be achieved, and the pharmacodynamics associated with each compound in the subject.

The dosage form of a disclosed pharmaceutical composition may be determined by the mode of administration chosen. For example, in addition to injectable fluids, topical or oral dosage forms may be employed. Topical preparations may include eye drops, ointments, sprays and the like. Oral formulations may be liquid (e.g., syrups, solutions or suspensions), or solid (e.g., powders, pills, tablets, or capsules). Methods of preparing such dosage forms are known, or will be apparent, to those skilled in the art.

Certain embodiments of the pharmaceutical compositions that include a subject compound may be formulated in unit dosage form suitable for individual administration of precise dosages. The amount of active ingredient administered may depend on the subject being treated, the severity of the affliction, and the manner of administration, and is known to those skilled in the art. In certain instances, the formulation to be administered contains a quantity of the compounds disclosed herein in an amount effective to achieve the desired effect in the subject being treated.

Each therapeutic compound can independently be in any dosage form, such as those described herein, and can also be administered in various ways, as described herein. For example, the compounds may be formulated together, in a single dosage unit (that is, combined together in one form such as capsule, tablet, powder, or liquid, etc.) as a combination product. Alternatively, when not formulated together in a single dosage unit, an individual compound may be administered at the same time as another therapeutic compound or sequentially, in any order thereof.

A disclosed compound can be administered alone, as the sole active pharmaceutical agent, or in combination with one or more additional compounds of the present disclosure or in conjunction with other agents. When administered as a combination, the therapeutic agents can be formulated as separate compositions that are administered simultaneously or at different times, or the therapeutic agents can be administered together as a single composition combining two or more therapeutic agents. Thus, the pharmaceutical compositions disclosed herein containing a compound of the present disclosure optionally include other therapeutic agents. Accordingly, certain embodiments are directed to such pharmaceutical compositions, where the composition further includes a therapeutically effective amount of an agent selected as is known to those of skill in the art.

Methods of Administration

The subject compounds find use for treating a disease or disorder in a subject. The route of administration may be selected according to a variety of factors including, but not limited to, the condition to be treated, the formulation and/or device used, the subject to be treated, and the like. Routes of administration useful in the disclosed methods include, but are not limited to, oral and parenteral routes, such as intravenous (iv), intraperitoneal (ip), rectal, topical, ophthalmic, nasal, intrathecal, and transdermal. Formulations for these dosage forms are described herein.

An effective amount of a subject compound may depend, at least, on the particular method of use, the subject being treated, the severity of the affliction, and the manner of administration of the therapeutic composition. A “therapeutically effective amount” of a composition is a quantity of a specified compound sufficient to achieve a desired effect in a subject (e.g., patient) being treated. For example, this may be the amount of a subject compound necessary to prevent, inhibit, reduce or relieve a disease or disorder in a subject. Ideally, a therapeutically effective amount of a compound is an amount sufficient to prevent, inhibit, reduce or relieve a disease or disorder in a subject without causing a substantial cytotoxic effect on host cells in the subject.

Therapeutically effective doses of a subject compound or pharmaceutical composition can be determined by one of skill in the art. For example, in some instances, a therapeutically effective dose of a compound or pharmaceutical composition is administered with a goal of achieving local (e.g., tissue) concentrations that are at least as high as the EC₅₀ of an applicable compound disclosed herein.

The specific dose level and frequency of dosage for any particular subject may be varied and may depend upon a variety of factors, including the activity of the subject compound, the metabolic stability and length of action of that compound, the age, body weight, general health, sex and diet of the subject, mode and time of administration, rate of excretion, drug combination, and severity of the condition of the host undergoing therapy.

In some embodiments, multiple doses of a compound are administered. The frequency of administration of a compound can vary depending on any of a variety of factors, e.g., severity of the symptoms, condition of the subject, etc. For example, in some embodiments, a compound is administered once per month, twice per month, three times per month, every other week, once per week (qwk), twice per week, three times per week, four times per week, five times per week, six times per week, every other day, daily (qd/od), twice a day (bds/bid), or three times a day (tds/tid), etc.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. By “average” is meant the arithmetic mean. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### General Synthetic Procedures

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Compounds as described herein can be purified by any purification protocol known in the art, including chromatography, such as HPLC, preparative thin layer chromatography, flash column chromatography and ion exchange chromatography. Any suitable stationary phase can be used, including normal and reversed phases as well as ionic resins. In certain embodiments, the disclosed compounds are purified via silica gel and/or alumina chromatography. See, e.g., Introduction to Modern Liquid Chromatography, 2nd Edition, ed. L. R. Snyder and J. J. Kirkland, John Wiley and Sons, 1979; and Thin Layer Chromatography, ed E. Stahl, Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4^{th} edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann, “Chemie der Kohlenhydrate: Monosaccharide and Derivate”, Georg Thieme Verlag, Stuttgart 1974. The protecting groups may be removed at a convenient subsequent stage using methods known from the art.

The subject compounds, including compounds that are not commercially available, can be synthesized via a variety of different synthetic routes using commercially available starting materials and/or starting materials prepared by conventional synthetic methods. A variety of examples of synthetic routes that can be used to synthesize the compounds disclosed herein are described in the schemes below.

In certain embodiments, compounds of the present disclosure (e.g., compounds of formula (I), (Ia) and (Ib)) are synthesized using conventional methods and conditions, as depicted in the combination of Scheme 1 and Scheme 2. Scheme 1 shows the formation of a component of a compound of the present disclosure, while Scheme 2 depicts the assembly of the complete compound. Components of compounds of the present disclosure are synthesized using conventional methods and conditions, as depicted in Scheme 1:

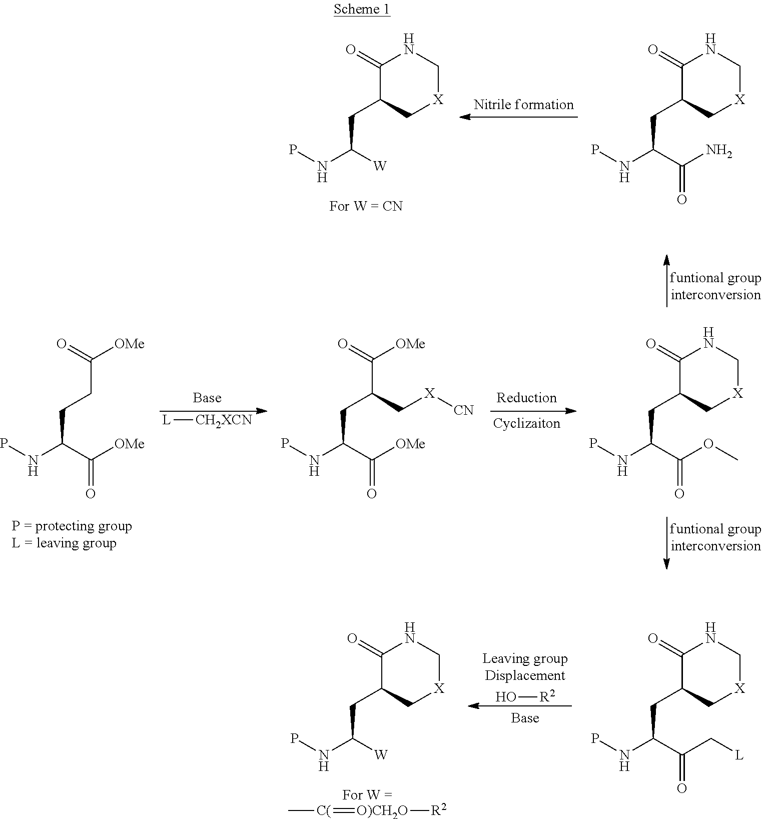

wherein R², W and X are as defined herein.

The starting materials and reagents employed in Scheme 1 may be obtained commercially or through conventional techniques. The scheme is an example of a method to generate components of compounds of the present disclosure where the exact steps and materials will depend on the functional groups present. The selection of the starting materials, reagent, substrates, base, protecting group, solvent and leaving group can be accomplished by one of ordinary skilled in the art. A nitrogen protected, a non-limiting example is a tert-butylcarbonate (Boc) group, diester of glutamic acid, a non-limiting example is the dimethyl ester, is reacted with base, such as lithium hexamethyldisilylamide, and then a nitrile containing electrophile, non-limiting examples are bromoacetonitrile and 3-bromopropionitrile. Reduction of the resulting nitrile, such as in the presence of cobalt (II) chloride and sodium borohydride, followed by cyclization to the corresponding lactam. The ester of the corresponding lactam can be converted to the primary amide, for example saponification with hydroxide, such as lithium hydroxide, and the resulting carboxylic acid coupled with ammonia using base and a coupling agent, such as with carbonyl diimidazole (CDI) or 1-[bis(dimethylamino)methylene-1H-1,2,3-triazolo[4,5-b]pyridinium 3-oxide hexafluorophosphate (HATU). Conversion of the amide to a nitrile, for W=CN, is accomplished using a dehydrating agent, such as POCl₃ or trifluoroacetic anhydride. Alternatively, for W=—C(═O)CH₂O—R² the ester intermediate can be interconverted to a substituted methyl ketone. There are numerous methods described in the literature that one skilled in the art could follow. One, non-limiting example, is to generate the methyl ketone with a leaving group (L), such as a chloro by contacting the ester with tert-butylmagnesium chloride and sodium chloroacetate in the presence of base. The resulting leaving group substituted methyl ketone, such as with a chloro, is reacted with the salt of the R²OH group, which is generated by reacting R²OH with a suitable base, such as sodium hydride or potassium tert-butoxide. The resulting displacement of the leaving group generates the intermediate where W=—C(═O)CH₂O—R².

Compounds of the present disclosure (e.g., compounds of formula (I), (Ia) and (Ib)) are synthesized using the intermediate generated in Scheme 1 with conventional methods and conditions, as depicted in Scheme 2:

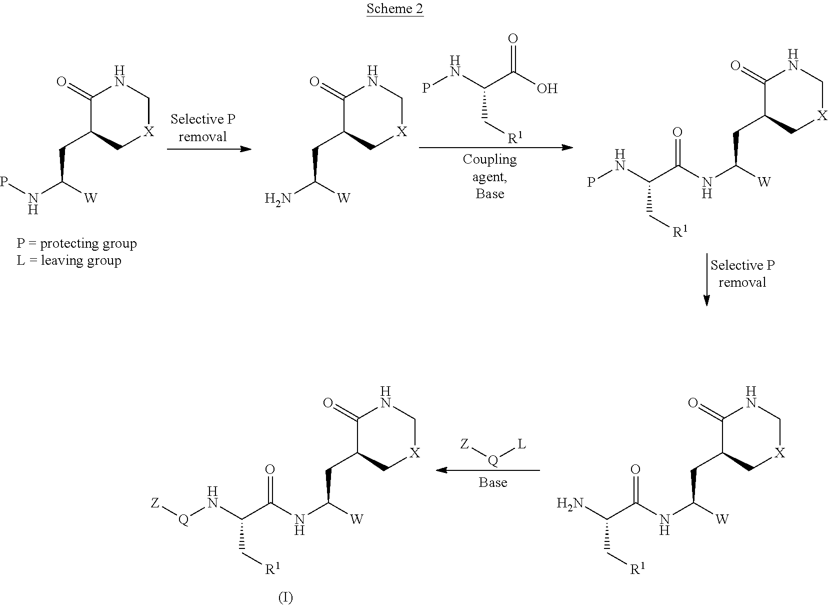

wherein R¹, Q, W, X and Z are as defined herein.

The starting materials and reagents employed in Scheme 2 may be obtained commercially or through techniques known to one of ordinary skill in the art. The scheme is an example of a method to generate compounds of the present disclosure where the exact steps and materials will depend on the functional groups present. The selection of the starting materials, reagent, substrates, base, protecting group, solvent and leaving group can be accomplished by one of ordinary skilled in the art. Removal of the nitrogen protecting group is well documented in the literature and is of common knowledge. For example, removal of a Boc group can be accomplished with acid, such as trifluoroacetic acid or hydrochloric acid. Coupling the resulting amine to a protected amino acid can be accomplished with a coupling agent, such as HATU, and an appropriate base, for example triethylamine or N-methylmorpholine. The resulting coupled product then has the protecting group selectively removed as is of common knowledge. For compounds of the present disclosure the resulting amine is then treated with Z-Q-L in the presence of reagent that correspond to the identity of Q and L. For Q, where L is for example chloride or bromide in the presence of an appropriate base, such as triethylamine or diisopropylethylamine, will facilitate the nitrogen to Q bonding reaction to generate the compound of the present disclosure. For Q=—C(═O)— and L is hydroxyl, contact with the amine and a coupling agent, as a non-limiting examples HATU, in the presence of an appropriate base, such as trimethylamine or N-methylmorpholine will form the amide bond of the compound of the present disclosure.

Schemes 1 and 2 are meant to be by way of non-limiting examples only, and one of ordinary skill in the art will understand that alternate reagents, solvents, order of reactions or starting materials can be used to make compounds of the present disclosure and/or other intermediates or compounds contained herein.

### Example 1: Synthesis of Compounds

All reagents and solvents were used as purchased from commercial sources. Moisture sensitive reactions were carried out under a nitrogen atmosphere. Reactions were monitored by TLC using pre-coated silica gel aluminum plates containing a fluorescent indicator (F-254). Detection was done with UV (254 nm). Alternatively, the progress of a reaction was monitored by LC/MS. Specifically, but without limitation, the following abbreviations were used, in addition to the other ones described herein, in the examples: Boc (tert-butoxycarbonyl); Boc₂O (di-tert-butyl dicarbonate); cat. (catalytic amount); DCM (dichloromethane); dioxane (1,4-dioxane); DMF (N,N-dimethylformamide); CDI (carbonyldiimidazole); EDCI (N-ethyl-N′-carbodiimide); EtOH (ethanol); ether or Et₂O (diethyl ether); Et₃N (triethylamine); EtOAc (ethyl acetate); HATU (1-[bis(dimethylamino)methylene]-1H-1,2,3-triazolo[4,5-b]pyridinium 3-oxide hexafluorophosphate or N-[(dimethylamino)-1H-1,2,3-triazolo-[4,5-b]pyridin-1-ylmethylene]-N-methylmethanaminium hexafluorophosphate N-oxide); KOtBu (potassium tert-butoxide); hex (hexanes); MeCN (acetonitrile); MeOH (methanol); μW (microwave); N-methylmorpholine (NMM); NaOtBu (sodium tert-butoxide); O/N (overnight); RT or rt (room or ambient temperature); TBS (tert-butyldimethylsilyl); t-BuMgCl (tert-butylmagnesium chloride); TFA (trifluoroacetic acid); TFAA (trifluoroacetic anhydride); THF (tetrahydrofuran). ¹H NMR spectra were recorded at RT with a Bruker Avance III 600 MHz NMR spectrometer equipped with a Bruker's 5 mm PABBO probe. Chemical shifts are reported in ppm downfield from tetramethylsilane using residual solvent signals as internal reference. NMR data were processed utilizing ACD/Spectrus processor (v2016.1.1, ACD/Labs Inc.). Nomenclature for the naming of compounds, such as for Compound Examples and intermediate compounds, were performed using ACD/Name (Chemists' Version from ACD/Labs Inc.) or Bruker TopSpin 4.0.6 to generate the IUPAC-style names. Naming of commercial or literature compounds utilized SciFinder, ACD/Names, and common or trivial names known to those skilled in the art.

The LC/MS system used for monitoring the progress of reactions, assessing the purity (absorbance at 254 nm) and identity of the product consisted of Dionex ULTIMATE 3000 uHPLC module and Thermo Scientific LTQ XL mass-spectrometer with electrospray ionization and Ion-Trap type of detector (alternating positive-negative mode). Separation was performed with Thermo Scientific™ Accucore™ aQ C18 Polar Endcapped LC column (100 mm×2.1 mm; particle size 2.6 μm, 80 Å). The column was maintained at 35° C. Commercial HPLC-grade methanol and domestic ‘millipore (Milli-Q)’ water used for chromatography were modified by adding 0.1% (v/v) of formic acid. The eluent was delivered with constant flow rate of 0.4 mL/min, column was equilibrated for 5 min with the corresponding eluent prior to injection of the sample (1 μL) and one of the following separation conditions were used:

Eluent Systems:
A—Gradient of Methanol-Water, 45 to 95% in 5.25 min, followed by 5 min of isocratic MeOH-water 95%; and
B—Gradient of Methanol-Water, 30 to 65% in 4.75 min, then to 95% in 2.5 min, followed by 4 min of isocratic MeOH-water 95%.

### Compound 1

### Synthesis of (3S)-3-{[N-(4-methoxy-1H-indole-2-carbonyl)-L-leucyl]amino}-2-oxo-4-[(3S)-2-oxopiperidin-3-yl]butyl 2,2-dimethylpropanoate, 1

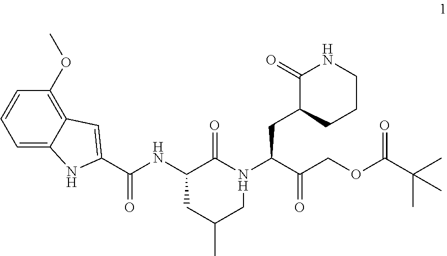

Compound 1 was synthesized as in Scheme 3.

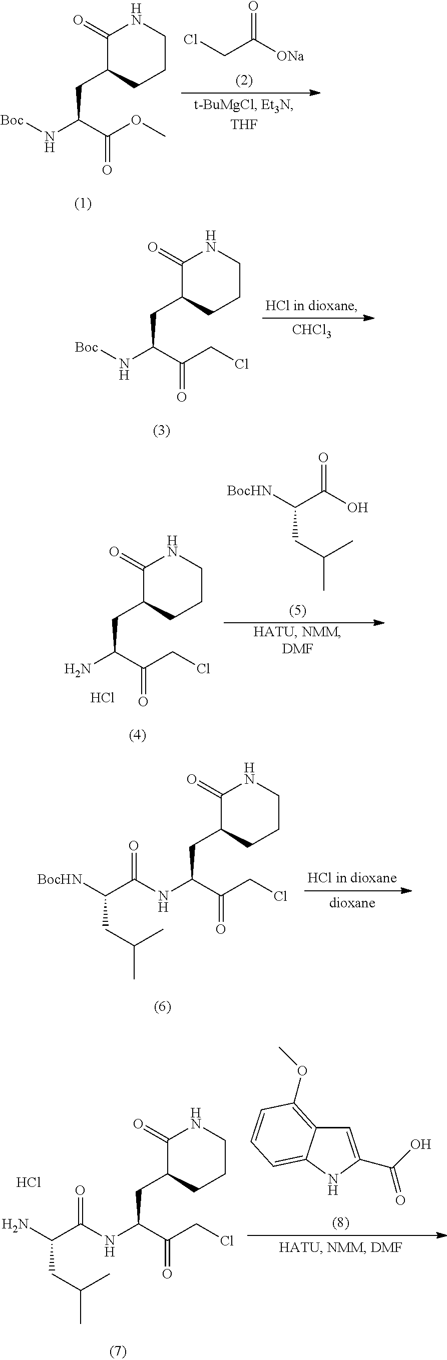

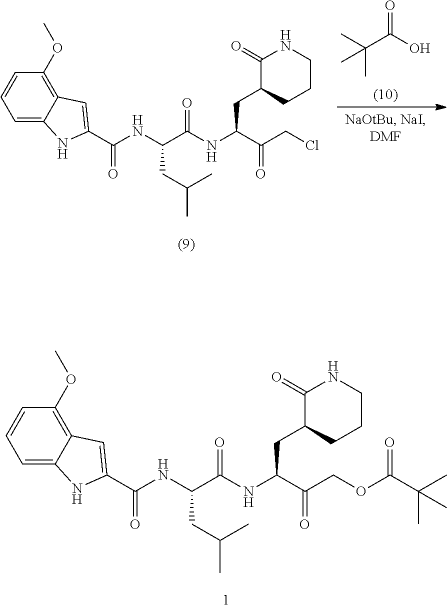

Preparation of tert-butyl {(2S)-4-chloro-3-oxo-1-[(3S)-2-oxopiperidin-3-yl]butan-2-yl}carbamate, (3). To a solution of methyl N-(tert-butoxycarbonyl)-3-[(3S)-2-oxopiperidin-3-yl]-L-alaninate (1) (7.0 g, 23.3 mmol) [prepared using method in Journal of Medicinal Chemistry (2015), 58, p 9414-9420 and references within], sodium chloroacetate (8.96 g, 76.9 mmol) and Et₃N (10.7 mL, 76.9 mmol) in anhydrous THF (200 mL) cooled in an ice bath was added a solution of 2.0 M tert-butylmagnesium chloride solution in THF (116.5 mL, 233 mmol) via a dropping funnel over 1.5 h. After 30 min, the ice bath was removed. After 18 h at room temperature, ice was then added, followed by addition of a precooled 4 N HCl aqueous solution to carefully adjust pH to 7. The two layers were separated and the aqueous layer was extracted with EtOAc (3×150 mL). The combined organic phase was washed with brine (100 mL), dried over Na₂SO₄, filtered and concentrated under reduced pressure. The crude residue was purified by column chromatography on silica gel with a gradient of 50 to 100% EtOAc in hexanes, which generated (3) (4.7 g, 63% yield) as a white foam.

Preparation of (3S)-3-[(2S)-2-amino-4-chloro-3-oxobutyl]piperidin-2-one hydrochloride salt, (4). A solution of tert-butyl {(2S)-4-chloro-3-oxo-1-[(3S)-2-oxopiperidin-3-yl]butan-2-yl}carbamate (3) (3.70 g, 11.60 mmol) in CHCl₃ (50 mL) was cooled using an ice-water bath and then 4 M HCl in 1,4-dioxane (14.5 mL) was added. After 30 min the ice bath was removed and the mixture warmed to room temperature. After overnight, an off-white precipitate formed and the mixture was concentrated under reduced pressure (30° C. water bath temperature). Co-evaporated with acetonitrile (3×20 mL), DCM (1×20 mL), and additional drying for 1 h under reduced pressure afforded (4) (2.96 g) as a cream color solid. This material was used without further purification in the next step. ¹H NMR (600 MHz, DMSO-d₆) δ 8.55 (br s, 3H), 7.80 (br s, 1H), 4.90 (d, J=16.9 Hz, 1H), 4.74 (d, J=16.9 Hz, 1H), 4.37-4.29 (m, 1H), 3.17-3.07 (m, 2H), 2.47-2.38 (m, 1H), 2.12-2.05 (m, 1H), 2.01-1.88 (m, 2H), 1.83-1.74 (m, 1H), 1.68-1.55 (m, 2H), 1.50-1.36 (m, 1H).

Preparation of N²-(tert-butoxycarbonyl)-N-{(2S)-4-chloro-3-oxo-1-[(3S)-2-oxopiperidin-3-yl]butan-2-yl}-L-leucinamide, (6). To a solution of (4) (2.96 g, 11.60 mmol) and N-(tert-butylcarbonyl)-L-leucine (5) (2.95 g, 12.76 mmol) in anhydrous DMF (50 mL) cooled in an ice bath was added HATU (4.85 g, 12.76 mmol). Then NMM (3.83 mL, 34.80 mmoL) was added dropwise. After 45 min, ice/saturated aqueous NaHCO₃ mixture (1:1, 200 mL) was added and the resulting mixture was extracted with ethyl acetate (3×250 mL). The combined organic layer was washed with saturated brine solution (1×250 mL), dried over anhydrous Na₂SO₄, filtered, and concentrated under reduced pressure. The resulting residue was dissolved in CHCl₃ (20 mL) and loaded on a 120 g silica gel column (Silicycle) and product purified by Biotage® with a gradient of 0 to 4% MeOH in CHCl₃, which generated (6) (2.70 g, 54% yield) as a white foamy solid. ¹H NMR (600 MHz, DMSO-d₆) δ 8.45 (d, J=7.9 Hz, 1H), 7.46 (br s, 1H), 7.04 (br d, J=7.2 Hz, 1H), 4.61 (d, J=16.7 Hz, 1H), 4.54 (d, J=16.7 Hz, 1H), 4.50-4.39 (m, 1H), 3.93-3.86 (m, 1H), 3.14-3.06 (m, 2H), 2.22-2.09 (m, 2H), 1.90-1.83 (m, 1H), 1.80-1.57 (m, 4H), 1.57-1.48 (m, 1H), 1.48-1.41 (m, 1H), 1.38 (s, 9H), 1.34-1.23 (m, 1H), 0.89 (d, J=6.6 Hz, 3H), 0.85 (d, J=6.6 Hz, 3H).

Preparation of N-{(2S)-4-chloro-3-oxo-1-[(3S)-2-oxopiperidin-3-yl]butan-2-yl}-L-leucinamide hydrochloride salt, (7). A solution of (6) (2.70 g, 6.25 mmol) in CHCl₃ (25 mL) was cooled using an ice-water bath and then 4 M HCl in 1,4-dioxane (10 mL) was added. After 30 min, the ice bath was removed. After overnight, a gummy precipitate formed. The mixture was then concentrated under reduced pressure (bath temperature at 35° C.). Co-evaporated with acetonitrile (3×20 mL), DCM (1×20 mL), and drying under reduced pressure for an 1 h afforded (7) (2.30 g, quantitative yield) as an off-white color solid. This material was used without further purification in the next step. ¹H NMR (600 MHz, DMSO-d₆) δ 9.13 (d, J=7.5 Hz, 1H), 8.33 (br d, J=3.8 Hz, 3H), 7.53 (br s, 1H), 4.68 (s, 2H), 4.56 (ddd, J=4.0, 7.3, 11.1 Hz, 1H), 3.86-3.76 (m, 1H), 3.76-3.57 (m, 1H), 3.18-3.05 (m, 2H), 2.29-2.20 (m, 1H), 2.14 (ddd, J=4.7, 11.2, 14.1 Hz, 1H), 1.97-1.89 (m, 1H), 1.78-1.64 (m, 4H), 1.64-1.51 (m, 1H), 1.43-1.35 (m, 1H), 0.93 (d, J=6.4 Hz, 3H), 0.91 (d, J=6.4 Hz, 3H).

Preparation of N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide, (9). To a solution of (7) (2.30 g, 6.24 mmol) and 4-methoxy-1H-indole-2-carboxylic acid (8) (1.31 g, 6.87 mmol) in anhydrous DMF (25 mL) cooled in an ice bath was added HATU (2.61 g, 6.87 mmol). Then NMM (2.06 mL, 18.74 mmol) was added dropwise. After 45 min, ice/saturated aqueous NaHCO₃ mixture (1:1, 150 mL) was added and the resulting mixture was extracted with EtOAc (3×150 mL). The combined organic layer was washed with saturated brine solution (1×150 mL), dried over anhydrous Na₂SO₄, filtered, and concentrated under reduced pressure. The residue was dissolved in CHCl₃ (25 mL) and loaded on 120 g silica gel column (Silicycle) and product purified by Biotage® with a gradient of 0 to 4% MeOH in CHCl₃. The resulting solid was triturated with EtOAc (10 mL), filtered, and the solid washed with EtOAc (2×3 mL), dried under vacuum, which generated (9) (1.71 g, 54% yield) as an off-white solid. ¹H NMR (600 MHz, DMSO-d₆) δ 11.57 (d, J=2.1 Hz, 1H), 8.60 (d, J=7.7 Hz, 1H), 8.44 (d, J=7.5 Hz, 1H), 7.46 (br s, 1H), 7.37 (dd, J=0.8, 2.3 Hz, 1H), 7.09 (t, J=8.1 Hz, 1H), 7.02 (d, J=8.3 Hz, 1H), 6.51 (d, J=7.3 Hz, 1H), 4.61 (d, J=16.0 Hz, 1H), 4.57 (d, J=16.0 Hz, 1H), 4.53-4.42 (m, 2H), 3.89 (s, 3H), 3.13-3.04 (m, 2H), 2.22-2.13 (m, 2H), 1.89-1.80 (m, 1H), 1.78-1.66 (m, 4H), 1.60-1.47 (m, 2H), 1.39-1.29 (m, 1H), 0.95 (d, J=6.4 Hz, 3H), 0.89 (d, J=6.4 Hz, 3H). LC/MS: Eluent system A (retention time: 5.19 min); ESI-MS: 505 [M+H]⁺.

Preparation of (3S)-3-{[N-(4-methoxy-1H-indole-2-carbonyl)-L-leucyl]amino}-2-oxo-4-[(3S)-2-oxopiperidin-3-yl]butyl 2,2-dimethylpropanoate, 1. A mixture of 2,2-dimethylpropanoic acid (10) (48.54 mg, 0.48 mmol) and sodium tert-butoxide (22.8 mg, 0.24 mmol) in anhydrous DMF (3 mL) was stirred at room temperature for 30 min. To this N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (40 mg, 0.08 mmol) and NaI (23.8 mg, 0.16 mmol) were added. After 24 h, saturated brine solution (10 mL) was added and the mixture extracted with CHCl₃ (3×10 mL). The combined organic layer was washed with saturated brine solution (10 mL), dried over anhydrous Na₂SO₄, filtered, and concentrated reduced pressure. The residue was dissolved in CHCl₃ (5 mL) and loaded on silica gel column (3×4 g Silicycle siliasep columns) and the product was purified by Biotage® with a gradient of 0 to 1% MeOH in CHCl₃, which afforded 1 (14.9 mg, 33% yield) as an off-white solid. ¹H NMR (600 MHz, DMSO-d₆) δ 11.58 (d, J=2.3 Hz, 1H), 8.57 (d, J=8.1 Hz, 1H), 8.44 (d, J=7.7 Hz, 1H), 7.46 (br s, 1H), 7.37 (dd, J=0.8, 2.3 Hz, 1H), 7.11 (t, J=8.0 Hz, 1H), 7.01 (d, J=8.3 Hz, 1H), 6.51 (d, J=7.5 Hz, 1H), 4.87 (d, J=17.1 Hz, 1H), 4.84 (d, J=17.1 Hz, 1H), 4.51-4.42 (m, 2H), 3.89 (s, 3H), 3.14-3.04 (m, 2H), 2.24-2.15 (m, 2H), 1.88-1.80 (m, 1H), 1.77-1.64 (m, 4H), 1.60-1.47 (m, 2H), 1.38-1.27 (m, 1H), 1.18 (s, 9H), 0.95 (d, J=6.4 Hz, 3H), 0.90 (d, J=6.4 Hz, 3H). LC/MS: Eluent system A (retention time: 6.68 min); ESI-MS: 571 [M+H]⁺.

### Compound 2

### Synthesis of (3S)-3-{[N-(4-methoxy-1H-indole-2-carbonyl)-L-leucyl]amino}-2-oxo-4-[(3S)-2-oxopiperidin-3-yl]butyl benzoate, 2

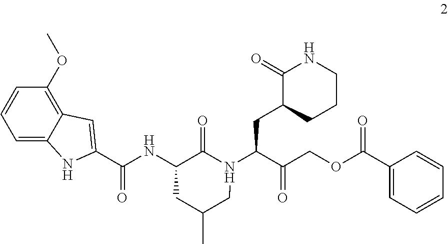

Compound 2 was synthesized as in Scheme 4.

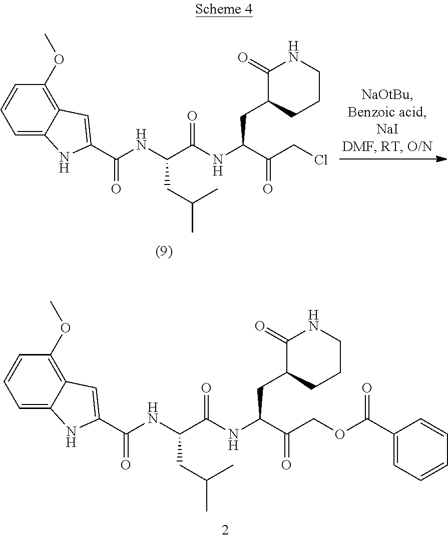 [SMILES: COc1cccc2[nH]c(C(=O)N[C@@H](CC(C)C)C(=O)N[C@@H](C[C@@H]3CCCNC3=O)C(=O)CCl)cc12 ; COc1cccc2[nH]c(C(=O)N[C@@H](CC(C)C)C(=O)N[C@@H](C[C@@H]3CCCNC3=O)C(=O)COC(=O)c3ccccc3)cc12]

Preparation of (3S)-3-{[N-(4-methoxy-1H-indole-2-carbonyl)-L-leucyl]amino}-2-oxo-4-[(3S)-2-oxopiperidin-3-yl]butyl benzoate, 2. To a solution of benzoic acid (37 mg, 0.30 mmol) in DMF (3 mL) was added NaOtBu (19 mg, 0.20 mmol). After 15 min, a solution of N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (50 mg, 0.10 mmol) in DMF (2 mL) was added, followed by NaI (15 mg, 0.10 mmol). After overnight, water (5 mL) was added and the resulting mixture was extracted with DCM (3×25 mL). The combined organic phase was washed with brine (10 mL), dried over Na₂SO₄, filtered and concentrated under reduced pressure. The product was purified by column chromatography on silica gel with a gradient of 0 to 10% MeOH in EtOAc, which generated 2 (15 mg, 25% yield) as a white solid. ¹H NMR (600 MHz, DMSO-d₆) δ 11.58 (d, J=2.3 Hz, 1H), 8.64 (d, J=7.9 Hz, 1H), 8.45 (d, J=7.5 Hz, 1H), 8.01-7.96 (m, 2H), 7.71-7.68 (m, 1H), 7.59-7.52 (m, 2H), 7.47 (br s, 1H), 7.39-7.36 (m, 1H), 7.12-7.07 (m, 1H), 7.03-6.99 (m, 1H), 6.51 (d, J=7.5 Hz, 1H), 5.16-5.11 (m, 2H), 4.56-4.46 (m, 2H), 3.88 (s, 3H), 3.13-3.06 (m, 2H), 2.27-2.19 (m, 2H), 1.88-1.82 (m, 1H), 1.79-1.66 (m, 4H), 1.62-1.48 (m, 2H), 1.38-1.32 (m, 1H), 0.95 (d, J=6.4 Hz, 3H), 0.90 (d, J=6.4 Hz, 3H). LC/MS: Eluent system B (retention time: 6.19 min); ESI-MS 591 [M+H]⁺.

### Compound 3

### Synthesis of (3S)-3-{[N-(4-methoxy-1H-indole-2-carbonyl)-L-leucyl]amino}-2-oxo-4-[(3S)-2-oxopiperidin-3-yl]butyl pyridine-2-carboxylate, 3

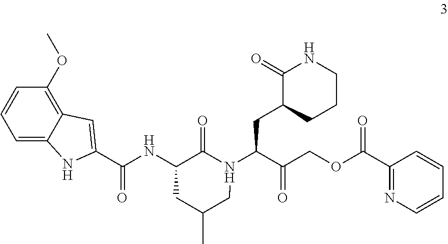

Compound 3 was synthesized as in Scheme 5.

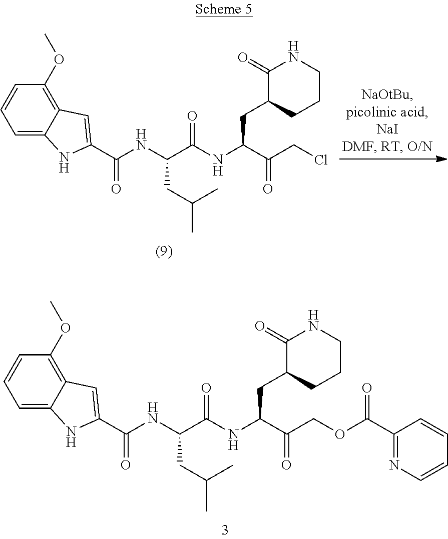 [SMILES: COc1cccc2[nH]c(C(=O)N[C@@H](CC(C)C)C(=O)N[C@@H](C[C@@H]3CCCNC3=O)C(=O)CCl)cc12 ; COc1cccc2[nH]c(C(=O)N[C@@H](CC(C)C)C(=O)N[C@@H](C[C@@H]3CCCNC3=O)C(=O)COC(=O)c3ccccn3)cc12]

Preparation of (3S)-3-{[N-(4-methoxy-1H-indole-2-carbonyl)-L-leucyl]amino}-2-oxo-4-[(3S)-2-oxopiperidin-3-yl]butyl pyridine-2-carboxylate, 3. To a solution of picolinic acid (41 mg, 0.33 mmol) in DMF (3 mL) was added NaOtBu (21 mg, 0.22 mmol). After 15 min, a solution of N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (55 mg, 0.11 mmol) in DMF (2 mL), followed by addition of NaI (16 mg, 0.11 mmol). The reaction mixture was stirred at room temperature for overnight. Then it was quenched by adding water (5 mL). The mixture was extracted with DCM (3×25 mL), and the combined organic phase was washed with brine (10 mL), dried over Na₂SO₄, filtered and concentrated. The product was purified by column chromatography on silica gel with a gradient of 0 to 10% MeOH in EtOAc, which generated 3 (12 mg, 20% yield) as a white solid. ¹H NMR (600 MHz, DMSO-d₆) δ 11.59 (d, J=1.9 Hz, 1H), 8.77-8.73 (m, 1H), 8.65 (d, J=8.3 Hz, 1H), 8.46 (d, J=7.5 Hz, 1H), 8.11-8.07 (m, 1H), 8.05-8.00 (m, 1H), 7.69 (ddd, J=1.1, 4.7, 7.7 Hz, 1H), 7.47 (br s, 1H), 7.39-7.37 (m, 1H), 7.12-7.07 (m, 1H), 7.03-6.99 (m, 1H), 6.51 (d, J=7.5 Hz, 1H), 5.22-5.13 (m, 2H), 4.58-4.53 (m, 1H), 4.52-4.46 (m, 1H), 3.89 (s, 3H), 3.13-3.06 (m, 2H), 2.28-2.21 (m, 2H), 1.89-1.83 (m, 1H), 1.78-1.68 (m, 4H), 1.62-1.48 (m, 2H), 1.40-1.33 (m, 1H), 0.96 (d, J=6.4 Hz, 3H), 0.91 (d, J=6.4 Hz, 3H). LC/MS: Eluent system B (retention time: 5.19 min); ESI-MS 592 [M+H]⁺.

### Compound 4

### Synthesis of (3S)-3-{[N-(4-methoxy-1H-indole-2-carbonyl)-L-leucyl]amino}-2-oxo-4-[(3S)-2-oxopiperidin-3-yl]butyl pyridine-3-carboxylate, 4

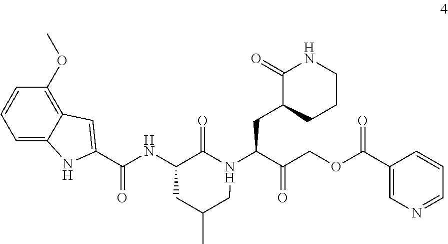

Compound 4 was synthesized as in Scheme 6.

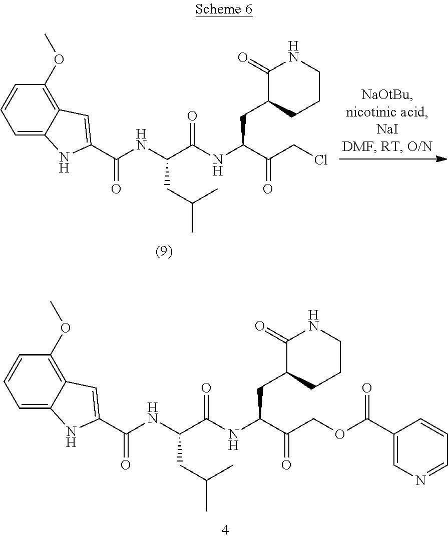 [SMILES: COc1cccc2[nH]c(C(=O)N[C@@H](CC(C)C)C(=O)N[C@@H](C[C@@H]3CCCNC3=O)C(=O)CCl)cc12 ; COc1cccc2[nH]c(C(=O)N[C@@H](CC(C)C)C(=O)N[C@@H](C[C@@H]3CCCNC3=O)C(=O)COC(=O)c3cccnc3)cc12]

Preparation of (3S)-3-{[N-(4-methoxy-1H-indole-2-carbonyl)-L-leucyl]amino}-2-oxo-4-[(3S)-2-oxopiperidin-3-yl]butyl pyridine-3-carboxylate, 4. To a solution of nicotinic acid (37 mg, 0.30 mmol) in DMF (3 mL) was added NaOtBu (19 mg, 0.20 mmol). After 15 min, a solution of N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (50 mg, 0.10 mmol) in DMF (2 mL) was added followed by addition of NaI (15 mg, 0.10 mmol). After overnight, water (5 mL) was added and the resulting mixture was extracted with DCM (3×25 mL). The combined organic layer was washed with brine (10 mL), dried over Na₂SO₄, filtered and concentrated under reduced pressure. The product was purified by column chromatography on silica gel with a gradient of 0 to 10% MeOH in EtOAc, which generated 4 (28 mg, 47% yield) as an off-white solid. ¹H NMR (600 MHz, DMSO-d₆) δ 11.58 (d, J=2.2 Hz, 1H), 9.12 (dd, J=0.8, 2.3 Hz, 1H), 8.86 (dd, J=1.5, 4.9 Hz, 1H), 8.65 (d, J=8.3 Hz, 1H), 8.46 (d, J=7.5 Hz, 1H), 8.34-8.31 (m, 1H), 7.61 (ddd, J=1.1, 4.8, 8.0 Hz, 1H), 7.48 (br s, 1H), 7.40-7.37 (m, 1H), 7.13-7.08 (m, 1H), 7.03-6.99 (m, 1H), 6.51 (d, J=7.5 Hz, 1H), 5.21-5.13 (m, 2H), 4.59-4.52 (m, 1H), 4.52-4.47 (m, 1H), 3.89 (s, 3H), 3.13-3.06 (m, 2H), 2.27-2.21 (m, 2H), 1.89-1.83 (m, 1H), 1.78-1.67 (m, 4H), 1.61-1.48 (m, 2H), 1.39-1.31 (m, 1H), 0.95 (d, J=6.4 Hz, 3H), 0.90 (d, J=6.8 Hz, 3H). LC/MS: Eluent system B (retention time: 5.31 min); ESI-MS 592 [M+H]⁺.

### Compound 5

### Synthesis of (3S)-3-{[N-(4-methoxy-1H-indole-2-carbonyl)-L-leucyl]amino}-2-oxo-4-[(3S)-2-oxopiperidin-3-yl]butyl 2,6-dichlorobenzoate, 5

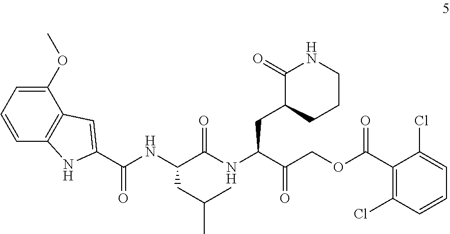 [SMILES: COc1cccc2[nH]c(C(=O)N[C@@H](CC(C)C)C(=O)N[C@@H](C[C@@H]3CCCNC3=O)C(=O)COC(=O)c3c(Cl)cccc3Cl)cc12]

Compound 5 was synthesized as in Scheme 8.

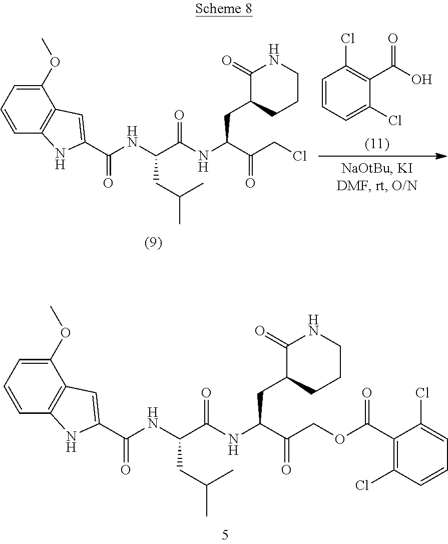

Preparation of (3S)-3-{[N-(4-methoxy-1H-indole-2-carbonyl)-L-leucyl]amino}-2-oxo-4-[(3S)-2-oxopiperidin-3-yl]butyl 2,6-dichlorobenzoate, 5. After 30 min, to a solution of 2,6-dichlorobenzoic acid (11) (101 mg, 0.53 mmol) and sodium tert-butoxide (30 mg, 0.31 mmol) in DMF (3 mL) was added N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (50 mg, 0.099 mmol) followed by potassium iodide (42 mg, 0.25 mmol). After overnight, the mixture was diluted with DCM (15 mL) and washed with water (15 mL). Organic layer was poured into Petri dish and left in the fume hood to dry. The residue was dissolved in a minimal amount of ethyl acetate and loaded on silica. Purification of the product by silica gel column chromatography (eluted with gradient of 0 to 12% methanol in 1:1 chloroform:ethyl acetate mixture), followed by recrystallization from ethyl acetate provided 5 (17.8 mg, 27% yield) as a white powder. ¹H NMR (600 MHz, DMSO-d6) δ 11.55 (d, J=2.1 Hz, 1H), 8.62 (d, J=7.6 Hz, 1H), 8.45 (d, J=7.6 Hz, 1H), 7.62-7.59 (m, 2H), 7.57-7.54 (m, 1H), 7.47-7.44 (m, 1H), 7.36 (dd, J=0.9, 2.3 Hz, 1H), 7.10 (t, J=8.0 Hz, 1H), 7.01 (d, J=8.3 Hz, 1H), 6.51 (d, J=8.0 Hz, 1H), 5.19 (d, J=17.0 Hz, 1H), 5.16 (d, J=17.0 Hz, 1H), 4.58-4.48 (m, 2H), 3.88 (s, 3H), 3.12-3.04 (m, 2H), 2.25-2.20 (m, 2H), 1.87-1.66 (m, 4H), 1.61-1.47 (m, 2H), 1.38-1.31 (m, 2H), 0.95 (d, J=6.3 Hz, 3H), 0.90 (d, J=6.6 Hz, 3H). LC/MS: Eluent system A (retention time: 6.33 min); ESI-MS 659 [M+H]⁺, 657 [M−H]⁻.

### Compound 6

### Synthesis of (3S)-3-{[N-(4-methoxy-1H-indole-2-carbonyl)-L-leucyl]amino}-2-oxo-4-[(3S)-2-oxopiperidin-3-yl]butyl 2,6-bis(trifluoromethyl)benzoate, 6

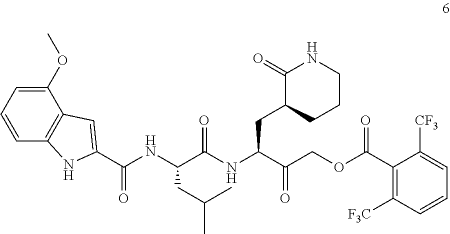 [SMILES: COc1cccc2[nH]c(C(=O)N[C@@H](CC(C)C)C(=O)N[C@@H](C[C@@H]3CCCNC3=O)C(=O)COC(=O)c3c(C(F)(F)F)cccc3C(F)(F)F)cc12]

Compound 6 was synthesized as in scheme 9.

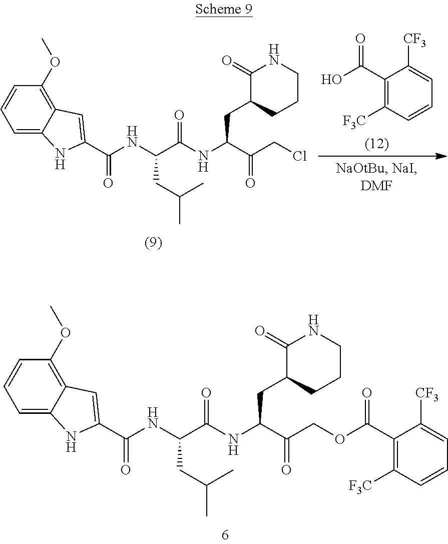

Preparation of (3S)-3-{[N-(4-methoxy-1H-indole-2-carbonyl)-L-leucyl]amino}-2-oxo-4-[(3S)-2-oxopiperidin-3-yl]butyl 2,6-bis(trifluoromethyl)benzoate, 6. A mixture of 2,6-bis(trifluoromethyl)benzoic acid (12) (122.7 mg, 0.48 mmol) and sodium tert-butoxide (22.8 mg, 0.24 mmol) in anhydrous DMF (3 mL) was stirred at room temperature for 30 min. To this N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (40 mg, 0.08 mmol) and NaI (23.8 mg, 0.16 mmol) were added successively. After 48 h, a saturated brine solution (10 mL) was added and the mixture was extracted with CHCl₃ (3×10 mL). The combined organic layer was washed with saturated brine solution (10 mL), dried over anhydrous Na₂SO₄, filtered, and concentrated under reduced pressure. The resulting residue was dissolved in CHCl₃ (5 mL) and loaded on silica gel column (3×4 g Silicycle siliasep columns) and the product was purified by Biotage® with a gradient of 0 to 2% MeOH in CHCl₃, which afforded 6 (31.9 mg, 55% yield) as an off-white solid. ¹H NMR (600 MHz, DMSO-d₆) δ 11.55 (d, J=2.1 Hz, 1H), 8.65 (d, J=7.9 Hz, 1H), 8.45 (d, J=7.7 Hz, 1H), 8.26 (d, J=7.9 Hz, 2H), 8.02 (t, J=8.0 Hz, 1H), 7.46 (br s, 1H), 7.37 (dd, J=0.7, 2.2 Hz, 1H), 7.11 (t, J=8.1 Hz, 1H), 7.02 (d, J=8.2 Hz, 1H), 6.51 (d, J=7.5 Hz, 1H), 5.21 (d, J=17.1 Hz, 1H), 5.16 (d, J=17.1 Hz, 1H), 4.60-4.49 (m, 2H), 3.89 (s, 3H), 3.15-3.03 (m, 2H), 2.28-2.19 (m, 2H), 1.87-1.79 (m, 1H), 1.77-1.63 (m, 4H), 1.62-1.55 (m, 1H), 1.55-1.46 (m, 1H), 1.37-1.28 (m, 1H), 0.95 (d, J=6.4 Hz, 3H), 0.90 (d, J=6.4 Hz, 3H). ¹⁹F NMR (565 MHz, DMSO-d₆) δ −58.28-−58.36 (m, 6F). LC/MS: Eluent system A (retention time: 6.49 min); ESI-MS: 727 [M+H]⁺.

### Compound 7

### Synthesis of (3S)-3-{[N-(4-methoxy-1H-indole-2-carbonyl)-L-leucyl]amino}-2-oxo-4-[(3S)-2-oxopiperidin-3-yl]butyl pentafluorobenzoate, 7

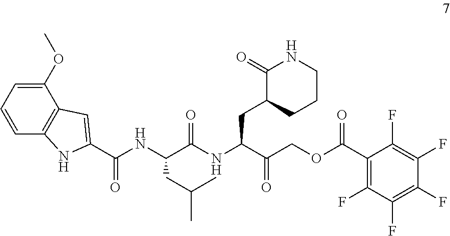 [SMILES: COc1cccc2[nH]c(C(=O)N[C@@H](CC(C)C)C(=O)N[C@@H](C[C@@H]3CCCNC3=O)C(=O)COC(=O)c3c(F)c(F)c(F)c(F)c3F)cc12]

Compound 7 was synthesized as in scheme 10.

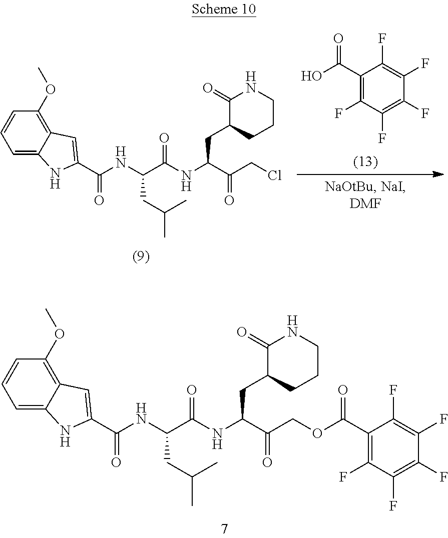 [SMILES: COc1cccc2[nH]c(C(=O)N[C@@H](CC(C)C)C(=O)N[C@@H](C[C@@H]3CCCNC3=O)C(=O)CCl)cc12 ; O=C(O)c1c(F)c(F)c(F)c(F)c1F ; COc1cccc2[nH]c(C(=O)N[C@@H](CC(C)C)C(=O)N[C@@H](C[C@@H]3CCCNC3=O)C(=O)COC(=O)c3c(F)c(F)c(F)c(F)c3F)cc12]

Preparation of (3S)-3-{[N-(4-methoxy-1H-indole-2-carbonyl)-L-leucyl]amino}-2-oxo-4-[(3S)-2-oxopiperidin-3-yl]butyl pentafluorobenzoate, 7. A mixture of pentafluorobenzoic acid (13) (100.8 mg, 0.475 mmol) and sodium tert-butoxide (22.8 mg, 0.237 mmol) in anhydrous DMF (3 mL) was stirred at room temperature for 30 min. To this N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (40 mg, 0.079 mmol) and NaI (23.8 mg, 0.158 mmol) were added successively. After 48 h, saturated brine solution (10 mL) was added and the resulting mixture was extracted with CHCl₃ (3×10 mL). The combined organic layer was washed with saturated brine solution (10 mL), dried over anhydrous Na₂SO₄, filtered, and concentrated under reduced pressure. The crude was dissolved in CHCl₃ (5 mL) and loaded on silica gel column (3×4 g Silicycle siliasep columns) and the product was purified by Biotage® with a gradient of 0 to 2% MeOH in CHCl₃, which afforded 7 (11.1 mg, 20% yield) as an off-white solid. ¹H NMR (600 MHz, DMSO-d₆) δ 11.58 (d, J=2.1 Hz, 1H), 8.63 (d, J=8.1 Hz, 1H), 8.46 (d, J=7.5 Hz, 1H), 7.47 (br s, 1H), 7.37 (dd, J=0.7, 2.1 Hz, 1H), 7.11 (t, J=8.1 Hz, 1H), 7.01 (d, J=8.3 Hz, 1H), 6.51 (d, J=7.5 Hz, 1H), 5.26 (d, J=17.0 Hz, 1H), 5.22 (d, J=17.0 Hz, 1H), 4.56-4.51 (m, 1H), 4.50-4.45 (m, 1H), 3.89 (s, 3H), 3.15-3.05 (m, 2H), 2.28-2.17 (m, 2H), 1.89-1.81 (m, 1H), 1.78-1.66 (m, 4H), 1.63-1.46 (m, 2H), 1.44-1.30 (m, 1H), 0.95 (d, J=6.4 Hz, 3H), 0.90 (d, J=6.4 Hz, 3H). ¹⁹F NMR (565 MHz, DMSO-d₆) δ −138.40-−138.66 (m, 2F), −147.94-−148.25 (m, 1F), −160.63-−161.00 (m, 2F). LC/MS: Eluent system A (retention time: 6.71 min); ESI-MS: 681 [M+H]⁺.

### Compound 8

### Synthesis of (3S)-3-{[N-(4-methoxy-1H-indole-2-carbonyl)-L-leucyl]amino}-2-oxo-4-[(3S)-2-oxopiperidin-3-yl]butyl pyridine-4-carboxylate, 8

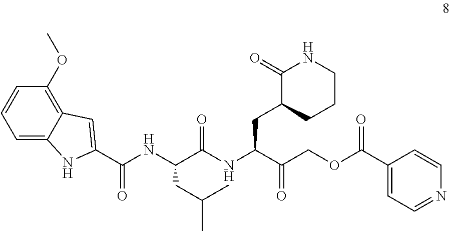 [SMILES: COc1cccc2[nH]c(C(=O)N[C@@H](CC(C)C)C(=O)N[C@@H](C[C@@H]3CCCNC3=O)C(=O)COC(=O)c3ccncc3)cc12]

Compound 8 was synthesized as in Scheme 11.

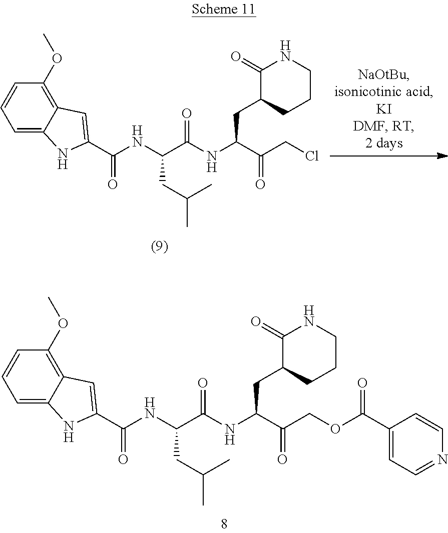 [SMILES: COc1cccc2[nH]c(C(=O)N[C@@H](CC(C)C)C(=O)N[C@@H](C[C@@H]3CCCNC3=O)C(=O)CCl)cc12 ; COc1cccc2[nH]c(C(=O)N[C@@H](CC(C)C)C(=O)N[C@@H](C[C@@H]3CCCNC3=O)C(=O)COC(=O)c3ccncc3)cc12]

Preparation of (3S)-3-{[N-(4-methoxy-1H-indole-2-carbonyl)-L-leucyl]amino}-2-oxo-4-[(3S)-2-oxopiperidin-3-yl]butyl pyridine-4-carboxylate, 8. To a solution of isonicotinic acid (37 mg, 0.30 mmol) in DMF (5 mL) was added NaOtBu (19 mg, 0.20 mmol). After 15 min, N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (50 mg, 0.10 mmol) was added followed by KI (16.6 mg, 0.10 mmol). After 2 days, water (5 mL) was added and the resulting mixture was extracted with EtOAc (3×20 mL), the combined organic phase was washed with brine (10 mL), dried over Na₂SO₄, filtered and concentrated under reduced pressure. The product was purified by column chromatography on silica gel with a gradient of 0 to 10% MeOH in EtOAc, which generated 8 (31 mg, 52% yield) as a white solid. ¹H NMR (600 MHz, DMSO-d₆) δ 11.58 (d, J=2.3 Hz, 1H), 8.88-8.81 (m, 2H), 8.65 (d, J=7.9 Hz, 1H), 8.46 (d, J=7.9 Hz, 1H), 7.89-7.84 (m, 2H), 7.48 (br s, 1H), 7.39-7.37 (m, 1H), 7.13-7.08 (m, 1H), 7.04-6.99 (m, 1H), 6.52 (d, J=7.5 Hz, 1H), 5.23-5.15 (m, 2H), 4.58-4.52 (m, 1H), 4.52-4.46 (m, 1H), 3.89 (s, 3H), 3.14-3.06 (m, 2H), 2.27-2.20 (m, 2H), 1.89-1.83 (m, 1H), 1.80-1.64 (m, 4H), 1.61-1.50 (m, 2H), 1.39-1.31 (m, 1H), 0.95 (d, J=6.4 Hz, 3H), 0.90 (d, J=6.4 Hz, 3H). LC/MS: Eluent system A (retention time: 5.36 min); ESI-MS 592 [M+H]⁺.

### Compound 9

### Synthesis of ((3S)-3-{[N-(4-methoxy-1H-indole-2-carbonyl)-L-leucyl]amino}-2-oxo-4-[(3S)-2-oxopiperidin-3-yl]butyl 2,2,5-trimethyl-1,3-dioxane-5-carboxylate, 9

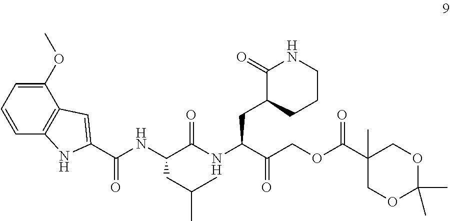 [SMILES: COc1cccc2[nH]c(C(=O)N[C@@H](CC(C)C)C(=O)N[C@@H](C[C@@H]3CCCNC3=O)C(=O)COC(=O)C3(C)COC(C)(C)OC3)cc12]

Compound 9 was synthesized as in Scheme 12.

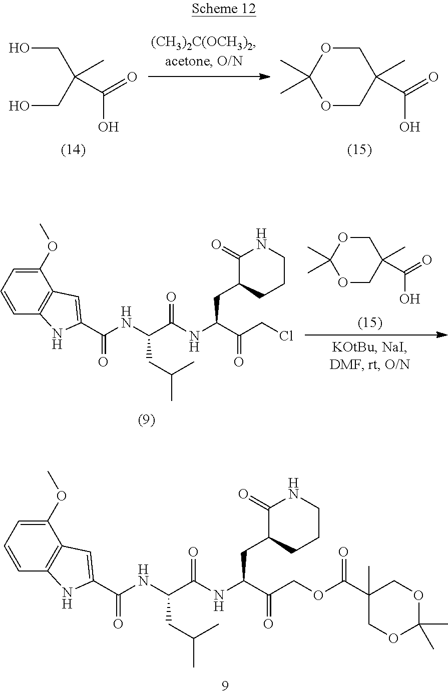

Preparation of 2,2,5-trimethyl-1,3-dioxane-5-carboxylic acid, (15). To an ice cooled solution of 3-hydroxy-2-(hydroxymethyl)-2-methylpropanoic acid (14) (1.0 g, 7.45 mmol) in acetone (100 mL) was added 2,2-dimethoxypropane (0.9 g, 8.21 mmol) slowly. The ice bath was removed and after overnight the mixture was concentrated under reduced pressure. The resulting gum was suspended in ether (25 mL) to form a solid and after filtration and drying generated (15) (1.0 g, 79% yield) as a white solid.

Preparation of (3S)-3-{[N-(4-methoxy-1H-indole-2-carbonyl)-L-leucyl]amino}-2-oxo-4-[(3S)-2-oxopiperidin-3-yl]butyl 2,2,5-trimethyl-1,3-dioxane-5-carboxylate, 9. To a solution of 2,2,5-trimethyl-1,3-dioxane-5-carboxylic acid (15) (69 mg, 0.396 mmol) in DMF (1 mL) was added KOtBu (31 mg, 0.273 mmol). After 10 min, N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (40 mg, 0.079 mmol) was added to the mixture followed by sodium iodide (15 mg, 0.100 mmol). After overnight, ethyl acetate/chloroform (75 mL/25 mL) was added and the resulting mixture was washed with water (25 mL) and brine (25 mL), and the organic layer was dried over MgSO₄, concentrated under reduce pressure and the product purified by column chromatography on silica (0-2% methanol in chloroform), which produced 9 (29 mg, 56% yield). ¹H NMR (600 MHz, DMSO-d₆) δ 11.59 (d, J=2.3 Hz, 1H), 8.58 (d, J=8.1 Hz, 1H), 8.45 (d, J=7.7 Hz, 1H), 7.46 (br s, 1H), 7.39-7.36 (m, 1H), 7.13-7.07 (m, 1H), 7.04-6.99 (m, 1H), 6.51 (d, J=7.5 Hz, 1H), 4.95-4.87 (m, 2H), 4.51-4.43 (m, 2H), 4.09-4.02 (m, 2H), 3.89 (s, 3H), 3.67-3.60 (m, 2H), 3.14-3.04 (m, 2H), 2.25-2.14 (m, 2H), 1.89-1.79 (m, 1H), 1.77-1.64 (m, 4H), 1.61-1.45 (m, 1H), 1.36 (s, 3H), 1.35-1.30 (m, 2H), 1.27 (s, 3H), 1.15 (s, 3H), 0.95 (d, J=6.4 Hz, 3H), 0.90 (d, J=6.4 Hz, 3H). LC/MS: Eluent system A (retention time: 5.91 min); ESI-MS: 643 [M+H]⁺.

### Compound 10

### Synthesis of (3S)-3-{[N-(4-methoxy-1H-indole-2-carbonyl)-L-leucyl]amino}-2-oxo-4-[(3S)-2-oxopiperidin-3-yl]butyl 2-(trifluoromethyl)pyridine-3-carboxylate, 10

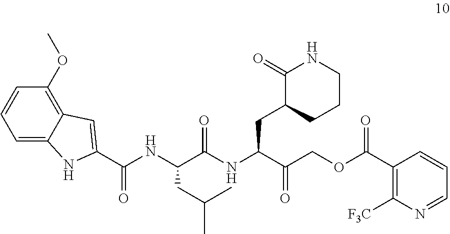 [SMILES: COc1cccc2[nH]c(C(=O)N[C@@H](CC(C)C)C(=O)N[C@@H](C[C@@H]3CCCNC3=O)C(=O)COC(=O)c3cccnc3C(F)(F)F)cc12]

Compound 10 was synthesized as in Scheme 13.

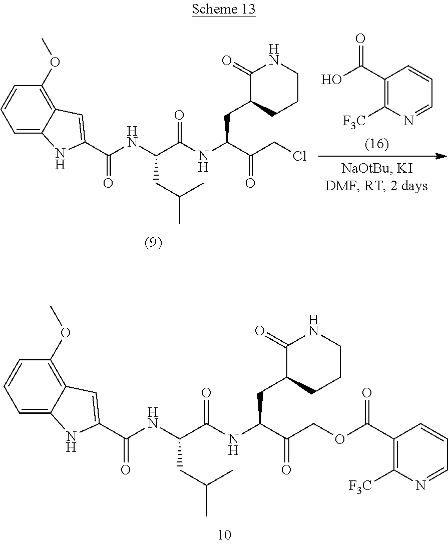

Preparation of (3S)-3-{[N-(4-methoxy-1H-indole-2-carbonyl)-L-leucyl]amino}-2-oxo-4-[(3S)-2-oxopiperidin-3-yl]butyl 2-(trifluoromethyl)pyridine-3-carboxylate, 10. To a solution of 2-(trifluoromethyl)nicotinic acid (16) (57 mg, 0.30 mmol) in DMF (5 mL) was added NaOtBu (19 mg, 0.20 mmol). After 15 min, (9) (50 mg, 0.10 mmol) was added followed by KI (16.6 mg, 0.10 mmol). After 2 days, water (5 mL) was added and the resulting mixture was extracted with EtOAc (3×20 mL). The combined organic phase was washed with brine (10 mL), dried over Na₂SO₄, filtered and concentrated under reduced pressure. The product was purified by column chromatography on silica gel with a gradient of 0 to 5% MeOH in DCM, which generated 10 (22 mg, 33% yield) as a white solid. ¹H NMR (600 MHz, DMSO-d₆) δ 11.58 (d, J=2.3 Hz, 1H), 8.95 (dd, J=1.5, 4.9 Hz, 1H), 8.65 (d, J=7.9 Hz, 1H), 8.46 (d, J=7.5 Hz, 1H), 8.38 (dd, J=1.3, 8.1 Hz, 1H), 7.92 (dd, J=4.9, 7.9 Hz, 1H), 7.47 (br s, 1H), 7.39-7.37 (m, 1H), 7.12-7.08 (m, 1H), 7.04-6.99 (m, 1H), 6.51 (d, J=7.5 Hz, 1H), 5.24-5.16 (m, 2H), 4.58-4.53 (m, 1H), 4.53-4.48 (m, 1H), 3.89 (s, 3H), 3.13-3.05 (m, 2H), 2.27-2.20 (m, 2H), 1.89-1.81 (m, 1H), 1.79-1.66 (m, 4H), 1.62-1.49 (m, 2H), 1.39-1.32 (m, 1H), 0.95 (d, J=6.4 Hz, 3H), 0.91 (d, J=6.4 Hz, 3H). ¹⁹F NMR (565 MHz, DMSO-d₆) δ −63.16 (s, 3F). LC/MS: Eluent system A (retention time: 5.81 min); ESI-MS 660 [M+H]⁺.

### Compound 11

### Synthesis of (3S)-3-{[N-(4-methoxy-1H-indole-2-carbonyl)-L-leucyl]amino}-2-oxo-4-[(3S)-2-oxopiperidin-3-yl]butyl 6-tert-butylpyridine-3-carboxylate, 11

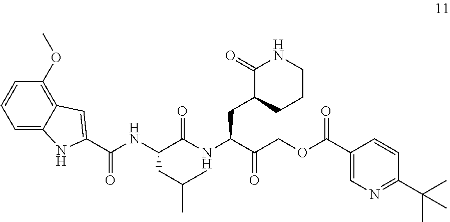 [SMILES: COc1cccc2[nH]c(C(=O)N[C@@H](CC(C)C)C(=O)N[C@@H](C[C@@H]3CCCNC3=O)C(=O)COC(=O)c3ccc(C(C)(C)C)nc3)cc12]

Compound 11 was synthesized as in Scheme 14.

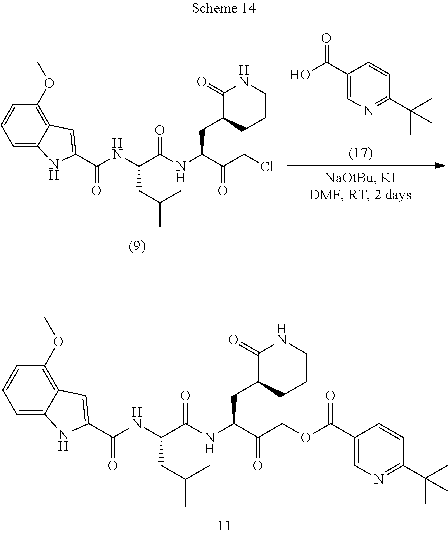 [SMILES: COc1cccc2[nH]c(C(=O)N[C@@H](CC(C)C)C(=O)N[C@@H](C[C@@H]3CCCNC3=O)C(=O)CCl)cc12 ; CC(C)(C)c1ccc(C(=O)O)cn1 ; COc1cccc2[nH]c(C(=O)N[C@@H](CC(C)C)C(=O)N[C@@H](C[C@@H]3CCCNC3=O)C(=O)COC(=O)c3ccc(C(C)(C)C)nc3)cc12]

Preparation of (3S)-3-{[N-(4-methoxy-1H-indole-2-carbonyl)-L-leucyl]amino}-2-oxo-4-[(3S)-2-oxopiperidin-3-yl]butyl 6-tert-butylpyridine-3-carboxylate, 11. To a solution of 6-tert-butylnicotinic acid (18) (54 mg, 0.30 mmol) in DMF (5 mL) was added NaOtBu (19 mg, 0.20 mmol) at room temperature. After 15 min, (9) (50 mg, 0.10 mmol) was added followed by KI (16.6 mg, 0.10 mmol). After 2 days, water (5 mL) was added and the mixture was extracted with EtOAc (3×20 mL). The combined organic phase was washed with brine (10 mL), dried over Na₂SO₄, filtered and concentrated under reduced pressure. The product was purified by column chromatography on silica gel with a gradient of 0 to 10% MeOH in EtOAc, which generated 11 (32 mg, 49% yield) as a white solid. ¹H NMR (600 MHz, DMSO-d₆) δ 11.58 (d, J=2.3 Hz, 1H), 9.06 (dd, J=0.8, 2.3 Hz, 1H), 8.64 (d, J=8.3 Hz, 1H), 8.46 (d, J=7.5 Hz, 1H), 8.26-8.23 (m, 1H), 7.64-7.61 (m, 1H), 7.47 (br s, 1H), 7.39-7.36 (m, 1H), 7.13-7.08 (m, 1H), 7.04-6.99 (m, 1H), 6.51 (d, J=7.2 Hz, 1H), 5.20-5.11 (m, 2H), 4.57-4.46 (m, 2H), 3.89 (s, 3H), 3.13-3.06 (m, 2H), 2.27-2.20 (m, 2H), 1.89-1.83 (m, 1H), 1.77-1.68 (m, 4H), 1.61-1.48 (m, 2H), 1.39-1.32 (m, 1H), 1.35 (s, 9H), 0.95 (d, J=6.0 Hz, 3H), 0.90 (d, J=6.4 Hz, 3H). LC/MS: Eluent system A (retention time: 6.60 min); ESI-MS 648 [M+H]⁺.

### Compound 12

### Synthesis of (3S)-3-{[N-(4-methoxy-1H-indole-2-carbonyl)-L-leucyl]amino}-2-oxo-4-[(3S)-2-oxopiperidin-3-yl]butyl 1-cyanocyclopropane-1-carboxylate, 12

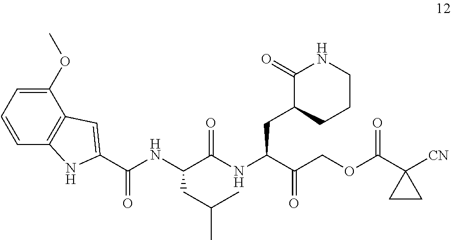 [SMILES: COc1cccc2[nH]c(C(=O)N[C@@H](CC(C)C)C(=O)N[C@@H](C[C@@H]3CCCNC3=O)C(=O)COC(=O)C3(C#N)CC3)cc12]

Compound 12 was synthesized as in Scheme 15.

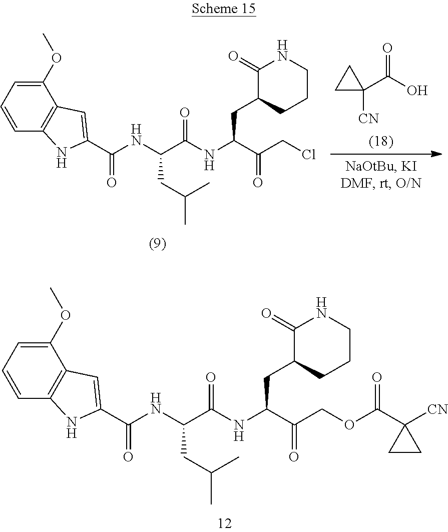

Preparation of (3S)-3-{[N-(4-methoxy-1H-indole-2-carbonyl)-L-leucyl]amino}-2-oxo-4-[(3S)-2-oxopiperidin-3-yl]butyl 1-cyanocyclopropane-1-carboxylate, 12. The procedure for 5 in Scheme 8 was followed with (9) (40 mg, 0.079 mmol), 1-cyanocyclopropanecarboxylic acid (18) (60 mg, 0.54 mmol), sodium tert-butoxide (32 mg, 0.33 mmol), potassium iodide (24 mg, 0.145 mmol) and DMF (3 mL). The product was purified by column chromatography (eluted with gradient of 0 to 12% methanol in 1:1 chloroform:ethyl acetate mixture), followed by trituration with ether provided 12 (28.6 mg, 62% yield) as a light pink powder. ¹H NMR (600 MHz, DMSO-d6) δ 11.57 (d, J=1.9 Hz, 1H), 8.68-8.57 (m, 1H), 8.45 (d, J=7.7 Hz, 1H), 7.47-7.40 (m, 1H), 7.37 (dd, J=0.7, 2.2 Hz, 1H), 7.10 (t, J=8.0 Hz, 1H), 7.01 (d, J=8.3 Hz, 1H), 6.51 (d, J=7.7 Hz, 1H), 5.09-4.97 (m, 2H), 4.91-4.43 (m, 2H), 3.89 (s, 3H), 3.12-3.05 (m, 2H), 2.23-2.15 (m, 2H), 1.88-1.80 (m, 3H), 1.75-1.63 (m, 6H), 1.59-1.47 (m, 2H), 1.37-1.29 (m, 1H), 0.95 (d, J=6.3 Hz, 3H), 0.90 (d, J=6.4 Hz, 3H). LC/MS: Eluent system A (retention time: 5.21 min); ESI-MS 580 [M+H]⁺, 578 [M−H]⁻.

### Compound 13

### Synthesis of (3S)-3-{[N-(4-methoxy-1H-indole-2-carbonyl)-L-leucyl]amino}-2-oxo-4-[(3S)-2-oxopiperidin-3-yl]butyl 3,3,3-trifluoro-2,2-dimethylpropanoate, 13

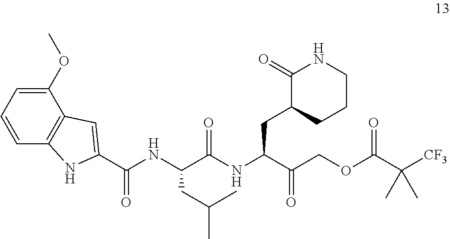 [SMILES: COc1cccc2[nH]c(C(=O)N[C@@H](CC(C)C)C(=O)N[C@@H](C[C@@H]3CCCNC3=O)C(=O)COC(=O)C(C)(C)C(F)(F)F)cc12]

Compound 13 was synthesized as in Scheme 16

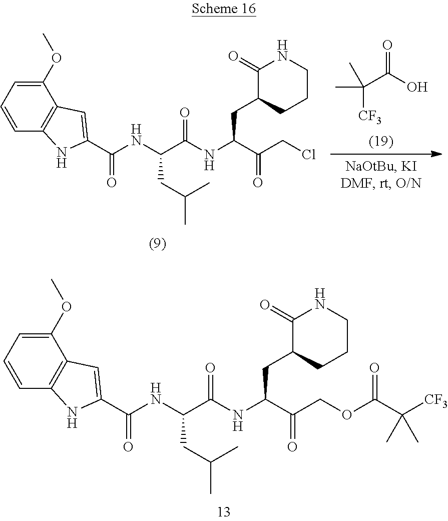

Preparation of (3S)-3-{[N-(4-methoxy-1H-indole-2-carbonyl)-L-leucyl]amino}-2-oxo-4-[(3S)-2-oxopiperidin-3-yl]butyl 3,3,3-trifluoro-2,2-dimethylpropanoate, 13. The procedure for 5 in Scheme 8 was followed with (9) (40 mg, 0.079 mmol), 3,3,3-trifluoro-2,2-dimethylpropanoic acid (19) (91 mg, 0.58 mmol), sodium tert-butoxide (42 mg, 0.43 mmol), potassium iodide (23 mg, 0.139 mmol) and DMF (3 mL). The product was purified by column chromatography on silica gel (eluted with gradient of 0 to 12% methanol in 1:1 chloroform:ethyl acetate mixture), followed by trituration with ether which provided 13 (27.0 mg, 55% yield) as an off-white powder. ¹H NMR (600 MHz, DMSO-d6) δ 11.58 (d, J=2.0 Hz, 1H), 8.59 (d, J=8.1 Hz, 1H), 8.45 (d, J=7.7 Hz, 1H), 7.46 (br.s, 1H), 7.37 (dd, J=0.7, 2.0 Hz, 1H), 7.10 (dd, J=7.5, 8.2 Hz, 1H), 7.01 (d, J=8.2 Hz, 1H), 6.51 (d, J=7.6 Hz, 1H), 5.00 (d, J=17.0 Hz, 1H), 4.97 (d, J=17.0 Hz, 1H), 4.50-4.44 (m, 2H), 3.89 (s, 3H), 3.12-3.05 (m, 2H), 2.24-2.16 (m, 2H), 1.87-1.81 (m, 1H), 1.76-1.66 (m, 4H), 1.59-1.48 (m, 2H), 1.42 (s, 6H), 1.37-1.30 (m, 1H), 0.95 (d, J=6.4 Hz, 3H), 0.90 (d, J=6.4 Hz, 3H). ¹⁹F NMR (565 MHz, DMSO-d6) δ −73.76 (s, 3F). LC/MS: Eluent system A (retention time: 6.48 min); ESI-MS 625 [M+H]⁺, 623 [M−H]⁻.

### Compound 14

### Synthesis of (3S)-3-{[N-(4-methoxy-1H-indole-2-carbonyl)-L-leucyl]amino}-2-oxo-4-[(3S)-2-oxopiperidin-3-yl]butyl 2-cyano-2-methylpropanoate, 14

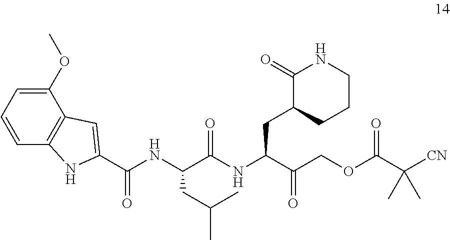 [SMILES: COc1cccc2[nH]c(C(=O)N[C@@H](CC(C)C)C(=O)N[C@@H](C[C@@H]3CCCNC3=O)C(=O)COC(=O)C(C)(C)C#N)cc12]

Compound 14 was synthesized as in Scheme 17.

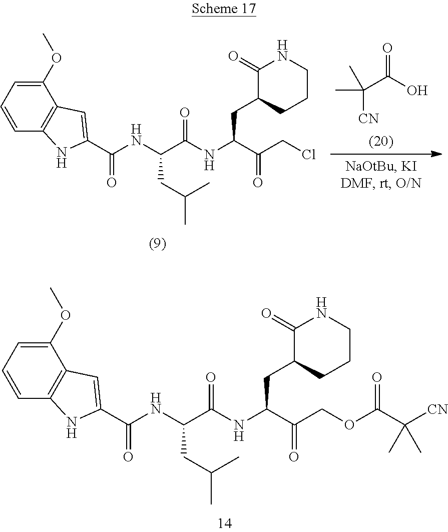

Preparation of (3S)-3-{[N-(4-methoxy-1H-indole-2-carbonyl)-L-leucyl]amino}-2-oxo-4-[(3S)-2-oxopiperidin-3-yl]butyl 2-cyano-2-methylpropanoate, 14. The procedure for 5 in Scheme 8 was followed with (9) (40 mg, 0.079 mmol), 2-cyano-2-methylpropanoic acid (20) (61 mg, 0.58 mmol), sodium tert-butoxide (36 mg, 0.37 mmol), potassium iodide (47 mg, 0.28 mmol) and DMF (3 mL). The product was purified by column chromatography on silica gel (eluted with gradient of 0 to 12% methanol in 1:1 chloroform:ethyl acetate mixture), followed by trituration with ether, which provided 14 (25.9 mg, 56% yield) as a white powder. ¹H NMR (600 MHz, DMSO-d6) δ 11.58 (d, J=2.0 Hz, 1H), 8.68-8.58 (m, 1H), 8.45 (d, J=7.6 Hz, 1H), 7.48-7.41 (m, 1H), 7.37 (dd, J=0.7, 2.1 Hz, 1H), 7.10 (t, J=8.1 Hz, 1H), 7.01 (d, J=8.2 Hz, 1H), 6.51 (d, J=7.6 Hz, 1H), 5.07 (d, J=17.0 Hz, 1H), 5.01 (d, J=17.0 Hz, 1H), 4.51-4.45 (m, 2H), 3.89 (s, 3H), 3.13-3.05 (m, 2H), 2.24-2.16 (m, 2H), 1.87-1.81 (m, 1H), 1.77-1.66 (m, 4H), 1.603 (s, 3H), 1.600 (s, 3H), 1.59-1.48 (m, 2H), 1.37-1.30 (m, 1H), 0.95 (d, J=6.2 Hz, 3H), 0.90 (d, J=6.2 Hz, 3H). LC/MS: Eluent system A (retention time: 5.51 min); ESI-MS 82 [M+H]⁺, 580 [M−H]⁻.

### Compound 15

### Synthesis of (3S)-3-{[N-(4-methoxy-1H-indole-2-carbonyl)-L-leucyl]amino}-2-oxo-4-[(3S)-2-oxopiperidin-3-yl]butyl 1-(trifluoromethyl)cyclopropane-1-carboxylate, 15

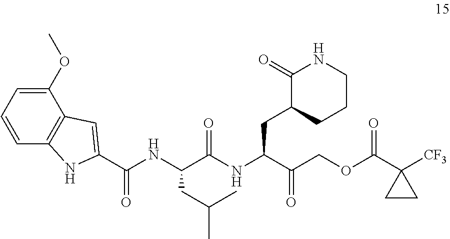 [SMILES: COc1cccc2[nH]c(C(=O)N[C@@H](CC(C)C)C(=O)N[C@@H](C[C@@H]3CCCNC3=O)C(=O)COC(=O)C3(C(F)(F)F)CC3)cc12]

Compound 15 was synthesized as in Scheme 18.

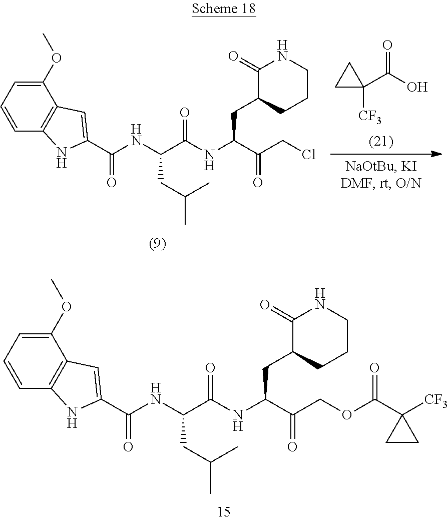

Preparation of (3S)-3-{[N-(4-methoxy-1H-indole-2-carbonyl)-L-leucyl]amino}-2-oxo-4-[(3S)-2-oxopiperidin-3-yl]butyl 1-(trifluoromethyl)cyclopropane-1-carboxylate, 15. The procedure for 5 in Scheme 8 was followed with (9) (40 mg, 0.079 mmol) with 1-(trifluoromethyl)cyclopropanecarboxylic acid (21) (84 mg, 0.54 mmol), sodium tert-butoxide (32 mg, 0.33 mmol), potassium iodide (32 mg, 0.19 mmol) and DMF (3 mL). The product was purified by column chromatography on silica gel (eluted with gradient of 0 to 12% methanol in 1:1 chloroform:ethyl acetate mixture), followed by trituration with ether which provided 15 (25.8 mg, 56% yield) as a white powder. ¹H NMR (600 MHz, DMSO-d6) δ 11.58 (d, J=2.0 Hz, 1H), 8.58 (d, J=7.9 Hz, 1H), 8.44 (d, J=7.7 Hz, 1H), 7.46 (br.s, 1H), 7.37 (dd, J=0.7, 2.2 Hz, 1H), 7.10 (t, J=8.0 Hz, 1H), 7.01 (d, J=8.2 Hz, 1H), 6.51 (d, J=7.6 Hz, 1H), 4.99 (d, J=17.2 Hz, 1H), 4.96 (d, J=17.2 Hz, 1H), 4.49-4.43 (m, 2H), 3.89 (s, 3H), 3.12-3.05 (m, 2H), 2.23-2.15 (m, 2H), 1.86-1.80 (m, 1H), 1.75-1.64 (m, 4H), 1.58-1.47 (m, 6H), 1.36-1.29 (m, 1H), 0.95 (d, J=6.3 Hz, 3H), 0.90 (d, J=6.3 Hz, 3H). ¹⁹F NMR (565 MHz, DMSO-d6) δ −66.03 (s, 3F). LC/MS: Eluent system A (retention time: 6.21 min); ESI-MS 623 [M+H]⁺, 621 [M−H]⁻.

### Compound 16

### Synthesis of (3S)-3-{[N-(4-methoxy-1H-indole-2-carbonyl)-L-leucyl]amino}-2-oxo-4-[(3S)-2-oxopiperidin-3-yl]butyl 2-methyl-1,3-dioxane-2-carboxylate, 16

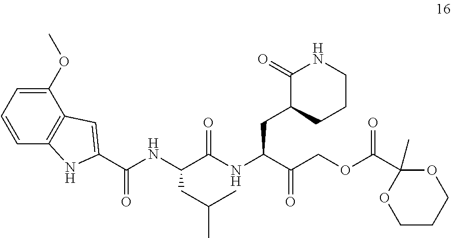 [SMILES: COc1cccc2[nH]c(C(=O)N[C@@H](CC(C)C)C(=O)N[C@@H](C[C@@H]3CCCNC3=O)C(=O)COC(=O)C3(C)OCCCO3)cc12]

Compound 16 was synthesized as in Scheme 19.

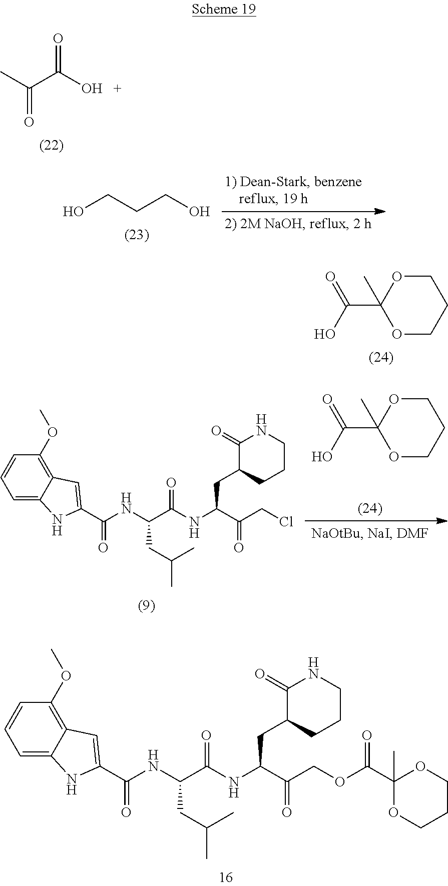

Preparation of 2-methyl-1,3-dioxane-2-carboxylic acid, (24). Following a procedure by Wardrop and coworkers reported in Organic Letters, 2001, 3(15), 2261-2264; A mixture of propane-1,3-diol (23) (2.00 g, 26.3 mmol), 2-oxopropanoic acid (22) (1.54 g, 17.5 mmol) and Amberlite IR-120 (plus) resin (200 mg) in benzene (50 mL) were heated on an oil bath at reflux in a Dean-Stark apparatus. After overnight, the mixture was cooled to room temperature, filtered, and concentrated under reduced pressure. The resulting residue was dissolved in 2 M aqueous NaOH (20 mL), then heated at reflux. After 2 h, the mixture was cooled to room temperature, acidified to pH=1 with ice cold 6 M aqueous H₃PO₄ (25 mL) and extracted with EtOAc (4×25 mL). The combined organic layer was dried over anhydrous Na₂SO₄, filtered, concentrated under reduced pressure. The product was purified by column chromatography on silica gel (Biotage®, 25 g silicycle column) with a gradient of 0% to 100% EtOAc in hexanes, which generated (24) (1.35 g, 53% yield) as a white solid.

Preparation of (3S)-3-{[N-(4-methoxy-1H-indole-2-carbonyl)-L-leucyl]amino}-2-oxo-4-[(3S)-2-oxopiperidin-3-yl]butyl 2-methyl-1,3-dioxane-2-carboxylate, 16. A mixture of 2-methyl-1,3-dioxane-2-carboxylic acid (24) (69.5 mg, 0.48 mmol) and sodium tert-butoxide (22.8 mg, 0.24 mmol) in anhydrous DMF (3 mL) was stirred at room temperature for 30 min. To this (9) (40 mg, 0.08 mmol) and NaI (23.8 mg, 0.16 mmol) were added successively. After 24 h, saturated brine solution (10 mL) was added and the resulting mixture was extracted with CHCl₃ (3×10 mL). The combined organic layer was washed with saturated brine solution (1×10 mL), dried over anhydrous Na₂SO₄, filtered, and concentrated reduced pressure. The residue was dissolved in CHCl₃ (5 mL) and loaded on silica gel column (3×4 g Silicycle siliasep columns) and the product was purified by Biotage® with a gradient of 0 to 4% MeOH in DCM, which afforded 16 (24.9 mg, 51% yield) as an off-white solid. ¹H NMR (600 MHz, DMSO-d₆) δ 11.58 (d, J=2.1 Hz, 1H), 8.60 (d, J=8.1 Hz, 1H), 8.45 (d, J=7.5 Hz, 1H), 7.46 (br s, 1H), 7.38 (dd, J=0.8, 2.3 Hz, 1H), 7.11 (t, J=8.1 Hz, 1H), 7.01 (d, J=8.3 Hz, 1H), 6.51 (d, J=7.3 Hz, 1H), 5.05 (d, J=17.1 Hz, 1H), 5.01 (d, J=17.1 Hz, 1H), 4.54-4.45 (m, 2H), 3.89 (s, 3H), 3.88-3.79 (m, 4H), 3.14-3.05 (m, 2H), 2.26-2.17 (m, 2H), 1.94-1.81 (m, 2H), 1.79-1.66 (m, 4H), 1.62-1.46 (m, 2H), 1.38 (s, 3H), 1.36-1.19 (m, 2H), 0.95 (d, J=6.4 Hz, 3H), 0.90 (d, J=6.4 Hz, 3H). LC/MS: Eluent system A (retention time: 6.69 min); ESI-MS: 615 [M+H]⁺.

### Compound 17

### Synthesis of (3S)-3-{[N-(cyclopentanecarbonyl)-L-leucyl]amino}-2-oxo-4-[(3S)-2-oxopiperidin-3-yl]butyl pyridine-3-carboxylate, 17

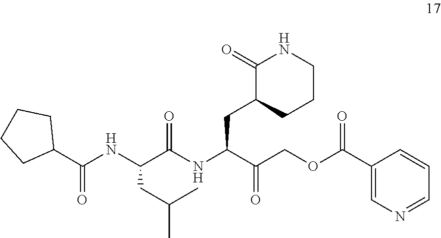 [SMILES: CC(C)C[C@H](NC(=O)C1CCCC1)C(=O)N[C@@H](C[C@@H]1CCCNC1=O)C(=O)COC(=O)c1cccnc1]

Compound 17 was synthesized as in Scheme 20.

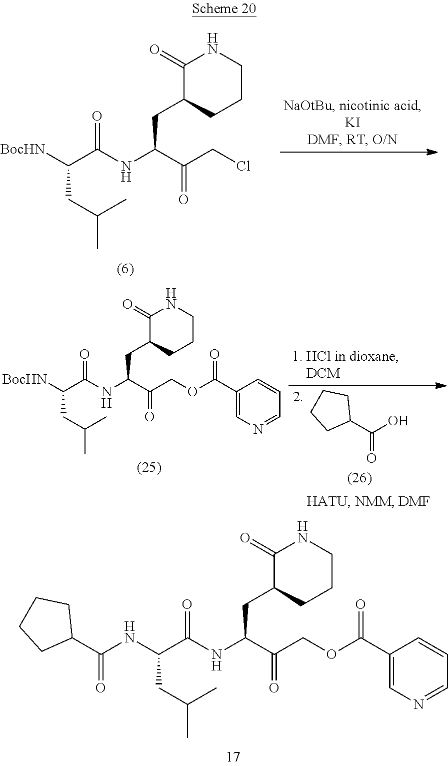

Preparation of (3S)-3-{[N-(tert-butoxycarbonyl)-L-leucyl]amino}-2-oxo-4-[(3S)-2-oxopiperidin-3-yl]butyl pyridine-3-carboxylate, (25). To a solution of nicotinic acid (103 mg, 0.84 mmol) in DMF (5 mL) was added NaOtBu (54 mg, 0.56 mmol). After 15 min, (6) (120 mg, 0.28 mmol) followed by KI (46.5 mg, 0.28 mmol) were added. After overnight, water (5 mL) was added and the resulting mixture was extracted with EtOAc (3×25 mL). The combined organic phase was washed with brine (10 mL), dried over Na₂SO₄, filtered and concentrated under reduced pressure. The product was purified by column chromatography on silica gel with a gradient of 0 to 10% MeOH in EtOAc, which generated (25) (110 mg, 76% yield) as a white solid.

Preparation of (3S)-3-{[N-(cyclopentanecarbonyl)-L-leucyl]amino}-2-oxo-4-[(3S)-2-oxopiperidin-3-yl]butyl pyridine-3-carboxylate, 17. To a solution of (25) (65 mg, 0.125 mmol) in DCM (2 mL) cooled in an ice bath was added a 4N HCl solution in dioxane (0.5 mL, 2.0 mmol). After 1 hour, the volatiles were removed under reduced pressure. The resulting residue was co-evaporated with CH₃CN/ether (1:1, 2×20 mL) and dried completely under reduced pressure. To the residue was added DMF (5 mL) and after cooling in an ice bath then cyclopentanecarboxylic acid (26) (14 mg, 0.125 mmol), HATU (52.5 mg, 0.138 mmol) and NMM (0.041 mL, 0.375 mmol) were added. After 45 min, a mixture of ice and sat NaHCO₃ (aq) were added. The two layers were separated and the aqueous layer was extracted with EtOAc (3×15 mL). The organic layers were combined and dried over Na₂SO₄, filtered and concentrated under reduced pressure. The product was purified by column chromatography on silica gel with a gradient of 0 to 10% MeOH in CHCl₃, which generated 17 (18 mg, 28% yield) as a light yellow solid. ¹H NMR (600 MHz, DMSO-d₆) δ 9.12 (dd, J=0.8, 2.3 Hz, 1H), 8.86 (dd, J=1.7, 4.7 Hz, 1H), 8.56 (d, J=7.9 Hz, 1H), 8.35-8.31 (m, 1H), 7.94 (d, J=7.5 Hz, 1H), 7.64-7.58 (m, 1H), 7.48 (br s, 1H), 5.18-5.09 (m, 2H), 4.53-4.47 (m, 1H), 4.29-4.24 (m, 1H), 3.14-3.08 (m, 2H), 2.67-2.63 (m, 1H), 2.25-2.17 (m, 2H), 1.88-1.83 (m, 1H), 1.78-1.66 (m, 4H), 1.66-1.53 (m, 6H), 1.52-1.44 (m, 4H), 1.38-1.32 (m, 1H), 0.91 (d, J=6.8 Hz, 3H), 0.85 (d, J=6.4 Hz, 3H). LC/MS: Eluent system A (retention time: 4.36 min); ESI-MS 515 [M+H]⁺.

### Compound 18

### Synthesis of (3S)-3-{[N-(4-methoxy-1H-indole-2-carbonyl)-L-leucyl]amino}-2-oxo-4-[(3S)-2-oxopiperidin-3-yl]butyl 2,6-dimethyl-4-(trifluoromethyl)pyridine-3-carboxylate, 18

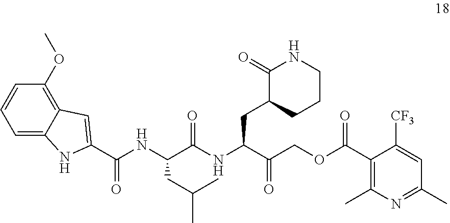 [SMILES: COc1cccc2[nH]c(C(=O)N[C@@H](CC(C)C)C(=O)N[C@@H](C[C@@H]3CCCNC3=O)C(=O)COC(=O)c3c(C(F)(F)F)cc(C)nc3C)cc12]

Compound 18 was synthesized as in Scheme 21.

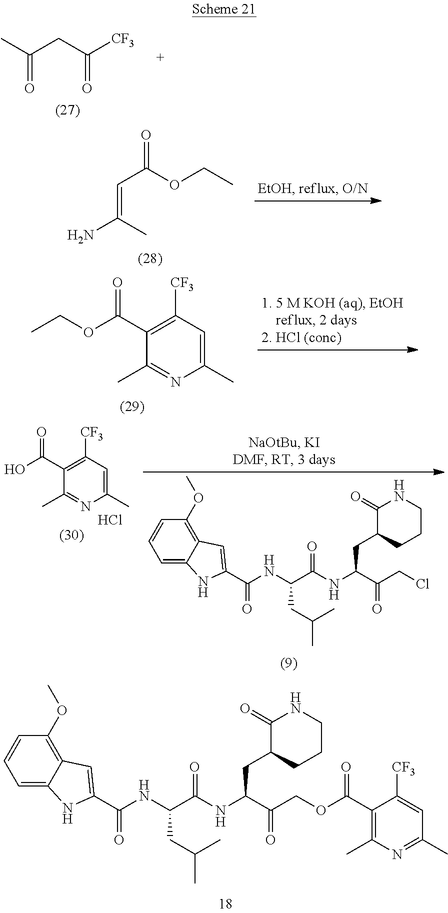

Preparation of ethyl 2,6-dimethyl-4-(trifluoromethyl)pyridine-3-carboxylate, (29). A mixture of 1,1,1-trifluoro-2,4-pentanedione (27) (3.0 g, 19.5 mmol) and ethyl 3-aminocrotonate (28) (2.5 g, 19.5 mmol) in EtOH (40 mL) was heated at reflux. After overnight, the reaction mixture was cooled to room temperature and the volatiles were removed under reduced pressure. The product was purified by column chromatography on silica gel with an eluent of DCM, which generated (29) (2.1 g, 44% yield) as a light yellow liquid.

Preparation of 2,6-dimethyl-4-(trifluoromethyl)pyridine-3-carboxylic acid hydrochloride, (30). To a solution of ethyl 2,6-dimethyl-4-(trifluoromethyl)pyridine-3-carboxylate (29) (2.05 g, 8.3 mmol) in EtOH (15 mL) was added a 5M aqueous KOH solution (5.0 mL, 25.0 mmol) and the mixture was heated to reflux. After 2 days, the mixture was cooled to room temperature and a concentrated HCl solution (3.0 mL, 36 mmol) was added and the volatiles were removed under reduced pressure. The resulting residue was dissolved in EtOH (30 mL) and heated to 60° C. The insoluble solid was filtered off and the filtrate was concentrated under reduced pressure. To the residue was added ether (50 mL) and the solidified product was collected by filtration and dried, which provided (30) (2.1 g, 99% yield) as a pale yellow solid.

Preparation of (3S)-3-{[N-(4-methoxy-1H-indole-2-carbonyl)-L-leucyl]amino}-2-oxo-4-[(3S)-2-oxopiperidin-3-yl]butyl 2,6-dimethyl-4-(trifluoromethyl)pyridine-3-carboxylate, 18. To a solution of 2,6-dimethyl-4-(trifluoromethyl)pyridine-3-carboxylic acid hydrochloride (30) (102 mg, 0.40 mmol) in DMF (5 mL) was added NaOtBu (58 mg, 0.60 mmol). After 30 min, (9) (50 mg, 0.10 mmol) followed by KI (16.6 mg, 0.10 mmol) were added. After 3 days, EtOAc (40 mL) was added and the mixture was washed with water (3×10 mL), brine (10 mL), dried over Na₂SO₄, filtered and concentrated under reduced pressure. The product was purified by column chromatography on silica gel with a gradient of 0 to 10% MeOH in CHCl₃, which generated 18 (23 mg, 33% yield) as a white solid. ¹H NMR (600 MHz, DMSO-d₆) δ 11.56 (d, J=2.3 Hz, 1H), 8.64 (d, J=8.3 Hz, 1H), 8.45 (d, J=7.5 Hz, 1H), 7.63 (s, 1H), 7.47 (br s, 1H), 7.38-7.36 (m, 1H), 7.12-7.08 (m, 1H), 7.03-6.99 (m, 1H), 6.51 (d, J=7.2 Hz, 1H), 5.23-5.13 (m, 2H), 4.60-4.48 (m, 2H), 3.89 (s, 3H), 3.13-3.05 (m, 2H), 2.63 (s, 3H), 2.59 (s, 3H), 2.29-2.20 (m, 2H), 1.89-1.82 (m, 1H), 1.79-1.66 (m, 4H), 1.63-1.48 (m, 2H), 1.40-1.32 (m, 1H), 0.95 (d, J=6.4 Hz, 3H), 0.90 (d, J=6.4 Hz, 3H). ¹⁹F NMR (565 MHz, DMSO-d₆) δ −60.74 (s, 3F). LC/MS: Eluent system A (retention time: 6.37 min); ESI-MS 688 [M+H]⁺.

### Compound 19

### Synthesis of (3S)-3-{[N-(4-methoxy-1H-indole-2-carbonyl)-L-leucyl]amino}-2-oxo-4-[(3S)-2-oxopiperidin-3-yl]butyl 1-cyanocyclopentane-1-carboxylate, 19

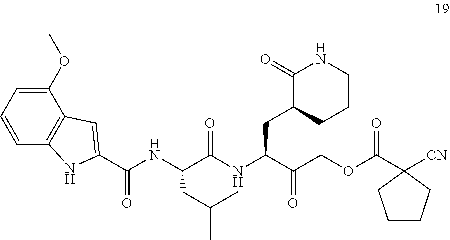 [SMILES: COc1cccc2[nH]c(C(=O)N[C@@H](CC(C)C)C(=O)N[C@@H](C[C@@H]3CCCNC3=O)C(=O)COC(=O)C3(C#N)CCCC3)cc12]

Compound 19 was synthesized as in Scheme 22.

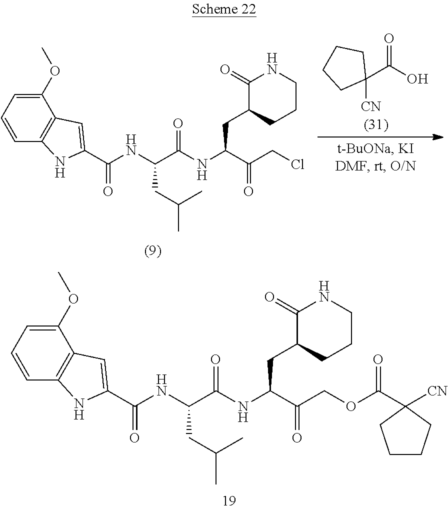

Preparation of (3S)-3-{[N-(4-methoxy-1H-indole-2-carbonyl)-L-leucyl]amino}-2-oxo-4-[(3S)-2-oxopiperidin-3-yl]butyl 1-cyanocyclopentane-1-carboxylate, 19. The procedure for 5 in Scheme 8 was followed with (9) (40 mg, 0.079 mmol), 1-cyanocyclopentanecarboxylic acid (31) (70 mg, 0.50 mmol), sodium tert-butoxide (32 mg, 0.33 mmol), potassium iodide (34 mg, 0.20 mmol) and DMF (3 mL). The product was purified by column chromatography (eluted with gradient of 0 to 12% methanol in 1:1 chloroform:ethyl acetate mixture), followed by trituration with ether that provided 19 (6.0 mg, 10% yield) as a white powder. ¹H NMR (600 MHz, DMSO-d₆) δ 11.56 (br s, 1H), 8.68-8.27 (m, 2H), 7.47-7.31 (m, 2H), 7.10 (t, J=7.9 Hz, 1H), 7.01 (d, J=8.0 Hz, 1H), 6.51 (d, J=7.6 Hz, 1H), 5.10-4.98 (m, 2H), 4.61-4.43 (m, 2H), 3.89 (s, 3H), 3.12-3.04 (m, 2H), 2.31-2.13 (m, 6H), 1.85-1.65 (m, 9H), 1.59-1.48 (m, 2H), 1.37-1.30 (m, 1H), 0.95 (d, J=6.2 Hz, 3H), 0.90 (d, J=6.2 Hz, 3H). LC/MS: Eluent system A (retention time: 5.91 min); ESI-MS 608 [M+H]⁺, 606 [M−H]⁻.

### Compound 20

### Synthesis of (3S)-3-{[N-(4-methoxy-1H-indole-2-carbonyl)-L-leucyl]amino}-2-oxo-4-[(3S)-2-oxopiperidin-3-yl]butyl 1-(trifluoromethyl)cyclohexane-1-carboxylate, 20

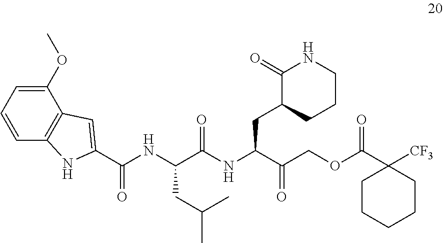 [SMILES: COc1cccc2[nH]c(C(=O)N[C@@H](CC(C)C)C(=O)N[C@@H](C[C@@H]3CCCNC3=O)C(=O)COC(=O)C3(C(F)(F)F)CCCCC3)cc12]

Compound 20 was synthesized as in Scheme 23.

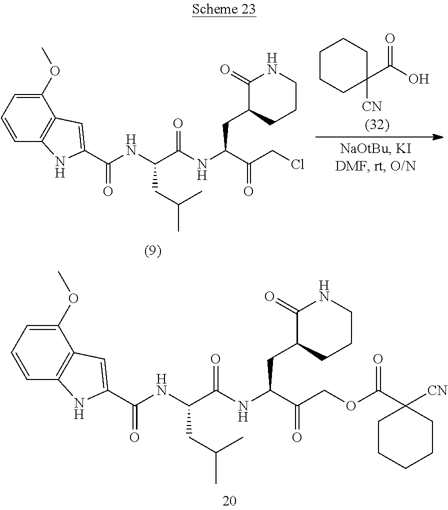

Preparation of (3S)-3-{[N-(4-methoxy-1H-indole-2-carbonyl)-L-leucyl]amino}-2-oxo-4-[(3S)-2-oxopiperidin-3-yl]butyl 1-(trifluoromethyl)cyclohexane-1-carboxylate, 20. The procedure for 5 in Scheme 8 was followed with (9) (50 mg, 0.099 mmol), 1-(trifluoromethyl)cyclohexanecarboxylic acid (32) (105 mg, 0.54 mmol), sodium tert-butoxide (44 mg, 0.46 mmol), potassium iodide (102 mg, 0.61 mmol) and DMF (3 mL). The product was purified by column chromatography (eluted with gradient of 0 to 12% methanol in 1:1 chloroform:ethyl acetate mixture), followed by trituration with ether that provided 20 (9.6 mg, 15% yield) as yellow powder. ¹H NMR (600 MHz, DMSO-d₆) δ 11.56 (br s, 1H), 8.65-8.30 (m, 2H), 7.48-7.32 (m, 2H), 7.10 (dd, J=7.8, 8.0 Hz, 1H), 7.01 (d, J=8.1 Hz, 1H), 6.51 (d, J=7.6 Hz, 1H), 5.10-4.98 (m, 2H), 4.63-4.44 (m, 2H), 3.89 (s, 3H), 3.13-3.04 (m, 2H), 2.25-2.12 (m, 4H), 1.87-1.15 (m, 16H), 0.94 (d, J=6.0 Hz, 3H), 0.89 (d, J=6.0 Hz, 3H). ¹⁹F NMR (565 MHz, DMSO-d₆) δ −73.56 (s, 3F). LC/MS: Eluent system A (retention time: 7.05 min); ESI-MS 665 [M+H]⁺, 663 [M−H]⁻.

### Compound 21

### Synthesis of (3S)-3-{[N-(4-methoxy-1H-indole-2-carbonyl)-L-leucyl]amino}-2-oxo-4-[(3S)-2-oxopiperidin-3-yl]butyl 2-methyl-4-(trifluoromethyl)-1,3-thiazole-5-carboxylate, 21

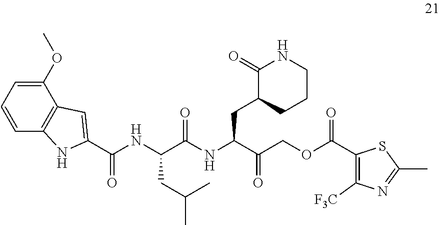 [SMILES: COc1cccc2[nH]c(C(=O)N[C@@H](CC(C)C)C(=O)N[C@@H](C[C@@H]3CCCNC3=O)C(=O)COC(=O)c3sc(C)nc3C(F)(F)F)cc12]

Compound 21 was synthesized as in Scheme 24.

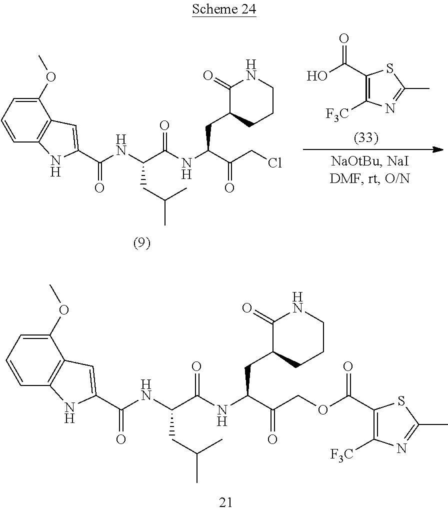 [SMILES: COc1cccc2[nH]c(C(=O)N[C@@H](CC(C)C)C(=O)N[C@@H](C[C@@H]3CCCNC3=O)C(=O)CCl)cc12 ; Cc1nc(C(F)(F)F)c(C(=O)O)s1 ; COc1cccc2[nH]c(C(=O)N[C@@H](CC(C)C)C(=O)N[C@@H](C[C@@H]3CCCNC3=O)C(=O)COC(=O)c3sc(C)nc3C(F)(F)F)cc12]

Preparation of (3S)-3-{[N-(4-methoxy-1H-indole-2-carbonyl)-L-leucyl]amino}-2-oxo-4-[(3S)-2-oxopiperidin-3-yl]butyl 2-methyl-4-(trifluoromethyl)-1,3-thiazole-5-carboxylate, 21. Following a similar procedure as described for compound 9 in Scheme 12 with 2-methyl-4-(trifluoromethyl)-1,3-thiazole-5-carboxylic acid (33) (80 mg, 0.379 mmol), NaOtBu (19 mg, 0.189 mmol), (9) (23 mg, 0.046 mmol), sodium iodide (27 mg, 0.184 mmol) and DMF (1 mL). The product was purified by column chromatography on silica gel (0 to 20% methanol in 1:1 chloroform:ethyl acetate), which generated 21 (12 mg, 38% yield) as a white solid. ¹H NMR (600 MHz, DMSO-d₆) δ 11.57 (br s, 1H), 8.62 (br d, J=7.9 Hz, 1H), 8.45 (br d, J=7.9 Hz, 1H), 7.47 (br s, 1H), 7.40-7.34 (m, 1H), 7.13-7.07 (m, 1H), 7.03-6.97 (m, 1H), 6.54-6.48 (m, 1H), 5.21-5.10 (m, 2H), 4.55-4.43 (m, 2H), 3.89 (s, 3H), 3.14-3.05 (m, 2H), 2.75 (s, 3H), 2.27-2.14 (m, 2H), 1.89-1.80 (m, 1H), 1.78-1.65 (m, 4H), 1.62-1.48 (m, 2H), 1.39-1.30 (m, 1H), 0.95 (d, J=6.4 Hz, 3H), 0.90 (d, J=6.8 Hz, 3H). ¹⁹F NMR (565 MHz, DMSO-d₆) δ −59.84 (s, 3F). LC/MS: Eluent system A (retention time: 6.04 min); ESI-MS: 680 [M+H]⁺.

### Compound 22

### Synthesis of (3S)-3-{[N-(4-methoxy-1H-indole-2-carbonyl)-L-leucyl]amino}-2-oxo-4-[(3S)-2-oxopiperidin-3-yl]butyl 4,6-dimethylpyridine-3-carboxylate, 22

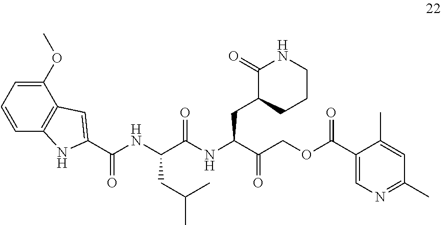 [SMILES: COc1cccc2[nH]c(C(=O)N[C@@H](CC(C)C)C(=O)N[C@@H](C[C@@H]3CCCNC3=O)C(=O)COC(=O)c3cnc(C)cc3C)cc12]

Compound 22 was synthesized as in Scheme 25.

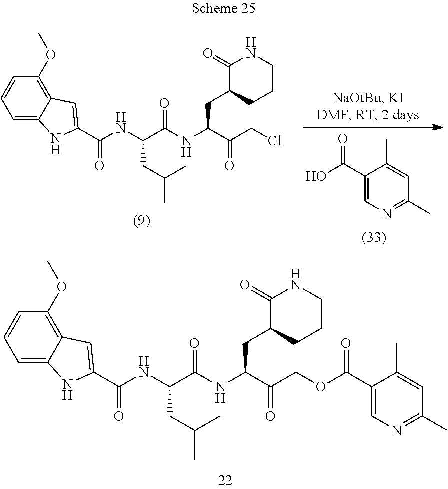 [SMILES: COc1cccc2[nH]c(C(=O)N[C@@H](CC(C)C)C(=O)N[C@@H](C[C@@H]3CCCNC3=O)C(=O)CCl)cc12 ; Cc1cc(C)c(C(=O)O)cn1 ; COc1cccc2[nH]c(C(=O)N[C@@H](CC(C)C)C(=O)N[C@@H](C[C@@H]3CCCNC3=O)C(=O)COC(=O)c3cnc(C)cc3C)cc12]

Preparation (3S)-3-{[N-(4-methoxy-1H-indole-2-carbonyl)-L-leucyl]amino}-2-oxo-4-[(3S)-2-oxopiperidin-3-yl]butyl 4,6-dimethylpyridine-3-carboxylate, 22. To a solution of 4,6-dimethylnicotinic acid (27) (34.5 mg, 0.228 mmol) in DMF (3 mL) was added NaOtBu (13.2 mg, 0.137 mmol). After 30 min, (9) (23.1 mg, 0.046 mmol) followed by KI (7.6 mg, 0.046 mmol) were added. After 2 days, the mixture was diluted with EtOAc (25 mL) was washed with water (3×5 mL), brine (10 mL), dried over Na₂SO₄, filtered and concentrated under reduced pressure. The product was purified by column chromatography on silica gel with a gradient of 0 to 10% MeOH in CHCl₃, which generated 22 (15.0 mg, 51% yield) as an off white solid. ¹H NMR (600 MHz, DMSO-d₆) δ 11.57 (d, J=2.2 Hz, 1H), 8.87 (s, 1H), 8.63 (d, J=7.9 Hz, 1H), 8.45 (d, J=7.7 Hz, 1H), 7.47 (br s, 1H), 7.38-7.36 (m, 1H), 7.28-7.26 (m, 1H), 7.11-7.07 (m, 1H), 7.02-6.99 (m, 1H), 6.51 (d, J=7.7 Hz, 1H), 5.17-5.07 (m, 2H), 4.57-4.47 (m, 2H), 3.89 (s, 3H), 3.14-3.03 (m, 2H), 2.5 (s, 3H, hidden under DMSO-d₅), 2.49 (s, 3H), 2.27-2.19 (m, 2H), 1.88-1.81 (m, 1H), 1.78-1.65 (m, 4H), 1.61-1.47 (m, 2H), 1.39-1.30 (m, 1H), 0.95 (d, J=6.4 Hz, 3H), 0.90 (d, J=6.4 Hz, 3H). LC/MS: Eluent system A (retention time: 5.37 min); ESI-MS 619 [M+H]⁺.

### Compound 23

### Synthesis of (3S)-3-{[N-(4-methoxy-1H-indole-2-carbonyl)-L-leucyl]amino}-2-oxo-4-[(3S)-2-oxopiperidin-3-yl]butyl 2,4,6-trimethylpyridine-3-carboxylate, 23

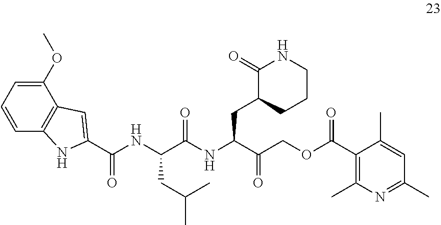 [SMILES: COc1cccc2[nH]c(C(=O)N[C@@H](CC(C)C)C(=O)N[C@@H](C[C@@H]3CCCNC3=O)C(=O)COC(=O)c3c(C)cc(C)nc3C)cc12]

Compound 23 was synthesized as in Scheme 26.

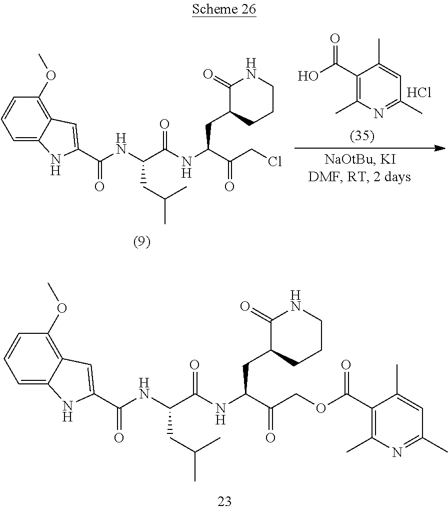 [SMILES: COc1cccc2[nH]c(C(=O)N[C@@H](CC(C)C)C(=O)N[C@@H](C[C@@H]3CCCNC3=O)C(=O)CCl)cc12 ; Cc1cc(C)c(C(=O)O)c(C)n1 ; Cl ; COc1cccc2[nH]c(C(=O)N[C@@H](CC(C)C)C(=O)N[C@@H](C[C@@H]3CCCNC3=O)C(=O)COC(=O)c3c(C)cc(C)nc3C)cc12]

Preparation of (3S)-3-{[N-(4-methoxy-1H-indole-2-carbonyl)-L-leucyl]amino}-2-oxo-4-[(3S)-2-oxopiperidin-3-yl]butyl 2,4,6-trimethylpyridine-3-carboxylate, 23. To a solution of 2,4,6-trimethylpyridine-3-carboxylic acid hydrochloride (35) (81 mg, 0.40 mmol) in DMF (5 mL) was added NaOtBu (58 mg, 0.60 mmol). After 30 min, (9) (51 mg, 0.10 mmol) and then KI (16.6 mg, 0.10 mmol) were added. After 2 days, EtOAc (40 mL) was added and the mixture was washed with water (3×10 mL), brine (10 mL), dried over Na₂SO₄, filtered and concentrated under reduced pressure. The product was purified by column chromatography on silica gel with a gradient of 0 to 10% MeOH in CHCl₃, which generated 23 (38 mg, 60% yield) as a white solid. ¹H NMR (600 MHz, DMSO-d₆) δ 11.57 (d, J=2.3 Hz, 1H), 8.63 (d, J=8.3 Hz, 1H), 8.45 (d, J=7.9 Hz, 1H), 7.46 (br s, 1H), 7.39-7.36 (m, 1H), 7.12-7.08 (m, 1H), 7.05-7.00 (m, 2H), 6.51 (d, J=7.2 Hz, 1H), 5.18-5.09 (m, 2H), 4.57-4.49 (m, 2H), 3.89 (s, 3H), 3.13-3.06 (m, 2H), 2.46 (s, 3H), 2.41 (s, 3H), 2.30 (s, 3H), 2.28-2.21 (m, 2H), 1.89-1.83 (m, 1H), 1.78-1.68 (m, 4H), 1.63-1.49 (m, 2H), 1.39-1.32 (m, 1H), 0.96 (d, J=6.4 Hz, 3H), 0.91 (d, J=6.4 Hz, 3H). LC/MS: Eluent system A (retention time: 4.39 min); ESI-MS 634 [M+H]⁺.

### Compound 24

### Synthesis of N-[(2S)-1-({(1S)-1-cyano-2-[(3S)-2-oxopyrrolidin-3-yl]ethyl}amino)-4-methyl-1-oxopentan-2-yl]-4-methoxy-1H-indole-2-carboxamide, 24

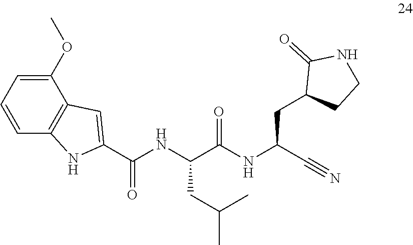 [SMILES: COc1cccc2[nH]c(C(=O)N[C@@H](CC(C)C)C(=O)N[C@H](C#N)C[C@@H]3CCNC3=O)cc12]

Compound 24 was synthesized as in Scheme 27.

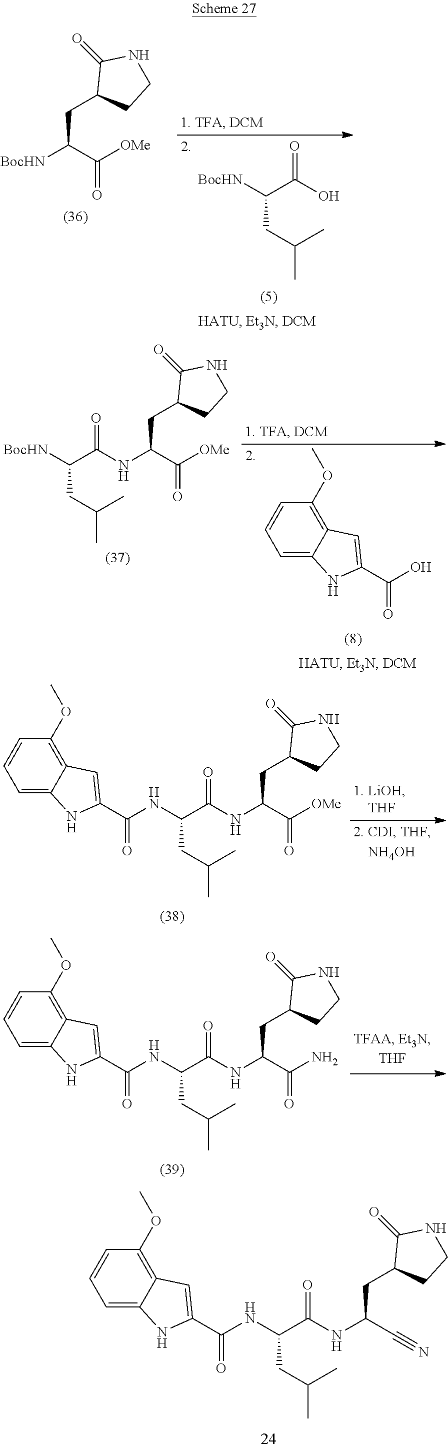

Preparation of methyl N-(tert-butoxycarbonyl)-L-leucyl-3-[(3S)-2-oxopyrrolidin-3-yl]-L-alaninate, (37). To a solution of N-(tert-butoxycarbonyl)-3-[(3S)-2-oxopyrrolidin-3-yl]-L-alaninate (36) (220 mg, 0.77 mmol) [prepared using method in Journal of Medicinal Chemistry (2015), 58, p 9414-9420 and references within] in DCM (5 mL) cooled in an ice bath was added TFA (5 mL). After 1 hour, the solvent was removed under reduced pressure. The resulting residue was co-evaporated with DCM/ether (1:1, 2×20 mL) and dried under reduced pressure. To the resulting residue was added DCM (10 mL) and the mixture was cooled and then (5) (187 mg, 0.81 mmol), HATU (456 mg, 1.20 mmol) and then Et₃N (0.5 mL, 3.85 mmol) were added. After 45 min, a mixture of ice and saturate NaHCO₃ solution were added. The two layers were separated and the aqueous layer was extracted with DCM (2×15 mL). The organic layers were combined, dried over Na₂SO₄, filtered and concentrated under reduced pressure. The product was purified by column chromatography on silica gel with a gradient of 0 to 10% MeOH in CHCl₃, which generated (37) (200 mg, 65% yield) as a white solid.

Preparation of methyl N-(4-methoxy-1H-indole-2-carbonyl)-L-leucyl-3-[(3S)-2-oxopyrrolidin-3-yl]-L-alaninate, (38). To a solution of methyl N-(tert-butoxycarbonyl)-L-leucyl-3-[(3S)-2-oxopyrrolidin-3-yl]-L-alaninate (37) (200 mg, 0.50 mmol) in DCM (5 mL) cooled in an ice bath was added TFA (5 mL). After 1 hour, the mixture was concentrated under reduced pressure. The resulting residue was co-evaporated with DCM/ether (1:1, 2×20 mL) and dried under reduced pressure. To the was added DCM (10 mL) and the mixture was cooled in an ice bath upon which 4-methoxy-1H-indole-2-carboxylic acid (14) (105 mg, 0.55 mmol), HATU (304 mg, 0.80 mmol) and the Et₃N (0.21 mL, 1.50 mmol) were added. After 45 min, a mixture of ice and saturated NaHCO₃ (aq) was added. The two layers were separated and the aqueous layer was extracted with DCM (2×15 mL). The organic layers were combined, dried over Na₂SO₄, filtered and concentrated under reduced pressure. The product was purified by column chromatography on silica gel with a gradient of 0 to 10% MeOH in CHCl₃, which generated (38) (189 mg, 80% yield) as a white solid.

Preparation of N-(4-methoxy-1H-indole-2-carbonyl)-L-leucyl-3-[(3S)-2-oxopyrrolidin-3-yl]-L-alaninamide, (39). To a solution of methyl N-(4-methoxy-1H-indole-2-carbonyl)-L-leucyl-3-[(3S)-2-oxopyrrolidin-3-yl]-L-alaninate (38) (189 mg, 0.40 mmol) in THF (5 mL) cooled in an ice bath was added a 1.0 M aqueous LiOH solution. After 1 h, a 1.0 M aqueous HCl solution was added until the pH of the mixture was 3, and then the mixture was extracted with EtOAc (3×20 mL). The combined organic layer was washed with brine (10 mL), dried over Na₂SO₄, filtered and concentrated under reduced pressure. The resulting residue was dissolved in anhydrous THF (5 mL) and then carbonyldiimidazole (CDI) (78 mg, 0.48 mmol) was added. After 15 min, an aqueous ammonium hydroxide solution (28-30%) (0.2 mL, 4.8 mmol) was added. After 2 h, another batch of CDI (78 mg, 0.48 mmol) and ammonium hydroxide solution (0.2 mL, 4.8 mmol) were added. After an additional 1 h, water (5 mL) was added. The mixture was then extracted with EtOAc (3×20 mL), and the combined organic phase was dried over Na₂SO₄, filtered and concentrated under reduced pressure. The product was purified by column chromatography on silica gel with a gradient of 0 to 10% MeOH in CHCl₃, which generated (39) (90 mg, 49% yield) as a white sticky solid.

Preparation of N-[(2S)-1-({(1S)-1-cyano-2-[(3S)-2-oxopyrrolidin-3-yl]ethyl}amino)-4-methyl-1-oxopentan-2-yl]-4-methoxy-1H-indole-2-carboxamide, 24. To a solution of N-(4-methoxy-1H-indole-2-carbonyl)-L-leucyl-3-[(3S)-2-oxopyrrolidin-3-yl]-L-alaninamide (39) (90 mg, 0.20 mmol) and Et₃N (0.083 mL, 0.60 mmol) in THF cooled in an ice bath was added trifluoroacetic anhydride (TFAA) (0.056 mL, 0.40 mmol). After 2 h, water (5 mL) was added and the resulting mixture was extracted with EtOAc (3×20 mL). The combined organic layers were dried over Na₂SO₄, filtered and concentrated under reduced pressure. The product was purified by column chromatography on silica gel with a gradient of 0 to 10% MeOH in CHCl₃, which generated 24 (75 mg, 85% yield) as a white solid. ¹H NMR (600 MHz, DMSO-d₆) δ 11.58 (d, J=2.3 Hz, 1H), 8.91 (d, J=7.9 Hz, 1H), 8.48 (d, J=7.9 Hz, 1H), 7.72 (s, 1H), 7.39-7.36 (m, 1H), 7.13-7.08 (m, 1H), 7.03-7.00 (m, 1H), 6.51 (d, J=7.5 Hz, 1H), 5.01-4.95 (m, 1H), 4.49-4.41 (m, 1H), 3.89 (s, 3H), 3.18-3.08 (m, 2H), 2.41-2.33 (m, 1H), 2.19-2.09 (m, 2H), 1.84-1.76 (m, 1H), 1.76-1.65 (m, 3H), 1.57-1.49 (m, 1H), 0.95 (d, J=6.4 Hz, 3H), 0.90 (d, J=6.8 Hz, 3H). LC/MS: Eluent system A (retention time: 4.56 min); ESI-MS 440 [M+H]⁺.

### Compound 25

### Synthesis of (3S)-3-{[N-(4-methoxy-1H-indole-2-carbonyl)-L-leucyl]amino}-2-oxo-4-[(3S)-2-oxopyrrolidin-3-yl]butyl 2,6-bis(trifluoromethyl)benzoate, 25

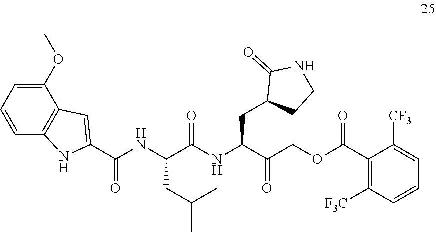 [SMILES: COc1cccc2[nH]c(C(=O)N[C@@H](CC(C)C)C(=O)N[C@@H](C[C@@H]3CCNC3=O)C(=O)COC(=O)c3c(C(F)(F)F)cccc3C(F)(F)F)cc12]

Compound 25 was synthesized as in Scheme 28.

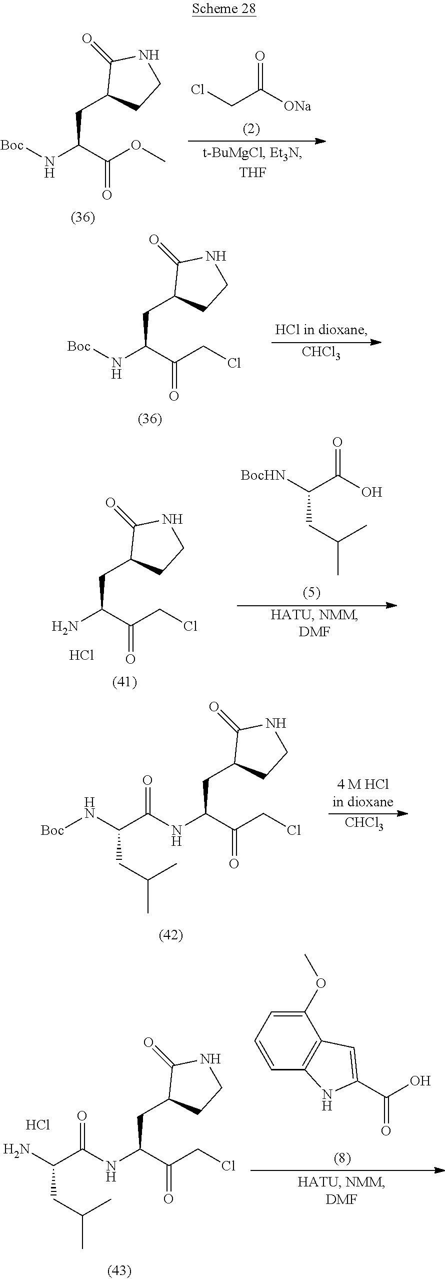

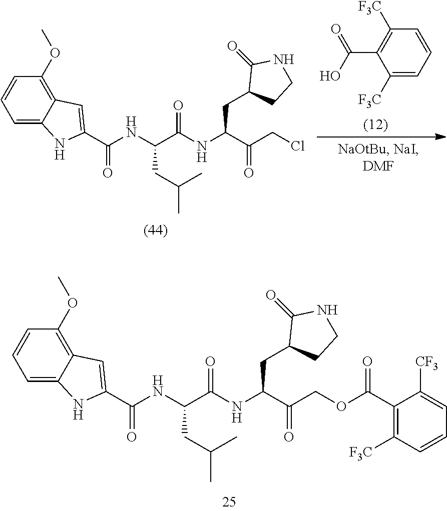 [SMILES: COc1cccc2[nH]c(C(=O)N[C@@H](CC(C)C)C(=O)N[C@@H](C[C@@H]3CCNC3=O)C(=O)CCl)cc12 ; O=C(O)c1c(C(F)(F)F)cccc1C(F)(F)F ; COc1cccc2[nH]c(C(=O)N[C@@H](CC(C)C)C(=O)N[C@@H](C[C@@H]3CCNC3=O)C(=O)COC(=O)c3c(C(F)(F)F)cccc3C(F)(F)F)cc12]

Preparation of tert-butyl {(2S)-4-chloro-3-oxo-1-[(3S)-2-oxopyrrolidin-3-yl]butan-2-yl}carbamate, (40). To a solution of methyl N-(tert-butoxycarbonyl)-3-[(3S)-2-oxopyrrolidin-3-yl]-L-alaninate (36) (8.5 g, 29.7 mmol) [prepared using method in Journal of Medicinal Chemistry (2015), 58, p 9414-9420 and references within], sodium chloroacetate (11.4 g, 98.0 mmol) and Et₃N (13.6 mL, 98.0 mmol) in anhydrous THF (250 mL) cooled in an ice bath was added a solution of 2.0 M tert-butylmagnesium chloride solution in THF (148.5 mL, 297 mmol) via a dropping funnel over 1.5 h. After 30 min, the ice bath was removed. After 18 h at room temperature, ice was then added, followed by a precooled 4 N HCl aqueous solution to carefully adjust pH to 7. The two layers were separated and the aqueous layer was extracted with EtOAc (3×150 mL). The combined organic phase was washed with brine (100 mL), dried over Na₂SO₄, filtered and concentrated under reduced pressure. The product was purified by column chromatography on silica gel with a gradient of 0 to 1% MeOH in EtOAc, which generated (40) (5.45 g, 60% yield) as a white foam.

Preparation of (3S)-3-[(2S)-2-amino-4-chloro-3-oxobutyl]pyrrolidin-2-one hydrochloride salt, (41). A solution of tert-butyl {(2S)-4-chloro-3-oxo-1-[(3S)-2-oxopyrrolidin-3-yl]butan-2-yl}carbamate (40) (3.25 g, 10.66 mmol) in CHCl₃ (50 mL) was cooled using ice-water bath and 4 M HCl in 1,4-dioxane (15.0 mL) was added. After 30 min, the bath was removed. After overnight, an off-white precipitate had formed. The mixture was concentrated under reduced pressure (bath at 35° C.). The residue was then co-evaporated with acetonitrile (3×20 mL), DCM (20 mL), and then dried under reduce pressure for an additional 1 h, which afforded (41) (2.58 g) as a pale yellow color solid. This material was used in the next step without further purification. ¹H NMR (600 MHz, DMSO-d₆) δ 8.61 (br s, 3H), 8.01 (br s, 1H), 4.93 (d, J=17.1 Hz, 1H), 4.77 (d, J=17.1 Hz, 1H), 4.36-4.28 (m, 1H), 3.33-3.16 (m, 2H), 2.64-2.54 (m, 1H), 2.34-2.27 (m, 1H), 2.03-1.97 (m, 1H), 1.93-1.86 (m, 1H), 1.85-1.77 (m, 1H).

Preparation of N²-(tert-butoxycarbonyl)-N-{(2S)-4-chloro-3-oxo-1-[(3S)-2-oxopyrrolidin-3-yl]butan-2-yl}-L-leucinamide (42). To a solution of (41) (2.58 g, 10.7 mmol) and N-(tert-butylcarbonyl)-L-leucine (5) (2.72 g, 11.77 mmol) in anhydrous DMF (50 mL) cooled in an ice bath was added HATU (4.48 g, 11.77 mmol). Then NMM (3.53 mL, 32.10 mmol) was added dropwise. After 45 min, a mixture of ice/saturated aqueous NaHCO₃ (1:1, 200 mL) was added. The resulting mixture was extracted with EtOAc (3×200 mL). The combined organic layer was washed with saturated brine solution (200 mL), dried over anhydrous Na₂SO₄, filtered, and concentrated under reduced pressure. The resulting residue was dissolved in CHCl₃ (20 mL) and loaded on 120 g silica gel column (Silicycle) and the product was purified by Biotage® with a gradient of 0 to 4% MeOH in CHCl₃, which generated (42) (2.10 g, 47% yield) as a white foamy solid. ¹H NMR (600 MHz, DMSO-d₆) δ 8.47 (d, J=7.9 Hz, 1H), 7.64 (br s, 1H), 7.04 (d, J=7.3 Hz, 1H), 4.61 (d, J=16.7 Hz, 1H), 4.55 (d, J=16.7 HZ, 1H), 4.44-4.37 (m, 1H), 3.95-3.86 (m, 1H), 3.22-3.12 (m, 1H), 3.11-3.03 (m, 1H), 2.32-2.21 (m, 1H), 2.16-2.07 (m, 1H), 2.03-1.94 (m, 1H), 1.70-1.57 (m, 3H), 1.51-1.40 (m, 2H), 1.38 (s, 9H), 0.89 (d, J=6.6 Hz, 3H), 0.86 (d, J=6.6 Hz, 3H).

Preparation of N-{(2S)-4-chloro-3-oxo-1-[(3S)-2-oxopyrrolidin-3-yl]butan-2-yl}-L-leucinamide hydrochloride salt (43). A solution of (42) (2.10 g, 5.03 mmol) in CHCl₃ (25 mL) was cooled using an ice-water bath and then 4 M HCl in 1,4-dioxane (10.0 mL) was added. After 30 min, the ice bath was removed. After overnight at room temperature, a gummy precipitate had formed. The mixture was concentrated under reduced pressure (bath at 35° C.). The resulting residue was co-evaporated with acetonitrile (3×20 mL), DCM (20 mL), and then dried under reduced pressure for additional 1 h, which afforded (43) (1.7 g) as an off-white color solid. This material was used in the next step without further purification. ¹H NMR (600 MHz, DMSO-d₆) δ 9.13 (d, J=7.3 Hz, 1H), 8.33 (br d, J=3.8 Hz, 3H), 7.71 (s, 1H), 4.70 (s, 2H), 4.56-4.49 (m, 1H), 3.94-3.76 (m, 1H), 3.21-3.10 (m, 2H), 2.45-2.35 (m, 1H), 2.28-2.09 (m, 1H), 1.96-1.90 (m, 1H), 1.76-1.65 (m, 3H), 1.64-1.45 (m, 2H), 0.94 (d, J=6.6 Hz, 3H), 0.91 (d, J=6.6 Hz, 3H).

Preparation of N-[(2S)-1-({(2S)-4-chloro-3-oxo-1-[(3S)-2-oxopyrrolidin-3-yl]butan-2-yl}amino)-4-methyl-1-oxopentan-2-yl]-4-methoxy-1H-indole-2-carboxamide, (44). To a solution of (43) (1.7 g, 4.8 mmol) and 4-methoxy-1H-indole-2-carboxylic acid (8) (1.0 g, 5.28 mmol) in anhydrous DMF (30 mL) cooled in an ice bath was added HATU (2.0 g, 5.28 mmol). Then NMM (1.58 mL, 14.40 mmol) was added dropwise. After 45 min, an ice/saturated aqueous NaHCO₃ (1:1, 150 mL) was added. The mixture was extracted with EtOAc (3×150 mL). The combined organic layer was washed with saturated brine solution (150 mL), dried over anhydrous Na₂SO₄, filtered, and concentrated under reduced pressure. The resulting residue was dissolved in CHCl₃ (25 mL) and loaded on 120 g silica gel column (Silicycle) and the product purified by Biotage® with a gradient of 0 to 6% MeOH in CHCl₃. The resulting solid was triturated with EtOAc (10 mL), filtered, washed with EtOAc (2×2 mL), and dried under reduced pressure, which generated (44) (1.0 g, 42% yield) as an off-white solid. ¹H NMR (600 MHz, DMSO-d₆) δ 11.59 (d, J=2.1 Hz, 1H), 8.62 (d, J=7.7 Hz, 1H), 8.45 (d, J=7.5 Hz, 1H), 7.65 (s, 1H), 7.37 (dd, J=0.8, 2.3 Hz, 1H), 7.10 (t, J=8.1 Hz, 1H) 7.01 (d, J=8.3 Hz, 1H), 6.51 (d, J=7.5 Hz, 1H), 4.62 (d, J=16.7 Hz, 1H), 4.59 (d, J=16.7 Hz, 1H), 4.49-4.42 (m, 2H), 3.89 (s, 3H), 3.18-3.06 (m, 2H), 2.33-2.25 (m, 1H), 2.15-2.06 (m, 1H), 2.00-1.93 (m, 1H), 1.79-1.52 (m, 5H), 0.95 (d, J=6.4 Hz, 3H), 0.90 (d, J=6.4 Hz, 3H).

Preparation of (3S)-3-{[N-(4-methoxy-1H-indole-2-carbonyl)-L-leucyl]amino}-2-oxo-4-[(3S)-2-oxopyrrolidin-3-yl]butyl 2,6-bis(trifluoromethyl)benzoate, 25. A mixture of 2,6-bis(trifluoromethyl)benzoic acid (12) (131.4 mg, 0.50 mmol) and sodium tert-butoxide (24.5 mg, 0.25 mmol) in anhydrous DMF (3 mL) was stirred at room temperature for 30 min. To this (9) (50 mg, 0.10 mmol) and NaI (30.5 mg, 0.20 mmol) were added successively. After 72 h, a saturated brine solution (10 mL) was added and the mixture was extracted with CHCl₃ (3×10 mL). The combined organic layer was washed with saturated brine solution (10 mL), dried over anhydrous Na₂SO₄, filtered, and concentrated under reduced pressure. The crude was dissolved in CHCl₃ (5 mL) and loaded on silica gel column (3×4 g Silicycle siliasep). The product was purified by Biotage® with a gradient of 0 to 2% MeOH in CHCl₃, which afforded 25 (58.3 mg, 80% yield) as an off-white solid. ¹H NMR (600 MHz, DMSO-d₆) δ 11.56 (br d, J=1.5 Hz, 1H), 8.65 (br d, J=8.1 Hz, 1H), 8.46 (br d, J=7.7 Hz, 1H), 8.26 (d, J=8.1 Hz, 2H), 8.02 (t, J=8.1 Hz, 1H), 7.65 (s, 1H), 7.37 (d, J=1.5 Hz, 1H), 7.11 (t, J=8.0 Hz, 1H), 7.01 (d, J=8.3 Hz, 1H), 6.51 (d, J=7.7 Hz, 1H), 5.21 (d, J=17.0 Hz, 1H), 5.16 (d, J=17.0 Hz, 1H), 4.57-4.49 (m, 2H), 3.89 (s, 3H), 3.16-3.05 (m, 2H), 2.37-2.30 (m, 1H), 2.12-2.00 (m, 2H), 1.78-1.54 (m, 5H), 0.95 (d, J=6.2 Hz, 3H), 0.90 (d, J=6.2 Hz, 3H). ¹⁹F NMR (565 MHz, DMSO-d₆) δ −58.3-−58.33 (m, 6F). LC/MS: Eluent system A (retention time: 5.12 min); ESI-MS: 713 [M+H]⁺.

### Compound 26

### Synthesis of (3S)-3-{[N-(4-methoxy-1H-indole-2-carbonyl)-L-leucyl]amino}-2-oxo-4-[(3S)-2-oxopyrrolidin-3-yl]butyl 2,6-dimethyl-4-(trifluoromethyl)pyridine-3-carboxylate, 26

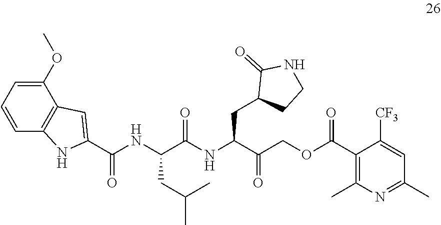 [SMILES: COc1cccc2[nH]c(C(=O)N[C@@H](CC(C)C)C(=O)N[C@@H](C[C@@H]3CCNC3=O)C(=O)COC(=O)c3c(C(F)(F)F)cc(C)nc3C)cc12]

Compound 26 was synthesized as in Scheme 29.

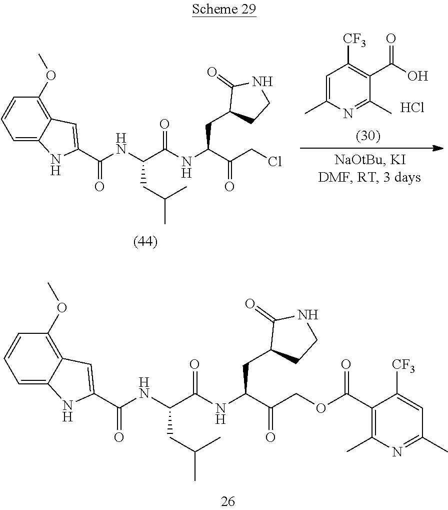

Preparation of (3S)-3-{[N-(4-methoxy-1H-indole-2-carbonyl)-L-leucyl]amino}-2-oxo-4-[(3S)-2-oxopyrrolidin-3-yl]butyl 2,6-dimethyl-4-(trifluoromethyl)pyridine-3-carboxylate, 26. To a solution of 2,6-dimethyl-4-(trifluoromethyl)pyridine-3-carboxylic acid hydrochloride (30) (102 mg, 0.40 mmol) in DMF (5 mL) was added NaOtBu (58 mg, 0.60 mmol). After 30 min, N-[(2S)-1-({(2S)-4-chloro-3-oxo-1-[(3S)-2-oxopyrrolidin-3-yl]butan-2-yl}amino)-4-methyl-1-oxopentan-2-yl]-4-methoxy-1H-indole-2-carboxamide (44) (50 mg, 0.10 mmol) followed by KI (16.6 mg, 0.10 mmol) were added. After 3 days, EtOAc (40 mL) was added and the resulting mixture was washed with water (3×10 mL), brine (10 mL), dried over Na₂SO₄, filtered and concentrated under reduced pressure. The product was purified by column chromatography on silica gel with a gradient of 0 to 10% MeOH in CHCl₃, which generated 26 (32 mg, 47% yield) as a white solid. ¹H NMR (600 MHz, DMSO-d₆) δ 11.57 (d, J=2.3 Hz, 1H), 8.65 (d, J=7.9 Hz, 1H), 8.46 (d, J=7.9 Hz, 1H), 7.66 (s, 1H), 7.63 (s, 1H). 7.39-7.36 (m, 1H), 7.14-7.07 (m, 1H), 7.04-6.98 (m, 1H), 6.51 (d, J=7.2 Hz, 1H), 5.23-5.14 (m, 2H), 4.57-4.49 (m, 2H), 3.89 (s, 3H), 3.18-3.06 (m, 2H), 2.64 (s, 3H), 2.59 (s, 3H), 2.39-2.31 (m, 1H), 2.14-2.01 (m, 2H), 1.77-1.57 (m, 5H), 0.95 (d, J=6.4 Hz, 3H), 0.91 (d, J=6.4 Hz, 3H). ¹⁹F NMR (565 MHz, DMSO-d₆) δ −60.73 (s, 3F). LC/MS: Eluent system A (retention time: 6.29 min); ESI-MS 674 [M+H]⁺.

### Compound 27

### Synthesis of (3S)-3-{[N-(4-methoxy-1H-indole-2-carbonyl)-L-leucyl]amino}-2-oxo-4-[(3S)-2-oxopyrrolidin-3-yl]butyl 3,3,3-trifluoro-2,2-dimethylpropanoate, 27

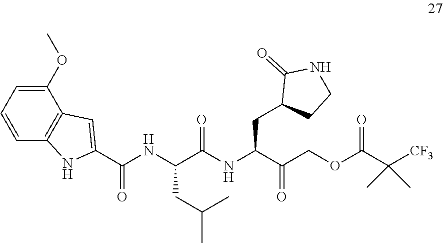 [SMILES: COc1cccc2[nH]c(C(=O)N[C@@H](CC(C)C)C(=O)N[C@@H](C[C@@H]3CCNC3=O)C(=O)COC(=O)C(C)(C)C(F)(F)F)cc12]

Compound 27 was synthesized as in Scheme 30.

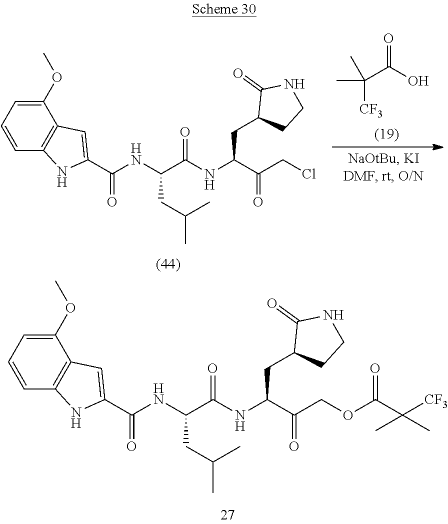

Preparation of (3S)-3-{[N-(4-methoxy-1H-indole-2-carbonyl)-L-leucyl]amino}-2-oxo-4-[(3S)-2-oxopyrrolidin-3-yl]butyl 3,3,3-trifluoro-2,2-dimethylpropanoate, 27. The procedure for 5 in Scheme 8 was followed with N-[(2S)-1-({(2S)-4-chloro-3-oxo-1-[(3S)-2-oxopyrrolidin-3-yl]butan-2-yl}amino)-4-methyl-1-oxopentan-2-yl]-4-methoxy-1H-indole-2-carboxamide (44) (40 mg, 0.081 mmol), 3,3,3-trifluoro-2,2-dimethylpropanoic acid (19) (77 mg, 0.49 mmol), sodium tert-butoxide (25 mg, 0.26 mmol), potassium iodide (20 mg, 0.120 mmol) and DMF (3 mL). The product was purified by column chromatography (eluted with gradient of 0 to 12% methanol in 1:1 chloroform:ethyl acetate mixture), followed by trituration with ether which provided 27 (10.0 mg, 20% yield) as a white powder. ¹H NMR (600 MHz, DMSO-d₆) δ 11.58 (d, J=1.9 Hz, 1H), 8.58 (d, J=8.2 Hz, 1H), 8.45 (d, J=7.6 Hz, 1H), 7.65 (br.s, 1H), 7.37 (dd, J=0.7, 2.3 Hz, 1H), 7.10 (t, J=8.0 Hz, 1H), 7.01 (d, J=8.2 Hz, 1H), 6.51 (d, J=7.6 Hz, 1H), 5.01 (d, J=17.0 Hz, 1H), 4.98 (d, J=17.0 Hz, 1H), 4.50-4.41 (m, 2H), 3.89 (s, 3H), 3.17-3.06 (m, 2H), 2.35-2.28 (m, 1H), 2.11-2.06 (m, 1H), 2.02-1.96 (m, 1H), 1.78-1.70 (m, 2H), 1.68-1.61 (m, 1H), 1.59-1.53 (m, 2H), 1.43 (s, 6H), 0.95 (d, J=6.4 Hz, 3H), 0.90 (d, J=6.4 Hz, 3H). LC/MS: Eluent system A (retention time: 6.31 min); ESI-MS 611 [M+H]⁺, 609 [M−H]⁻.

### Compound 28

### Synthesis of (3S)-3-{[N-(4-methoxy-1H-indole-2-carbonyl)-L-leucyl]amino}-2-oxo-4-[(3S)-2-oxopiperidin-3-yl]butyl 1,5-dimethyl-3-(trifluoromethyl)-1H-pyrazole-4-carboxylate, 28

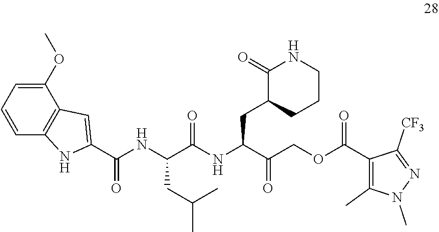 [SMILES: COc1cccc2[nH]c(C(=O)N[C@@H](CC(C)C)C(=O)N[C@@H](C[C@@H]3CCCNC3=O)C(=O)COC(=O)c3c(C(F)(F)F)nn(C)c3C)cc12]

Compound 28 was synthesized as in Scheme 31.

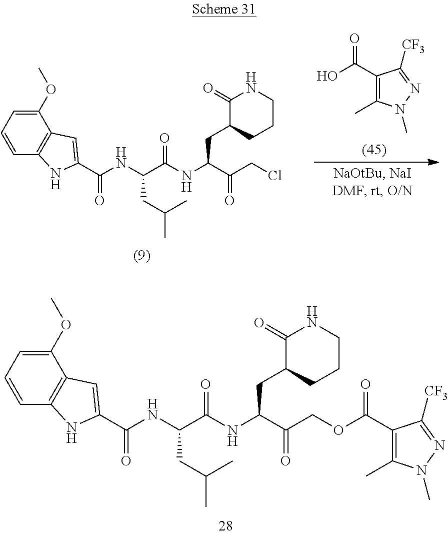 [SMILES: COc1cccc2[nH]c(C(=O)N[C@@H](CC(C)C)C(=O)N[C@@H](C[C@@H]3CCCNC3=O)C(=O)CCl)cc12 ; Cc1c(C(=O)O)c(C(F)(F)F)nn1C ; COc1cccc2[nH]c(C(=O)N[C@@H](CC(C)C)C(=O)N[C@@H](C[C@@H]3CCCNC3=O)C(=O)COC(=O)c3c(C(F)(F)F)nn(C)c3C)cc12]

Preparation of (3S)-3-{[N-(4-methoxy-1H-indole-2-carbonyl)-L-leucyl]amino}-2-oxo-4-[(3S)-2-oxopiperidin-3-yl]butyl 1,5-dimethyl-3-(trifluoromethyl)-1H-pyrazole-4-carboxylate, 28. The procedure for 9 in Scheme 12 was followed with 1,5-dimethyl-3-(trifluoromethyl)-1H-pyrazole-4-carboxylic acid (45) (66 mg, 0.316 mmol), NaOtBu (15 mg, 0.158 mmol), N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (40 mg, 0.0792 mmol), sodium iodide (24 mg, 0.158 mmol) and DMF (1 mL). The product was purified by column chromatography on silica (0 to 2% methanol in 1:1 chloroform:ethyl acetate), which generated 28 (20 mg, 37% yield) as a white solid. ¹H NMR (600 MHz, DMSO-d₆) δ 11.57 (br s, 1H), 8.61 (d, J=7.9 Hz, 1H), 8.45 (d, J=7.5 Hz, 1H), 7.48-7.44 (m, 1H), 7.39-7.36 (m, 1H), 7.13-7.08 (m, 1H), 7.04-6.99 (m, 1H), 6.54-6.49 (m, 1H), 5.12-5.02 (m, 2H), 4.55-4.45 (m, 2H), 3.91 (s, 3H), 3.86 (s, 3H), 3.12-3.04 (m, 2H), 2.54 (s, 3H), 2.29-2.17 (m, 2H), 1.89-1.79 (m, 1H), 1.77-1.64 (m, 4H), 1.63-1.47 (m, 2H), 1.44-1.28 (m, 1H), 0.95 (d, J=6.4 Hz, 3H), 0.90 (d, J=6.4 Hz, 3H). ¹⁹F NMR (565 MHz, DMSO-d₆) δ −60.80 (s, 3F). LC/MS: Eluent system A (retention time: 5.87 min); ESI-MS: 677 [M+H]⁺.

### Compound 29

### Synthesis of (3S)-3-{[N-(4-methoxy-1H-indole-2-carbonyl)-L-leucyl]amino}-2-oxo-4-[(3S)-2-oxopiperidin-3-yl]butyl 6-methylimidazo[2,1-b][1,3]thiazole-5-carboxylate, 29

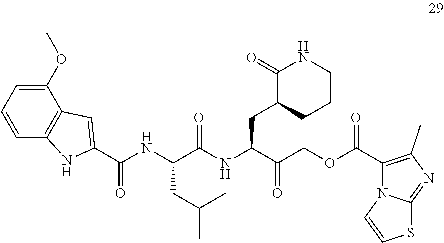 [SMILES: COc1cccc2[nH]c(C(=O)N[C@@H](CC(C)C)C(=O)N[C@@H](C[C@@H]3CCCNC3=O)C(=O)COC(=O)c3c(C)nc4sccn34)cc12]

Compound 29 was synthesized as in Scheme 32.

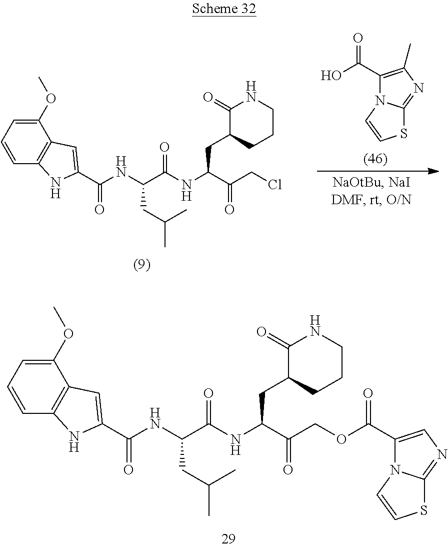 [SMILES: COc1cccc2[nH]c(C(=O)N[C@@H](CC(C)C)C(=O)N[C@@H](C[C@@H]3CCCNC3=O)C(=O)CCl)cc12 ; Cc1nc2sccn2c1C(=O)O ; COc1cccc2[nH]c(C(=O)N[C@@H](CC(C)C)C(=O)N[C@@H](C[C@@H]3CCCNC3=O)C(=O)COC(=O)c3cnc4sccn34)cc12]

Preparation of (3S)-3-{[N-(4-methoxy-1H-indole-2-carbonyl)-L-leucyl]amino}-2-oxo-4-[(3S)-2-oxopiperidin-3-yl]butyl 6-methylimidazo[2,1-b][1,3]thiazole-5-carboxylate, 29. The procedure for 9 in Scheme 12 was followed with 6-methylimidazo[2,1-b][1,3]thiazole-5-carboxylic acid (46) (60 mg, 0.332 mmol), NaOtBu (16 mg, 0.166 mmol), N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (42 mg, 0.083 mmol), sodium iodide (25 mg, 0.166 mmol) and DMF (1 mL). The product was purified by column chromatography on silica (0 to 2% methanol in 1:1 chloroform:ethyl acetate), which generated 29 (11 mg, 20% yield) as a white solid. ¹H NMR (600 MHz, DMSO-d₆) δ 11.57 (br s, 1H), 8.64 (d, J=7.9 Hz, 1H), 8.45 (d, J=7.9 Hz, 1H), 8.13 (d, J=4.1 Hz, 1H), 7.48-7.41 (m, 2H), 7.38 (br s, 1H), 7.12-7.08 (m, 1H), 7.03-6.99 (m, 1H), 6.51 (d, J=7.2 Hz, 1H), 5.23-5.09 (m, 2H), 4.58-4.47 (m, 2H), 3.89 (s, 3H), 3.15-3.04 (m, 2H), 2.50 (s, 3H), 2.32-2.16 (m, 2H), 1.88-1.80 (m, 1H), 1.77-1.66 (m, 4H), 1.62-1.47 (m, 2H), 1.45-1.30 (m, 1H), 0.96 (d, J=6.4 Hz, 3H), 0.91 (d, J=6.4 Hz, 3H). LC/MS: Eluent system A (retention time: 5.81 min); ESI-MS: 651 [M+H]⁺.

### Compound 30

### Synthesis of N-[(2S)-1-({(2S)-4-[4-(methanesulfonyl)phenoxy]-3-oxo-1-[(3S)-2-oxopiperidin-3-yl]butan-2-yl}amino)-4-methyl-1-oxopentan-2-yl]-4-methoxy-1H-indole-2-carboxamide, 30

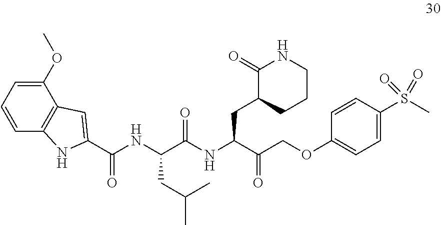 [SMILES: COc1cccc2[nH]c(C(=O)N[C@@H](CC(C)C)C(=O)N[C@@H](C[C@@H]3CCCNC3=O)C(=O)COc3ccc(S(C)(=O)=O)cc3)cc12]

Compound 30 was synthesized as in Scheme 33.

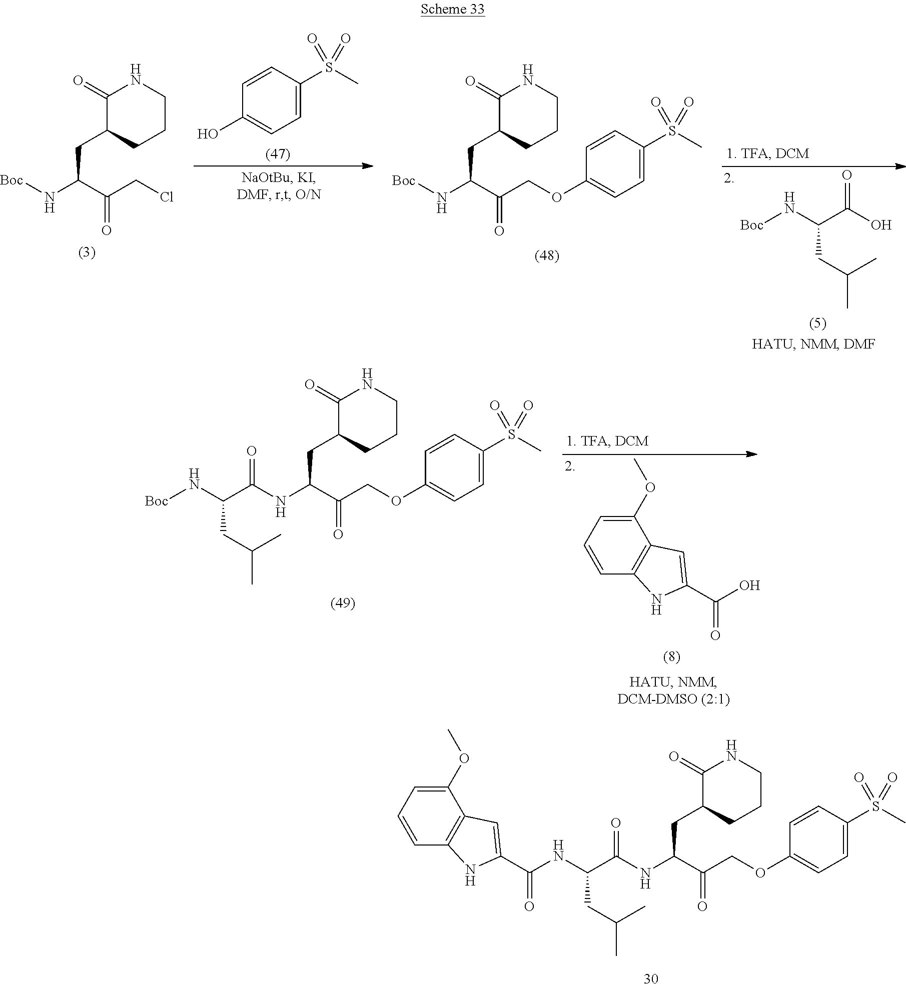

Preparation of tert-butyl {(2S)-4-[4-(methanesulfonyl)phenoxy]-3-oxo-1-[(3S)-2-oxopiperidin-3-yl]butan-2-yl}carbamate, (48). The procedure for 5 in Scheme 8 was followed with tert-butyl ((S)-4-chloro-3-oxo-1-((S)-2-oxopiperidin-3-yl)butan-2-yl)carbamate (3) (100 mg, 0.31 mmol), 4-(methylsulfonyl)phenol (47) (92 mg, 0.53 mmol), sodium tert-butoxide (33 mg, 0.35 mmol), potassium iodide (36 mg, 0.22 mmol) and DMF (5 mL). After overnight at room temperature, an additional portion of 4-(methylsulfonyl)phenol (47) (36 mg, 0.21 mmol) and sodium tert-butoxide (12 mg, 0.125 mmol) in DMF (2 mL) that was pre-mixed for 30 minutes was added. After 2 hours, the resulting mixture was partitioned between DCM (15 mL) and water (6 mL). The organic layer was poured into a petri dish and left in the fume hood to evaporate at room temperature. The product was purified by silica gel column chromatography (eluted with 0% to 12% methanol in chloroform) provided (48) (55 mg, 39% yield) as a clear oil.

Preparation of N²-(tert-butoxycarbonyl)-N-{(2S)-4-[4-(methanesulfonyl)phenoxy]-3-oxo-1-[(3S)-2-oxopiperidin-3-yl]butan-2-yl}-L-leucinamide, (49). To a solution of (48) (55 mg, 0.121 mmol) in DCM (5 mL) cooled in an ice bath was added TFA (3 mL). After 30 min, the mixture was concentrated under reduced pressure (bath temperature 36° C.). The resulting residue was dissolved in DMF (6 mL), placed in the ice bath and N-(tert-butylcarbonyl)-L-leucine (5) (35 mg, 0.151 mmol) was added 10 minutes later, followed by HATU (61 mg, 0.160 mmol) and NMM (53 mg, 0.52 mmol). After 45 min, the mixture was partitioned between chloroform (15 mL) and water (5 mL). The separated organic layer was poured onto petri dish and left in the fume hood to evaporate at room temperature. The product was purified by silica gel column chromatography (eluted with 0 to 8% methanol in chloroform), which provided (49) (53 mg, 77% yield over two steps) as a clear oil.

Preparation of N-[(2S)-1-({(2S)-4-[4-(methanesulfonyl)phenoxy]-3-oxo-1-[(3S)-2-oxopiperidin-3-yl]butan-2-yl}amino)-4-methyl-1-oxopentan-2-yl]-4-methoxy-1H-indole-2-carboxamide, 30. A solution of (49) (53 mg, 0.093 mmol) in DCM (5 mL) was treated with TFA (3 mL). After 30 min, the mixture was concentrated under reduced pressure (bath temperature 36° C.). The residue obtained after evaporation was dissolved in a DCM (10 mL)-DMSO (5 mL) mixture, and the resulting solution was cooled in an ice bath. After 15 min, 4-methoxy-1H-indole-2-carboxylic acid (8) (23 mg, 0.121 mmol) was added, followed by HATU (50 mg, 0.131 mmol) and NMM (48 mg, 0.47 mmol). After 1 hour, the resulting mixture was diluted with DCM (20 mL), washed with 1% aqueous sodium bicarbonate solution (3 mL), followed by water (3 mL). The organic layer was poured onto petri dish and left in the fume hood to evaporate at room temperature. The product was purified by silica gel column chromatography (eluted with gradient of 0 to 12% methanol in 1:1 chloroform:ethyl acetate mixture) provided 30 (12.0 mg, 20% yield over two steps) as an off-white powder. LC/MS: Eluent system A (retention time: 4.97 min); ESI-MS 641 [M+H]⁺, 639 [M−H]⁻.

### Compound 31

### Synthesis of (3S)-3-{[N-(4-methoxy-1H-indole-2-carbonyl)-L-leucyl]amino}-2-oxo-4-[(3S)-2-oxopiperidin-3-yl]butyl 1,3,5-trimethyl-1H-pyrazole-4-carboxylate, 31

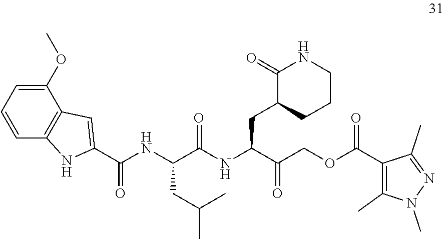 [SMILES: COc1cccc2[nH]c(C(=O)N[C@@H](CC(C)C)C(=O)N[C@@H](C[C@@H]3CCCNC3=O)C(=O)COC(=O)c3c(C)nn(C)c3C)cc12]

Compound 31 was synthesized as in Scheme 34.

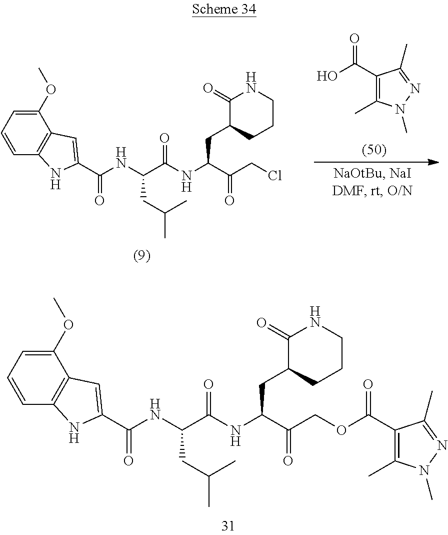 [SMILES: COc1cccc2[nH]c(C(=O)N[C@@H](CC(C)C)C(=O)N[C@@H](C[C@@H]3CCCNC3=O)C(=O)CCl)cc12 ; Cc1nn(C)c(C)c1C(=O)O ; COc1cccc2[nH]c(C(=O)N[C@@H](CC(C)C)C(=O)N[C@@H](C[C@@H]3CCCNC3=O)C(=O)COC(=O)c3c(C)nn(C)c3C)cc12]

Preparation of (3S)-3-{[N-(4-methoxy-1H-indole-2-carbonyl)-L-leucyl]amino}-2-oxo-4-[(3S)-2-oxopiperidin-3-yl]butyl 1,3,5-trimethyl-1H-pyrazole-4-carboxylate, 31. The procedure for 9 in Scheme 12 was followed with 1,3,5-trimethyl-1H-pyrazole-4-carboxylic acid (50) (55 mg, 0.357 mmol), NaOtBu (17 mg, 0.178 mmol), N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (45 mg, 0.089 mmol), sodium iodide (27 mg, 0.178 mmol) and DMF (1 mL). The product was purified by column chromatography on silica (0 to 2% methanol in 1:1 chloroform:ethyl acetate), which generated 31 (13 mg, 23% yield) as a white solid. ¹H NMR (600 MHz, DMSO-d₆) δ 11.57 (br s, 1H), 8.60 (d, J=8.3 Hz, 1H), 8.44 (d, J=8.3 Hz, 1H), 7.45 (br s, 1H), 7.37 (dd, J=0.8, 2.3 Hz, 1H), 7.15-7.07 (m, 1H), 7.04-6.97 (m, 1H), 6.51 (d, J=7.9 Hz, 1H), 5.09-4.94 (m, 2H), 4.56-4.44 (m, 2H), 3.89 (s, 3H), 3.69 (s, 3H), 2.44 (s, 3H), 2.26 (s, 3H), 1.92-1.78 (m, 2H), 1.78-1.65 (m, 4H), 1.62-1.46 (m, 4H), 1.43-1.25 (m, 2H), 0.95 (d, J=6.0 Hz, 3H), 0.91 (d, J=6.0 Hz, 3H). LC/MS: Eluent system A (retention time: 5.53 min); ESI-MS: 623 [M+H]⁺.

### Compound 32

### Synthesis of (3S)-3-{[N-(4-methoxy-1H-indole-2-carbonyl)-L-leucyl]amino}-2-oxo-4-[(3S)-2-oxopiperidin-3-yl]butyl isoquinoline-4-carboxylate, 32

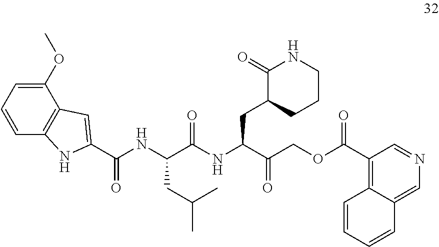 [SMILES: COc1cccc2[nH]c(C(=O)N[C@@H](CC(C)C)C(=O)N[C@@H](C[C@@H]3CCCNC3=O)C(=O)COC(=O)c3cncc4ccccc34)cc12]

Compound 32 was synthesized as in Scheme 35

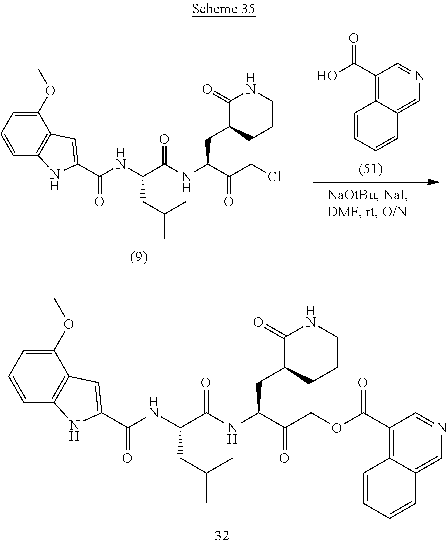 [SMILES: COc1cccc2[nH]c(C(=O)N[C@@H](CC(C)C)C(=O)N[C@@H](C[C@@H]3CCCNC3=O)C(=O)CCl)cc12 ; O=C(O)c1cncc2ccccc12 ; COc1cccc2[nH]c(C(=O)N[C@@H](CC(C)C)C(=O)N[C@@H](C[C@@H]3CCCNC3=O)C(=O)COC(=O)c3cncc4ccccc34)cc12]

Preparation of (3S)-3-{[N-(4-methoxy-1H-indole-2-carbonyl)-L-leucyl]amino}-2-oxo-4-[(3S)-2-oxopiperidin-3-yl]butyl isoquinoline-4-carboxylate, 32. The procedure for 9 in Scheme 12 was followed with isoquinoline-4-carboxylic acid (51) (59 mg, 0.341 mmol), NaOtBu (17 mg, 0.171 mmol), N-[(2S)-1-({(2S)-4-chloro-3-oxo-1-[(3S)-2-oxopiperidin-3-yl]butan-2-yl}amino)-4-methyl-1-oxopentan-2-yl]-4-methoxy-1H-indole-2-carboxamide (9) (43 mg, 0.085 mmol), sodium iodide (26 mg, 0.171 mmol) and DMF (1 mL). The product was purified by column chromatography on silica (0 to 2% methanol in 1:1 chloroform:ethyl acetate), which generated 32 (18 mg, 32% yield) as a white solid. ¹H NMR (600 MHz, DMSO-d₆) δ 11.58 (br s, 1H), 9.58 (s, 1H), 9.12 (s, 1H), 8.77 (dd, J=0.8, 8.7 Hz, 1H), 8.68 (d, J=8.1 Hz, 1H), 8.47 (d, J=7.7 Hz, 1H), 8.30 (d, J=8.1 Hz, 1H), 7.98-7.94 (m, 1H), 7.84-7.80 (m, 1H), 7.48 (br s, 1H), 7.39 (d, J=2.3 Hz, 1H), 7.12-7.07 (m, 1H), 7.02 (d, J=8.3 Hz, 1H), 6.51 (d, J=7.5 Hz, 1H), 5.34-5.22 (m, 2H), 4.65-4.56 (m, 1H), 4.56-4.46 (m, 1H), 3.89 (s, 3H), 3.17-3.02 (m, 2H), 2.36-2.19 (m, 2H), 1.94-1.83 (m, 1H), 1.82-1.66 (m, 4H), 1.65-1.50 (m, 2H), 1.45-1.32 (m, 1H), 0.96 (d, J=6.2 Hz, 3H), 0.92 (d, J=6.4 Hz, 3H). LC/MS: Eluent system A (retention time: 6.09 min); ESI-MS: 642 [M+H]⁺.

### Compound 34

### Synthesis of N-[(2S)-1-({(1S)-1-cyano-2-[(3S)-2-oxopiperidin-3-yl]ethyl}amino)-4-methyl-1-oxopentan-2-yl]-4-methoxy-1H-indole-2-carboxamide, 34

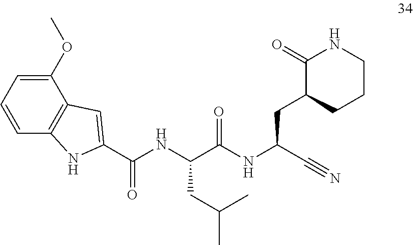 [SMILES: COc1cccc2[nH]c(C(=O)N[C@@H](CC(C)C)C(=O)N[C@H](C#N)C[C@@H]3CCCNC3=O)cc12]

Compound 34 was synthesized as in Scheme 36.

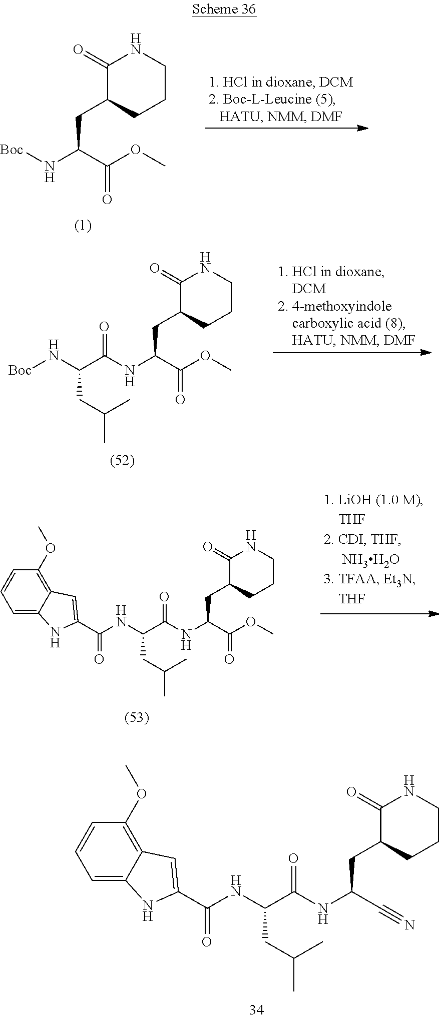 [SMILES: COC(=O)[C@H](C[C@@H]1CCCNC1=O)NC(=O)OC(C)(C)C ; COC(=O)[C@H](C[C@@H]1CCCNC1=O)NC(=O)[C@H](CC(C)C)NC(=O)OC(C)(C)C ; COC(=O)[C@H](C[C@@H]1CCCNC1=O)NC(=O)[C@H](CC(C)C)NC(=O)c1cc2c(OC)cccc2[nH]1 ; COc1cccc2[nH]c(C(=O)N[C@@H](CC(C)C)C(=O)N[C@H](C#N)C[C@@H]3CCCNC3=O)cc12]

Preparation of methyl N-(tert-butoxycarbonyl)-L-leucyl-3-[(3S)-2-oxopiperidin-3-yl]-L-alaninate, (52). Prepared as described in Y. Zhai et al. Eur. J. Med. Chem. 2016, 124, 559. To a solution of methyl N-(tert-butoxycarbonyl)-3-[(3S)-2-oxopiperidin-3-yl]-L-alaninate (1) (501 mg, 1.67 mmol) in DCM (10 mL) was cooled using an ice-water bath and then 4 M HCl in 1,4-dioxane (10 mL) was added. After 30 min, the ice bath was removed. After overnight, the mixture was concentrated under reduced pressure. Treatment with DCM/ether (1:1, 3×20 mL), and drying under reduced pressure for 1 h afforded a white solid. This material was used without further purification in the next step. To a solution of hydrochloride salt and N-(tert-butoxycarbonyl)-L-leucine (5) (425 mg, 1.84 mmol) in anhydrous DMF (20 mL) cooled in an ice bath was added HATU (700 mg, 1.84 mmol), followed by addition of NMM (0.551 mL, 5.01 mmol) dropwise. After 45 min, an ice/saturated aqueous NaHCO₃ mixture (1:1, 20 mL) was added and the resulting mixture was extracted with EtOAc (3×50 mL). The combined organic layer was washed with saturated brine solution (20 mL), dried over anhydrous Na₂SO₄, filtered, and concentrated under reduced pressure. The product was purified by column chromatography on silica gel with a gradient of 0 to 10% MeOH in CHCl₃, which generated (52) (650 mg, 94% yield) as a white solid. ¹HNMR (600 MHz, DMSO-d₆) δ 8.31 (d, J=8.1 Hz, 1H), 7.42 (s, 1H), 6.85 (d, J=8.0 Hz, 1H), 4.41-4.31 (m, 1H), 3.98-3.87 (m, 1H), 3.60 (s, 3H), 3.13-3.04 (m, 2H), 2.28-2.13 (m, 2H), 1.89-1.80 (m, 1H), 1.77-1.68 (m, 1H), 1.69-1.56 (m, 2H), 1.56-1.48 (m, 1H), 1.36 (s, 9H), 1.42-1.28 (m, 3H), 0.87 (d, J=6.6 Hz, 1H), 0.84 (d, J=6.6 Hz, 1H).

Preparation of methyl N-(4-methoxy-1H-indole-2-carbonyl)-L-leucyl-3-[(3S)-2-oxopiperidin-3-yl]-L-alaninate, (53). A solution of methyl N-(tert-butoxycarbonyl)-L-leucyl-3-[(3S)-2-oxopiperidin-3-yl]-L-alaninate (52) (200 mg, 0.484 mmol) in DCM (5 mL) was cooled using an ice-water bath and then 4 M HCl in 1,4-dioxane (5 mL) was added. After 30 min, the ice bath was removed. After overnight, the mixture was concentrated under reduced pressure. The residue was treated with DCM/ether (1:1, 3×20 mL), and dried under reduced pressure to afford a white solid. The resulting residue was dissolved in in anhydrous DMF (20 mL) and the solution cooled in an ice-bath. To the solution was added 4-methoxy-1H-indole-2-carboxylic acid (8) (101 mg, 0.528 mmol), HATU (202 mg, 0.531 mmol), and then dropwise NMM (0.162 mL, 1.44 mmol). After 45 min, an ice/saturated aqueous NaHCO₃ mixture (1:1, 20 mL) was added and the resulting mixture was extracted with EtOAc (3×25 mL). The combined organic layer was washed with saturated brine solution (10 mL), dried over anhydrous Na₂SO₄, filtered, and concentrated under reduced pressure. The product was purified by column chromatography on silica gel with a gradient of 0 to 10% MeOH in CHCl₃, which generated (53) (231 mg, 98% yield) as a white solid. ¹HNMR (600 MHz, DMSO-d₆) δ 11.54 (d, J=2.1 Hz, 1H), 8.52 (d, J=8.0 Hz, 1H), 8.35 (d, J=8.1 Hz, 1H), 7.43 (s, 1H), 7.34 (dd, J=0.8, 2.3 Hz, 1H), 7.09 (dd, J=7.7, 8.2 Hz, 1H), 7.00 (d, J=8.2 Hz, 1H), 6.50 (d, J=7.7 Hz, 1H), 4.55-4.49 (m, 1H), 4.43-4.37 (m, 1H), 3.88 (s, 3H), 3.60 (s, 3H), 3.14-3.03 (m, 2H), 2.30-2.20 (m, 2H), 1.87-1.80 (m, 1H), 1.75-1.62 (m, 4H), 1.58-1.49 (m, 2H), 1.38-1.30 (m, 1H), 0.93 (d, J=6.5 Hz, 3H), 0.89 (d, J=6.5 Hz, 3H). LC/MS: Eluent system A (retention time: 4.95 min); ESI-MS 487 [M+H]⁺.

Preparation of N-[(2S)-1-({(1S)-1-cyano-2-[(3S)-2-oxopiperidin-3-yl]ethyl}amino)-4-methyl-1-oxopentan-2-yl]-4-methoxy-1H-indole-2-carboxamide, 34. To a solution of methyl N-(4-methoxy-1H-indole-2-carbonyl)-L-leucyl-3-[(3S)-2-oxopiperidin-3-yl]-L-alaninate (53) (200 mg, 0.411 mmol) in THF (5 mL) in an ice bath was added slowly a 1.0 M LiOH aqueous solution (5 mL). After 2 h, the pH of the reaction mixture was adjusted to 3 by adding 1.0 M HCl aqueous solution. The two layers were separated and the aqueous layer was extracted with EtOAc (3×25 mL). The combined organic phase was washed with brine (10 mL), dried over anhydrous Na₂SO₄, filtered, and concentrated under reduced pressure. The residue was dried under reduced pressure. To the residue was added anhydrous THF (10 mL) and then CDI (100 mg, 0.617 mmol). The mixture was stirred for 15 min before NH₃ (aq., 28%, 0.260 mL, 1.89 mmol) was added. The reaction mixture was then stirred for 1 h before it was quenched with water (10 mL). The mixture was extracted with EtOAc (3×25 mL). The combined organic phase was washed with brine (10 mL), dried over anhydrous Na₂SO₄, filtered, and concentrated under reduced pressure. Purification was accomplished by column chromatography on silica gel with a gradient of 0 to 10% MeOH in CHCl₃, which generated the amide intermediate (116 mg) as a white solid. To this amide (115 mg, 0.243 mmol) in anhydrous THF (10 mL) was added Et₃N (0.10 mL, 0.729 mmol) at 0° C., followed by addition of TFAA (102 mg, 0.486 mmol) slowly at this temperature. The progress of the reaction was monitored by TLC and once the intermediate disappeared after 1 h, water (10 mL) was added. The two layers were separated and the aqueous layer was extracted with EtOAc (3×25 mL). The combined organic phase was washed with brine (10 mL), dried over anhydrous Na₂SO₄, filtered, and concentrated under reduced pressure. The product was purified by column chromatography on silica gel with a gradient of 0 to 5% MeOH in CHCl₃, which generated 34 (28 mg, 15% yield for 3 steps) as a white solid. ¹HNMR (600 MHz, DMSO-d₆) δ 11.55 (d, J=2.1 Hz, 1H), 8.89 (d, J=8.1 Hz, 1H), 8.45 (d, J=7.8 Hz, 1H), 7.52 (s, 1H), 7.36 (dd, J=0.8, 2.3 Hz, 1H), 7.09 (dd, J=7.7, 8.2 Hz, 1H), 7.00 (d, J=8.2 Hz, 1H), 6.51 (d, J=7.7 Hz, 1H), 5.08-5.01 (m, 1H), 4.48-4.41 (m, 1H), 3.88 (s, 3H), 3.13-3.03 (m, 2H), 2.31-2.20 (m, 2H), 1.87-1.76 (m, 2H), 1.75-1.64 (m, 3H), 1.60-1.46 (m, 2H), 1.44-1.34 (m, 1H), 0.93 (d, J=6.4 Hz, 3H), 0.89 (d, J=6.5 Hz, 3H); ¹³CNMR (151 MHz, DMSO-d₆) δ 172.4, 172.0, 161.1, 153.6, 137.8, 129.8, 124.4, 119.8, 118.0, 105.4, 101.3, 99.2, 55.1, 51.3, 41.1, 40.1, 38.4, 36.9, 34.0, 26.0, 24.4, 23.0, 21.3, 21.1; LC/MS: Eluent system A (retention time: 4.75 min); ESI-MS: 454 [M+H]⁺; IR: 2244 cm⁻¹; HRMS (ESI+) calcd for C₂₄H₃₁N₅O₄+H 454.2454, found 454.2470.

### Compound 35

### Synthesis of 6-chloro-N-[(2S)-1-({(1S)-1-cyano-2-[(3S)-2-oxopiperidin-3-yl]ethyl}amino)-4,4-dimethyl-1-oxopentan-2-yl]-4-methoxy-1H-indole-2-carboxamide, 35

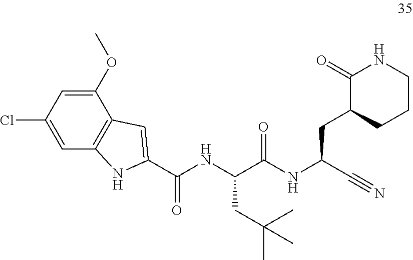 [SMILES: COc1cc(Cl)cc2[nH]c(C(=O)N[C@@H](CC(C)(C)C)C(=O)N[C@H](C#N)C[C@@H]3CCCNC3=O)cc12]

Compound 35 was synthesized as in Scheme 37.

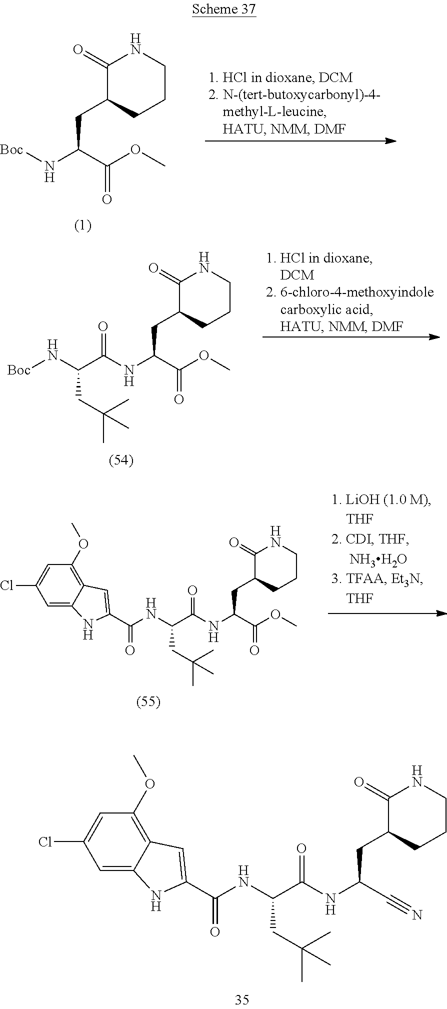 [SMILES: COC(=O)[C@H](C[C@@H]1CCCNC1=O)NC(=O)OC(C)(C)C ; COC(=O)[C@H](C[C@@H]1CCCNC1=O)NC(=O)[C@H](CC(C)(C)C)NC(=O)OC(C)(C)C ; COC(=O)[C@H](C[C@@H]1CCCNC1=O)NC(=O)[C@H](CC(C)(C)C)NC(=O)c1cc2c(OC)cc(Cl)cc2[nH]1 ; COc1cc(Cl)cc2[nH]c(C(=O)N[C@@H](CC(C)(C)C)C(=O)N[C@H](C#N)C[C@@H]3CCCNC3=O)cc12]

Preparation of methyl N-(tert-butoxycarbonyl)-4-methyl-L-leucyl-3-[(3S)-2-oxopiperidin-3-yl]-L-alaninate, (54). To a solution of methyl N-(tert-butoxycarbonyl)-3-[(3S)-2-oxopiperidin-3-yl]-L-alaninate (1) (200 mg, 0.667 mmol) in DCM (5 mL) cooled using an ice-water bath was added 4 M HCl in 1,4-dioxane (5 mL). After 30 min, the ice bath was removed. After overnight, the mixture was concentrated under reduced pressure. The residue was treated with DCM/ether (1:1, 3×20 mL), and dried under reduced pressure, which afforded 157 mg of a white solid. To the solid was added N-(tert-butoxycarbonyl)-4-methyl-L-leucine (182 mg, 0.742 mmol) and anhydrous DMF (10 mL) and the resulting solution was cooled in an ice bath. Then HATU (282 mg, 0.741 mmol) was added, followed by dropwise addition of NMM (0.220 mL, 2.01 mmol) dropwise. After 45 min, an ice/saturated aqueous NaHCO₃ mixture (1:1, 10 mL) was added and the resulting mixture was extracted with EtOAc (3×25 mL). The combined organic layer was washed with saturated brine solution (10 mL), dried over anhydrous Na₂SO₄, filtered, and concentrated under reduced pressure. The product was purified by column chromatography on silica gel with a gradient of 0 to 10% MeOH in CHCl₃, which generated (54) (260 mg, 91% yield) as a white solid. ¹HNMR (600 MHz, DMSO-d₆) δ 8.22 (d, J=8.0 Hz, 1H), 7.43 (s, 1H), 6.87 (d, J=8.5 Hz, 1H), 4.38-4.32 (m, 1H), 3.99 (td, J=8.5, 3.8 Hz, 1H), 3.59 (s, 3H), 3.14-3.04 (m, 2H), 2.28-2.15 (m, 2H), 1.88-1.80 (m, 1H), 1.77-1.69 (m, 1H), 1.67-1.61 (m, 1H), 1.56-1.41 (m, 3H), 1.37 (s, 9H), 1.36-1.31 (m, 1H), 0.89 (s, 9H).

Preparation of methyl N-(6-chloro-4-methoxy-1H-indole-2-carbonyl)-4-methyl-L-leucyl-3-[(3S)-2-oxopiperidin-3-yl]-L-alaninate, (55). A solution of methyl N-(tert-butoxycarbonyl)-4-methyl-L-leucyl-3-[(3S)-2-oxopiperidin-3-yl]-L-alaninate (54) (186 mg, 0.435 mmol) in DCM (5 mL) was cooled using an ice-water bath and then 4 M HCl in 1,4-dioxane (5 mL) was added. After 30 min, the ice bath was removed. After overnight, the mixture was concentrated under reduced pressure. The residue was treated with DCM/ether (1:1, 3×20 mL), and drying under reduced pressure. To the residue was added 6-chloro-4-methoxy-1H-indole-2-carboxylic acid (108 mg, 0.480 mmol) and anhydrous DMF (10 mL) and the solution was cooled in an ice bath. Then HATU (183 mg, 0.481 mmol) was added, followed by dropwise addition of NMM (0.150 mL, 1.32 mmol). After 45 min, an ice/saturated aqueous NaHCO₃ mixture (1:1, 10 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in EtOAc, which generated (55) (175 mg, 75% yield) as a white solid. ¹HNMR (600 MHz, DMSO-d₆) δ 11.70 (d, J=2.2 Hz, 1H), 8.48 (d, J=7.9 Hz, 1H), 8.45 (d, J=8.5 Hz, 1H), 7.43 (s, 1H), 7.34 (dd, J=0.8, 2.3 Hz, 1H), 7.04 (dd, J=0.8, 1.6 Hz, 1H), 6.55 (d, J=1.6 Hz, 1H), 4.57 (td, J=8.9, 3.4 Hz, 1H), 4.41-4.34 (m, 1H), 3.90 (s, 3H), 3.59 (s, 3H), 3.10-3.02 (m, 2H), 2.29-2.17 (m, 2H), 1.85-1.78 (m, 1H), 1.77-1.71 (m, 1H), 1.70-1.63 (m, 3H), 1.54-1.44 (m, 1H), 1.38-1.28 (m, 1H), 0.93 (s, 9H). LC/MS: Eluent system A (retention time: 6.28 min); ESI-MS 535 [M+H]⁺.

Preparation of 6-chloro-N-[(2S)-1-({(1S)-1-cyano-2-[(3S)-2-oxopiperidin-3-yl]ethyl}amino)-4,4-dimethyl-1-oxopentan-2-yl]-4-methoxy-1H-indole-2-carboxamide, 35. To a solution of methyl N-(6-chloro-4-methoxy-1H-indole-2-carbonyl)-4-methyl-L-leucyl-3-[(3S)-2-oxopiperidin-3-yl]-L-alaninate (55) (150 mg, 0.280 mmol) in THF (5 mL) in an ice bath was added a 1.0 M LiOH aqueous solution (5 mL). After 2 h, the pH of the reaction mixture was adjusted to 3 by adding 1.0 M HCl aqueous solution. The two layers were separated and the aqueous layer was extracted with EtOAc (3×25 mL). The combined organic phase was washed with brine (10 mL), dried over anhydrous Na₂SO₄, filtered, concentrated and dried under reduced pressure. To the residue was added THF (10 mL) and CDI (91.0 mg, 0.561 mmol). After 15 min, NH₃ (aq., 28%, 0.20 mL, 1.46 mmol) was added. After 1 h, water (10 mL) was added and the resulting mixture was extracted with EtOAc (3×25 mL). The combined organic phase was washed with brine (10 mL), dried over anhydrous Na₂SO₄, filtered, and concentrated under reduced pressure. Purification was accomplished by column chromatography on silica gel with a gradient of 0 to 10% MeOH in CHCl₃, which generated the intermediate (124 mg) as a white solid. To this solid (124 mg, 0.238 mmol) dissolved in anhydrous THF (10 mL) and cooled in an ice bath was added Et₃N (0.101 mL, 0.714 mmol), followed by slow addition of TFAA (101 mg, 0.480 mmol). The progress of the reaction was carefully monitored by TLC and once the intermediate disappeared after 1 h, water (10 mL) was added. The two layers were separated and the aqueous layer was extracted with EtOAc (3×25 mL). The combined organic phase was washed with brine (10 mL), dried over anhydrous Na₂SO₄, filtered, and concentrated under reduced pressure. The product was purified by column chromatography on silica gel with a gradient of 0 to 5% MeOH in CHCl₃, which generated 35 (59.4 mg, 43% yield over 3 steps) as a white solid. ¹HNMR (600 MHz, DMSO-d₆) δ 11.71 (d, J=2.2 Hz, 1H), 8.88 (d, J=8.0 Hz, 1H), 8.53 (d, J=8.0 Hz, 1H), 7.52 (s, 1H), 7.35 (dd, J=0.8, 2.3 Hz, 1H), 7.04 (dd, J=0.8, 1.6 Hz, 1H), 6.55 (d, J=1.6 Hz, 1H), 5.07-5.00 (m, 1H), 4.49 (td, J=8.6, 3.6 Hz, 1H), 3.91 (s, 3H), 3.12-3.01 (m, 2H), 2.30-2.20 (m, 2H), 1.84-1.73 (m, 3H), 1.71-1.63 (m, 2H), 1.57-1.48 (m, 1H), 1.42-1.33 (m, 1H), 0.93 (s, 9H); ¹³CNMR (151 MHz, DMSO-d₆) δ 172.5, 172.0, 160.4, 154.1, 137.3, 130.7, 128.7, 119.6, 116.8, 105.0, 101.3, 100.8, 55.6, 50.6, 44.2, 41.1, 38.5, 36.9, 33.9, 30.3, 29.5 (3C), 26.0, 21.1; LC/MS: Eluent system A (retention time: 6.05 min); ESI-MS: 502 [M+H]⁺; IR: 2245 cm⁻¹; HRMS (ESI+) calcd for C₂₅H₃₂ClN₅O₄+H 502.2221, found 502.2249.

### Compound 40

### Synthesis of N-[(2S)-1-({(1S)-1-cyano-2-[(3S)-2-oxopiperidin-3-yl]ethyl}amino)-4-methyl-1-oxopentan-2-yl]-4-(trifluoromethoxy)-1H-indole-2-carboxamide, 40

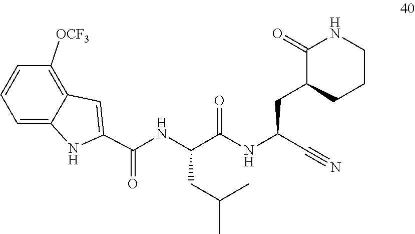 [SMILES: CC(C)C[C@H](NC(=O)c1cc2c(OC(F)(F)F)cccc2[nH]1)C(=O)N[C@H](C#N)C[C@@H]1CCCNC1=O]

Compound 40 was synthesized as in Scheme 38.

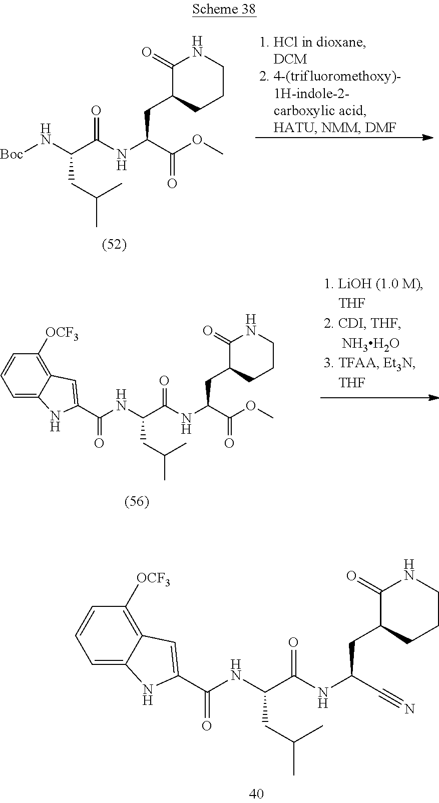 [SMILES: COC(=O)[C@H](C[C@@H]1CCCNC1=O)NC(=O)[C@H](CC(C)C)NC(=O)OC(C)(C)C ; COC(=O)[C@H](C[C@@H]1CCCNC1=O)NC(=O)[C@H](CC(C)C)NC(=O)c1cc2c(OC(F)(F)F)cccc2[nH]1 ; CC(C)C[C@H](NC(=O)c1cc2c(OC(F)(F)F)cccc2[nH]1)C(=O)N[C@H](C#N)C[C@@H]1CCCNC1=O]

Preparation of methyl N-[4-(trifluoromethoxy)-1H-indole-2-carbonyl]-L-leucyl-3-[(3S)-2-oxopiperidin-3-yl]-L-alaninate, (56). A solution of methyl N-(tert-butoxycarbonyl)-L-leucyl-3-[(3S)-2-oxopiperidin-3-yl]-L-alaninate (52) (177 mg, 0.428 mmol) in DCM (5 mL) was cooled using an ice-water bath and then 4 M HCl in 1,4-dioxane (5 mL) was added. After 30 min, the ice bath was removed. After overnight, the mixture was concentrated under reduced pressure. The residue was treated with DCM/ether (1:1, 3×20 mL), and dried under reduced pressure for 1 h. To the residue was added anhydrous DMF (10 mL) and 4-(trifluoromethoxy)-1H-indole-2-carboxylic acid (116 mg, 0.471 mmol) and the solution was cooled in an ice bath, after which HATU (179 mg, 0.471 mmol) was added followed by dropwise addition of NMM (0.141 mL, 1.28 mmol). After 45 min, an ice/saturated aqueous NaHCO₃ mixture (1:1, 10 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in CHCl₃, which generated (56) (165 mg, 71% yield) as a white solid. ¹HNMR (600 MHz, DMSO-d₆) δ 12.00 (d, J=1.8 Hz, 1H), 8.62 (d, J=8.1 Hz, 1H), 8.60 (d, J=8.0 Hz, 1H), 7.47-7.43 (m, 2H), 7.43 (s, 1H), 7.24 (dd, J=7.8, 8.0 Hz, 1H), 7.05-7.01 (m, 1H), 4.59-4.52 (m, 1H), 4.43-4.36 (m, 1H), 3.61 (s, 3H), 3.12-3.04 (m, 2H), 2.30-2.21 (m, 2H), 1.87-1.80 (m, 1H), 1.75-1.64 (m, 4H), 1.60-1.48 (m, 2H), 1.38-1.30 (m, 1H), 0.94 (d, J=6.5 Hz, 3H), 0.90 (d, J=6.5 Hz, 3H). LC/MS: Eluent system A (retention time: 6.40 min); ESI-MS: 541 [M+H]⁺.

Preparation of N-[(2S)-1-({(1S)-1-cyano-2-[(3S)-2-oxopiperidin-3-yl]ethyl}amino)-4-methyl-1-oxopentan-2-yl]-4-(trifluoromethoxy)-1H-indole-2-carboxamide, 40. To a solution of methyl N-[4-(trifluoromethoxy)-1H-indole-2-carbonyl]-L-leucyl-3-[(3S)-2-oxopiperidin-3-yl]-L-alaninate (56) (161 mg, 0.298 mmol) in THF (5 mL) cooled in an ice bath was slowly added a 1.0 M LiOH aqueous solution (5 mL). After 2 h, the pH of the reaction mixture was adjusted to 3 by adding 1.0 M HCl aqueous solution. The two layers were separated and the aqueous layer was extracted with EtOAc (3×25 mL). The combined organic phase was washed with brine (10 mL), dried over anhydrous Na₂SO₄, filtered, concentrated and dried under reduced pressure. To the residue was added THF (10 mL) and CDI (96.6 mg, 0.596 mmol). After 15 min, NH₃ (aq., 28%, 0.202 mL, 1.46 mmol) was added. After 1 h, water (10 mL) was added. The resulting mixture was extracted with EtOAc (3×25 mL). The combined organic phase was washed with brine (10 mL), dried over anhydrous Na₂SO₄, filtered, and concentrated under reduced pressure. Purification was accomplished by column chromatography on silica gel with a gradient of 0 to 10% MeOH in CHCl₃, which generated the intermediate (127 mg) as a white solid. To this intermediate (127 mg, 0.242 mmol) in anhydrous THF (10 mL) cooled in an ice bath was added Et₃N (0.101 mL, 0.726 mmol), followed by slow addition of a solution of TFAA (102 mg, 0.484 mmol) in anhydrous THF (2 mL). The progress of the reaction was monitored by TLC, and once the intermediate disappeared after 45 min, water (10 mL) was added. The two layers were separated and the aqueous layer was extracted with EtOAc (3×25 mL). The combined organic phase was washed with brine (10 mL), dried over anhydrous Na₂SO₄, filtered, and concentrated under reduced pressure. The product was purified by column chromatography on silica gel with a gradient of 0 to 5% MeOH in CHCl₃, which generated 40 (58.2 mg, 42% yield for 3 steps) as a white solid. ¹HNMR (600 MHz, DMSO-d₆) δ 12.02 (d, J=1.8 Hz, 1H), 8.95 (d, J=8.1 Hz, 1H), 8.71 (d, J=7.8 Hz, 1H), 7.52 (s, 1H), 7.47-7.44 (m, 2H), 7.25 (dd, J=7.8, 8.3 Hz, 1H), 7.03 (d, J=7.6 Hz, 1H), 5.08-5.02 (m, 1H), 4.51-4.44 (m, 1H), 3.14-3.02 (m, 2H), 2.31-2.21 (m, 2H), 1.87-1.76 (m, 2H), 1.76-1.64 (m, 3H), 1.61-1.50 (m, 2H), 1.44-1.34 (m, 1H), 0.94 (d, J=6.4 Hz, 3H), 0.89 (d, J=6.5 Hz, 3H); ¹⁹FNMR (565 MHz, DMSO-d₆) δ −56.44 (s, 3F); ¹³CNMR (151 MHz, DMSO-d6) δ 172.2, 172.0, 160.6, 141.7, 138.3, 132.3, 123.7, 121.4, 120.2 (q, J=123.5 Hz), 119.7, 112.0, 111.2, 99.6, 51.4, 41.1, 40.0, 38.4, 36.9, 34.0, 26.0, 24.4, 23.0, 21.3, 21.1; LC/MS: Eluent system A (retention time: 6.29 min); ESI-MS: 508 [M+H]⁺; IR: 2246 cm⁻¹; HRMS (ESI+) calcd for C₂₄H₂₈F₃N₅O₄+H 508.2172, found 508.2195.

### Compound 66

### Synthesis of N²-[(benzyloxy)carbonyl]-N-{(1S)-1-cyano-2-[(3S)-2-oxopiperidin-3-yl]ethyl}-L-leucinamide, 66

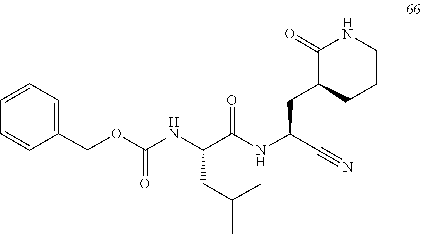 [SMILES: CC(C)C[C@H](NC(=O)OCc1ccccc1)C(=O)N[C@H](C#N)C[C@@H]1CCCNC1=O]

Compound 66 was synthesized as in Scheme 39.

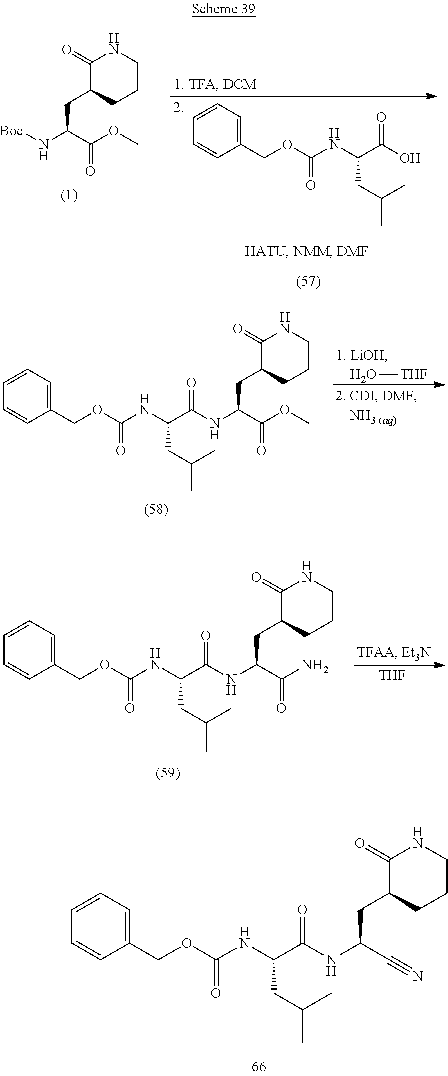

Preparation of methyl N-[(benzyloxy)carbonyl]-L-leucyl-3-[(3S)-2-oxopiperidin-3-yl]-L-alaninate, (58). To the ice-cooled solution of methyl N-(tert-butoxycarbonyl)-3-[(3S)-2-oxopiperidin-3-yl]-L-alaninate (1) (277 mg, 0.924 mmol) in DCM (5 mL) was added TFA (2 mL) and after 45 min the mixture was concentrated under reduced pressure. The residue was dissolved in DMF (5 mL), mixed with N-[(benzyloxy)carbonyl]-L-leucine (57) (524 mg, 1.98 mmol) and cooled in and ice bath. Then HATU (391 mg, 1.03 mmol) was added, followed by NMM (690 mg, 6.82 mmol). After 45 minutes the mixture was diluted with ethyl acetate (15 mL) and washed with 5% aqueous sodium bicarbonate solution (10 mL). The aqueous layer was extracted with ethyl acetate (15 mL), then the combined organic layer was washed with brine (10 mL), poured into a Petri dish and left overnight to evaporate in the fume hood. The resulting residue was dissolved in chloroform and loaded on silica gel column, the product was eluted with the 0% to 7% gradient of methanol in 1:1 mixture ethyl acetate-chloroform providing (58) (297 mg, 72% yield over two steps) as clear oil which solidified upon standing.

Preparation of N-[(benzyloxy)carbonyl]-L-leucyl-3-[(3S)-2-oxopiperidin-3-yl]-L-alaninamide, (59). A solution of methyl N-[(benzyloxy)carbonyl]-L-leucyl-3-[(3S)-2-oxopiperidin-3-yl]-L-alaninate (58) (272 mg, 0.608 mmol) in THF (5 mL) cooled in the ice bath was treated with 1M aqueous lithium hydroxide (3 mL). After 90 min, the mixture was acidified with 1M sodium bisulfate to pH of 2 and then extracted with ethyl acetate (3×15 mL). The combined organic layer was dried over sodium sulfate, filtered and concentrated under reduced pressure. The residue was dissolved in DMF (8 mL) and the resulting solution was cooled in an ice bath, after 15 minutes CDI (554 mg, 3.42 mmol) was added followed 25 min later by saturated ammonium hydroxide solution (442 mg, 9.26 mmol). After stirring for additional 45 minutes the mixture was diluted with ethyl acetate (25 mL), washed with water (10 mL) then saturated ammonium chloride solution (10 mL). The separated organic layer was dried with sodium sulfate, filtered and concentrated under reduced pressure. The product was purified by silica gel column chromatography (eluted with gradient of 0% to 20% of methanol in 1:1 chloroform:ethyl acetate mixture), which provided (59) (151 mg, 57% yield over two steps) as a white solid.

Preparation of N²-[(benzyloxy)carbonyl]-N-{(1S)-1-cyano-2-[(3S)-2-oxopiperidin-3-yl]ethyl}-L-leucinamide, 66. The conversion of the primary amide (59) (143 mg, 0.331 mmol) into the corresponding nitrile was performed as described for 24. Purification by silica gel column chromatography (eluted with gradient of 0% to 9% of methanol in 1:1 mixture ethyl acetate-chloroform) provided 66 (113 mg, 82% yield) as clear oil which solidified upon standing. ¹H NMR (600 MHz, DMSO-d₆) δ 8.85 (d, J=8.0 Hz, 1H), 7.56-7.50 (m, 2H), 7.39-7.26 (m, 5H), 5.04-4.98 (m, 3H), 4.01-3.96 (m, 1H), 3.13-3.04 (m, 2H), 2.31-2.18 (m, 2H), 1.85-1.67 (m, 3H), 1.65-1.45 (m, 3H), 1.42-1.34 (m, 2H), 0.89 (d, J=6.6 Hz, 3H), 0.86 (d, J=6.6 Hz, 3H). LC/MS: Eluent system B (retention time: 7.17 min); ESI-MS 415 [M+H]⁺, 413 [M−H]⁻.

### Compound 67

### Synthesis of N-{(1S)-1-cyano-2-[(3S)-2-oxopiperidin-3-yl]ethyl}-N²-{[(9H-fluoren-9-yl)methoxy]carbonyl}-L-leucinamide, 67

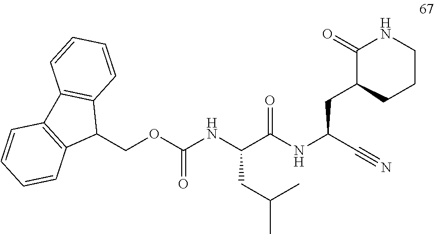 [SMILES: CC(C)C[C@H](NC(=O)OCC1c2ccccc2-c2ccccc21)C(=O)N[C@H](C#N)C[C@@H]1CCCNC1=O]

Compound 67 was synthesized as in Scheme 40.

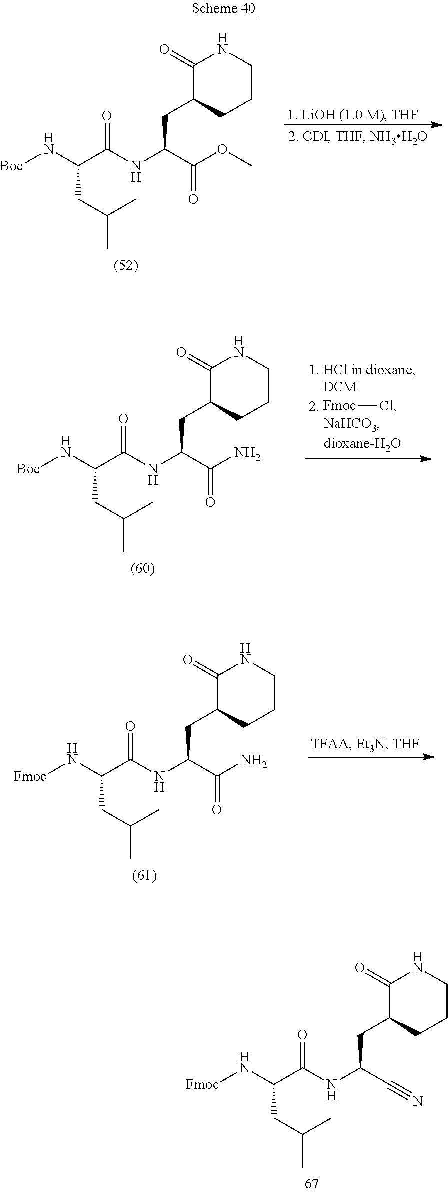

Preparation of N-(tert-butoxycarbonyl)-L-leucyl-3-[(3S)-2-oxopiperidin-3-yl]-L-alaninamide, (60). To a solution of methyl N-(tert-butoxycarbonyl)-L-leucyl-3-[(3S)-2-oxopiperidin-3-yl]-L-alaninate (52) (1.01 g, 2.44 mmol) in THF (20 mL) cooled in an ice bath was added a 1.0 M LiOH aqueous solution (20 mL). After 2 h, the pH of the reaction mixture was adjusted to 3 by adding 1.0 M HCl aqueous solution. The two layers were separated and the aqueous layer was extracted with EtOAc (3×50 mL). The combined organic phase was washed with brine (20 mL), dried over anhydrous Na₂SO₄, filtered, and concentrated under reduced pressure. To the residue was added anhydrous THF (30 mL) and CDI (791 mg, 4.88 mmol). After 15 min, NH₃ (aq., 28%, 3.81 mL, 27.6 mmol) was added. After 2 h, water (10 mL) was added, followed by addition of a 1.0 M HCl aqueous solution to adjust to pH of 6. The mixture was extracted with EtOAc (3×50 mL). The combined organic phase was washed with brine (15 mL), dried over anhydrous Na₂SO₄, filtered, and concentrated under reduced pressure. Purification was accomplished by silica gel column chromatography with a gradient of 0 to 10% MeOH in CHCl₃, which generated (60) (913 mg, 94% yield) as a white solid.

Preparation of N-{[(9H-fluoren-9-yl)methoxy]carbonyl}-L-leucyl-3-[(3S)-2-oxopiperidin-3-yl]-L-alaninamide, (61). A solution of N-(tert-butoxycarbonyl)-L-leucyl-3-[(3S)-2-oxopiperidin-3-yl]-L-alaninamide (60) (350 mg, 0.879 mmol) in DCM (7 mL) was cooled using an ice-water bath and then 4 M HCl in 1,4-dioxane (7 mL) was added. After 30 min, the ice bath was removed. After overnight, the mixture was concentrated under reduced pressure. The residue was treated with DCM/ether (1:1, 3×25 mL), and dried under reduced pressure for 1 h. To the residue was added dioxane (15 mL) and a solution of NaHCO₃ (222 mg, 2.64 mmol) in water (15 mL). To the mixture was slowly added a solution of fluorenylmethyloxycarbonyl chloride (Fmoc-Cl) (341 mg, 1.32 mmol) in dioxane (15 mL). After stirring for 15 min, the reaction mixture was diluted with water (10 mL) and extracted with EtOAc (3×25 mL). The combined organic phase was washed with brine (25 mL), dried over anhydrous Na₂SO₄, filtered, and concentrated under reduced pressure. The product was purified by column chromatography on silica gel with a gradient of 0 to 5% MeOH in CHCl₃, which generated (61) (406 mg, 89% yield) as a white solid.

Preparation of N-{(1S)-1-cyano-2-[(3S)-2-oxopiperidin-3-yl]ethyl}-N²-{[(9H-fluoren-9-yl)methoxy]carbonyl}-L-leucinamide, 67. To a solution of N-{[(9H-fluoren-9-yl)methoxy]carbonyl}-L-leucyl-3-[(3S)-2-oxopiperidin-3-yl]-L-alaninamide (61) (401 mg, 0.770 mmol) in anhydrous THF (20 mL) cooled in an ice bath was added Et₃N (0.322 mL, 2.31 mmol), followed by slow addition of TFAA (323 mg, 1.54 mmol). The progress of the reaction was monitored by TLC, and once the intermediate disappeared, after 15 min, water (10 mL) was added. The two layers were separated and the aqueous layer was extracted with EtOAc (3×25 mL). The combined organic phase was washed with brine (10 mL), dried over anhydrous Na₂SO₄, filtered, and concentrated under reduced pressure. The product was purified by column chromatography on silica gel with a gradient of 0 to 5% MeOH in CHCl₃, which generated 67 (353 mg, 91% yield) as a white solid. ¹HNMR (600 MHz, DMSO-d₆) δ 8.83 (d, J=8.0 Hz, 1H), 7.89 (d, J=7.6 Hz, 2H), 7.71 (t, J=7.1 Hz, 2H), 7.60 (d, J=8.0 Hz, 1H), 7.51 (s, 1H), 7.43-7.40 (m, 2H), 7.34-7.30 (m, 2H), 5.04-4.98 (m, 1H), 4.32-4.27 (m, 1H), 4.26-4.19 (m, 2H), 4.02-3.95 (m, 1H), 3.10-3.00 (m, 2H), 2.29-2.16 (m, 2H), 1.84-1.72 (m, 2H), 1.70-1.57 (m, 2H), 1.55-1.46 (m, 2H), 1.43-1.33 (m, 2H), 0.90 (d, J=6.6 Hz, 3H), 0.86 (d, J=6.5 Hz, 3H); LC/MS: Eluent system A (retention time: 6.43 min); ESI-MS: 503 [M+H]⁺.

### Compound 68

### Synthesis of 6-chloro-N-[(2S)-1-({(1S)-1-cyano-2-[(3S)-2-oxopiperidin-3-yl]ethyl}amino)-4-methyl-1-oxopentan-2-yl]-4-methoxy-1H-indole-2-carboxamide, 68

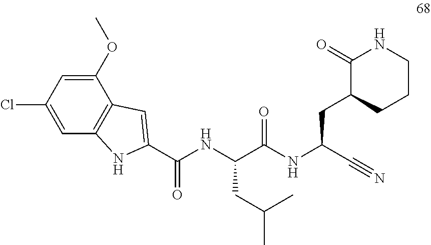 [SMILES: COc1cc(Cl)cc2[nH]c(C(=O)N[C@@H](CC(C)C)C(=O)N[C@H](C#N)C[C@@H]3CCCNC3=O)cc12]

Compound 68 was synthesized as in Scheme 41.

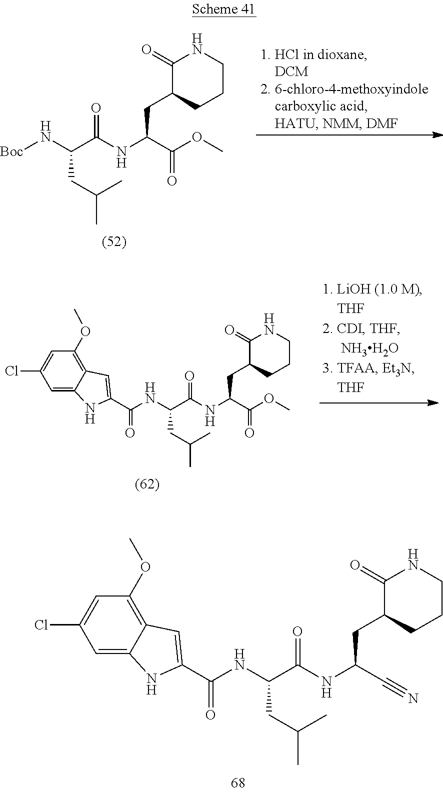 [SMILES: COC(=O)[C@H](C[C@@H]1CCCNC1=O)NC(=O)[C@H](CC(C)C)NC(=O)OC(C)(C)C ; COC(=O)[C@H](C[C@@H]1CCCNC1=O)NC(=O)[C@H](CC(C)C)NC(=O)c1cc2c(OC)cc(Cl)cc2[nH]1 ; COc1cc(Cl)cc2[nH]c(C(=O)N[C@@H](CC(C)C)C(=O)N[C@H](C#N)C[C@@H]3CCCNC3=O)cc12]

Preparation of methyl N-(6-chloro-4-methoxy-1H-indole-2-carbonyl)-L-leucyl-3-[(3S)-2-oxopiperidin-3-yl]-L-alaninate, (62). A solution of methyl N-(tert-butoxycarbonyl)-L-leucyl-3-[(3S)-2-oxopiperidin-3-yl]-L-alaninate (52) (201 mg, 0.486 mmol) in DCM (5 mL) was cooled using an ice-water bath and then 4 M HCl in 1,4-dioxane (5 mL) was added. After 30 min, the ice bath was removed. After overnight, the mixture was concentrated under reduced pressure. The residue was treated with DCM/ether (1:1, 3×20 mL), and dried under reduced pressure for 1 h. To the residue was added anhydrous DMF (10 mL) and 6-chloro-4-methoxy-1H-indole-2-carboxylic acid (120 mg, 0.532 mmol) and the solution was cooled in an ice bath. To the cooled solution was added HATU (202 mg, 0.531 mmol), followed by dropwise addition of NMM (0.160 mL, 1.46 mmol). After 45 min, an ice/saturated aqueous NaHCO₃ mixture (1:1, 10 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in CHCl₃, which generated (62) (185 mg, 73% yield) as a white solid. ¹HNMR (600 MHz, DMSO-d₆) δ 11.69 (d, J=2.1 Hz, 1H), 8.54 (d, J=8.0 Hz, 1H), 8.42 (d, J=8.1 Hz, 1H), 7.43 (s, 1H), 7.36 (dd, J=0.8, 2.3 Hz, 1H), 7.03 (dd, J=0.8, 1.6 Hz, 1H), 6.55 (d, J=1.6 Hz, 1H), 4.54-4.48 (m, 1H), 4.42-4.36 (m, 1H), 3.91 (s, 3H), 3.60 (s, 3H), 3.13-3.03 (m, 2H), 2.29-2.20 (m, 2H), 1.87-1.80 (m, 1H), 1.75-1.62 (m, 4H), 1.57-1.46 (m, 2H), 1.38-1.29 (m, 1H), 0.93 (d, J=6.4 Hz, 3H), 0.89 (d, J=6.5 Hz, 3H). LC/MS: Eluent system A (retention time: 6.01 min); ESI-MS: 521 [M+H]⁺.

Preparation of 6-chloro-N-[(2S)-1-({(1S)-1-cyano-2-[(3S)-2-oxopiperidin-3-yl]ethyl}amino)-4-methyl-1-oxopentan-2-yl]-4-methoxy-1H-indole-2-carboxamide, 68. To a solution of N-(6-chloro-4-methoxy-1H-indole-2-carbonyl)-L-leucyl-3-[(3S)-2-oxopiperidin-3-yl]-L-alaninate (62) (140 mg, 0.270 mmol) in THF (5 mL) cooled in an ice bath was slowly added a 1.0 M LiOH aqueous solution (5 mL). After 2 h, the pH of the reaction mixture was adjusted to 3 by adding 1.0 M HCl aqueous solution. The two layers were separated and the aqueous layer was extracted with EtOAc (3×25 mL). The combined organic phase was washed with brine (10 mL), dried over anhydrous Na₂SO₄, filtered, and concentrated under reduced pressure. To the residue was added THF (10 mL) and CDI (88.2 mg, 0.544 mmol). After 15 min, NH₃ (aq., 28%, 0.201 mL, 1.46 mmol) was added. After 1 h, water (10 mL) was added and the resulting mixture was extracted with EtOAc (3×25 mL). The combined organic phase was washed with brine (10 mL), dried over anhydrous Na₂SO₄, filtered, and concentrated under reduced pressure. Purification was accomplished by column chromatography on silica gel with a gradient of 0 to 10% MeOH in CHCl₃, which generated the intermediate (108 mg) as a white solid. To this intermediate (106 mg, 0.238 mmol) in anhydrous THF (10 mL) cooled in an ice bath was added Et₃N (88.0 μL, 0.631 mmol), followed by slow addition of TFAA (88.4 mg, 0.421 mmol). The progress of the reaction was monitored by TLC and once the intermediate disappeared after 1 h, water (10 mL) was added. The two layers were separated and the aqueous layer was extracted with EtOAc (3×25 mL). The combined organic phase was washed with brine (10 mL), dried over anhydrous Na₂SO₄, filtered, and concentrated under reduced pressure. The product was purified by column chromatography on silica gel with a gradient of 0 to 5% MeOH in CHCl₃, which generated 68 (16.3 mg, 12% yield for 3 steps) as a white solid. ¹HNMR (600 MHz, DMSO-d₆) δ 11.71 (d, J=2.1 Hz, 1H), 8.90 (d, J=8.1 Hz, 1H), 8.52 (d, J=7.8 Hz, 1H), 7.52 (s, 1H), 7.38 (dd, J=0.8, 2.3 Hz, 1H), 7.04 (dd, J=0.9, 1.5 Hz, 1H), 6.56 (d, J=1.5 Hz, 1H), 5.07-5.01 (m, 1H), 4.47-4.41 (m, 1H), 3.91 (s, 3H), 3.14-3.03 (m, 2H), 2.30-2.20 (m, 2H), 1.86-1.75 (m, 2H), 1.74-1.63 (m, 3H), 1.60-1.46 (m, 2H), 1.44-1.34 (m, 1H), 0.93 (d, J=6.4 Hz, 3H), 0.88 (d, J=6.5 Hz, 3H); ¹³CNMR (151 MHz, DMSO-d₆) δ 172.3, 172.0, 160.8, 154.1, 137.3, 130.5, 128.7, 119.8, 116.8, 105.0, 101.3, 100.8, 55.6, 51.4, 41.1, 40.0, 38.4, 36.9, 34.0, 26.0, 24.4, 23.0, 21.3, 21.1; LC/MS: Eluent system A (retention time: 5.87 min); ESI-MS: 488 [M+H]⁺; IR: 2244 cm⁻¹; HRMS (ESI+) calcd for C₂₄H₃₀ClN₅O₄+H 488.2065, found 488.2079.

### Compound 69

### Synthesis of N-[(2S)-1-({(1S)-1-cyano-2-[(3S)-2-oxopiperidin-3-yl]ethyl}amino)-4-methyl-1-oxopentan-2-yl]-4-ethoxy-1H-indole-2-carboxamide, 69

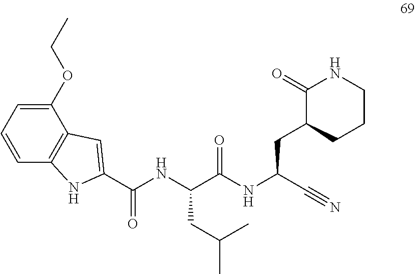 [SMILES: CCOc1cccc2[nH]c(C(=O)N[C@@H](CC(C)C)C(=O)N[C@H](C#N)C[C@@H]3CCCNC3=O)cc12]

Compound 69 was synthesized as in Scheme 42.

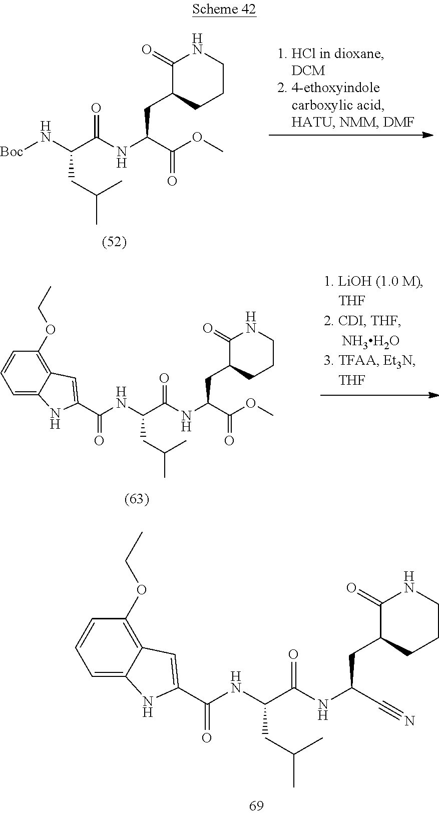 [SMILES: COC(=O)[C@H](C[C@@H]1CCCNC1=O)NC(=O)[C@H](CC(C)C)NC(=O)OC(C)(C)C ; CCOc1cccc2[nH]c(C(=O)N[C@@H](CC(C)C)C(=O)N[C@@H](C[C@@H]3CCCNC3=O)C(=O)OC)cc12 ; CCOc1cccc2[nH]c(C(=O)N[C@@H](CC(C)C)C(=O)N[C@H](C#N)C[C@@H]3CCCNC3=O)cc12]

Preparation of methyl N-(4-ethoxy-1H-indole-2-carbonyl)-L-leucyl-3-[(3S)-2-oxopiperidin-3-yl]-L-alaninate, (63). A solution of methyl N-(tert-butoxycarbonyl)-L-leucyl-3-[(3S)-2-oxopiperidin-3-yl]-L-alaninate (52) (175 mg, 0.423 mmol) in DCM (5 mL) was cooled using an ice-water bath and then 4 M HCl in 1,4-dioxane (5 mL) was added. After 30 min, the ice bath was removed. After overnight, the mixture was concentrated under reduced pressure. The residue was treated with DCM/ether (1:1, 3×20 mL), and dried under reduced pressure for 1 h. To the residue was added anhydrous DMF (10 mL) and 4-ethoxy-1H-indole-2-carboxylic acid (94.4 mg, 0.460 mmol) and the solution was cooled in an ice bath upon which HATU (175 mg, 0.459 mmol) was added, followed by dropwise addition of NMM (0.140 mL, 1.26 mmol). After 45 min, an ice/saturated aqueous NaHCO₃ mixture (1:1, 10 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in EtOAc, which generated (63) (165 mg, 78% yield) as a white solid. ¹HNMR (600 MHz, DMSO-d₆) δ 11.51 (d, J=2.1 Hz, 1H), 8.49 (d, J=8.0 Hz, 1H), 8.39 (d, J=8.2 Hz, 1H), 7.43 (s, 1H), 7.36 (dd, J=0.8, 2.3 Hz, 1H), 7.06 (dd, J=7.7, 8.3 Hz, 1H), 6.99 (d, J=8.3 Hz, 1H), 6.48 (d, J=7.6 Hz, 1H), 4.55-4.49 (m, 1H), 4.43-4.37 (m, 1H), 4.14 (q, J=7.0 Hz, 2H), 3.60 (s, 3H), 3.13-3.03 (m, 2H), 2.30-2.20 (m, 2H), 1.87-1.80 (m, 1H), 1.73-1.63 (m, 4H), 1.57-1.50 (m, 2H), 1.41 (t, J=7.0 Hz, 3H), 1.38-1.30 (m, 1H), 0.93 (d, J=6.4 Hz, 3H), 0.89 (d, J=6.5 Hz, 3H). LC/MS: Eluent system A (retention time: 5.71 min); ESI-MS: 501 [M+H]⁺.

Preparation of N-[(2S)-1-({(1S)-1-cyano-2-[(3S)-2-oxopiperidin-3-yl]ethyl}amino)-4-methyl-1-oxopentan-2-yl]-4-ethoxy-1H-indole-2-carboxamide, 69. To a solution of methyl N-(4-ethoxy-1H-indole-2-carbonyl)-L-leucyl-3-[(3S)-2-oxopiperidin-3-yl]-L-alaninate (63) (145 mg, 0.290 mmol) in THF (5 mL) cooled in an ice bath was slowly added a 1.0 M LiOH aqueous solution (5 mL). After 2 h, the pH of the reaction mixture was adjusted to 3 by adding 1.0 M HCl aqueous solution. The two layers were separated and the aqueous layer was extracted with EtOAc (3×25 mL). The combined organic phase was washed with brine (10 mL), dried over anhydrous Na₂SO₄, filtered, concentrated and dried under reduced pressure. To residue was added THF (10 mL) and CDI (94.0 mg, 0.580 mmol). After 15 min, NH₃ (aq., 28%, 0.220 mL, 1.60 mmol) was added. After 1 h, water (10 mL) was added and the resulting mixture was extracted with EtOAc (3×25 mL). The combined organic phase was washed with brine (10 mL), dried over anhydrous Na₂SO₄, filtered, and concentrated under reduced pressure. Purification was accomplished by column chromatography on silica gel with a gradient of 0 to 10% MeOH in CHCl₃, which generated the intermediate (121 mg) as a white solid. To this intermediate (120 mg, 0.247 mmol) in anhydrous THF (10 mL) cooled in an ice bath was added Et₃N (0.101 mL, 0.741 mmol), followed by slow addition of TFAA (104 mg, 0.494 mmol). After 1 h, water (10 mL) was added. The two layers were separated and the aqueous layer was extracted with EtOAc (3×25 mL). The combined organic phase was washed with brine (10 mL), dried over anhydrous Na₂SO₄, filtered, and concentrated under reduced pressure. The product was purified by column chromatography on silica gel with a gradient of 0 to 5% MeOH in CHCl₃, which generated 69 (36.8 mg, 27% yield for 3 steps) as a light yellow solid. ¹HNMR (600 MHz, DMSO-d₆) δ 11.53 (d, J=2.1 Hz, 1H), 8.87 (d, J=8.0 Hz, 1H), 8.49 (d, J=7.8 Hz, 1H), 7.52 (s, 1H), 7.38 (dd, J=0.8, 2.3 Hz, 1H), 7.07 (dd, J=7.8, 8.2 Hz, 1H), 6.99 (d, J=8.2 Hz, 1H), 6.48 (d, J=7.6 Hz, 1H), 5.08-5.01 (m, 1H), 4.48-4.41 (m, 1H), 4.14 (q, J=7.0 Hz, 2H), 3.13-3.03 (m, 2H), 2.32-2.20 (m, 2H), 1.86-1.76 (m, 2H), 1.75-1.64 (m, 3H), 1.60-1.46 (m, 2H), 1.41 (t, J=7.0 Hz, 3H), 1.44-1.34 (m, 1H), 0.93 (d, J=6.5 Hz, 3H), 0.89 (d, J=6.5 Hz, 3H); ¹³CNMR (151 MHz, DMSO-d₆) δ 172.4, 172.0, 161.1, 152.9, 137.9, 129.7, 124.4, 119.8, 118.2, 105.3, 101.3, 100.0, 62.9, 51.3, 41.1, 40.0, 38.4, 36.9, 34.0, 26.0, 24.4, 23.0, 21.3, 21.1, 14.9; LC/MS: Eluent system A (retention time: 5.57 min); ESI-MS: 468 [M+H]⁺; IR: 2247 cm⁻¹; HRMS (ESI+) calcd for C₂₅H₃₃N₅O₄+H 468.2611, found 468.2629.

### Compound 70

### Synthesis of N-[(2S)-1-({(1S)-1-cyano-2-[(3S)-2-oxopiperidin-3-yl]ethyl}amino)-4,4-dimethyl-1-oxopentan-2-yl]-4-methoxy-1H-indole-2-carboxamide, 70

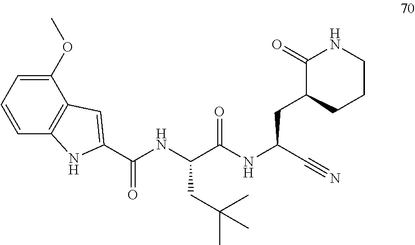 [SMILES: COc1cccc2[nH]c(C(=O)N[C@@H](CC(C)(C)C)C(=O)N[C@H](C#N)C[C@@H]3CCCNC3=O)cc12]

Compound 70 was synthesized as in Scheme 43.

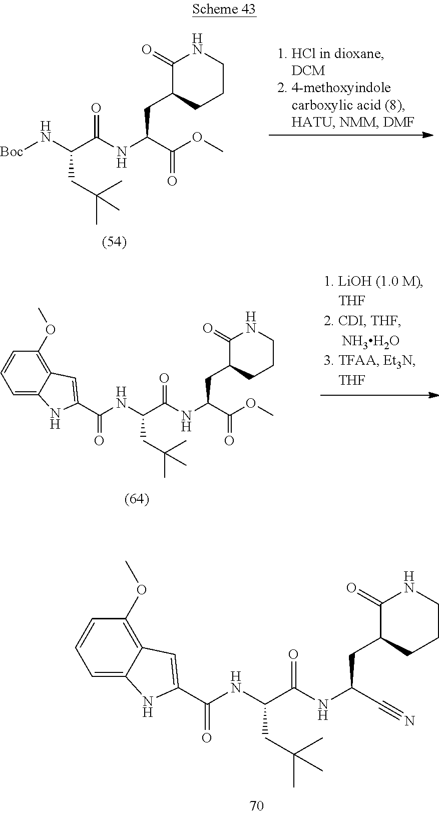 [SMILES: COC(=O)[C@H](C[C@@H]1CCCNC1=O)NC(=O)[C@H](CC(C)(C)C)NC(=O)OC(C)(C)C ; COC(=O)[C@H](C[C@@H]1CCCNC1=O)NC(=O)[C@H](CC(C)(C)C)NC(=O)c1cc2c(OC)cccc2[nH]1 ; COc1cccc2[nH]c(C(=O)N[C@@H](CC(C)(C)C)C(=O)N[C@H](C#N)C[C@@H]3CCCNC3=O)cc12]

Preparation of methyl N-(4-methoxy-1H-indole-2-carbonyl)-4-methyl-L-leucyl-3-[(3S)-2-oxopiperidin-3-yl]-L-alaninate, (64). A solution of methyl N-(tert-butoxycarbonyl)-4-methyl-L-leucyl-3-[(3S)-2-oxopiperidin-3-yl]-L-alaninate (54) (200 mg, 0.467 mmol) in DCM (5 mL) was cooled using an ice-water bath and then 4 M HCl in 1,4-dioxane (5 mL) was added. After 30 min, the ice bath was removed. After overnight, the mixture was concentrated under reduced pressure. The residue was treated with DCM/ether (1:1, 3×20 mL), and dried under reduced pressure for 1 h. To the resulting residue was added anhydrous DMF (10 mL) followed by 4-methoxy-1H-indole-2-carboxylic acid (8) (99.9 mg, 0.523 mmol) and after the solution was cooled in an ice bath, HATU (198 mg, 0.521 mmol) was added followed by dropwise addition of NMM (0.163 mL, 1.48 mmol). After 45 min, an ice/saturated aqueous NaHCO₃ mixture (1:1, 10 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in CHCl₃, which generated (64) (207 mg, 89% yield) as a white solid. ¹HNMR (600 MHz, DMSO-d₆) δ 11.55 (d, J=2.1 Hz, 1H), 8.45 (d, J=8.0 Hz, 1H), 8.38 (d, J=8.4 Hz, 1H), 7.43 (s, 1H), 7.31 (dd, J=0.8, 2.3 Hz, 1H), 7.09 (dd, J=7.7, 8.3 Hz, 1H), 7.00 (d, J=8.2 Hz, 1H), 6.50 (d, J=7.7 Hz, 1H), 4.58 (td, J=8.9, 3.3 Hz, 1H), 4.41-4.35 (m, 1H), 3.88 (s, 3H), 3.59 (s, 3H), 3.12-3.02 (m, 2H), 2.29-2.18 (m, 2H), 1.86-1.79 (m, 1H), 1.79-1.71 (m, 1H), 1.71-1.63 (m, 3H), 1.55-1.44 (m, 1H), 1.38-1.29 (m, 1H), 0.94 (s, 9H). LC/MS: Eluent system A (retention time: 5.31 min); ESI-MS: 501 [M+H]⁺.

Preparation of N-[(2S)-1-({(1S)-1-cyano-2-[(3S)-2-oxopiperidin-3-yl]ethyl}amino)-4,4-dimethyl-1-oxopentan-2-yl]-4-methoxy-1H-indole-2-carboxamide, 70. To a solution of methyl N-(4-methoxy-1H-indole-2-carbonyl)-4-methyl-L-leucyl-3-[(3S)-2-oxopiperidin-3-yl]-L-alaninate (64) (192 mg, 0.384 mmol) in THF (5 mL) cooled in an ice bath was slowly added a 1.0 M LiOH aqueous solution (5 mL). After 2 h, the pH of the reaction mixture was adjusted to 3 by adding 1.0 M HCl aqueous solution. The two layers were separated and the aqueous layer was extracted with EtOAc (3×25 mL). The combined organic phase was washed with brine (10 mL), dried over anhydrous Na₂SO₄, filtered, concentrated and dried under reduced pressure. To the residue was added THF (10 mL) and CDI (124 mg, 0.765 mmol). After 15 min, NH₃ (aq., 28%, 0.260 mL, 1.89 mmol) was added. After 1 h, water (10 mL) was added and the mixture was extracted with EtOAc (3×25 mL). The combined organic phase was washed with brine (10 mL), dried over anhydrous Na₂SO₄, filtered, and concentrated under reduced pressure. Purification was accomplished by column chromatography on silica gel with a gradient of 0 to 10% MeOH in CHCl₃, which generated the intermediate (161 mg, 86% yield) as a white solid. To this intermediate (159 mg, 0.327 mmol) was added anhydrous THF (10 mL) and the solution was cooled in an icebath and then Et₃N (0.142 mL, 1.02 mmol) was added, followed by slow addition of TFAA (137 mg, 0.654 mmol). The progress of the reaction mixture was monitored by TLC, and once the intermediate disappeared after 1 h, water (10 mL) was added. The two layers were separated and the aqueous layer was extracted with EtOAc (3×25 mL). The combined organic phase was washed with brine (10 mL), dried over anhydrous Na₂SO₄, filtered, and concentrated under reduced pressure. The product was purified by column chromatography on silica gel with a gradient of 0 to 5% MeOH in CHCl₃, which generated the 70 (26.7 mg, 17% yield) as a white solid. ¹HNMR (600 MHz, DMSO-d₆) δ 11.56 (d, J=2.1 Hz, 1H), 8.87 (d, J=8.1 Hz, 1H), 8.45 (d, J=8.1 Hz, 1H), 7.51 (s, 1H), 7.33 (dd, J=0.8, 2.3 Hz, 1H), 7.09 (dd, J=7.8, 8.2 Hz, 1H), 7.00 (d, J=8.2 Hz, 1H), 6.50 (d, J=7.8 Hz, 1H), 5.07-5.00 (m, 1H), 4.50 (td, J=8.7, 3.6 Hz, 1H), 3.88 (s, 3H), 3.12-3.02 (m, 2H), 2.30-2.20 (m, 2H), 1.84-1.74 (m, 3H), 1.71-1.63 (m, 2H), 1.58-1.49 (m, 1H), 1.42-1.33 (m, 1H), 0.94 (s, 9H). LC/MS: Eluent system A (retention time: 5.31 min); ESI-MS: 468 [M+H]⁺.

### Compound 71

### Synthesis of N-[(2S)-1-({(1S)-1-cyano-2-[(3S)-2-oxopiperidin-3-yl]ethyl}amino)-4-methyl-1-oxopentan-2-yl]-3,6-dihydro-2H-furo[2,3-e]indole-7-carboxamide, 71

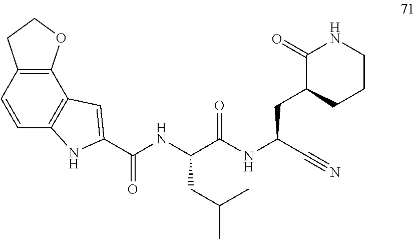 [SMILES: CC(C)C[C@H](NC(=O)c1cc2c3c(ccc2[nH]1)CCO3)C(=O)N[C@H](C#N)C[C@@H]1CCCNC1=O]

Compound 71 was synthesized as in Scheme 44.

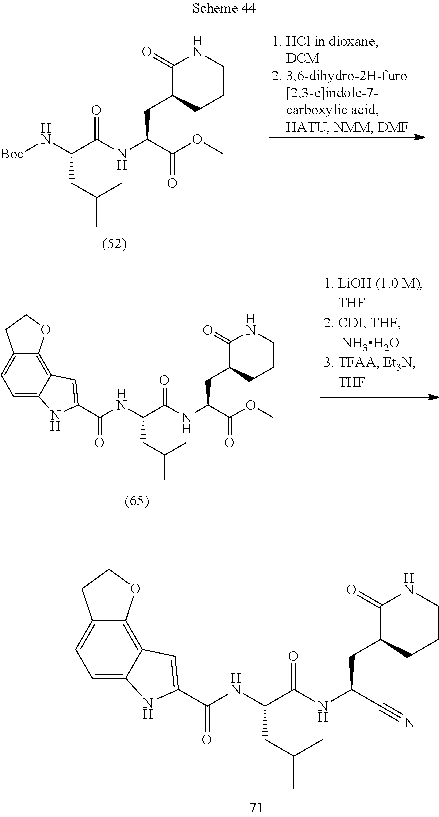 [SMILES: COC(=O)[C@H](C[C@@H]1CCCNC1=O)NC(=O)[C@H](CC(C)C)NC(=O)OC(C)(C)C ; COC(=O)[C@H](C[C@@H]1CCCNC1=O)NC(=O)[C@H](CC(C)C)NC(=O)c1cc2c3c(ccc2[nH]1)CCO3 ; CC(C)C[C@H](NC(=O)c1cc2c3c(ccc2[nH]1)CCO3)C(=O)N[C@H](C#N)C[C@@H]1CCCNC1=O]

Preparation of methyl N-(3,6-dihydro-2H-furo[2,3-e]indole-7-carbonyl)-L-leucyl-3-[(3S)-2-oxopiperidin-3-yl]-L-alaninate, (65). A solution of methyl N-(tert-butoxycarbonyl)-L-leucyl-3-[(3S)-2-oxopiperidin-3-yl]-L-alaninate (52) (178 mg, 0.430 mmol) in DCM (5 mL) was cooled using an ice-water bath and then 4 M HCl in 1,4-dioxane (5 mL) was added. After 30 min, the ice bath was removed. After overnight, the mixture was concentrated under reduced pressure. The residue was treated with DCM/ether (1:1, 3×20 mL), and dried under reduced pressure for 1 h. To the residue was added anhydrous DMF (10 mL) and 3,6-dihydro-2H-furo[2,3-e]indole-7-carboxylic acid (78.9 mg, 0.388 mmol) [prepared using procedures in Velazquez, F. et al Org. Lett. 2012, 14 (2), 556-559 and Patent US2005/0026987] and the solution was cooled in an ice bath after which HATU (221 mg, 0.582 mmol) was added followed by dropwise addition of NMM (0.130 mL, 1.17 mmol). After 45 min, an ice/saturated aqueous NaHCO₃ mixture (1:1, 10 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in CHCl₃, which generated (65) (65 mg, 30% yield) as a white solid. LC/MS: Eluent system A (retention time: 5.11 min); ESI-MS: 499 [M+H]⁺.

Preparation of N-[(2S)-1-({(1S)-1-cyano-2-[(3S)-2-oxopiperidin-3-yl]ethyl}amino)-4-methyl-1-oxopentan-2-yl]-3,6-dihydro-2H-furo[2,3-e]indole-7-carboxamide, 71. To a solution of methyl N-(3,6-dihydro-2H-furo[2,3-e]indole-7-carbonyl)-L-leucyl-3-[(3S)-2-oxopiperidin-3-yl]-L-alaninate (65) (65.0 mg, 0.130 mmol) in THF (4 mL) cooled in an ice bath was added a 1.0 M LiOH aqueous solution (4 mL). After 2 h, the pH of the reaction mixture was adjusted to 3 by adding 1.0 M HCl aqueous solution. The two layers were separated and the aqueous layer was extracted with EtOAc (3×25 mL). The combined organic phase was washed with brine (10 mL), dried over anhydrous Na₂SO₄, filtered, concentrated and dried under reduced pressure. To the residue was added THF (10 mL) and CDI (42.2 mg, 0.260 mmol). After 15 min, NH₃ (aq., 28%, 0.100 mL, 0.728 mmol) was added. After 1 h, water (10 mL) was added and the mixture was extracted with EtOAc (3×25 mL). The combined organic phase was washed with brine (10 mL), dried over anhydrous Na₂SO₄, filtered, and concentrated under reduced pressure. Purification was accomplished by column chromatography on silica gel with a gradient of 0 to 10% MeOH in CHCl₃, which generated the intermediate (49.2 mg) as a white solid. To this intermediate (49.2 mg, 0.101 mmol) was added THF (5 mL) and the solution was cooled in an ice bath. Then Et₃N (42.0 μL, 0.303 mmol) was added followed by slow addition of a solution of TFAA (42.4 mg, 0.202 mmol) in anhydrous THF (2 mL). The progress of the reaction was monitored by TLC, and once the intermediate disappeared after 30 min, water (10 mL) was added. The two layers were separated and the aqueous layer was extracted with EtOAc (3×25 mL). The combined organic phase was washed with brine (10 mL), dried over anhydrous Na₂SO₄, filtered, and concentrated under reduced pressure. The product was purified by column chromatography on silica gel with a gradient of 0 to 10% MeOH in EtOAc, which generated 71 (38.2 mg, 63% yield for 3 steps) as a white solid. ¹HNMR (600 MHz, DMSO-d₆) δ 11.52 (d, J=2.1 Hz, 1H), 8.91 (d, J=8.1 Hz, 1H), 8.46 (d, J=7.8 Hz, 1H), 7.52 (s, 1H), 7.17 (dd, J=0.8, 2.3 Hz, 1H), 7.05 (d, J=8.2 Hz, 1H), 6.91 (dd, J=0.8, 8.1 Hz, 1H), 5.08-5.02 (m, 1H), 4.69-4.60 (m, 2H), 4.50-4.41 (m, 1H), 3.22 (t, J=8.7 Hz, 2H), 3.13-3.03 (m, 2H), 2.32-2.20 (m, 2H), 1.87-1.75 (m, 2H), 1.74-1.64 (m, 3H), 1.61-1.47 (m, 2H), 1.44-1.35 (m, 1H), 0.94 (d, J=6.4 Hz, 3H), 0.89 (d, J=6.5 Hz, 3H); ¹³CNMR (151 MHz, DMSO-d6) δ 172.4, 172.0, 161.0, 152.5, 138.2, 130.7, 120.5, 119.8, 114.3, 113.1, 104.1, 99.7, 71.6, 51.3, 41.1, 40.1, 38.5, 36.9, 34.0, 29.4, 26.0, 24.4, 23.0, 21.4, 21.1; LC/MS: Eluent system A (retention time: 4.93 min); ESI-MS: 466 [M+H]⁺; IR: 2247 cm⁻¹; HRMS (ESI+) calcd for C₂₅H₃₁N₅O₄+H 466.2454, found 466.2467.

### Compound 72

### Synthesis of 3-{[N-(4-methoxy-1H-indole-2-carbonyl)leucyl]amino}-2-oxo-4-(2-oxopiperidin-3-yl)butyl 2,4,6-trimethylpyrimidine-5-carboxylate, 72

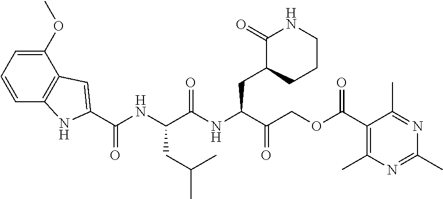 [SMILES: COc1cccc2[nH]c(C(=O)N[C@@H](CC(C)C)C(=O)N[C@@H](C[C@@H]3CCCNC3=O)C(=O)COC(=O)c3c(C)nc(C)nc3C)cc12]

Compound 72 was synthesized as in Scheme 45.

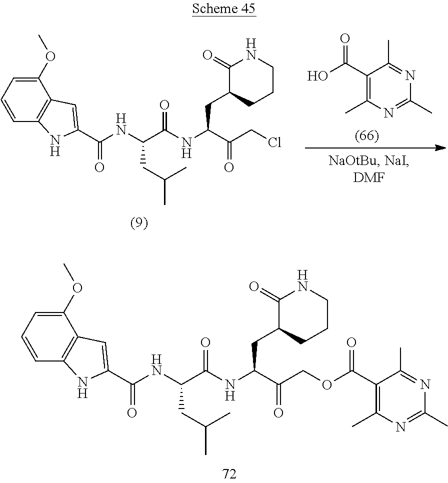 [SMILES: COc1cccc2[nH]c(C(=O)N[C@@H](CC(C)C)C(=O)N[C@@H](C[C@@H]3CCCNC3=O)C(=O)CCl)cc12 ; Cc1nc(C)c(C(=O)O)c(C)n1 ; COc1cccc2[nH]c(C(=O)N[C@@H](CC(C)C)C(=O)N[C@@H](C[C@@H]3CCCNC3=O)C(=O)COC(=O)c3c(C)nc(C)nc3C)cc12]

Preparation of 3-{[N-(4-methoxy-1H-indole-2-carbonyl)leucyl]amino}-2-oxo-4-(2-oxopiperidin-3-yl)butyl 2,4,6-trimethylpyrimidine-5-carboxylate, 72. To 2,4,6-trimethylpyrimidine-5-carboxylic acid, (66) (82.3 mg, 0.495 mmol) in anhydrous DMF (3 mL) was added sodium tert-butoxide (23.8 mg, 0.247 mmol). After 30 min, N-(1-{[4-chloro-3-oxo-1-(2-oxopiperidin-3-yl)butan-2-yl]amino}-4-methyl-1-oxopentan-2-yl)-4-methoxy-1H-indole-2-carboxamide, (9) (50.1 mg, 0.0992 mmol) and NaI (29.7 mg, 0.198 mmol) were added. After overnight, saturated brine solution (10 mL) was added, and the resulting mixture was extracted with CHCl₃ (3×10 mL). The combined organic layer was washed with saturated brine solution (1×10 mL), dried over anhydrous Na₂SO₄, filtered, and concentrated under reduced pressure. The residue was dissolved in CHCl₃ (5 mL) and loaded on a silica gel column, and the product was purified by Biotage® with a gradient of 0 to 6% MeOH in CHCl₃, which afforded 72 (38.1 mg, 61% yield) as an off-white solid. R_{f}=0.21 (5% MeOH in DCM). ¹H NMR (600 MHz, DMSO-d₆) δ 11.57 (d, J=2.3 Hz, 1H), 8.64 (d, J=8.1 Hz, 1H), 8.46 (d, J=7.5 Hz, 1H), 7.48-7.45 (m, 1H), 7.38 (dd, J=0.8, 2.3 Hz, 1H), 7.12-7.08 (m, 1H), 7.01 (d, J=8.1 Hz, 1H), 6.51 (d, J=7.3 Hz, 1H), 5.23-5.14 (m, 2H), 4.59-4.53 (m, 1H), 4.53-4.48 (m, 1H), 3.89 (s, 3H), 3.14-3.05 (m, 2H), 2.57 (s, 3H), 2.49 (s, 6H), 2.28-2.20 (m, 2H), 1.90-1.81 (m, 1H), 1.79-1.67 (m, 4H), 1.63-1.48 (m, 2H), 1.41-1.31 (m, 1H), 0.96 (d, J=6.4 Hz, 3H), 0.91 (d, J=6.4 Hz, 3H). ¹³C NMR (151 MHz, DMSO-d₆) δ 202.7, 173.0, 172.6, 167.2, 166.3, 164.2, 161.2, 153.6, 137.8, 129.8, 124.4, 122.3, 118.0, 105.4, 101.2, 99.2, 67.7, 55.1, 53.3, 51.7, 41.2, 40.1, 40.0, 36.8, 31.2, 25.6, 25.5, 24.4, 23.0, 22.43 (2C), 21.37, 21.2. LC/MS: Eluent system A (retention time: 5.71 min); ESI-MS: 635 [M+H]⁺; Anal. Calcd for C₃₃H₄₃N₆O₇.35H₂O: C, 61.83; H, 6.71; N, 13.11. Found: C, 61.80; H, 6.61; N, 12.92. HRMS (ESI+) calcd for C₃₃H₄₂N₆O₇+H 635.3193, found 635.3210.

### Compound 73

### Synthesis of (3S)-2-oxo-3-({N-[(2R)-oxolane-2-carbonyl]-L-leucyl}amino)-4-[(3S)-2-oxopiperidin-3-yl]butyl 2,4,6-trimethylpyridine-3-carboxylate, 73

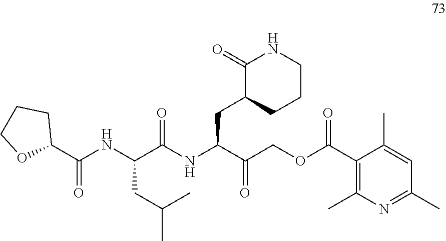 [SMILES: Cc1cc(C)c(C(=O)OCC(=O)[C@H](C[C@@H]2CCCNC2=O)NC(=O)[C@H](CC(C)C)NC(=O)[C@H]2CCCO2)c(C)n1]

Compound 73 was synthesized as in Scheme 46.

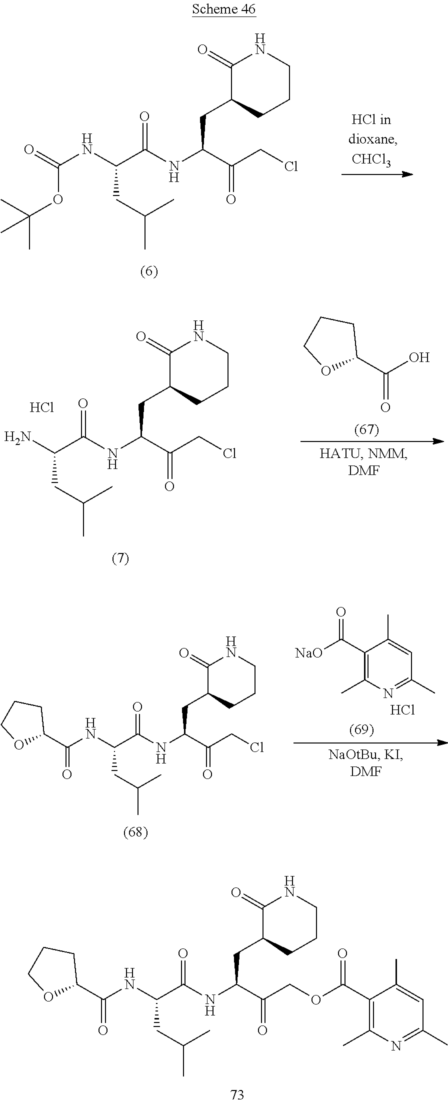

Preparation of N-{(2S)-4-chloro-3-oxo-1-[(3S)-2-oxopiperidin-3-yl]butan-2-yl}-L-leucinamide hydrogen chloride salt, (7). A solution of N²-(tert-butoxycarbonyl)-N-{(2S)-4-chloro-3-oxo-1-[(3S)-2-oxopiperidin-3-yl]butan-2-yl}-L-leucinamide (6) (277 mg, 0.642 mmol) in CHCl₃ (30 mL) was cooled using an ice-water bath and then 4 M HCl in 1,4-dioxane (3 mL) was added. After 30 min, the ice bath was removed, the mixture was warmed to room temperature. After overnight, the mixture was concentrated under reduced pressure at 35° C. (water bath temperature), treated with DCM (3×10 mL), and dried for an additional 1 h under reduced pressure to afford (7) (236 mg, quantitative yield) as an off-white solid. This material was used without further purification in the next step.

Preparation of N-{(2S)-4-chloro-3-oxo-1-[(3S)-2-oxopiperidin-3-yl]butan-2-yl}-N²-[(2R)-oxolane-2-carbonyl]-L-leucinamide, (68). To a solution of N-{(2S)-4-chloro-3-oxo-1-[(3S)-2-oxopiperidin-3-yl]butan-2-yl}-L-leucinamide hydrogen chloride salt (7) (236 mg, 0.641 mmol) and (2R)-oxolane-2-carboxylic acid (67) (82.1 mg, 0.706 mmol) in anhydrous DMF (10 mL) cooled in an ice bath was added HATU (268 mg, 0.706 mmol). Then, NMM (212 μL, 1.93 mmol) was added dropwise. After 45 min, a saturated aqueous brine solution (25 mL) was added, and the resulting mixture was extracted with ethyl acetate (3×25 mL). The combined organic layer was washed with saturated brine solution (1×25 mL), dried over anhydrous Na₂SO₄, filtered, and concentrated under reduced pressure. The resulting residue was dissolved in DCM (10 mL) and loaded on silica gel column, and the product was purified by Biotage® with a gradient of 0 to 2% MeOH in DCM, which generated (68) (191 mg, 69% yield) as an off-white solid.

Preparation of (3S)-2-oxo-3-({N-[(2R)-oxolane-2-carbonyl]-L-leucyl}amino)-4-[(3S)-2-oxopiperidin-3-yl]butyl 2,4,6-trimethylpyridine-3-carboxylate, 73. To sodium 2,4,6-trimethylpyridine-3-carboxylate hydrogen chloride salt (69) (597 mg, 2.67 mmol) in anhydrous DMF (3 mL) was added sodium tert-butoxide (171 mg, 1.78 mmol). After 30 min, N-{(2S)-4-chloro-3-oxo-1-[(3S)-2-oxopiperidin-3-yl]butan-2-yl}-N²-[(2R)-oxolane-2-carbonyl]-L-leucinamide, (68) (191 mg, 0.445 mmol) and KI (73.9 mg, 0.445 mmol) were added. After overnight, saturated brine solution (50 mL) was added, and the resulting mixture was extracted with CHCl₃ (3×10 mL). The combined organic layer was washed with saturated brine solution (1×50 mL), dried over anhydrous Na₂SO₄, filtered, and concentrated under reduced pressure. The residue was dissolved in CHCl₃ (10 mL) and loaded on a silica gel column, and the product was purified by Biotage® with a gradient of 0 to 10% MeOH in EtOAc, which afforded 73 (116.6 mg, 47% yield) as an off-white solid. ¹H NMR (600 MHz, DMSO-d₆) δ 8.56 (d, J=7.9 Hz, 1H), 7.73 (d, J=8.5 Hz, 1H), 7.50 (br s, 1H), 7.05 (s, 1H), 5.24-4.95 (m, 2H), 4.56-4.48 (m, 1H), 4.39-4.29 (m, 1H), 4.28-4.24 (m, 1H), 3.95-3.90 (m, 1H), 3.79-3.74 (m, 1H), 3.18-3.05 (m, 2H), 2.46 (s, 3H), 2.41 (s, 3H), 2.30 (s, 3H), 2.24-2.14 (m, 2H), 2.14-2.05 (m, 1H), 1.91-1.69 (m, 6H), 1.63-1.46 (m, 4H), 1.42-1.33 (m, 1H), 0.91 (d, J=6.2 Hz, 3H), 0.86 (d, J=6.2 Hz, 3H). LC/MS: Eluent system B (retention time: 4.41 min); ESI-MS: 559 [M+H]⁺.

### Compound 74

### Synthesis of (3S)-2-oxo-3-({N-[(2R)-oxolane-2-carbonyl]-L-leucyl}amino)-4-[(3S)-2-oxopyrrolidin-3-yl]butyl 2,6-bis(trifluoromethyl)benzoate, 74

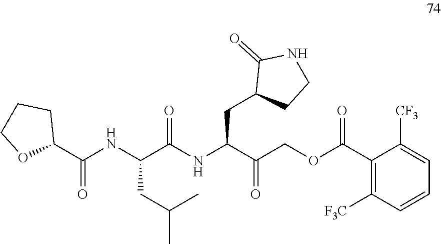 [SMILES: CC(C)C[C@H](NC(=O)[C@H]1CCCO1)C(=O)N[C@@H](C[C@@H]1CCNC1=O)C(=O)COC(=O)c1c(C(F)(F)F)cccc1C(F)(F)F]

Compound 74 was synthesized as in Scheme 47.

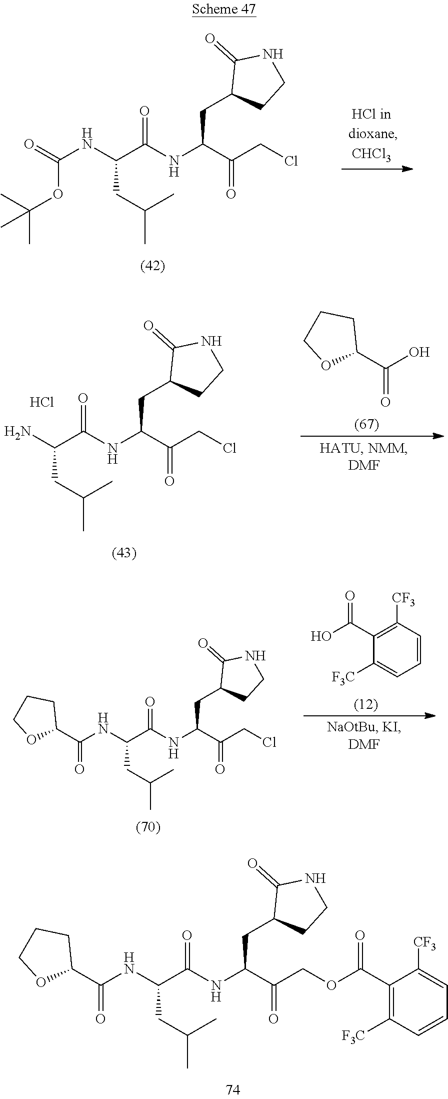

Preparation of N-{(2S)-4-chloro-3-oxo-1-[(3S)-2-oxopyrrolidin-3-yl]butan-2-yl}-L-leucinamide hydrogen chloride salt, (43). A solution of N²-(tert-butoxycarbonyl)-N-{(2S)-4-chloro-3-oxo-1-[(3S)-2-oxopyrrolidin-3-yl]butan-2-yl}-L-leucinamide (42) (200 mg, 0.479 mmol) in CHCl₃ (10 mL) was cooled using an ice-water bath and then 4 M HCl in 1,4-dioxane (2 mL) was added. After 30 min, the ice bath was removed, the mixture was warmed to room temperature. After overnight, the mixture was concentrated under reduced pressure at 35° C. (water bath temperature), treated with DCM (3×10 mL), and dried for an additional 1 h under reduced pressure to afford (43) (169 mg) as an off-white solid. This material was used without further purification in the next step.

Preparation of N-{(2S)-4-chloro-3-oxo-1-[(3S)-2-oxopyrrolidin-3-yl]butan-2-yl}-N²-[(2R)-oxolane-2-carbonyl]-L-leucinamide, (70). To a solution of N-{(2S)-4-chloro-3-oxo-1-[(3S)-2-oxopyrrolidin-3-yl]butan-2-yl}-L-leucinamide hydrogen chloride salt (43) (100 mg, 0.282 mmol) and (2R)-oxolane-2-carboxylic acid (67) (36.1 mg, 0.311 mmol) in anhydrous DMF (5 mL) cooled in an ice bath was added HATU (118 mg, 0.346 mmol). Then, NMM (93.1 μL, 0.847 mmol) was added dropwise. After 45 min, a saturated aqueous brine solution (25 mL) was added, and the resulting mixture was extracted with ethyl acetate (3×25 mL). The combined organic layer was washed with saturated brine solution (1×25 mL), dried over anhydrous Na₂SO₄, filtered, and concentrated under reduced pressure. The resulting residue was dissolved in CHCl₃ (10 mL) and loaded on silica gel column, and the product was purified by Biotage® with a gradient of 0 to 8% MeOH in EtOAc, which generated (70) (51.4 mg, 44% yield) as an off-white solid.

Preparation of (3S)-2-oxo-3-({N-[(2R)-oxolane-2-carbonyl]-L-leucyl}amino)-4-[(3S)-2-oxopyrrolidin-3-yl]butyl 2,6-bis(trifluoromethyl)benzoate, 74. To 2,6-bis(trifluoromethyl)benzoic acid, (12) (159 mg, 0.617 mmol) in anhydrous DMF (5 mL) was added sodium tert-butoxide (29.7 mg, 0.309 mmol). After 30 min, N-{(2S)-4-chloro-3-oxo-1-[(3S)-2-oxopyrrolidin-3-yl]butan-2-yl}-N²-[(2R)-oxolane-2-carbonyl]-L-leucinamide (70) (51.4 mg, 0.124 mmol) and KI (20.5 mg, 0.124 mmol) were added. After overnight, saturated brine solution (25 mL) was added, and the resulting mixture was extracted with CHCl₃ (3×25 mL). The combined organic layer was washed with saturated brine solution (1×25 mL), dried over anhydrous Na₂SO₄, filtered, and concentrated under reduced pressure. The residue was dissolved in CHCl₃ (5 mL) and loaded on a silica gel column, and the product was purified by Biotage® with a gradient of 0 to 10% MeOH in CHCl₃, which afforded 74 (22.1 mg, 28% yield) as an off-white solid. ¹H NMR (600 MHz, DMSO-d₆) δ 8.57 (d, J=7.9 Hz, 1H), 8.26 (d, J=7.9 Hz, 2H), 8.02 (t, J=8.0 Hz, 1H), 7.73 (d, J=8.5 Hz, 1H), 7.67 (s, 1H), 5.19-5.13 (m, 2H), 4.51-4.45 (m, 1H), 4.36-4.31 (m, 1H), 4.28-4.24 (m, 1H), 3.95-3.90 (m, 1H), 3.79-3.74 (m, 1H), 3.18-3.14 (m, 1H), 3.13-3.07 (m, 1H), 2.30-2.22 (m, 1H), 2.13-2.05 (m, 2H), 2.03-1.96 (m, 1H), 1.87-1.76 (m, 3H), 1.71-1.61 (m, 2H), 1.61-1.54 (m, 2H), 1.53-1.46 (m, 1H), 0.91 (d, J=6.2 Hz, 3H), 0.87 (d, J=6.2 Hz, 3H). ¹⁹F NMR (565 MHz, DMSO-d₆) δ −58.33 (s, 6F). LC/MS: Eluent system A (retention time: 5.73 min); ESI-MS: 638 [M+H]⁺.

### Compound 75

### Synthesis of N-{(1S)-1-cyano-2-[(3S)-2-oxopiperidin-3-yl]ethyl}-N²-(4-phenyl-1H-imidazole-2-carbonyl)-L-leucinamide, 75

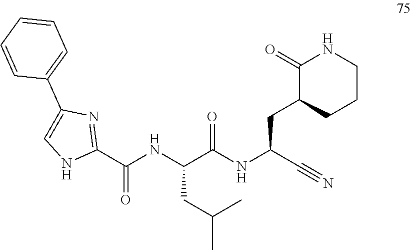 [SMILES: CC(C)C[C@H](NC(=O)c1nc(-c2ccccc2)c[nH]1)C(=O)N[C@H](C#N)C[C@@H]1CCCNC1=O]

Compound 75 was synthesized as in Scheme 48.

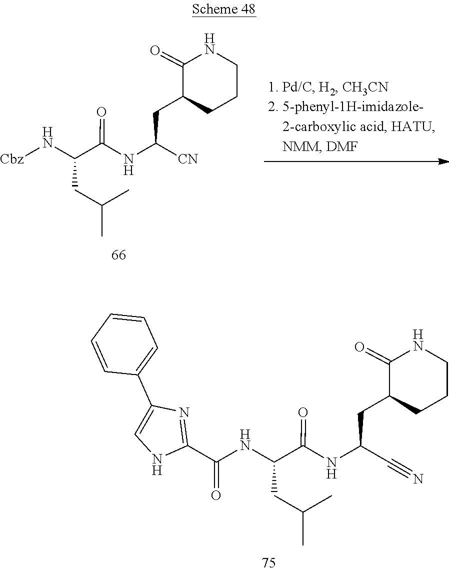 [SMILES: CC(C)C[C@H](NC(=O)OCc1ccccc1)C(=O)N[C@H](C#N)C[C@@H]1CCCNC1=O ; CC(C)C[C@H](NC(=O)c1nc(-c2ccccc2)c[nH]1)C(=O)N[C@H](C#N)C[C@@H]1CCCNC1=O]

Preparation of N-{(1S)-1-cyano-2-[(3S)-2-oxopiperidin-3-yl]ethyl}-N²-(4-phenyl-1H-imidazole-2-carbonyl)-L-leucinamide, 75. To a solution N²-[(benzyloxy)carbonyl]-N-{(1S)-1-cyano-2-[(3S)-2-oxopiperidin-3-yl]ethyl}-L-leucinamide 66 (89.8 mg, 0.217 mmol) in CH₃CN (10 mL) was added Pd/C (10% wt, 50.1 mg) and the mixture was stirred vigorously under a hydrogen atmosphere supplied by a hydrogen filled balloon. After 2 h, the mixture was filtered through a pad of Celite® and the volatiles in the filtrate were removed under reduced pressure. To the residue was added anhydrous DMF (5 mL) and 5-phenyl-1H-imidazole-2-carboxylic acid (48.9 mg, 0.260 mmol) and the solution was cooled in an ice bath, after which HATU (107 mg, 0.282 mmol) was added followed by dropwise addition of NMM (0.0590 mL, 0.537 mmol). After 45 min, an ice/saturated aqueous NaHCO₃ mixture (1:1, 20 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in CHCl₃, which generated 75 (11.7 mg, 12% yield) as a white solid. ¹HNMR (600 MHz, DMSO-d₆) δ 13.47 (s, 0.21H), 13.21 (s, 0.79H), 8.96 (d, J=8.0 Hz, 0.79H), 8.91 (d, J=8.0 Hz, 0.21H), 8.30 (d, J=8.5 Hz, 0.21H), 8.22 (d, J=8.5 Hz, 0.79H), 7.91-7.84 (m, 2H), 7.82 (s, 0.79H), 7.56-7.52 (m, 1.21H), 7.43-7.36 (m, 2H), 7.32-7.28 (m, 0.21H), 7.27-7.23 (m, 0.79H), 5.10-5.03 (m, 1H), 4.54-4.47 (m, 1H), 3.14-3.04 (m, 2H), 2.23-2.20 (m, 2H), 1.87-1.68 (m, 4H), 1.68-1.51 (m, 3H), 1.45-1.36 (m, 1H), 0.94 (d, J=6.6 Hz, 3H), 0.92 (d, J=6.6 Hz, 3H); LC/MS: Eluent system A (retention time: 5.17 min); ESI-MS: 451 [M+H]⁺.

### Compound 76

### Synthesis of N-{(1S)-1-cyano-2-[(3S)-2-oxopiperidin-3-yl]ethyl}-N²-(1-hydroxycyclopentane-1-carbonyl)-L-leucinamide, 76

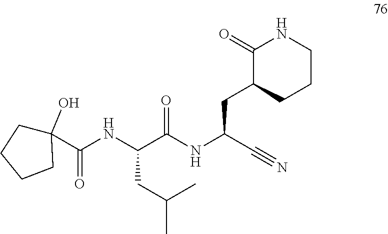 [SMILES: CC(C)C[C@H](NC(=O)C1(O)CCCC1)C(=O)N[C@H](C#N)C[C@@H]1CCCNC1=O]

Compound 76 was synthesized as in Scheme 49.

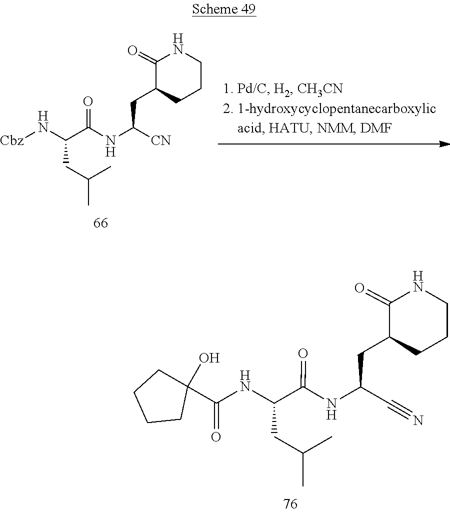 [SMILES: CC(C)C[C@H](NC(=O)OCc1ccccc1)C(=O)N[C@H](C#N)C[C@@H]1CCCNC1=O ; CC(C)C[C@H](NC(=O)C1(O)CCCC1)C(=O)N[C@H](C#N)C[C@@H]1CCCNC1=O]

Preparation of N-{(1S)-1-cyano-2-[(3S)-2-oxopiperidin-3-yl]ethyl}-N²-(1-hydroxycyclopentane-1-carbonyl)-L-leucinamide, 76. To a solution N²-[(benzyloxy)carbonyl]-N-{(1S)-1-cyano-2-[(3S)-2-oxopiperidin-3-yl]ethyl}-L-leucinamide 66 (95.1 mg, 0.229 mmol) in CH₃CN (10 mL) was added Pd/C (10% wt. 55.3 mg) and the mixture was stirred vigorously under a hydrogen atmosphere supplied by a hydrogen filled balloon. After 2 h, the mixture was filtered through a pad of Celite® and the volatiles in the filtrate was removed under reduced pressure. To the residue was added anhydrous DMF (5 mL) and 1-hydroxycyclopentanecarboxylic acid (35.8 mg, 0.275 mmol) and the solution was cooled in an ice bath, after which HATU (114 mg, 0.300 mmol) was added followed by dropwise addition of NMM (0.0755 mL, 0.687 mmol). After 45 min, an ice/saturated aqueous NaHCO₃ mixture (1:1, 20 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in EtOAc, which generated 76 (15.2 mg, 17% yield) as a white solid. ¹HNMR (600 MHz, DMSO-d₆) δ 8.88 (d, J=7.7 Hz, 1H), 7.65 (d, J=8.6 Hz, 1H), 7.56 (s, 1H), 5.4 (s, 1H), 5.03-4.97 (m, 1H), 4.35-4.28 (m, 1H), 3.14-3.04 (m, 2H), 2.28-2.17 (m, 2H), 1.94-1.80 (m, 3H), 1.80-1.67 (m, 4H), 1.66-1.50 (m, 7H), 1.49-1.36 (m, 2H), 0.90 (d, J=6.4 Hz, 3H), 0.86 (d, J=6.5 Hz, 3H); LC/MS: Eluent system A (retention time: 2.21 min); ESI-MS: 393 [M+H]⁺.

### Compound 77

### Synthesis of N-{(1S)-1-cyano-2-[(3S)-2-oxopiperidin-3-yl]ethyl}-N²-(cyclopropylmethyl)-L-leucinamide, 77

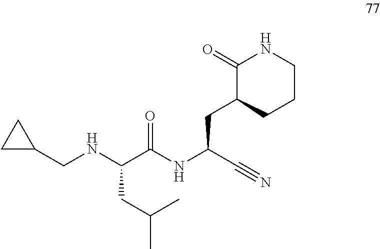 [SMILES: CC(C)C[C@H](NCC1CC1)C(=O)N[C@H](C#N)C[C@@H]1CCCNC1=O]

Compound 77 was synthesized as in Scheme 50.

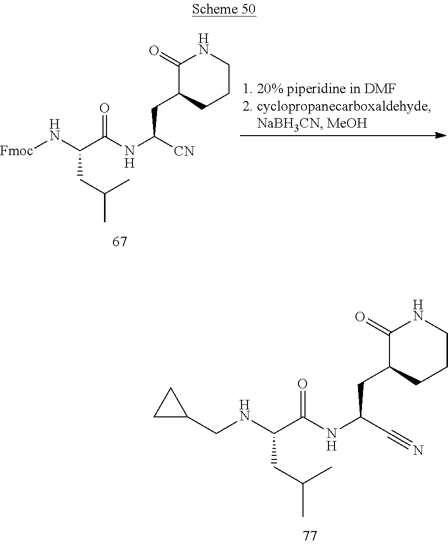

Preparation of N-{(1S)-1-cyano-2-[(3S)-2-oxopiperidin-3-yl]ethyl}-N²-(cyclopropylmethyl)-L-leucinamide, 77. To a round bottom flask was added N-{(1S)-1-cyano-2-[(3S)-2-oxopiperidin-3-yl]ethyl}-N²-{[(9H-fluoren-9-yl)methoxy]carbonyl}-L-leucinamide 67 (17.6 mg, 0.0350 mmol) and a 20% piperidine solution in DMF (1.0 mL). The reaction mixture was stirred for 15 min and the volatiles were removed under reduced pressure with a bath temperature at 37° C. To the residue was added anhydrous MeOH (5 mL) and cyclopropanecarboxaldehyde (2.5 mg, 0.0350 mmol), followed by addition of 1 drop of acetic acid. After 30 min, to the reaction mixture was added NaBH₃CN (3.3 mg, 0.0525 mmol). After 30 min, the volatiles were removed under reduced pressure and the product was purified by column chromatography on silica gel with a gradient of 0 to 10% MeOH in EtOAc, which generated 77 (3.5 mg, 30% yield) as a sticky solid. ¹HNMR (600 MHz, DMSO-d₆) δ 8.67 (d, J=8.0 Hz, 1H), 7.49 (s, 1H), 5.00-4.93 (m, 1H), 3.10-3.00 (m, 3H), 2.29 (dd, J=11.9, 6.2 Hz, 1H), 2.26-2.19 (m, 1H), 2.13-2.05 (m, 2H), 1.80-1.67 (m, 3H), 1.63-1.57 (m, 1H), 1.52-1.33 (m, 3H), 1.30-1.24 (m, 2H), 0.84 (d, J=6.6 Hz, 3H), 0.80 (d, J=6.6 Hz, 3H), 0.38-0.29 (m, 2H), 0.03-−0.06 (m, 2H); LC/MS: Eluent system C (Gradient of MeOH-Water, 15% to 65% in 5 min, 65% to 95% in 2.5 min, followed by 4 min of isocratic MeOH-water 95%) (retention time: 4.12 min); ESI-MS: 335 [M+H]⁺.

### Compound 78

### Synthesis of N-{(1S)-1-cyano-2-[(3S)-2-oxopiperidin-3-yl]ethyl}-2-[(2-oxo-1,2-dihydropyridin-3-yl)methyl]-L-leucinamide, 78

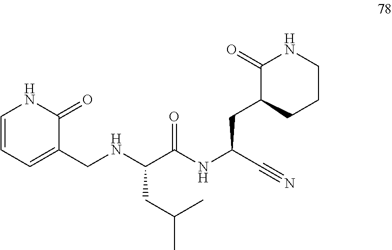 [SMILES: CC(C)C[C@H](NCc1ccc[nH]c1=O)C(=O)N[C@H](C#N)C[C@@H]1CCCNC1=O]

Compound 78 was synthesized as in Scheme 51.

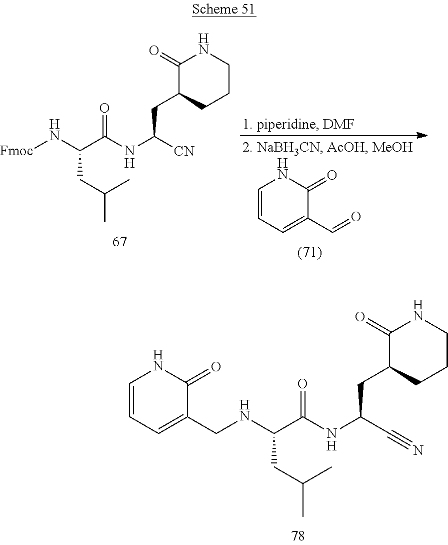

Preparation of N-{(1S)-1-cyano-2-[(3S)-2-oxopiperidin-3-yl]ethyl}-N²-[(2-oxo-1,2-dihydropyridin-3-yl)methyl]-L-leucinamide, 78. A mixture of N-{(1S)-1-cyano-2-[(3S)-2-oxopiperidin-3-yl]ethyl}-N²-{[(9H-fluoren-9-yl)methoxy]carbonyl}-L-leucinamide 67 (36 mg, 0.072 mmol) and piperidine (2 mL, 20% in DMF) was stirred for 5 min. The resulting mixture was concentrated under reduced pressure, then mixed with toluene (25 mL) and again concentrated under reduced pressure. The residue was mixed with 2-oxo-1,2-dihydropyridine-3-carbaldehyde (71) (11 mg, 0.079 mmol) and acetic acid (1 drop) in MeOH (5 mL). After 1 h at room temperature, NaBH₃CN (7 mg, 0.11 mmol) was added. After an additional 1 h, the mixture was concentrated under reduced pressure, the resulting residue mixed with water (10 mL) and extracted with ethyl acetate (3×25 mL). The combined organic layer was dried over Na₂SO₄, filtered and concentrated under reduced pressure. The product was purified by column chromatography on silica gel with a gradient of 0 to 1% MeOH in 1:1 CHCl₃/ethyl acetate, which generated 78 (11 mg, 39% yield) as a white solid. ¹H NMR (600 MHz, DMSO-d₆) δ 11.70-11.63 (m, 1H), 9.09 (d, J=7.9 Hz, 1H), 7.53 (br s, 1H), 7.40 (d, J=6.9 Hz, 1H), 7.32-7.25 (m, 1H), 6.17 (t, J=6.8 Hz, 1H), 5.09-5.02 (m, 1H), 3.43-3.34 (m, 1H), 3.20-3.04 (m, 2H), 1.88-1.81 (m, 2H), 1.76-1.71 (m, 2H), 1.71-1.65 (m, 2H), 1.65-1.56 (m, 2H), 1.46-1.39 (m, 2H), 1.38-1.32 (m, 2H), 1.31-1.20 (m, 1H), 0.88 (d, J=6.8 Hz, 3H), 0.82 (d, J=6.4 Hz, 3H). LC/MS: Eluent system C (Gradient of MeOH-Water, 15% to 65% in 5 min, 65% to 95% in 2.5 min, followed by 4 min of isocratic MeOH-water 95%) (retention time: 3.55 min); ESI-MS: 388 [M+H]⁺.

### Compound 79

### Synthesis of N-[(2S)-1-({(1S)-1-cyano-2-[(3S)-2-oxopiperidin-3-yl]ethyl}amino)-4-methyl-1-oxopentan-2-yl]-1-benzofuran-2-carboxamide, 79

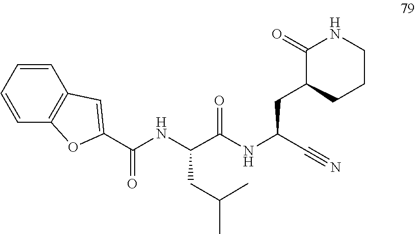 [SMILES: CC(C)C[C@H](NC(=O)c1cc2ccccc2o1)C(=O)N[C@H](C#N)C[C@@H]1CCCNC1=O]

Compound 79 was synthesized as in Scheme 52.

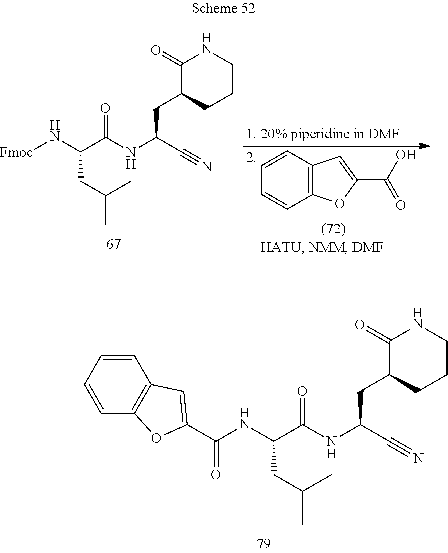

Preparation of N-[(2S)-1-({(1S)-1-cyano-2-[(3S)-2-oxopiperidin-3-yl]ethyl}amino)-4-methyl-1-oxopentan-2-yl]-1-benzofuran-2-carboxamide, 79. To a mixture of DMF:piperidine (1 mL, 8:2) was added N-{(1S)-1-cyano-2-[(3S)-2-oxopiperidin-3-yl]ethyl}-N²-{[(9H-fluoren-9-yl)methoxy]carbonyl}-L-leucinamide, 67 (34.2 mg, 0.068 mmol) and the solution was stirred at room temperature. After 15 min, the mixture was concentrated under reduced pressure, then treated with toluene (3×5 mL) and concentrated under reduced pressure. The residue was dissolved in DMF (3 mL) was cooled in an ice-water bath and then were successively added 1-benzofuran-2-carboxylic acid (72) (12.1 mg, 0.0748 mmol), HATU (28.5 mg, 0.0748 mmol) and NMM (22.4 μL, 0.204 mmol). After 1 h, saturated brine solution (25 mL) was added and the mixture extracted with CHCl₃ (3×25 mL). The combined organic layer was washed with saturated brine solution (1×25 mL), dried over anhydrous Na₂SO₄, filtered, and concentrated under reduced pressure. The residue was dissolved in CHCl₃ (5 mL) and loaded on a silica gel column, and the product was purified by Biotage® with a gradient of 0 to 4% MeOH in EtOAc, which afforded 79 (15.9 mg, 55% yield) as an off-white solid. ¹H NMR (600 MHz, DMSO-d₆) δ 8.92 (d, J=7.9 Hz, 1H), 8.78 (d, J=7.9 Hz, 1H), 7.81-7.78 (m, 1H), 7.69 (dd, J=0.8, 8.5 Hz, 1H), 7.64 (d, J=0.8 Hz, 1H), 7.54 (br s, 1H), 7.48 (ddd, J=1.2, 7.2, 8.4 Hz, 1H), 7.37-7.33 (m, 1H), 5.09-5.02 (m, 1H), 4.53-4.43 (m, 1H), 3.15-3.03 (m, 2H), 2.32-2.22 (m, 2H), 1.87-1.62 (m, 5H), 1.62-1.51 (m, 2H), 1.44-1.37 (m, 1H), 0.93 (d, J=6.6 Hz, 3H), 0.89 (d, J=6.6 Hz, 3H). LC/MS: Eluent system A (retention time: 4.67 min); ESI-MS: 425 [M+H]⁺.

### Compound 80

### Synthesis of N-{(1S)-1-cyano-2-[(3S)-2-oxopiperidin-3-yl]ethyl}-N²-[(2R)-oxolane-2-carbonyl]-L-leucinamide, 80

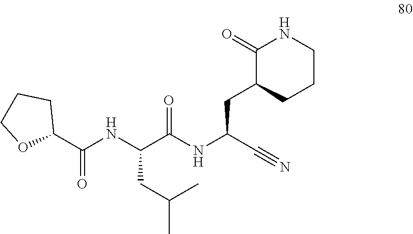 [SMILES: CC(C)C[C@H](NC(=O)[C@H]1CCCO1)C(=O)N[C@H](C#N)C[C@@H]1CCCNC1=O]

Compound 80 was synthesized as in Scheme 53.

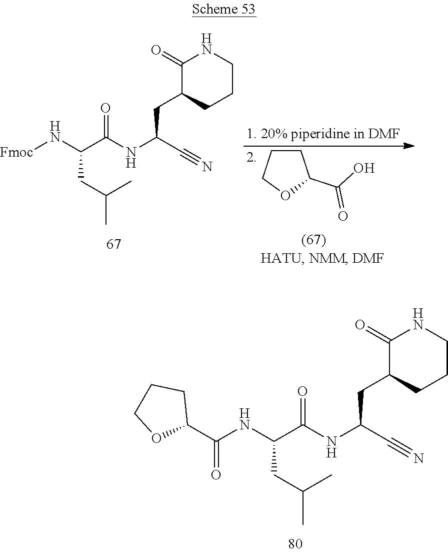

Preparation of N-{(1S)-1-cyano-2-[(3S)-2-oxopiperidin-3-yl]ethyl}-N²-[(2R)-oxolane-2-carbonyl]-L-leucinamide, 80. A similar procedure as described for compound 79 was followed: with N-{(1S)-1-cyano-2-[(3S)-2-oxopiperidin-3-yl]ethyl}-N²-{[(9H-fluoren-9-yl)methoxy]carbonyl}-L-leucinamide, 67 (33.4 mg, 0.0664 mmol), 20% piperidine in DMF and followed by (2R)-oxolane-2-carboxylic acid (67) (8.51 mg, 0.0731 mmol), HATU (27.8 mg, 0.0731 mmol) and NMM (22.4 μL, 0.199 mmol) in DMF (3 mL). The residue was dissolved in CHCl₃ (5 mL) and loaded on a silica gel column, and the product was purified by Biotage® with a gradient of 0 to 8% MeOH in EtOAc, which afforded 80 (17.3 mg, 69% yield) as an off-white solid. ¹H NMR (600 MHz, DMSO-d₆) δ 8.84 (d, J=7.9 Hz, 1H), 7.70 (d, J=8.3 Hz, 1H), 7.55 (br s, 1H), 5.03-4.98 (m, 1H), 4.30-4.23 (m, 2H), 3.95-3.88 (m, 1H), 3.79-3.73 (m, 1H), 3.15-3.05 (m, 2H), 2.30-2.17 (m, 2H), 2.13-2.04 (m, 1H), 1.87-1.70 (m, 6H), 1.61-1.51 (m, 3H), 1.48-1.37 (m, 2H), 0.90 (d, J=6.4 Hz, 3H), 0.86 (d, J=6.4 Hz, 3H). LC/MS: Eluent system B (retention time: 4.82 min); ESI-MS: 379 [M+H]⁺.

### Compound 81

### Synthesis of N-{(1S)-1-cyano-2-[(3S)-2-oxopiperidin-3-yl]ethyl}-N²-(6-methylpyridine-3-carbonyl)-L-leucinamide, 81

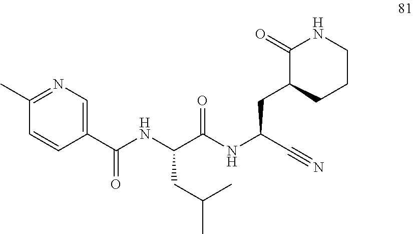 [SMILES: Cc1ccc(C(=O)N[C@@H](CC(C)C)C(=O)N[C@H](C#N)C[C@@H]2CCCNC2=O)cn1]

Compound 81 was synthesized as in Scheme 54.

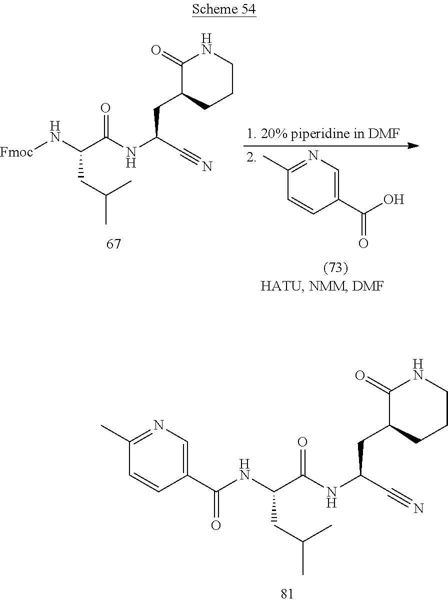

Preparation of N-{(1S)-1-cyano-2-[(3S)-2-oxopiperidin-3-yl]ethyl}-N²-(6-methylpyridine-3-carbonyl)-L-leucinamide, 81. A similar procedure as described for compound 79 was followed: with N-{(1S)-1-cyano-2-[(3S)-2-oxopiperidin-3-yl]ethyl}-N²-{[(9H-fluoren-9-yl)methoxy]carbonyl}-L-leucinamide 67 (32.7 mg, 0.0651 mmol), 20% piperidine in DMF (1 mL) and followed by 6-methylpyridine-3-carboxylic acid (73) (9.81 mg, 0.0716 mmol), HATU (27.2 mg, 0.0716 mmol) and NMM (21.5 μL, 0.195 mmol) in DMF (3 mL). The residue was dissolved in CHCl₃ (5 mL) and loaded on a silica gel column, and the product was purified by Biotage® with a gradient of 0 to 8% MeOH in EtOAc, which afforded 81 (15.3 mg, 59% yield) as an off-white solid. ¹H NMR (600 MHz, DMSO-d₆) δ 8.94-8.88 (m, 2H), 8.67 (d, J=7.5 Hz, 1H), 8.12 (dd, J=2.2, 8.0 Hz, 1H), 7.53 (br s, 1H), 7.36 (d, J=8.3 Hz, 1H), 5.06-5.00 (m, 1H), 4.48-4.40 (m, 1H), 3.15-3.02 (m, 2H), 2.52 (s, 3H) 2.31-2.21 (m, 2H), 1.86-1.75 (m, 2H), 1.75-1.61 (m, 3H), 1.60-1.49 (m, 2H), 1.40-1.34 (m, 1H), 0.93 (d, J=6.4 Hz, 3H), 0.88 (d, J=6.4 Hz, 3H). LC/MS: Eluent system C (see compound 78) (retention time: 5.64 min); ESI-MS: 400 [M+H]⁺.

### Compound 82

### Synthesis of N-{(1S)-1-cyano-2-[(3S)-2-oxopiperidin-3-yl]ethyl}-N²-(2,3-dihydro-1,4-benzodioxine-5-sulfonyl)-L-leucinamide, 82

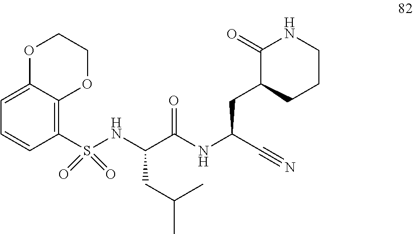 [SMILES: CC(C)C[C@H](NS(=O)(=O)c1cccc2c1OCCO2)C(=O)N[C@H](C#N)C[C@@H]1CCCNC1=O]

Compound 82 was synthesized as in Scheme 55.

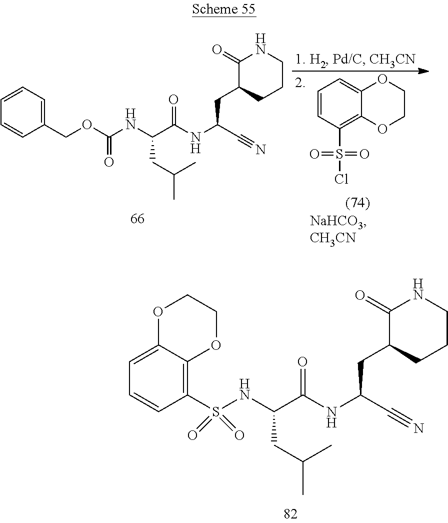 [SMILES: CC(C)C[C@H](NC(=O)OCc1ccccc1)C(=O)N[C@H](C#N)C[C@@H]1CCCNC1=O ; O=S(=O)(Cl)c1cccc2c1OCCO2 ; CC(C)C[C@H](NS(=O)(=O)c1cccc2c1OCCO2)C(=O)N[C@H](C#N)C[C@@H]1CCCNC1=O]

Preparation of N-{(1S)-1-cyano-2-[(3S)-2-oxopiperidin-3-yl]ethyl}-N²-(2,3-dihydro-1,4-benzodioxine-5-sulfonyl)-L-leucinamide, 82. To a solution of N²-[(benzyloxy)carbonyl]-N-{(1S)-1-cyano-2-[(3S)-2-oxopiperidin-3-yl]ethyl}-L-leucinamide 66 (32.1 mg, 0.0774 mmol) in acetonitrile (8 mL) was added 10% palladium on activated carbon (16.1 mg, 0.0152 mmol). The atmosphere in the flask was substituted with hydrogen by repeating cycle of applying vacuum followed by purging hydrogen gas three times and the mixture was stirred under slight positive pressure of hydrogen gas for 90 minutes. Then the resulting mixture was filtered through a Celite® plug, which was washed with acetonitrile (5 mL), and to the combined filtrate 2,3-dihydrobenzo[b][1,4]dioxine-5-sulfonyl chloride (74) (34.3 mg, 0.146 mg) was added, followed by sodium bicarbonate (84.2 mg, 1.00 mmol). After overnight, the mixture was diluted with ethyl acetate (25 mL) and washed with water (15 mL). The organic layer was dried with sodium sulfate, filtered and then concentrated under reduced pressure. The product was purified by column chromatography (eluted with gradient 0% to 12% of methanol in 1:1 mixture ethyl acetate-chloroform), which provided 82 (15.0 mg, 40% yield after two steps) as white powder. ¹H NMR (600 MHz, DMSO-d₆) δ 8.72 (d, J=7.8 Hz, 1H), 7.57-7.52 (m, 2H), 7.23 (dd, J=8.1, 1.3 Hz, 1H), 7.08 (dd, J=8.1, 1.6 Hz, 1H), 6.89 (t, J=8.1 Hz, 1H), 4.78 (ddd, J=8.3, 8.0, 7.8 Hz, 1H), 4.38-4.23 (m, 5H), 3.71-3.66 (m, 1H), 3.16-3.05 (m, 2H), 2.11-1.99 (m, 2H), 1.80-1.54 (m, 3H), 1.50-1.33 (m, 2H), 1.29-1.22 (m, 2H), 0.85 (d, J=6.6 Hz, 3H), 0.82 (d, J=6.6 Hz, 3H). LC/MS: Eluent system B (retention time: 5.86 min); ESI-MS 479 [M+H]⁺, 477 [M−H]⁻.

### Compound 83

### Synthesis of N-{(1S)-1-cyano-2-[(3S)-2-oxopiperidin-3-yl]ethyl}-N²-(piperidine-1-sulfonyl)-L-leucinamide, 83

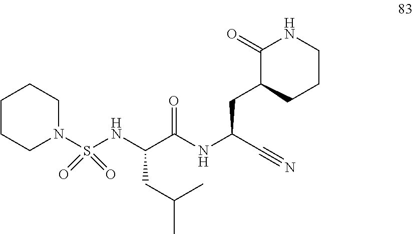 [SMILES: CC(C)C[C@H](NS(=O)(=O)N1CCCCC1)C(=O)N[C@H](C#N)C[C@@H]1CCCNC1=O]

Compound 83 was synthesized as in Scheme 56.

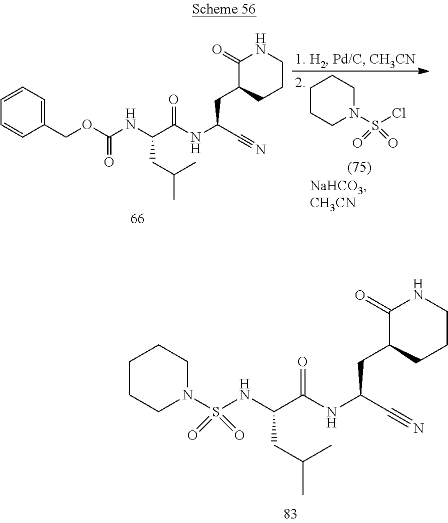

Preparation of N-{(1S)-1-cyano-2-[(3S)-2-oxopiperidin-3-yl]ethyl}-N²-(piperidine-1-sulfonyl)-L-leucinamide, 83. The product was synthesized as described for compound 82 starting with 66 (20.3 mg, 0.0490 mmol) except using piperidine-1-sulfonyl chloride (75) (70.6 mg, 0.384 mmol) in the presence of sodium bicarbonate (112 mg, 1.33 mmol). Purification by column chromatography (eluted with gradient of 0% to 12% methanol in 1:1 mixture ethyl acetate-chloroform) provided of 83 (2.9 mg, 14% yield over 2 steps) as a clear film. ¹H NMR (600 MHz, DMSO-d6) δ 8.91 (d, J=7.6 Hz, 1H), 7.60-7.50 (m, 2H), 5.02-4.91 (m, 1H), 3.72-3.67 (m, 1H), 3.15-2.91 (m, 4H), 2.29-2.18 (m, 2H), 1.90-1.26 (m, 14H), 0.94-0.85 (m, 8H). LC/MS: Eluent system A (retention time: 3.49 min); ESI-MS 428 [M+H]⁺, 426 [M−H]⁻.

### Compound 84

### Synthesis of N-{(1S)-1-cyano-2-[(3S)-2-oxopiperidin-3-yl]ethyl}-N²-(cyclohexanesulfonyl)-L-leucinamide, 84

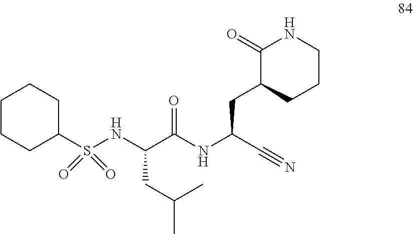 [SMILES: CC(C)C[C@H](NS(=O)(=O)C1CCCCC1)C(=O)N[C@H](C#N)C[C@@H]1CCCNC1=O]

Compound 84 was synthesized as in Scheme 57.

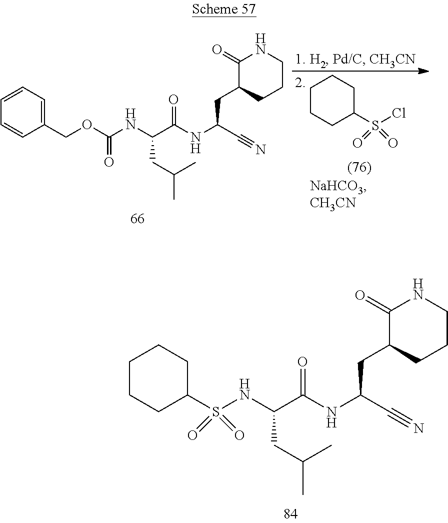

Preparation of N-{(1S)-1-cyano-2-[(3S)-2-oxopiperidin-3-yl]ethyl}-N²-(cyclohexanesulfonyl)-L-leucinamide, 84. The product was synthesized as described for compound 82 starting with 66 (20.3 mg, 0.0490 mmol) except with treatment of cyclohexanesulfonyl chloride (76) (81.2 mg, 0.444 mmol) in the presence of sodium bicarbonate (89.9 mg, 1.07 mmol). Purification by column chromatography (eluted with gradient 0% to 12% of methanol in 1:1 mixture ethyl acetate-chloroform) provided 84 (3.9 mg, 19% yield over 2 steps) as a clear film. LC/MS: Eluent system A (retention time: 3.96 min); ESI-MS 427 [M+H]⁺, 425 [M−H]⁻.

### Compound 85

### Synthesis of N-{(1S)-1-cyano-2-[(3S)-2-oxopiperidin-3-yl]ethyl}-N²-(6-methylpyridine-3-sulfonyl)-L-leucinamide, 85

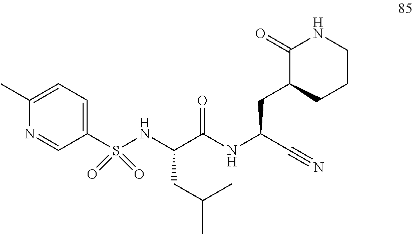 [SMILES: Cc1ccc(S(=O)(=O)N[C@@H](CC(C)C)C(=O)N[C@H](C#N)C[C@@H]2CCCNC2=O)cn1]

Compound 85 was synthesized as in Scheme 58.

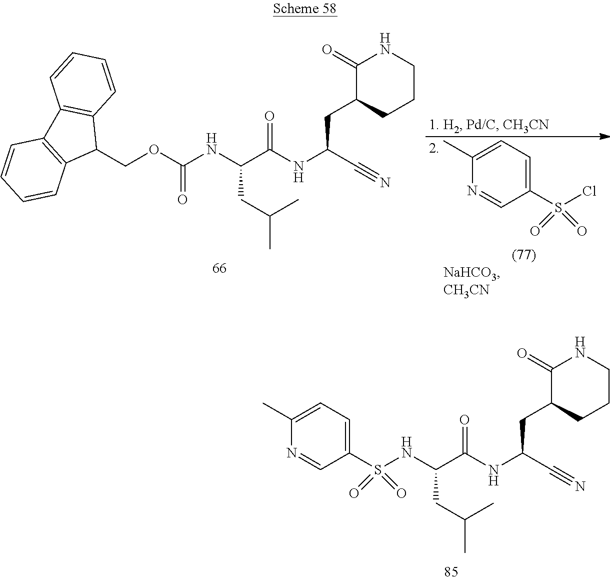 [SMILES: CC(C)C[C@H](NC(=O)OCC1c2ccccc2-c2ccccc21)C(=O)N[C@H](C#N)C[C@@H]1CCCNC1=O ; Cc1ccc(S(=O)(=O)Cl)cn1 ; Cc1ccc(S(=O)(=O)N[C@@H](CC(C)C)C(=O)N[C@H](C#N)C[C@@H]2CCCNC2=O)cn1]

Preparation of N-{(1S)-1-cyano-2-[(3S)-2-oxopiperidin-3-yl]ethyl}-N²-(6-methylpyridine-3-sulfonyl)-L-leucinamide, 85. To a 20% solution of piperidine in DMF (2 mL) was added N-{(1S)-1-cyano-2-[(3S)-2-oxopiperidin-3-yl]ethyl}-N²-{[(9H-fluoren-9-yl)methoxy]carbonyl}-L-leucinamide 67 (36.5 mg, 0.0726 mmol) and after 10 min the mixture was diluted with toluene (10 mL) and concentrated under reduced pressure. The resulting residue was dissolved in acetonitrile (8 mL) and treated with 6-methylpyridine-3-sulfonyl chloride (77) (33.5 mg, 0.175 mmol) in the presence of sodium bicarbonate (69.4 mg, 0.826 mmol). After stirring overnight, the reaction mixture was partitioned between ethyl acetate (25 mL) and water (10 mL). The separated organic layer was dried with sodium sulfate, filtered and concentrated under reduced pressure. The product was purified by column chromatography (eluted with gradient 0% to 8% of methanol in ethyl acetate), which provided 85 (21.0 mg, 66% yield over 2 steps) as off white solid. ¹H NMR (600 MHz, DMSO-d₆) δ 8.91 (d, J=7.7 Hz, 1H), 8.73 (d, J=2.1 Hz, 1H), 8.31 (d, J=8.4 Hz, 1H), 7.96 (dd, J=2.4, 8.2 Hz, 1H), 7.53 (br s, 1H), 7.42 (d, J=8.4 Hz, 1H), 4.71 (q, J=8.1 Hz, 1H), 3.15-3.06 (m, 2H), 2.52 (s, 3H), 2.14-1.97 (m, 2H), 1.82-1.48 (m, 5H), 1.41-1.21 (m, 4H), 0.82 (d, J=6.8 Hz, 3H), 0.72 (d, J=6.6 Hz, 3H). LC/MS: Eluent system B (retention time: 4.83 min); ESI-MS 436 [M+H]⁺, 434 [M−H]⁻.

### Compound 86

### Synthesis of N-{(1S)-1-cyano-2-[(3S)-2-oxopiperidin-3-yl]ethyl}-N²-{[(9H-fluoren-9-yl)methoxy]carbonyl}-4-methyl-L-leucinamide, 86

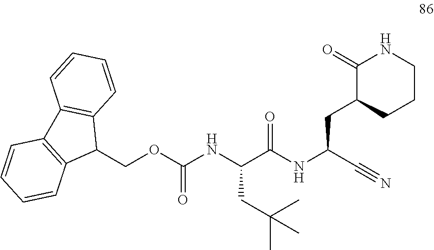 [SMILES: CC(C)(C)C[C@H](NC(=O)OCC1c2ccccc2-c2ccccc21)C(=O)N[C@H](C#N)C[C@@H]1CCCNC1=O]

Compound 86 was synthesized as in Scheme 59.

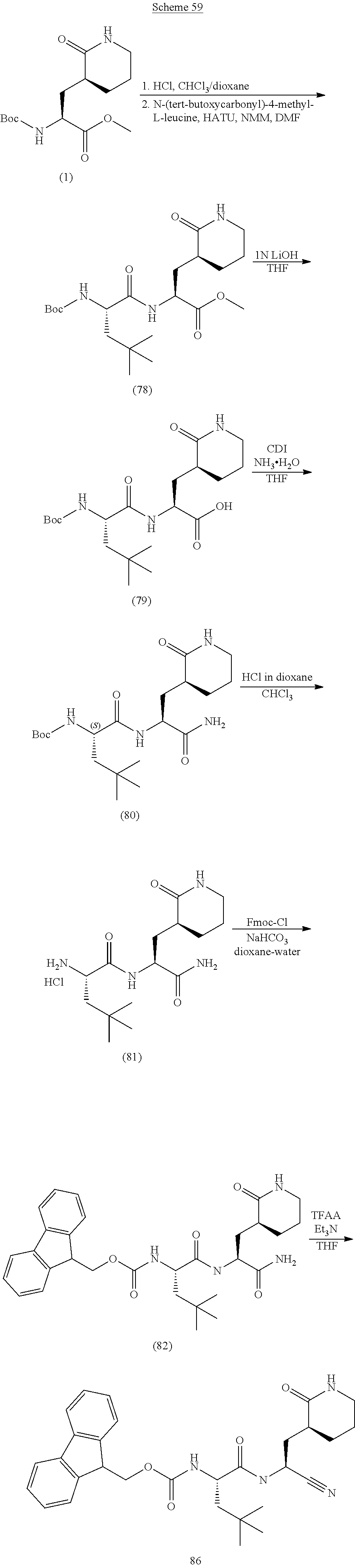

Preparation of methyl N-(tert-butoxycarbonyl)-4-methyl-L-leucyl-3-[(3S)-2-oxopiperidin-3-yl]-L-alaninate, (78). A solution of methyl N-(tert-butoxycarbonyl)-3-[(3S)-2-oxopiperidin-3-yl]-L-alaninate (1) (1.12 g, 3.73 mmol) in CHCl₃ (100 mL) was cooled using an ice-water bath and then 4 M HCl in 1,4-dioxane (11 mL) was added. After 30 min, the ice bath was removed. After overnight, the mixture was concentrated under reduced pressure. Treatment with DCM (3×25 mL), and drying under reduced pressure for additional 1 h. To the resulting residue was added N-(tert-butoxycarbonyl)-4-methyl-L-leucine (1.01 g, 4.10 mmol) and anhydrous DMF (25 mL) and the mixture was cooled in an ice bath, then HATU (1.56 g, 4.10 mmol) was added followed by dropwise addition of NMM (1.23 mL, 11.2 mmol). After 45 min, saturated aqueous brine solution (50 mL) was added and the resulting mixture was extracted with EtOAc (3×50 mL). The combined organic layer was washed with saturated brine solution (1×50 mL), dried over anhydrous Na₂SO₄, filtered, and concentrated under reduced pressure. The residue was dissolved in CHCl₃ (25 mL) and loaded on silica gel column (120 g Silicycle column) and the product was purified by Biotage® with a gradient of 0 to 100% EtOAC in hexanes then followed by a gradient of 0 to 4% MeOH in EtOAc, which generated (78) (1.13 g, 71% yield over two steps) as an off-white solid.

Preparation of N-(tert-butoxycarbonyl)-4-methyl-L-leucyl-3-[(3S)-2-oxopiperidin-3-yl]-L-alanine, (79). To a solution of N-(tert-butoxycarbonyl)-4-methyl-L-leucyl-3-[(3S)-2-oxopiperidin-3-yl]-L-alaninate (78) (1.13 g, 2.64 mmol) in THF (50 mL) cooled in an ice bath was added a 1.0 M LiOH aqueous solution (25 mL). After 1 h, the pH of the reaction mixture was adjusted to 4 by adding 1.0 M HCl aqueous solution while still cooling in the ice bath. The resulting two layers were separated and the aqueous layer was extracted with EtOAc (3×50 mL). The combined organic phase was washed with brine (1×50 mL), dried over anhydrous Na₂SO₄, filtered, and concentrated under reduced pressure, which afforded (79) (1.07 g, 97% yield) as an off-white solid. This material was used without further purification in the next step.

Preparation of N-(tert-butoxycarbonyl)-4-methyl-L-leucyl-3-[(3S)-2-oxopiperidin-3-yl]-L-alaninamide, (80). To N-(tert-butoxycarbonyl)-4-methyl-L-leucyl-3-[(3S)-2-oxopiperidin-3-yl]-L-alanine (79) (1.07 g, 2.59 mmol) was added anhydrous THF (30 mL) and CDI (840 mg, 5.18 mmol). After 15 min, NH₃ (aq., 28%, 4.10 mL, 60.6 mmol) was added. After a further 30 min, water (50 mL) was added and the mixture cooled in an ice-water bath. Then 1.0 M HCl aqueous solution was added until the pH was about 5. The mixture was extracted with EtOAc (3×50 mL). The combined organic phase was washed with brine (1×50 mL), dried over anhydrous Na₂SO₄, filtered, and concentrated under reduced pressure. The product was purified by column chromatography on silica gel using Biotage® with a gradient of 0 to 8% MeOH in CHCl₃, which generated (80) (1.07 g, quantitative yield) as a white solid.

Preparation of 4-methyl-L-leucyl-3-[(3S)-2-oxopiperidin-3-yl]-L-alaninamide hydrogen chloride salt, (81). A solution of N-(tert-butoxycarbonyl)-4-methyl-L-leucyl-3-[(3S)-2-oxopiperidin-3-yl]-L-alaninamide (80) (570 mg, 1.39 mmol) in CHCl₃ (20 mL) was cooled using an ice-water bath and then 4 M HCl in 1,4-dioxane (6 mL) was added. After 30 min, the ice bath was removed. After 4 h, the mixture was concentrated under reduced pressure. The residue was treated with DCM (3×25 mL), and further dried under reduced pressure for 1 h, which generated (81) (482 mg, quantitative yield) as a white solid. This material was used without further purification in the next step.

Preparation of N-{[(9H-fluoren-9-yl)methoxy]carbonyl}-4-methyl-L-leucyl-3-[(3S)-2-oxopiperidin-3-yl]-L-alaninamide, (82). To 4-methyl-L-leucyl-3-[(3S)-2-oxopiperidin-3-yl]-L-alaninamide hydrogen chloride salt (81) (480 mg, 1.38 mmol) was added dioxane (25 mL) and a solution of NaHCO₃ (348 mg, 4.14 mmol) in water (30 mL). To the mixture was slowly added a solution of fluorenylmethyloxycarbonyl chloride (Fmoc-Cl) (536 mg, 2.07 mmol) in dioxane (5 mL). After 15 min, the reaction mixture was diluted with water (20 mL) and extracted with EtOAc (3×50 mL). The combined organic phase was washed with brine (1×50 mL), dried over anhydrous Na₂SO₄, filtered, and concentrated under reduced pressure. The product was purified by column chromatography on silica gel using a Biotage® with a gradient of 0 to 6% MeOH in CHCl₃, which generated (82) (616 mg, 83% yield) as a white solid.

Preparation of N-{(1S)-1-cyano-2-[(3S)-2-oxopiperidin-3-yl]ethyl}-N²-{[(9H-fluoren-9-yl)methoxy]carbonyl}-4-methyl-L-leucinamide, 86. To a solution of N-{[(9H-fluoren-9-yl)methoxy]carbonyl}-4-methyl-L-leucyl-3-[(3S)-2-oxopiperidin-3-yl]-L-alaninamide (82) (616 mg, 1.51 mmol) in anhydrous THF (30 mL) cooled in an ice bath was added Et₃N (0.480 mL, 3.45 mmol), followed by dropwise addition of TFAA (0.320 mg, 2.30 mmol). After 15 min, water (30 mL) was added. The two layers were separated and the aqueous layer was extracted with EtOAc (3×30 mL). The combined organic phase was washed with brine (3×30 mL), dried over anhydrous Na₂SO₄, filtered, and concentrated under reduced pressure. The product was purified by column chromatography on silica gel using a Biotage® with a gradient of 0 to 2% MeOH in CHCl₃, which generated 86 (510 mg, 85% yield) as a white solid. ¹H NMR (600 MHz, DMSO-d₆) δ 8.82 (d, J=7.9 Hz, 1H), 7.89 (dd, J=0.8, 7.5 Hz, 2H), 7.71 (t, J=8.4 Hz, 2H), 7.62 (d, J=8.3 Hz, 1H), 7.52 (br s, 1H), 7.42 (t, J=7.4 Hz, 2H), 7.34-7.30 (m, 2H), 5.05-4.96 (m, 1H), 4.37-4.28 (m, 1H), 4.28-4.18 (m, 2H), 4.07-4.00 (m, 1H), 3.11-3.01 (m, 2H), 2.31-2.18 (m, 2H), 1.84-1.74 (m, 2H), 1.72-1.63 (m, 1H), 1.62-1.46 (m, 3H), 1.42-1.32 (m, 1H), 0.90 (s, 9H). LC/MS: Eluent system A (retention time: 6.65 min); ESI-MS: 517 [M+H]®.

### Compound 87

### Synthesis of (3S)-3-({4-methyl-N-[(2R)-oxolane-2-carbonyl]-L-leucyl}amino)-2-oxo-4-[(3S)-2-oxopiperidin-3-yl]butyl 2,4,6-trimethylpyridine-3-carboxylate, 87

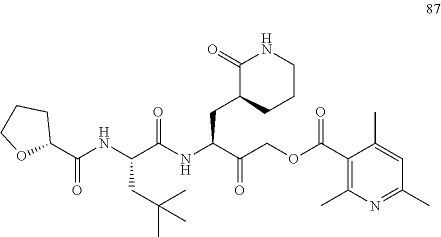 [SMILES: Cc1cc(C)c(C(=O)OCC(=O)[C@H](C[C@@H]2CCCNC2=O)NC(=O)[C@H](CC(C)(C)C)NC(=O)[C@H]2CCCO2)c(C)n1]

Compound 87 was synthesized as in Scheme 60.

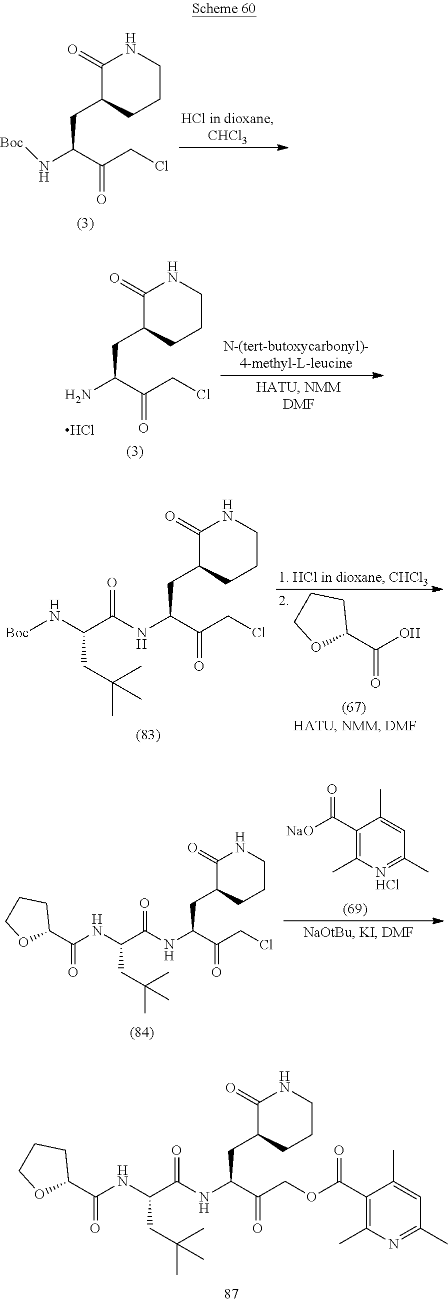

Preparation of (3S)-3-[(2S)-2-amino-4-chloro-3-oxobutyl]piperidin-2-one hydrogen chloride salt, (4). A solution of tert-butyl {(2S)-4-chloro-3-oxo-1-[(3S)-2-oxopiperidin-3-yl]butan-2-yl}carbamate, (3) (207.3 mg, 0.6503 mmol) in CHCl₃ (10 mL) was cooled using an ice-water bath and then 4 M HCl in 1,4-dioxane (2 mL) was added. After 30 min, the ice bath was removed. After overnight, the mixture was concentrated under reduced pressure at 35° C. (water bath temperature), treated with DCM (3×10 mL), and dried for an additional 1 h under reduced pressure to afford (4) (166 mg) as an off-white solid. This material was used without further purification in the next step.

Preparation of N²-(tert-butoxycarbonyl)-N-{(2S)-4-chloro-3-oxo-1-[(3S)-2-oxopiperidin-3-yl]butan-2-yl}-4-methyl-L-leucinamide, (83). To a solution (3S)-3-[(2S)-2-amino-4-chloro-3-oxobutyl]piperidin-2-one hydrogen chloride salt (4) (166 mg, 0.650 mmol) and N-(tert-butoxycarbonyl)-4-methyl-L-leucine (272 mg, 0.715 mmol) in anhydrous DMF (10 mL) cooled in an ice bath was added HATU (268 mg, 0.715 mmol). Then, NMM (215 μL, 1.95 mmol) was added dropwise. After 45 min, a saturated aqueous brine solution (30 mL) was added, and the resulting mixture was extracted with EtOAc (3×30 mL). The combined organic layer was washed with saturated brine solution (1×30 mL), dried over anhydrous Na₂SO₄, filtered, and concentrated under reduced pressure. The resulting residue was dissolved in CHCl₃ (10 mL) and loaded on silica gel column, and the product was purified by Biotage® with a gradient of 0 to 100% EtOAc in hexanes, which generated (83) (185.6 mg, 64% yield) as an off-white solid.

Preparation of N-{(2S)-4-chloro-3-oxo-1-[(3S)-2-oxopiperidin-3-yl]butan-2-yl}-4-methyl-N²-[(2R)-oxolane-2-carbonyl]-4-methyl-L-leucinamide, (84). To a solution of N²-(tert-butoxycarbonyl)-N-{(2S)-4-chloro-3-oxo-1-[(3S)-2-oxopiperidin-3-yl]butan-2-yl}-4-methyl-L-leucinamide (83) (185 mg, 0.415 mmol) in CHCl₃ (20 mL) was cooled using an ice-water bath and then 4 M HCl in 1,4-dioxane (2 mL) was added. After 30 min, the ice bath was removed. After overnight, the mixture was concentrated under reduced pressure. Treatment with DCM (3×10 mL), and then it was dried under reduced pressure for 1 h. To a solution of this residue and (2R)-oxolane-2-carboxylic acid (67) (53.2 mg, 0.457 mmol) in anhydrous DMF (10 mL) after cooling in an ice bath was added HATU (174 mg, 0.457 mmol), followed by dropwise addition of NMM (0.230 mL, 2.08 mmol). After 45 min, saturated aqueous brine solution (50 mL) was added and the resulting mixture was extracted with EtOAc (3×50 mL). The combined organic layer was washed with saturated brine solution (1×50 mL), dried over anhydrous Na₂SO₄, filtered, and concentrated under reduced pressure. The product was purified by column chromatography on silica gel using a Biotage® with a gradient of 0 to 10% MeOH in EtOAc, which generated (84) (117 mg, 94% yield) as a white solid.

Preparation of (3S)-3-({4-methyl-N-[(2R)-oxolane-2-carbonyl]-L-leucyl}amino)-2-oxo-4-[(3S)-2-oxopiperidin-3-yl]butyl 2,4,6-trimethylpyridine-3-carboxylate, 87. To sodium 2,4,6-trimethylpyridine-3-carboxylate hydrogen chloride salt (69) (355 mg, 1.59 mmol) in anhydrous DMF (10 mL) was added sodium tert-butoxide (102 mg, 1.06 mmol). After 30 min, a solution of N-{(2S)-4-chloro-3-oxo-1-[(3S)-2-oxopiperidin-3-yl]butan-2-yl}-4-methyl-N²-[(2R)-oxolane-2-carbonyl]-L-leucinamide (84) (117 mg, 0.264 mmol) in DMF (5 mL) and KI (43.9 mg, 0.264 mmol) were added. After overnight, saturated brine solution (50 mL) was then added, and the resulting mixture was extracted with CHCl₃ (3×10 mL). The combined organic layer was washed with saturated brine solution (1×50 mL), dried over anhydrous Na₂SO₄, filtered, and concentrated under reduced pressure. The residue was dissolved in CHCl₃ (10 mL) and loaded on a silica gel column, and the product was purified by Biotage® with a gradient of 0 to 8% MeOH in EtOAc, which afforded 87 (32.1 mg, 21% yield) as an off-white solid. ¹H NMR (600 MHz, DMSO-d₆) δ 8.50 (d, J=8.1 Hz, 1H), 7.77 (d, J=8.7 Hz, 1H), 7.51 (br s, 1H), 7.05 (s, 1H), 5.19-5.05 (m, 2H), 4.56-4.48 (m, 1H), 4.39-4.34 (m, 1H), 4.25-4.21 (m, 1H), 3.97-3.90 (m, 1H), 3.79-3.74 (m, 1H), 3.17-3.05 (m, 2H), 2.46 (s, 3H), 2.42 (s, 3H), 2.30 (s, 3H), 2.24-2.14 (m, 2H), 2.13-2.06 (m, 1H), 1.89-1.69 (m, 6H), 1.67-1.59 (m, 2H), 1.58-1.47 (m, 1H), 1.43-1.33 (m, 1H), 0.91 (s, 9H). LC/MS: Eluent system B (retention time: 5.61 min); ESI-MS: 573 [M+H]⁺.

### Compound 88

### Synthesis of N-[(2S)-1-({(1S)-1-cyano-2-[(3S)-2-oxopiperidin-3-yl]ethyl}amino)-4,4-dimethyl-1-oxopentan-2-yl]-1H-benzimidazole-2-carboxamide, 88

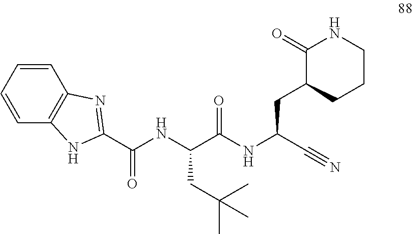 [SMILES: CC(C)(C)C[C@H](NC(=O)c1nc2ccccc2[nH]1)C(=O)N[C@H](C#N)C[C@@H]1CCCNC1=O]

Compound 88 was synthesized as in Scheme 61.

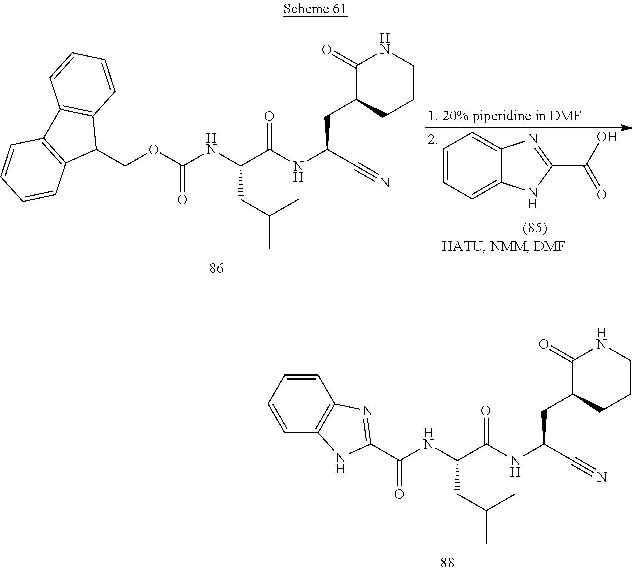 [SMILES: CC(C)C[C@H](NC(=O)OCC1c2ccccc2-c2ccccc21)C(=O)N[C@H](C#N)C[C@@H]1CCCNC1=O ; O=C(O)c1nc2ccccc2[nH]1 ; CC(C)C[C@H](NC(=O)c1nc2ccccc2[nH]1)C(=O)N[C@H](C#N)C[C@@H]1CCCNC1=O]

Preparation of N-[(2S)-1-({(1S)-1-cyano-2-[(3S)-2-oxopiperidin-3-yl]ethyl}amino)-4,4-dimethyl-1-oxopentan-2-yl]-1H-benzimidazole-2-carboxamide, 88. To a solution of 20% piperidine in DMF (1.0 mL) was added N-{(1S)-1-cyano-2-[(3S)-2-oxopiperidin-3-yl]ethyl}-N²-{[(9H-fluoren-9-yl)methoxy]carbonyl}-4-methyl-L-leucinamide 86 (43.4 mg, 0.084 mmol). After 15 min, volatiles were removed under reduced pressure at 40° C. (water bath temperature) and the residue was co-evaporated with toluene (3×10 mL). To the resulting residue was added anhydrous DMF (3 mL) and the mixture cooled in an ice-water bath. Then 1H-benzimidazole-2-carboxylic acid (85) (20.4 mg, 0.126 mmol) and HATU (47.9 mg, 0.126 mmol) were added followed by NMM (27.7 μL, 0.252 mmol). After 3 h, saturated brine solution (15 mL) was added and the mixture extracted with EtOAc (3×15 mL). The combined organic phase was washed with brine (1×30 mL), dried over anhydrous Na₂SO₄, filtered, and concentrated under reduced pressure. The product was purified by column chromatography on silica gel using Biotage® with a gradient of 0 to 4% MeOH in CHCl₃, which generated 88 (34.8 mg, 94% yield) as a white solid. ¹H NMR (600 MHz, DMSO-d₆) δ 13.32 (s, 1H), 8.86-8.82 (m, 2H), 7.76 (d, J=8.1 Hz, 1H), 7.55 (d, J=8.1 Hz, 1H), 7.52 (br s, 1H), 7.36-7.31 (m, 1H), 7.31-7.27 (m, 1H), 5.08-5.01 (m, 1H), 4.59-4.53 (m, 1H), 3.14-3.02 (m, 2H), 2.32-2.22 (m, 2H), 1.90-1.77 (m, 3H), 1.76-1.67 (m, 2H), 1.60-1.50 (m, 1H), 1.43-1.34 (m, 1H), 0.94 (s, 9H). LC/MS: Eluent system A (retention time: 4.96 min); ESI-MS: 439 [M+H]⁺.

### Compound 89

### Synthesis of 1-[(benzyloxy)carbonyl]prolyl-N-{(1S)-1-cyano-2-[(3S)-2-oxopiperidin-3-yl]ethyl}-4-methyl-L-leucinamide, 89

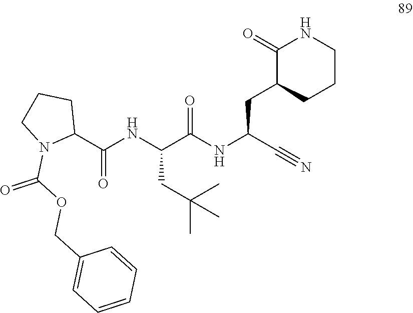 [SMILES: CC(C)(C)C[C@H](NC(=O)C1CCCN1C(=O)OCc1ccccc1)C(=O)N[C@H](C#N)C[C@@H]1CCCNC1=O]

Compound 89 was synthesized as in Scheme 62.

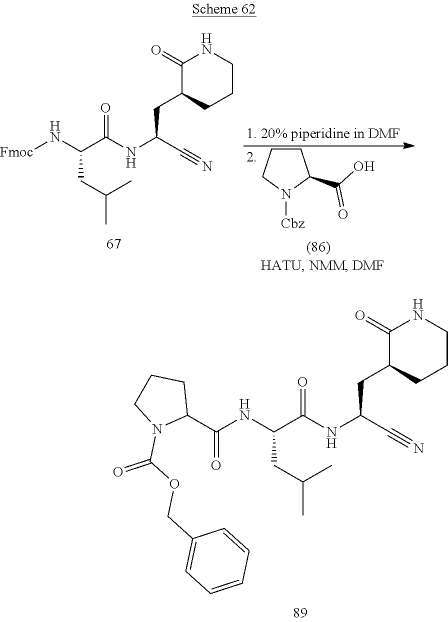

Preparation of 1-[(benzyloxy)carbonyl]prolyl-N-{(1S)-1-cyano-2-[(3S)-2-oxopiperidin-3-yl]ethyl}-4-methyl-L-leucinamide, 89. A similar procedure as described for compound 88 was followed with N-{(1S)-1-cyano-2-[(3S)-2-oxopiperidin-3-yl]ethyl}-N²-{[(9H-fluoren-9-yl)methoxy]carbonyl}-4-methyl-L-leucinamide 86 (53.5 mg, 0.104 mmol), 20% piperidine solution in DMF (1.0 mL), followed by 1-[(benzyloxy)carbonyl]-L-proline (86) (28.4 mg, 0.114 mmol), HATU (43.3 mg, 0.114 mmol) and NMM (24.2 μL, 0.311 mmol) in DMF (3 mL). The product was purified by column chromatography on silica gel using Biotage® with a gradient of 0 to 4% MeOH in CHCl₃, which generated 89 (48.6 mg, 89% yield) as a white solid. ¹H NMR (600 MHz, DMSO-d₆) δ 8.89 (d, J=7.9 Hz, 1H), 8.16 (t, J=8.7 Hz, 1H), 7.54 (br s, 1H), 7.41-7.35 (m, 2H), 7.35-7.26 (m, 3H), 5.18-5.03 (m, 1H), 5.02-4.91 (m, 2H). 4.31-4.24 (m, 2H), 3.50-3.39 (m, 2H), 3.15-3.04 (m, 2H), 2.37-2.06 (m, 3H), 1.85-1.63 (m, 7H), 1.61-1.48 (m, 2H), 1.45-1.32 (m, 1H), 0.90 (s, 9H). LC/MS: Eluent system A (retention time: 5.25 min); ESI-MS: 526 [M+H]⁺.

### Compound 90

### Synthesis of prolyl-N-{(1S)-1-cyano-2-[(3S)-2-oxopiperidin-3-yl]ethyl}-4-methyl-L-leucinamide, 90

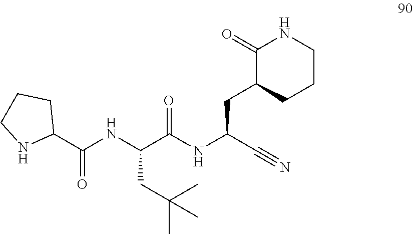 [SMILES: CC(C)(C)C[C@H](NC(=O)C1CCCN1)C(=O)N[C@H](C#N)C[C@@H]1CCCNC1=O]

Compound 90 was synthesized as in Scheme 63.

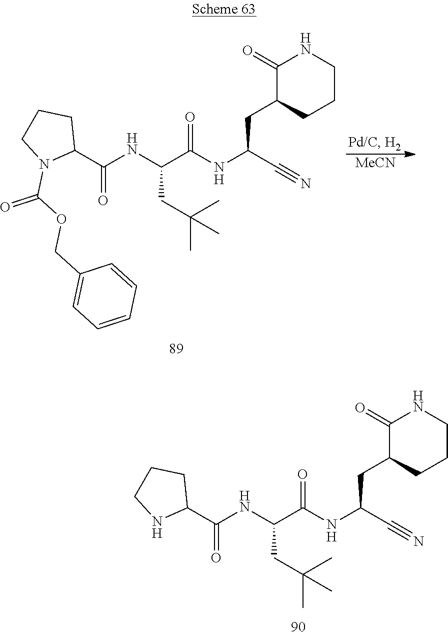 [SMILES: "MeCN" ; CC(C)(C)C[C@H](NC(=O)C1CCCN1C(=O)OCc1ccccc1)C(=O)N[C@H](C#N)C[C@@H]1CCCNC1=O ; CC(C)(C)C[C@H](NC(=O)C1CCCN1)C(=O)N[C@H](C#N)C[C@@H]1CCCNC1=O]

Preparation of prolyl-N-{(1S)-1-cyano-2-[(3S)-2-oxopiperidin-3-yl]ethyl}-4-methyl-L-leucinamide, 90. To a solution of 1-[(benzyloxy)carbonyl]prolyl-N-{(1S)-1-cyano-2-[(3S)-2-oxopiperidin-3-yl]ethyl}-4-methyl-L-leucinamide 89 (34.3 mg, 0.0652 mmol) in acetonitrile (10 mL) was degassed under vacuum and 5% Pd/C (68.5 mg, ˜50% w/w in water) was added. Hydrogen was added using a balloon. After overnight, the suspension was filtered through a pad of Celite®, that was also washed with acetonitrile (3×10 mL), followed by MeOH (3×10 mL). The combined organics were concentrated under reduced pressure. The product was purified by column chromatography on silica gel using Biotage® with a gradient of 0 to 10% MeOH in CHCl₃, which generated 90 (7.8 mg, 30% yield). ¹H NMR (600 MHz, DMSO-d₆) δ 8.95-8.89 (m, 1H), 8.15-8.05 (m, 1H), 7.56 (br s, 1H), 4.98 (br d, J=8.3 Hz, 1H), 4.49-4.37 (m, 1H), 4.35-4.27 (m, 1H), 3.16-3.04 (m, 2H), 2.93-2.84 (m, 1H), 2.82-2.72 (m, 1H), 2.31-2.14 (m, 2H), 2.02-1.90 (m, 2H), 1.87-1.70 (m, 4H), 1.69-1.52 (m, 5H), 1.44-1.35 (m, 1H), 0.90 (s, 9H). LC/MS: Eluent system A (retention time: 4.58 min); ESI-MS: 392 [M+H]⁺.

### Compound 91

### Synthesis of N-{(1S)-1-cyano-2-[(3S)-2-oxopiperidin-3-yl]ethyl}-N²-[(1,3-oxazol-2-yl)methyl]-L-leucinamide, 91

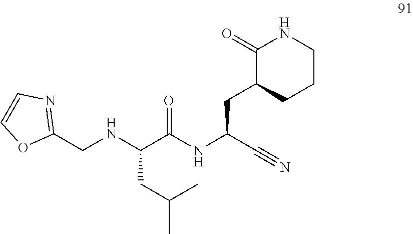 [SMILES: CC(C)C[C@H](NCc1ncco1)C(=O)N[C@H](C#N)C[C@@H]1CCCNC1=O]

Compound 91 was synthesized as in Scheme 64.

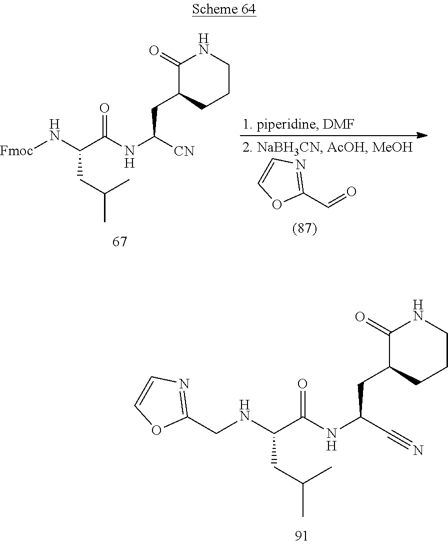

Preparation of N-{(1S)-1-cyano-2-[(3S)-2-oxopiperidin-3-yl]ethyl}-N²-[(1,3-oxazol-2-yl)methyl]-L-leucinamide, 91. Following a similar procedure as described for compound 78 in Scheme 51 with N-{(1S)-1-cyano-2-[(3S)-2-oxopiperidin-3-yl]ethyl}-N²-{[(9H-fluoren-9-yl)methoxy]carbonyl}-L-leucinamide 67 (30 mg, 0.060 mmol), piperidine (2 mL, 20% in DMF), 1,3-oxazole-2-carbaldehyde (87) (7.0 mg, 0.072 mmol), acetic acid (1 drop), NaBH₃CN (7.0 mg, 0.11 mmol) and methanol (5 mL), and purification by column chromatography on silica (0 to 1% methanol in 1:1 chloroform/ethyl acetate) generated 91 (8.0 mg, 37% yield) as a white solid. ¹H NMR (600 MHz, DMSO-d₆) δ 8.81 (d, J=8.3 Hz, 1H), 8.06-8.01 (m, 1H), 7.55 (br d, J=9.8 Hz, 1H), 7.16-7.11 (m, 1H), 5.16-4.85 (m, 2H), 3.15-3.31 (m, 2H), 2.35-2.24 (m, 2H), 2.24-2.14 (m, 2H), 1.83-1.86 (m, 1H), 1.74-1.83 (m, 2H), 1.71-1.60 (m, 1H), 1.60-1.50 (m, 2H), 1.39-1.48 (m, 2H), 1.24-1.28 (m, 1H), 0.86 (d, J=6.8 Hz, 3H), 0.80 (d, J=6.8 Hz, 3H). LC/MS: Eluent system C (see compound 78) (retention time: 4.25 min); ESI-MS: 362 [M+H]⁺.

### Compound 92

### Synthesis of N-{(1S)-1-cyano-2-[(3S)-2-oxopiperidin-3-yl]ethyl}-N²-{[(2S)-oxolan-2-yl]methyl}-L-leucinamide, 92

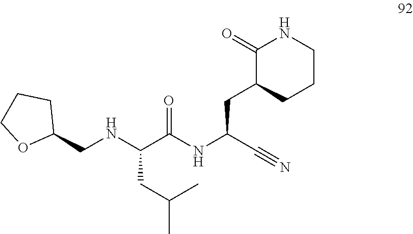 [SMILES: CC(C)C[C@H](NC[C@@H]1CCCO1)C(=O)N[C@H](C#N)C[C@@H]1CCCNC1=O]

Compound 92 was synthesized as in Scheme 65.

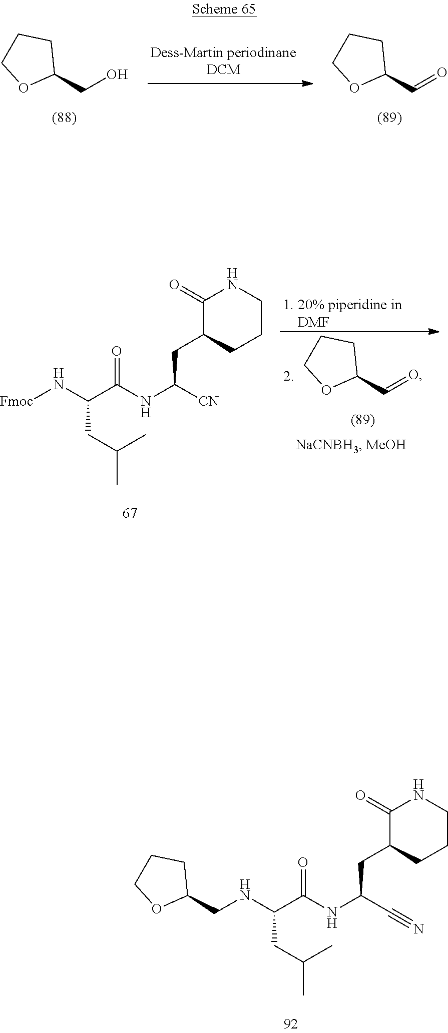

Preparation of (2S)-oxolane-2-carbaldehyde, (89). To a solution of [(2S)-oxolan-2-yl]methanol (88) (41.3 mg, 0.422 mmol) in DCM was added Dess-Martin periodinane (179 mg, 0.422 mmol). After 1 h the mixture was filtered through a pad of Celite® and silica gel. The volatiles of the filtrate were removed under reduced pressure to provide (89) (30.8 mg, 76% yield) as a colorless oil.

Preparation of N-{(1S)-1-cyano-2-[(3S)-2-oxopiperidin-3-yl]ethyl}-N²-{[(2S)-oxolan-2-yl]methyl}-L-leucinamide, 92. To a round bottom flask was added N-{(1S)-1-cyano-2-[(3S)-2-oxopiperidin-3-yl]ethyl}-N²-{[(9H-fluoren-9-yl)methoxy]carbonyl}-L-leucinamide 67 (45.3 mg, 0.0901 mmol) and a 20% piperidine solution in DMF (2.0 mL). After 15 min, the volatiles were removed under reduced pressure with a bath temperature at 37° C. To the residue was added anhydrous MeOH (10 mL) and (2S)-oxolane-2-carbaldehyde (89) (19.8 mg, 0.197 mmol), followed by addition of 1 drop of acetic acid. Then to the reaction mixture was added NaBH₃CN (9.3 mg, 0.148 mmol). After 1 h, the volatiles were removed under reduced pressure and the residue was added EtOAc (25 mL). The mixture was washed with a saturated aqueous NaHCO₃ solution (5 mL), brine (5 mL), dried over Na₂SO₄, filtered and concentrated under reduced pressure. The product was purified by column chromatography on silica gel with a gradient of 0 to 10% MeOH in EtOAc, which generated 92 (12.9 mg, 36% yield) as a sticky solid. ¹HNMR (600 MHz, DMSO-d₆) δ 8.71 (d, J=8.3 Hz, 1H), 7.54 (s, 1H), 5.05-4.99 (m, 1H), 3.84-3.78 (m, 1H), 3.74-3.68 (m, 1H), 3.61-3.55 (m, 1H), 3.15-3.04 (m, 3H), 2.46-2.37 (m, 2H), 2.28 (ddd, J=13.7, 9.5, 6.6 Hz, 1H), 2.18-2.11 (m, 1H), 1.89-1.71 (m, 7H), 1.65-1.59 (m, 1H), 1.58-1.49 (m, 2H), 1.45-1.37 (m, 1H), 1.34-1.29 (m, 2H), 0.88 (d, J=6.4 Hz, 3H), 0.85 (d, J=6.4 Hz, 3H); LC/MS: Eluent system C (see compound 78) (retention time: 4.28 min); ESI-MS: 365 [M+H]⁺.

### Compound 93

### Synthesis of N-[(2S)-1-({(1S)-1-cyano-2-[(3S)-2-oxopiperidin-3-yl]ethyl}amino)-4,4-dimethyl-1-oxopentan-2-yl]-4-methoxy-1-benzofuran-2-carboxamide, 93

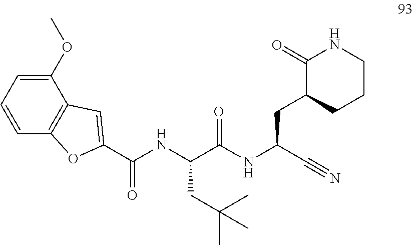 [SMILES: COc1cccc2oc(C(=O)N[C@@H](CC(C)(C)C)C(=O)N[C@H](C#N)C[C@@H]3CCCNC3=O)cc12]

Compound 93 was synthesized as in Scheme 66.

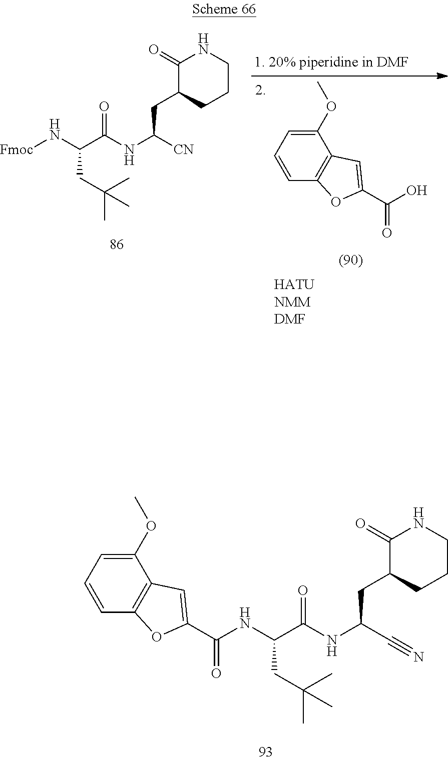

Preparation of N-[(2S)-1-({(1S)-1-cyano-2-[(3S)-2-oxopiperidin-3-yl]ethyl}amino)-4,4-dimethyl-1-oxopentan-2-yl]-4-methoxy-1-benzofuran-2-carboxamide, 93. To a round bottom flask was added N-{(1S)-1-cyano-2-[(3S)-2-oxopiperidin-3-yl]ethyl}-N²-{[(9H-fluoren-9-yl)methoxy]carbonyl}-4-methyl-L-leucinamide 86 (40.8 mg, 0.0790 mmol) and a 20% piperidine solution in DMF (2.0 mL). After 15 min, the volatiles were removed under reduced pressure with a bath temperature at 37° C. To the resulting residue was added anhydrous DMF (5 mL) and 4-methyoxybenzofuran-2-carboxylic acid (90) (18.2 mg, 0.0948 mmol) and the solution was cooled in an ice bath after which HATU (39.2 mg, 0.103 mmol) was added followed by dropwise addition of NMM (0.0261 mL, 0.237 mmol). After 45 min, an ice/saturated aqueous NaHCO₃ mixture (1:1, 20 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in EtOAc, which generated 93 (29.1 mg, 79% yield) as a white solid. ¹HNMR (600 MHz, DMSO-d₆) δ 8.89 (d, J=7.9 Hz, 1H), 8.71 (d, J=8.3 Hz, 1H), 7.65 (d, J=0.8 Hz, 1H), 7.52 (s, 1H), 7.41 (t, J=8.1 Hz, 1H), 7.25 (d, J=8.3 Hz, 1H), 6.86 (d, J=7.9 Hz, 1H), 5.06-5.00 (m, 1H), 4.50 (td, J=8.7, 3.8 Hz, 1H), 3.93 (s, 3H), 3.12-3.02 (m, 2H), 2.28-2.20 (m, 2H), 1.84-1.75 (m, 3H), 1.73-1.66 (m, 2H), 1.58-1.50 (m, 1H), 1.43-1.33 (m, 1H), 0.92 (s, 9H); LC/MS: Eluent system A (retention time: 5.58 min); ESI-MS: 469 [M+H]⁺.

### Compound 94

### Synthesis of (3-chlorophenyl)methyl [(2S)-1-({(1S)-1-cyano-2-[(3S)-2-oxopyrrolidin-3-yl]ethyl}amino)-3-cyclohexyl-1-oxopropan-2-yl]carbamate, 94

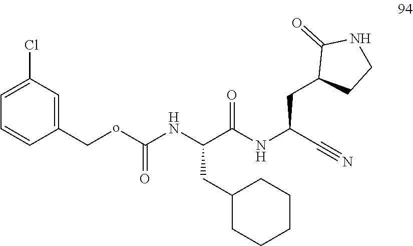

Compound 94 was synthesized as in Scheme 67.

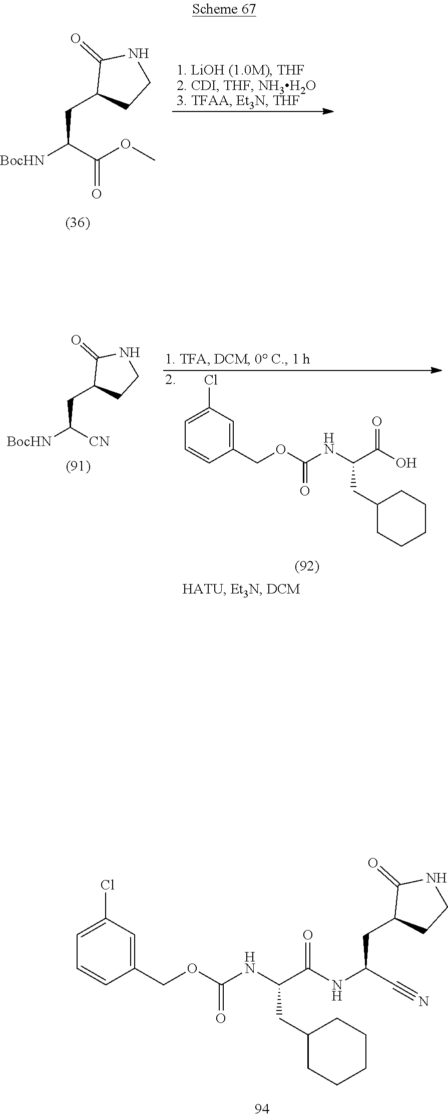

Preparation of tert-butyl {(1S)-1-cyano-2-[(3S)-2-oxopyrrolidin-3-yl]ethyl}carbamate, (91). To a solution of methyl N-(tert-butoxycarbonyl)-3-[(3S)-2-oxopyrrolidin-3-yl]-L-alaninate (36) (500 mg, 1.74 mmol) in THF (4 mL) cooled in an ice bath was added a 1.0 M LiOH aqueous solution (4 mL). After 2 h, the pH of the reaction mixture was adjusted to 3 by adding 1.0 M HCl aqueous solution. The volatiles were removed under reduced pressure and the reside was re-dissolved in DCM (50 mL). The mixture was washed with brine (10 mL), dried over anhydrous Na₂SO₄, filtered, and concentrated under reduced pressure. To the resulting residue was added anhydrous THF (20 mL) and CDI (282 mg, 1.74 mmol). After 15 min, NH₃ (aq., 28%, 1.20 mL, 8.73 mmol) was added. After 2 h, the volatiles were removed under reduced pressure. Purification was accomplished by column chromatography on silica gel with a gradient of 5 to 15% MeOH in CHCl₃, which generated the intermediate (230 mg, 49% yield) as a sticky solid. To this intermediate (110 mg, 0.405 mmol) was added anhydrous THF (5 mL) and the solution was cooled in an ice-bath. Then Et₃N (0.170 mL, 1.22 mmol) was added followed by slow addition of TFAA (172 mg, 0.810 mmol). After 90 min, water (10 mL) was added. The two layers were separated and the aqueous layer was extracted with EtOAc (2×15 mL). The combined organic phase was washed with brine (10 mL), dried over anhydrous Na₂SO₄, filtered, and concentrated under reduced pressure. The product was purified by column chromatography on silica gel with a gradient of 0 to 10% MeOH in CHCl₃, which generated (91) (60.0 mg, 58% yield) as a white solid. LC/MS: Eluent system B (retention time: 2.98 min); ESI-MS: 254 [M+H]⁺.

Preparation of (3-chlorophenyl)methyl [(2S)-1-({(1S)-1-cyano-2-[(3S)-2-oxopyrrolidin-3-yl]ethyl}amino)-3-cyclohexyl-1-oxopropan-2-yl]carbamate, 94. To a solution of tert-butyl {(1S)-1-cyano-2-[(3S)-2-oxopyrrolidin-3-yl]ethyl}carbamate (91) (120 mg, 0.474 mmol) in DCM (4 mL) cooled in an ice bath was added TFA (1.5 mL). After 1 hour, the mixture was concentrated under reduced pressure. The resulting residue was treated with DCM/hexanes (1:1, 2×20 mL) and concentrated under reduced pressure. This material and N-{[(3-chlorophenyl)methoxy]carbonyl}-3-cyclohexyl-L-alanine (92) (177 mg, 0.521 mmol) [prepared using procedures in Journal of Medicinal Chemistry, 2015, 58, page 3144-3155] in anhydrous DCM (8 mL) cooled in an ice bath was added HATU (270 mg, 0.710 mmol), followed by slow addition of Et₃N (0.200 mL, 1.42 mmol). After 45 min, an ice/saturated aqueous NaHCO₃ mixture (1:1, 20 mL) was added and the resulting mixture was extracted with DCM (2×20 mL). The combined organic layer was washed with saturated brine solution (10 mL), dried over anhydrous Na₂SO₄, filtered, and concentrated under reduced pressure. The product was purified by column chromatography on silica gel with a gradient of 60 to 100% EtOAc in hexane, which generated 94 (93.0 mg, 42% yield) as a white solid. ¹HNMR (600 MHz, DMSO-d₆) δ 8.85 (d, J=8.0 Hz, 1H), 7.71 (s, 1H), 7.63 (d, J=8.0 Hz, 1H), 7.43-7.36 (m, 3H), 7.32-7.29 (m, 1H), 5.08-5.01 (m, 2H), 4.97-4.92 (m, 1H), 4.06-3.99 (m, 1H), 3.17-3.10 (m, 1H), 3.09-3.02 (m, 1H), 2.36-2.29 (m, 1H), 2.16-2.07 (m, 2H), 1.82-1.74 (m, 1H), 1.73-1.62 (m, 6H), 1.52-1.40 (m, 2H), 1.34-1.26 (m, 1H), 1.21-1.09 (m, 3H), 0.95-0.81 (m, 2H); LC/MS: Eluent system A (retention time: 6.19 min); ESI-MS: 475 [M+H]⁺.

### Compound 95

### Synthesis of N-{(1S)-1-cyano-2-[(3S)-2-oxopiperidin-3-yl]ethyl}-N²-(cyclohexylmethyl)-L-leucinamide, 95

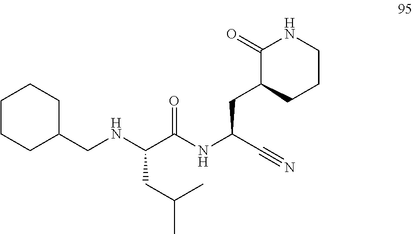 [SMILES: CC(C)C[C@H](NCC1CCCCC1)C(=O)N[C@H](C#N)C[C@@H]1CCCNC1=O]

Compound 95 was synthesized as in Scheme 68.

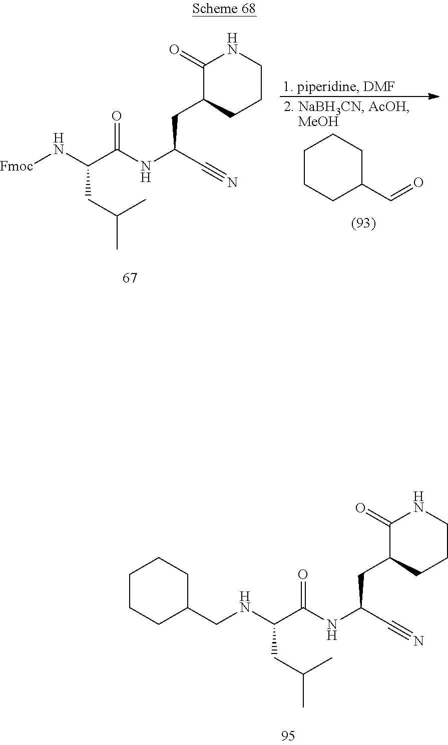

Preparation of N-{(1S)-1-cyano-2-[(3S)-2-oxopiperidin-3-yl]ethyl}-N²-(cyclohexylmethyl)-L-leucinamide, 95. Following a similar procedure as described for compound 78 in Scheme 51, but with N-{(1S)-1-cyano-2-[(3S)-2-oxopiperidin-3-yl]ethyl}-N²-{[(9H-fluoren-9-yl)methoxy]carbonyl}-L-leucinamide 67 33 mg, 0.066 mmol), piperidine (2 mL, 20% in DMF), cyclohexanecarbaldehyde (93) (11 mg, 0.099 mmol), acetic acid (1 drop), NaBH₃CN (8.9 mg, 0.14 mmol) and methanol (5 mL), and purification by column chromatography on silica (0 to 1% methanol in 1:1 chloroform/ethyl acetate) generated 95 (10 mg, 40% yield) as a white solid. ¹H NMR (600 MHz, DMSO-d₆) δ 8.69 (br d, J=8.3 Hz, 1H), 7.60-7.52 (m, 1H), 5.05-4.98 (m, 1H), 3.16-3.06 (m, 2H), 3.05-2.96 (m, 1H), 2.33-2.26 (m, 2H), 2.24-2.19 (m, 1H), 2.19-2.11 (m, 2H), 1.86-1.75 (m, 4H), 1.74-1.59 (m, 6H), 1.57-1.49 (m, 2H), 1.48-1.39 (m, 1H), 1.35-1.27 (m, 2H), 1.27-1.23 (m, 2H), 1.22-1.09 (m, 2H), 0.89 (d, J=6.8 Hz, 3H), 0.85 (d, J=6.4 Hz, 3H). LC/MS: Eluent system C (see compound 78) (retention time: 5.29 min); ESI-MS: 377 [M+H]⁺.

### Compound 96 and 97

### Syntheses of N-{(1S)-1-cyano-2-[(3S)-2-oxopiperidin-3-yl]ethyl}-N²-[(2,3-dihydro-1-benzofuran-2-yl)methyl]-L-leucinamide diastereomers, 96 and 97

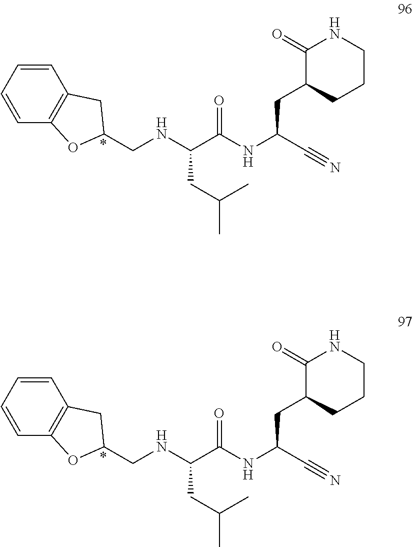

Compounds 96 and 97 were synthesized as in Scheme 69.

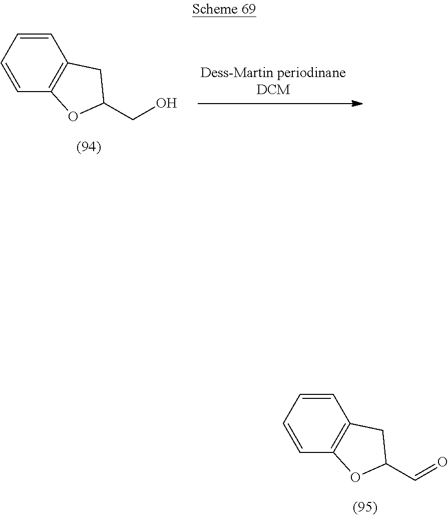 [SMILES: OCC1Cc2ccccc2O1 ; O=CC1Cc2ccccc2O1]

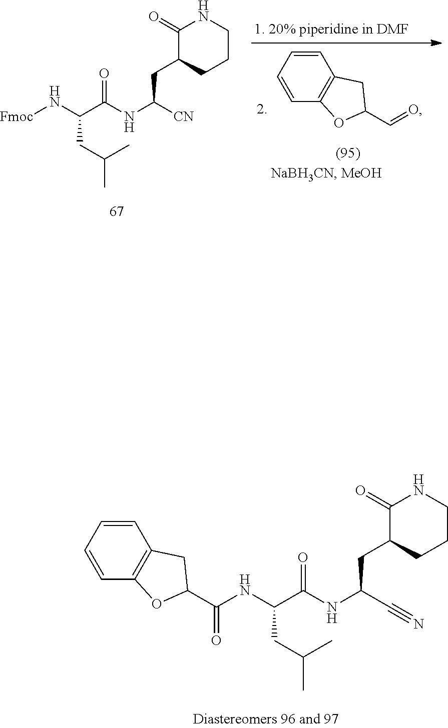

Preparation of 2,3-dihydro-1-benzofuran-2-carbaldehyde, (95). To a solution of (2,3-dihydro-1-benzofuran-2-yl)methanol (94) (51.3 mg, 0.342 mmol) in DCM was added Dess-Martin periodinane (145 mg, 0.342 mmol). After 1 h the mixture was filtered through a pad of Celite® and silica gel. The volatiles of the filtrate were removed under reduced pressure to provide (95) (40.3 mg, 80%) as a colorless oil.

Preparation of N-{(1S)-1-cyano-2-[(3S)-2-oxopiperidin-3-yl]ethyl}-N²-[(2,3-dihydro-1-benzofuran-2-yl)methyl]-L-leucinamide diastereomers, 96 and 97. To a round bottom flask was added N-{(1S)-1-cyano-2-[(3S)-2-oxopiperidin-3-yl]ethyl}-N²-{[(9H-fluoren-9-yl)methoxy]carbonyl}-L-leucinamide 67 (45.3 mg, 0.0901 mmol) and a 20% piperidine solution in DMF (2.0 mL). After 15 min the volatiles were removed under reduced pressure with a bath temperature at 37° C. To the residue was added anhydrous MeOH (10 mL) and 2,3-dihydro-1-benzofuran-2-carbaldehyde (95) (26.7 mg, 0.180 mmol), followed by addition of 1 drop of acetic acid. Then to the reaction mixture was added NaCNBH₃ (8.5 mg, 0.135 mmol). After 1 h, the volatiles were removed under reduced pressure and to the resulting residue was added EtOAc (25 mL). The mixture was washed with a saturated aqueous NaHCO₃ solution (5 mL), brine (5 mL), dried over Na₂SO₄, filtered and concentrated under reduced pressure. The diastereomer products were purified by column chromatography on silica gel with a gradient of 0 to 3% MeOH in EtOAc, which first provided diastereomer 96 (9.5 mg, 26% yield) as a sticky solid. ¹HNMR (600 MHz, DMSO-d₆) δ 8.77 (d, J=8.1 Hz, 1H), 7.53 (s, 1H), 7.18-7.16 (m, 1H), 7.07-7.03 (m, 1H), 6.78 (td, J=7.4, 1.0 Hz, 1H), 6.72 (d, J=7.9 Hz, 1H), 5.05-5.00 (m, 1H), 4.83-4.77 (m, 1H), 3.19 (dd, J=15.8, 9.2 Hz, 1H), 3.13 (t, J=7.2 Hz, 1H), 3.11-3.03 (m, 2H), 2.98 (dd, J=15.7, 7.4 Hz, 1H), 2.69-2.60 (m, 2H), 2.27 (ddd, J=13.6, 9.7, 6.5 Hz, 1H), 2.20-2.11 (m, 1H), 1.83-1.75 (m, 2H), 1.72-1.70 (m, 1H), 1.63 (sep, J=6.8 Hz, 1H), 1.50-1.31 (m, 5H), 0.88 (d, J=6.7 Hz, 3H), 0.86 (d, J=6.5 Hz, 3H); LC/MS: Eluent system C (see compound 78) (retention time: 5.59 min); ESI-MS: 413 [M+H]⁺.

Continuing the column elution at 3% MeOH in EtOAc generated the second diastereomer 97 as a sticky solid (5.2 mg, 14% yield). ¹HNMR (600 MHz, DMSO-d₆) δ 8.77 (d, J=8.1 Hz, 1H), 7.53 (s, 1H), 7.18-7.16 (m, 1H), 7.07-7.03 (m, 1H), 6.78 (td, J=7.4, 0.9 Hz, 1H), 6.72 (d, J=7.9 Hz, 1H), 5.04-4.98 (m, 1H), 4.78 (dtd, J=9.1, 7.0, 5.1 Hz, 1H), 3.21 (dd, J=15.8, 9.0 Hz, 1H), 3.11 (t, J=7.2 Hz, 1H), 3.08-3.04 (m, 2H), 2.89 (dd, J=15.8, 7.2 Hz, 1H), 2.69 (dd, J=12.4, 6.8 Hz, 1H), 2.55 (dd, J=12.4, 4.9 Hz, 1H), 2.27 (ddd, J=13.7, 9.7, 6.4 Hz, 1H), 2.17-2.10 (m, 1H), 1.82-1.75 (m, 2H), 1.71-1.65 (m, 1H), 1.63 (sep, J=6.7 Hz, 1H), 1.51-1.30 (m, 5H), 0.88 (d, J=6.7 Hz, 3H), 0.84 (d, J=6.6 Hz, 3H); LC/MS: Eluent system C (see compound 78) (retention time: 5.80 min); ESI-MS: 413 [M+H]⁺.

### Compound 98

### Synthesis of N-{(1S)-1-cyano-2-[(3S)-2-oxopiperidin-3-yl]ethyl}-N²-[(pyridin-2-yl)methyl]-L-leucinamide, 98

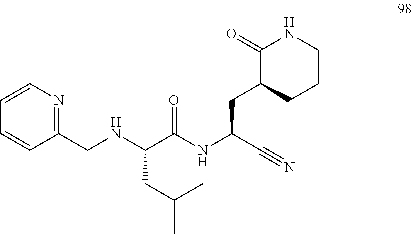 [SMILES: CC(C)C[C@H](NCc1ccccn1)C(=O)N[C@H](C#N)C[C@@H]1CCCNC1=O]

Compound 98 was synthesized as in Scheme 70.

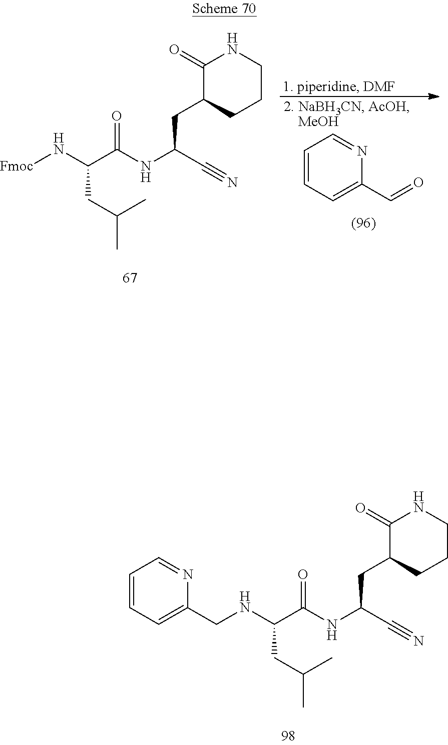

Preparation of N-{(1S)-1-cyano-2-[(3S)-2-oxopiperidin-3-yl]ethyl}-N²-[(pyridin-2-yl)methyl]-L-leucinamide, 98. Following a similar procedure as described for compound 78 in Scheme 51, but with N-{(1S)-1-cyano-2-[(3S)-2-oxopiperidin-3-yl]ethyl}-N²-{[(9H-fluoren-9-yl)methoxy]carbonyl}-L-leucinamide 67 (33 mg, 0.066 mmol), piperidine (2 mL, 20% in DMF), pyridine-2-carbaldehyde (96) (11 mg, 0.099 mmol), acetic acid (1 drop), NaBH₃CN (9 mg, 0.14 mmol) and methanol (5 mL) generated 98 (8 mg) as a gum. LC/MS: Eluent system C (see compound 78) (retention time: 4.62 min); ESI-MS: 372 [M+H]⁺.

### Compound 99

### Synthesis of N-[(2S)-1-({(1S)-1-cyano-2-[(3S)-2-oxopiperidin-3-yl]ethyl}amino)-4,4-dimethyl-1-oxopentan-2-yl]-4-methoxy-1H-pyrrolo[3,2-c]pyridine-2-carboxamide, 99

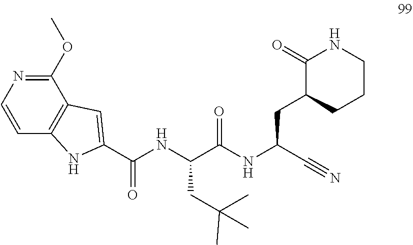 [SMILES: COc1nccc2[nH]c(C(=O)N[C@@H](CC(C)(C)C)C(=O)N[C@H](C#N)C[C@@H]3CCCNC3=O)cc12]

Compound 99 was synthesized as in Scheme 71.

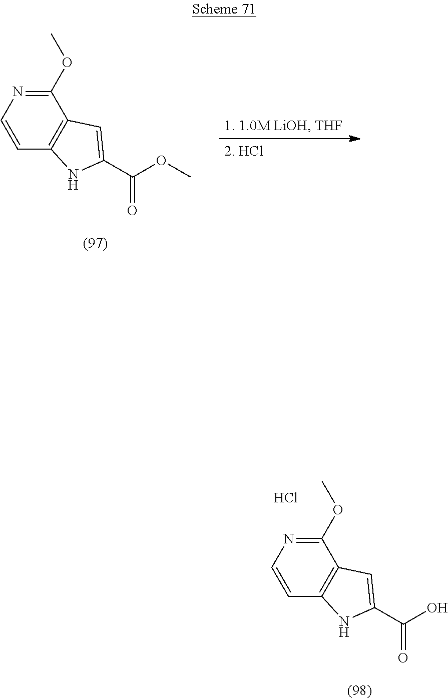

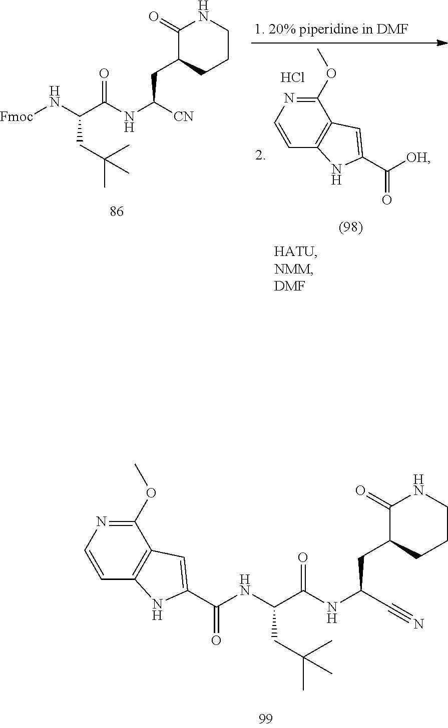

Preparation of 4-methoxy-1H-pyrrolo[3,2-c]pyridine-2-carboxylic acid hydrochloride, (98). To a solution of methyl 4-methoxy-1H-pyrrolo[3,2-c]pyridine-2-carboxylate (97) (24.3 mg, 0.118 mmol) in THF (2 mL) was added a 1.0 M LiOH aqueous solution (2 mL). After overnight the pH of the mixture was adjusted to 3 by adding a 1.0 M HCl aqueous solution. The volatiles were removed under reduced pressure, then the residue was treated with methanol (3×5 mL) and concentrated under reduced pressure. The residue of (98) was dried for overnight under reduced pressure and used in next step without purification.

Preparation of N-[(2S)-1-({(1S)-1-cyano-2-[(3S)-2-oxopiperidin-3-yl]ethyl}amino)-4,4-dimethyl-1-oxopentan-2-yl]-4-methoxy-1H-pyrrolo[3,2-c]pyridine-2-carboxamide, 99. To a round bottom flask was added N-{(1S)-1-cyano-2-[(3S)-2-oxopiperidin-3-yl]ethyl}-N²-{[(9H-fluoren-9-yl)methoxy]carbonyl}-4-methyl-L-leucinamide 86 (40.5 mg, 0.0784 mmol) and 20% piperidine solution in DMF (2.0 mL). After 15 min, the volatiles were removed under reduced pressure with a bath temperature at 37° C. To the residue was added anhydrous DMF (5 mL) and 4-methoxy-1H-pyrrolo[3,2-c]pyridine-2-carboxylic acid hydrochloride (98) (residue) and the solution was cooled in an ice bath, after which HATU (44.9 mg, 0.118 mmol) was added followed by dropwise addition of NMM (0.0431 mL, 0.392 mmol). After 45 min, an ice/saturated aqueous NaHCO₃ mixture (1:1, 20 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 15% MeOH in EtOAc, which generated 99 (21.1 mg, 57% yield) as a white solid. ¹HNMR (600 MHz, DMSO-d₆) δ 11.99 (d, J=1.9 Hz, 1H), 8.89 (d, J=8.0 Hz, 1H), 8.57 (d, J=8.1 Hz, 1H), 7.76 (d, J=6.0 Hz, 1H), 7.50 (s, 1H), 7.38 (dd, J=0.8, 2.3 Hz, 1H), 7.00 (dd, J=0.8, 6.0 Hz, 1H), 5.07-5.01 (m, 1H), 4.51 (td, J=8.7, 3.8 Hz, 1H), 3.97 (s, 3H), 3.12-3.02 (m, 2H), 2.29-2.20 (m, 2H), 1.85-1.74 (m, 3H), 1.71-1.64 (m, 2H), 1.58-1.48 (m, 1H), 1.42-1.34 (m, 1H), 0.94 (s, 9H);

LC/MS: Eluent system B (retention time: 5.67 min); ESI-MS: 469 [M+H]⁺.

### Compound 100

### Synthesis of N-[(2S)-1-({(1S)-1-cyano-2-[(3S)-2-oxopiperidin-3-yl]ethyl}amino)-4,4-dimethyl-1-oxopentan-2-yl]-4-methoxy-1-methyl-1H-pyrrolo[3,2-c]pyridine-2-carboxamide, 100

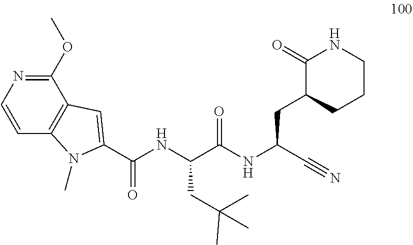 [SMILES: COc1nccc2c1cc(C(=O)N[C@@H](CC(C)(C)C)C(=O)N[C@H](C#N)C[C@@H]1CCCNC1=O)n2C]

Compound 100 was synthesized as in Scheme 72.

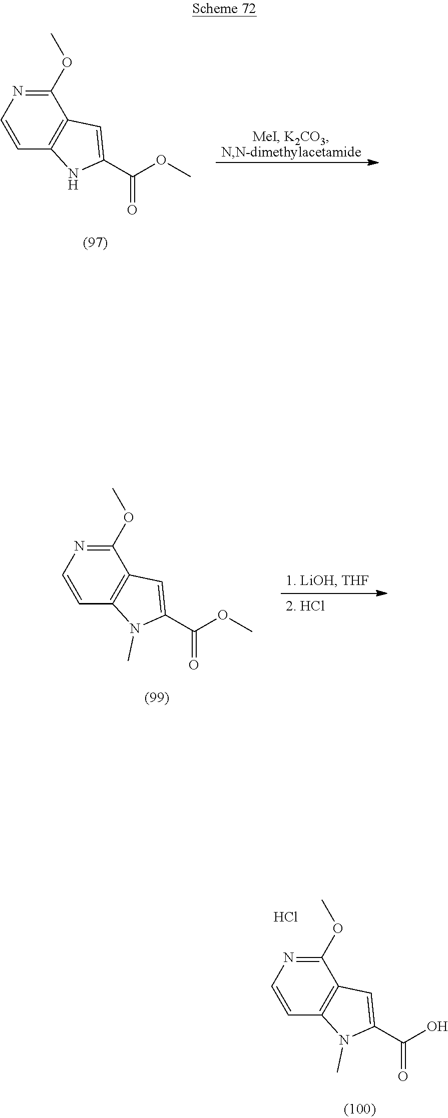 [SMILES: COC(=O)c1cc2c(OC)nccc2[nH]1 ; "MeI, K2CO3,N,N-dimethylacetamide" ; COC(=O)c1cc2c(OC)nccc2n1C ; COc1nccc2c1cc(C(=O)O)n2C ; Cl]

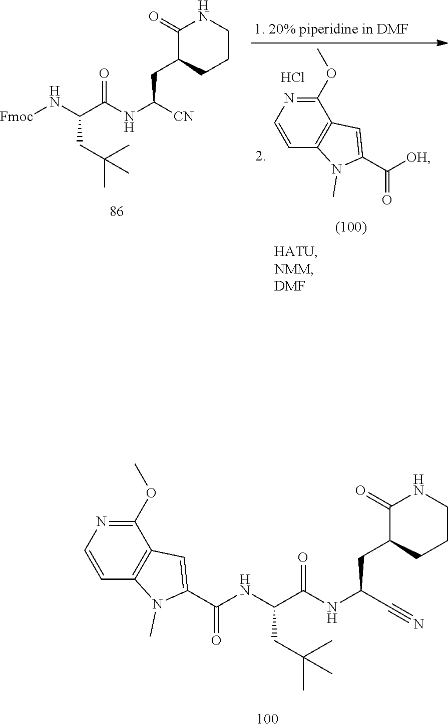

Preparation of methyl 4-methoxy-1-methyl-1H-pyrrolo[3,2-c]pyridine-2-carboxylate, (99). To a solution of methyl 4-methoxy-1H-pyrrolo[3,2-c]pyridine-2-carboxylate (97) (98.8 mg, 0.479 mmol) in anhydrous N,N-dimethylacetamide (3 mL) was added K₂CO₃ (132 mg, 0.958 mmol), followed by Mel (108 mg, 0.719 mmol). The reaction mixture was stirred for 2 days before it was diluted with EtOAc (60 mL). The organic solution was washed with water (3×5 mL), brine (10 mL), dried over anhydrous Na₂SO₄, filtered, and concentrated under reduced pressure. The product was purified by column chromatography on silica gel with a gradient of 0 to 30% EtOAc in hexanes, which generated (99) (75.6 mg, 72% yield) as a white solid. ¹HNMR (600 MHz, DMSO-d₆) δ 7.91 (d, J=6.1 Hz, 1H), 7.26 (dd, J=0.9, 6.1 Hz, 1H), 7.24 (d, J=0.9 Hz, 1H), 4.01 (s, 3H), 3.99 (s, 3H), 3.86 (s, 3H). LC/MS: Eluent system C (see compound 78) (retention time: 6.33 min); ESI-MS: 221 [M+H]⁺.

Preparation of 4-methoxy-1-methyl-1H-pyrrolo[3,2-c]pyridine-2-carboxylic acid hydrochloride, (100). To a solution of methyl 4-methoxy-1-methyl-1H-pyrrolo[3,2-c]pyridine-2-carboxylate (99) (24.2 mg, 0.110 mmol) in THF (2 mL) was added a 1.0 M LiOH aqueous solution (2 mL). After 2 h, the pH of the reaction mixture was adjusted to 3 by adding a 1.0 M HCl aqueous solution. The volatiles were removed under reduced pressure, then the residue was treated with methanol (3×5 mL) and concentrated under reduced pressure. The resulting (100) was dried for overnight and used in next step without further purification.

Preparation of N-[(2S)-1-({(1S)-1-cyano-2-[(3S)-2-oxopiperidin-3-yl]ethyl}amino)-4,4-dimethyl-1-oxopentan-2-yl]-4-methoxy-1-methyl-1H-pyrrolo[3,2-c]pyridine-2-carboxamide, 100. To a round bottom flask was added N-{(1S)-1-cyano-2-[(3S)-2-oxopiperidin-3-yl]ethyl}-N²-{[(9H-fluoren-9-yl)methoxy]carbonyl}-4-methyl-L-leucinamide 86 (38.0 mg, 0.0736 mmol) and a 20% piperidine solution in DMF (2.0 mL). After 15 min the volatiles were removed under reduced pressure with a bath temperature at 37° C. To the residue was added anhydrous DMF (5 mL) and 4-methoxy-1-methyl-1H-pyrrolo[3,2-c]pyridine-2-carboxylic acid hydrochloride (100) and the solution was cooled in an ice bath, after which HATU (41.8 mg, 0.110 mmol) was added followed by dropwise addition of NMM (0.0405 mL, 0.368 mmol). After 45 min, an ice/saturated aqueous NaHCO₃ mixture (1:1, 20 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in EtOAc, which generated 100 (26.8 mg, 75% yield) as a white solid. ¹HNMR (600 MHz, DMSO-d₆) δ 8.82 (d, J=8.0 Hz, 1H), 8.60 (d, J=8.0 Hz, 1H), 7.85 (d, J=6.0 Hz, 1H), 7.50 (s, 1H), 7.33 (d, J=0.8, 1H), 7.18 (dd, J=0.8, 6.0 Hz, 1H), 5.05-5.00 (m, 1H), 4.45 (td, J=8.7, 3.3 Hz, 1H), 3.99 (s, 3H), 3.96 (s, 3H), 3.12-3.02 (m, 2H), 2.33-2.20 (m, 2H), 1.86-1.76 (m, 3H), 1.74-1.69 (m, 1H), 1.67 (dd, J=3.4, 14.3 Hz, 1H), 1.57-1.48 (m, 1H), 1.43-1.35 (m, 1H), 0.95 (s, 9H). LC/MS: Eluent system A (retention time: 3.46 min); ESI-MS: 483 [M+H]⁺.

### Compound 101

### Synthesis of benzyl (2R)-2-{[(2S)-1-({(1S)-1-cyano-2-[(3S)-2-oxopiperidin-3-yl]ethyl}amino)-4,4-dimethyl-1-oxopentan-2-yl]carbamoyl}-2,3-dihydro-1H-indole-1-carboxylate, 101

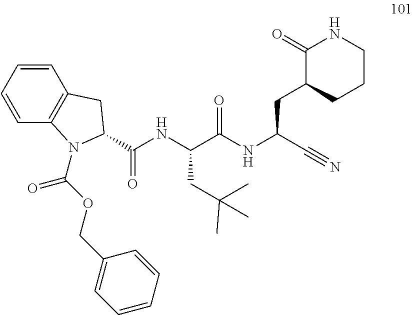 [SMILES: CC(C)(C)C[C@H](NC(=O)[C@H]1Cc2ccccc2N1C(=O)OCc1ccccc1)C(=O)N[C@H](C#N)C[C@@H]1CCCNC1=O]

Compound 101 was synthesized as in Scheme 73.

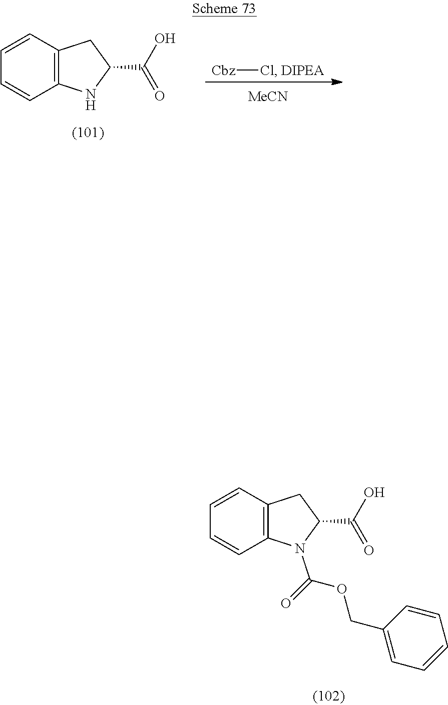

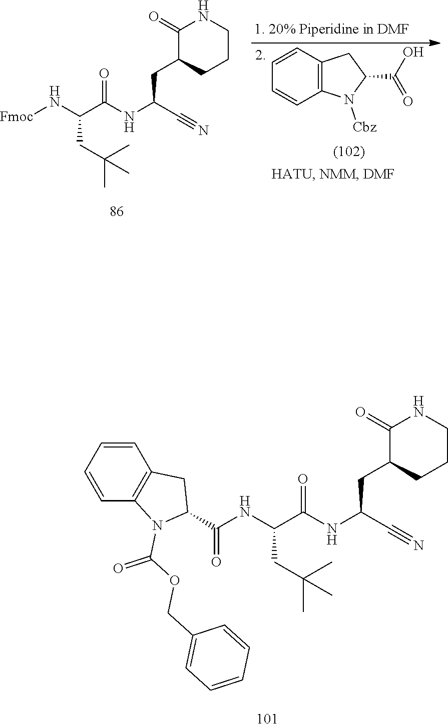

Preparation of (2R)-1-[(benzyloxy)carbonyl]-2,3-dihydro-1H-indole-2-carboxylic acid, (102). Prepared as described in reference Gordeev, Mikhail Fedorovich et al. WO 2008/108988. A solution of (R)-indoline-2-carboxylic acid (101) (1.05 g, 6.43 mmol) and DIPEA (2.3 mL, 12.9 mmol) in MeCN (30 mL) was cooled in an ice-water bath and then benzylchloroformate (Cbz-Cl) (1.0 mL, 7.08 mmol) was added dropwise over 10 min. After additional 30 min, the ice bath was removed. After 4 h, volatiles were removed under reduced pressure. The residue was dissolved in EtOAc (25 mL) and washed with 1% aq. HCl (1×25 mL), water (1×25 mL), saturated brine solution (1×25 mL), dried over anhydrous Na₂SO₄, filtered, and concentrated under reduced pressure, which generated (102) (1.41 g, 74% yield) as an oil that crystallized upon cooling into a brownish solid.

Preparation of benzyl (2R)-2-{[(2S)-1-({(1S)-1-cyano-2-[(3S)-2-oxopiperidin-3-yl]ethyl}amino)-4,4-dimethyl-1-oxopentan-2-yl]carbamoyl}-2,3-dihydro-1H-indole-1-carboxylate, 101. A similar procedure as described for compound 88 was followed with N-{(1S)-1-cyano-2-[(3S)-2-oxopiperidin-3-yl]ethyl}-N²-{[(9H-fluoren-9-yl)methoxy]carbonyl}-4-methyl-L-leucinamide, 86 (53.2 mg, 0.103 mmol), 20% piperidine solution in DMF (1.0 mL) and followed by (2R)-1-[(benzyloxy)carbonyl]-2,3-dihydro-1H-indole-2-carboxylic acid (102) (45.8 mg, 0.154 mmol), HATU (58.6 mg, 0.154 mmol) and NMM (33.9 μL, 0.309 mmol) in DMF (3 mL). The product was purified by column chromatography on silica gel using Biotage® with a gradient of 0 to 1% MeOH in EtOAc, which generated 101 (45.7 mg, 77% yield) as a white solid. ¹H NMR (600 MHz, DMSO-d₆) δ 8.97-8.90 (m, 1H), 8.71-8.45 (m, 1H), 7.78-7.69 (m, 1H), 7.54 (br s, 1H), 7.50-7.24 (m, 5H), 7.23-7.13 (m, 2H), 6.95 (dt, J=0.9, 7.4 Hz, 1H), 5.40-5.20 (m, 1H), 5.12-4.80 (m, 3H), 4.38-4.27 (m, 1H), 3.54-3.43 (m, 1H), 3.13-3.02 (m, 2H), 2.96-2.85 (m, 1H), 2.36-2.02 (m, 2H), 1.92-1.43 (m, 6H), 1.40-1.28 (m, 1H), 0.90 (s, 9H). LC/MS: Eluent system A (retention time: 6.21 min); ESI-MS: 574 [M+H]⁺.

### Compound 102

### Synthesis of (2R)—N-[(2S)-1-({(1S)-1-cyano-2-[(3S)-2-oxopiperidin-3-yl]ethyl}amino)-4,4-dimethyl-1-oxopentan-2-yl]-2,3-dihydro-1H-indole-2-carboxamide, 102

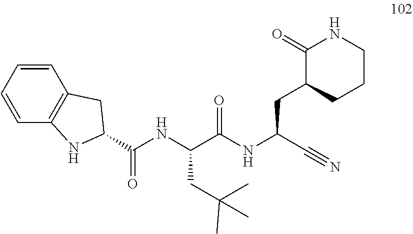 [SMILES: CC(C)(C)C[C@H](NC(=O)[C@H]1Cc2ccccc2N1)C(=O)N[C@H](C#N)C[C@@H]1CCCNC1=O]

Compound 102 was synthesized as in Scheme 74.

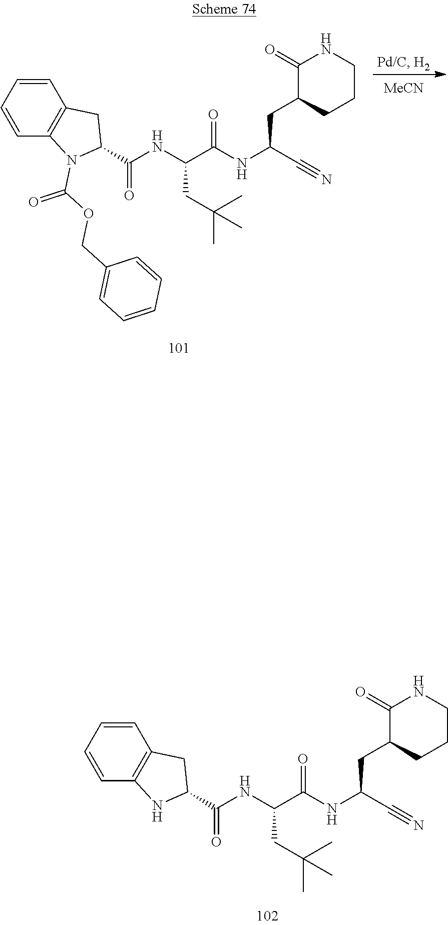 [SMILES: "MeCN" ; CC(C)(C)C[C@H](NC(=O)[C@H]1Cc2ccccc2N1C(=O)OCc1ccccc1)C(=O)N[C@H](C#N)C[C@@H]1CCCNC1=O ; "Pd/C, H2" ; CC(C)(C)C[C@H](NC(=O)[C@H]1Cc2ccccc2N1)C(=O)N[C@H](C#N)C[C@@H]1CCCNC1=O]

Preparation of (2R)—N-[(2S)-1-({(1S)-1-cyano-2-[(3S)-2-oxopiperidin-3-yl]ethyl}amino)-4,4-dimethyl-1-oxopentan-2-yl]-2,3-dihydro-1H-indole-2-carboxamide, 102. A similar procedure as described for compound 90 was followed with benzyl (2R)-2-{[(2S)-1-({(1S)-1-cyano-2-[(3S)-2-oxopiperidin-3-yl]ethyl}amino)-4,4-dimethyl-1-oxopentan-2-yl]carbamoyl}-2,3-dihydro-1H-indole-1-carboxylate 101 (42.6 mg, 0.074 mmol), 5% Pd/C (20.3 mg, ˜50% w/w in water) and acetonitrile (15 mL). The product was purified by column chromatography on silica gel using Biotage® with a gradient of 0 to 1% MeOH in CHCl₃, which generated 102 (18.7 mg, 57% yield) as an off-white solid. ¹H NMR (600 MHz, DMSO-d₆) δ 8.89 (d, J=7.9 Hz, 1H), 7.90 (d, J=8.3 Hz, 1H), 7.53 (br s, 1H), 6.99 (d, J=7.2 Hz, 1H), 6.95 (t, J=7.5 Hz, 1H), 6.59 (d, J=7.6 Hz, 2H), 5.89 (d, J=3.6 Hz, 1H), 5.02-4.92 (m, 1H), 4.36-4.29 (m, 1H), 4.27-4.20 (m, 1H), 3.30-3.23 (m, 2H), 3.13-3.02 (m, 2H), 2.87 (dd, J=8.4, 16.1 Hz, 1H), 2.29-2.15 (m, 2H), 1.84-1.73 (m, 2H), 1.73-1.66 (m, 1H), 1.66-1.61 (m, 1H), 1.59-1.47 (m, 2H), 1.42-1.33 (m, 1H), 0.92 (s, 9H). LC/MS: Eluent system A (retention time: 4.38 min); ESI-MS: 440 [M+H]⁺.

### Compound 103

### Synthesis of N-{(1S)-1-cyano-2-[(3S)-2-oxo(6,6-d₂)piperidin-3-yl]ethyl}-N²-{[(9H-fluoren-9-yl)methoxy]carbonyl}-4-methyl-L-leucinamide, 103

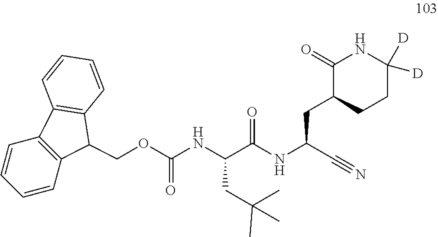 [SMILES: [2H]C1([2H])CC[C@@H](C[C@@H](C#N)NC(=O)[C@H](CC(C)(C)C)NC(=O)OCC2c3ccccc3-c3ccccc32)C(=O)N1]

Compound 103 was synthesized as in Scheme 75.

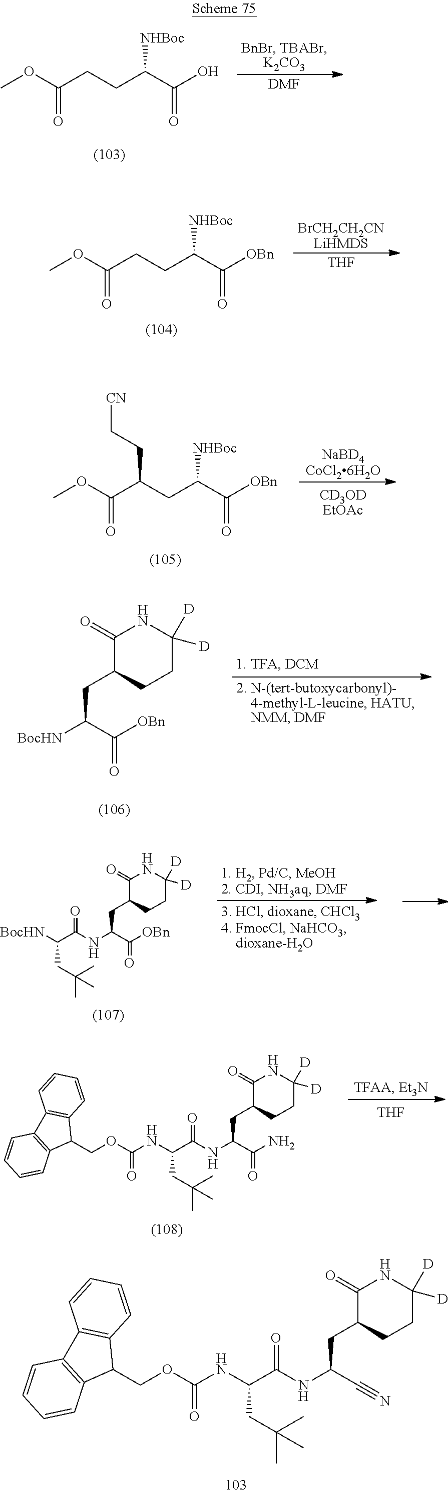

Preparation of 1-benzyl 5-methyl N-(tert-butoxycarbonyl)glutamate, (104). The mixture of the 5-methyl ester of N-(tert-butoxycarbonyl)glutamic acid (103) (1.07 g, 4.09 mmol), benzyl bromide (1.41 g, 8.25 mmol), tetrabutylammonium bromide (TBABr) (160 mg, 0.496 mmol), and potassium carbonate (1.19 g, 8.62 mmol) in DMF (15 mL) was warmed in a 65° C. bath for 8 h. It was then concentrated under reduced pressure. The residue was partitioned between chloroform (35 mL) and water (20 mL). The water layer was washed with chloroform (2×15 mL) and the combined organic layer was washed with brine (15 mL), dried with sodium sulfate, filtered and concentrated under reduced pressure. The product was purified by silica gel column chromatography (eluted with the gradient of 0% to 100% of ethyl acetate in hexanes), which provided (104) (1.22 g, 85% yield) as a clear oil. LC/MS: Eluent system B (retention time: 5.62 min); ESI-MS 352 [M+H]⁺.

Preparation of 1-benzyl 5-methyl (4S)—N-(tert-butoxycarbonyl)-4-(2-cyanoethyl)glutamate, (105). To the solution of 1-benzyl 5-methyl N-(tert-butoxycarbonyl)glutamate (104) (562 mg, 1.60 mmol) in anhydrous THF (12 mL) cooled in a −78° C. bath was added a 1M solution of LiHMDS (4.0 mL) over 15 minutes. After 90 minutes at −78° C., a solution of 3-bromopropionitrile (302 mg, 2.27 mmol) in anhydrous THF (2 mL) was added slowly and the mixture was stirred at −78° C. for an additional 3 h. Then to the resulting mixture was added methanol (1 mL) followed by a 20% solution of acetic acid in THF (1 mL), both pre-cooled to −78° C. prior to addition. After 20 minutes the dry ice/acetone bath was removed and after warming up to ambient temperature the mixture was concentrated under reduced pressure. The residue was partitioned between ethyl acetate (40 mL) and water (20 mL). The water layer was washed with ethyl acetate (2×15 mL) and the combined organic layer was washed with brine (15 mL), dried with sodium sulfate, filtered and concentrated under reduced pressure. The product was purified by silica gel column chromatography (eluted with the gradient of 0% to 40% of ethyl acetate in hexanes), which provided 1-benzyl 5-methyl (4S)—N-(tert-butoxycarbonyl)-4-(2-cyanoethyl)glutamate (105) (251 mg, 39% yield) as a clear oil. LC/MS: Eluent system B (retention time: 5.39 min); ESI-MS 405 [M+H]⁺.

Preparation of benzyl N-(tert-butoxycarbonyl)-3-[(3S)-2-oxo(6,6-d₂)piperidin-3-yl]-L-alaninate, (106). To a solution of 1-benzyl 5-methyl (4S)—N-(tert-butoxycarbonyl)-4-(2-cyanoethyl)glutamate (105) (202 mg, 0.500 mmol) in methanol-d₄ (10 mL) containing cobalt chloride hexahydrate (89.4 mg, 0.376 mmol) cooled in a −42° C. bath (acetonitrile/dry ice bath) was added sodium borodeuteride (201 mg, 4.81 mmol) portion-wise over 1 minute. The reaction flask was transferred into a −20° C. freezer. After overnight. Then the mixture was diluted with ethyl acetate (25 mL) and stirred at room temperature for additional 4 h. Saturated ammonium chloride solution (15 mL) was added and the pH was adjusted to between 6.8 and 7 with 1 M aqueous sodium hydrosulfate. The organic layer was dried with sodium sulfate, filtered and concentrated under reduced pressure. The produce was purified by column chromatography (eluted with the gradient of 0% to 8% methanol in DCM), which provided (106) (121 mg, 64% yield) as clear oil. LC/MS: Eluent system B (retention time: 5.21 min); ESI-MS 379 [M+H]⁺.

Preparation of benzyl N-(tert-butoxycarbonyl)-4-methyl-L-leucyl-3-[(3S)-2-oxo(6,6-d₂)piperidin-3-yl]-L-alaninate, (107). Removal of the t-butoxycarbonyl group of benzyl N-(tert-butoxycarbonyl)-3-[(3S)-2-oxo(6,6-d₂)piperidin-3-yl]-L-alaninate (106) (121 mg, 0.320 mmol) used TFA conditions described for intermediate (58) in Scheme 39 was followed by HATU mediated amide coupling as described for intermediate (78) in Scheme 59 with N-(tert-butoxycarbonyl)-4-methyl-L-leucine (83.2 mg, 0.340 mmol), HATU (141 mg, 0.37 mmol), and NMM (123 mg, 1.21 mmol) in DMF (5 mL). The resulting product was purified by silica gel column chromatography (eluted with the gradient of 0% to 12% methanol in DCM) provided (107) (102 mg, 63% yield) as a white foam. LC/MS: Eluent system B (retention time: 6.39 min); ESI-MS 506 [M+H]⁺.

Preparation of N-{[(9H-fluoren-9-yl)methoxy]carbonyl}-4-methyl-L-leucyl-3-[(3S)-2-oxo(6,6-d₂)piperidin-3-yl]-L-alaninamide, (108). The palladium catalyzed hydrogenolysis of the benzyl N-(tert-butoxycarbonyl)-4-methyl-L-leucyl-3-[(3S)-2-oxo(6,6-d₂)piperidin-3-yl]-L-alaninate (107) (86.1 mg, 0.161 mmol) was performed as described for the compound 82 (Scheme 55) except methanol was used instead of acetonitrile. The filtrate was concentrated under reduced pressure, the residue dissolved in DMF (6 mL) and after cooling in an ice bath CDI (482 mg, 2.97 mmol) was added, and after 30 min aqueous ammonia (416 mg, 8.71 mmol) was also added. After an additional 15 minutes, the mixture was diluted with ethyl acetate (25 mL) and washed with water (15 mL). The aqueous layer was extracted with ethyl acetate (2×15 mL), and the combined organic layer was washed with brine (10 mL), dried with sodium sulfate, filtered and concentrated under reduced pressure. The resulting residue was dissolved in chloroform (6 mL) cooled in an ice bath and then 4M hydrogen chloride solution in dioxane (2 mL) was added. After 90 minutes in the ice bath, the resulting precipitate was collected by decantation and drying under reduced pressure. The resulting material was dissolved in dioxane (15 mL), then an aqueous solution (10 mL) of sodium bicarbonate (1.59 g, 18.9 mmol) was added following by (9-fluorenylmethyl) chloroformate (368 mg, 1.42 mmol) and an additional portion of dioxane (10 mL). The suspension was sonicated for 10 minutes then diluted with ethyl acetate (60 mL) and washed with water. The aqueous layer was extracted with ethyl acetate (2×15 mL), and the combined organic layer was washed with brine (10 mL), dried with sodium sulfate, filtered and concentrated under reduced pressure. The product was purified by silica gel column chromatography (eluted with the gradient of 0% to 12% methanol in chloroform) provided (108) as clear oil (27.0 mg, 31% yield over 4 steps). LC/MS: Eluent system B (retention time: 6.43 min); ESI-MS 537 [M+H]⁺.

Preparation of N-{(1S)-1-cyano-2-[(3S)-2-oxo(6,6-d₂)piperidin-3-yl]ethyl}-N²-{[(9H-fluoren-9-yl)methoxy]carbonyl}-4-methyl-L-leucinamide, 103. Conversion of the primary amide to the nitrile for N-{[(9H-fluoren-9-yl)methoxy]carbonyl}-4-methyl-L-leucyl-3-[(3S)-2-oxo(6,6-d₂)piperidin-3-yl]-L-alaninamide (108) (27.0 mg, 0.0503 mmol) was performed as described for the compound 86 in Scheme 59. The product was purified by silica gel column chromatography (eluted with the gradient of 0% to 8% methanol in chloroform), which provided 103 (15.1 mg, 58% yield) as a white powder. LC/MS: Eluent system B (retention time: 6.65 min); ESI-MS 519 [M+H]⁺.

### Compound 104

### Synthesis of N-[(2S)-1-({(1S)-1-cyano-2-[(3S)-2-oxo(6,6-d₂)piperidin-3-yl]ethyl}amino)-4,4-dimethyl-1-oxopentan-2-yl]-4-methoxy-1H-indole-2-carboxamide, 104

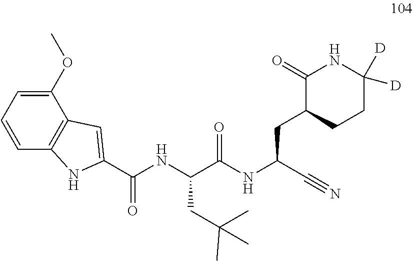 [SMILES: [2H]C1([2H])CC[C@@H](C[C@@H](C#N)NC(=O)[C@H](CC(C)(C)C)NC(=O)c2cc3c(OC)cccc3[nH]2)C(=O)N1]

Compound 104 was synthesized as in Scheme 76.

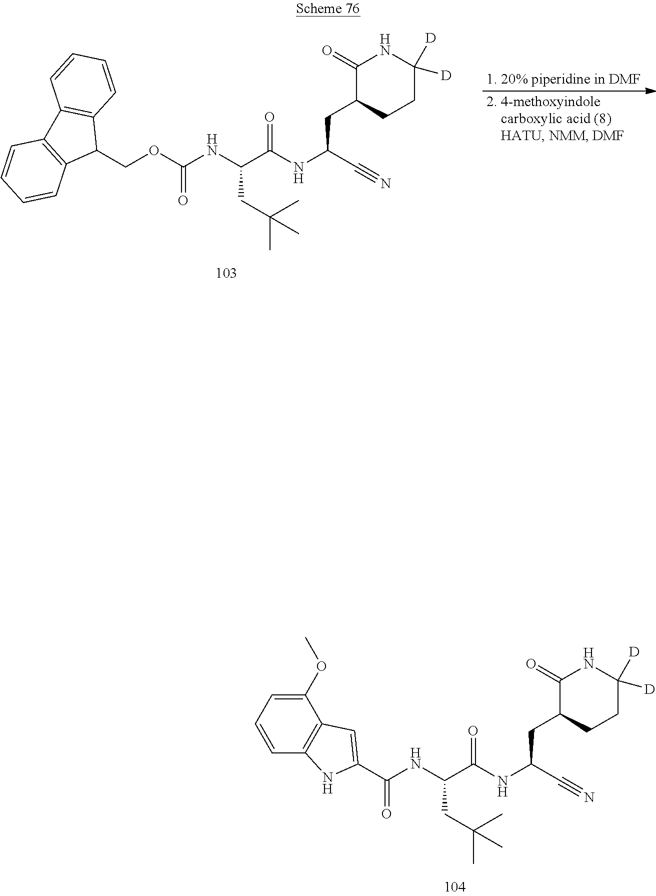 [SMILES: [2H]C1([2H])CC[C@@H](C[C@@H](C#N)NC(=O)[C@H](CC(C)(C)C)NC(=O)OCC2c3ccccc3-c3ccccc32)C(=O)N1 ; [2H]C1([2H])CC[C@@H](C[C@@H](C#N)NC(=O)[C@H](CC(C)(C)C)NC(=O)c2cc3c(OC)cccc3[nH]2)C(=O)N1]

Preparation of N-[(2S)-1-({(1S)-1-cyano-2-[(3S)-2-oxo(6,6-d₂)piperidin-3-yl]ethyl}amino)-4,4-dimethyl-1-oxopentan-2-yl]-4-methoxy-1H-indole-2-carboxamide, 104. Removal of the Fmoc protecting group and the subsequent amide formation reaction was performed as described for the compound 88 in Scheme 61 except starting with N-{(1S)-1-cyano-2-[(3S)-2-oxo(6,6-d₂)piperidin-3-yl]ethyl}-N²-{[(9H-fluoren-9-yl)methoxy]carbonyl}-4-methyl-L-leucinamide 103 (8.1 mg, 0.016 mmol) and using 4-methoxyindole carboxylic acid (8) (30.8 mg, 0.161 mmol). Purification was accomplished by silica gel column chromatography (eluted with gradient of 0% to 12% methanol in 1:1 mixture ethyl acetate-chloroform) provided 104 (6.9 mg, 92% yield) as white powder. ¹H NMR (600 MHz, DMSO-d₆) δ 11.51 (d, J=2.0 Hz, 1H), 8.87 (d, J=7.8 Hz, 1H), 8.44 (d, J=7.8 Hz, 1H), 7.45 (s, 1H), 7.31 (dd, J=0.7, 2.2 Hz, 1H), 7.10 (dd, J=7.9, 8.1 Hz, 1H), 7.02 (d, J=8.3 Hz, 1H), 6.51 (d, J=7.6 Hz, 1H), 5.00 (ddd, J=8.6, 8.2, 7.4 Hz, 1H), 4.50 (ddd, J=8.6, 8.4, 3.0 Hz, 1H), 3.87 (s, 3H), 2.30-2.20 (m, 2H), 1.84-1.74 (m, 3H), 1.68-1.63 (m, 2H), 1.55-1.46 (m, 1H), 1.42-1.35 (m, 1H), 0.93 (s, 9H). LC/MS: Eluent system B (retention time: 5.23 min); ESI-MS 470 [M+H]⁺, 468 [M−H]⁻.

### Compound 105

### Synthesis of N-[(2S)-1-({(1S)-1-cyano-2-[(3S)-2-oxo(6,6-d₂)piperidin-3-yl]ethyl}amino)-4,4-dimethyl-1-oxopentan-2-yl]-4-methoxy-1H-pyrrolo[3,2-c]pyridine-2-carboxamide, 105

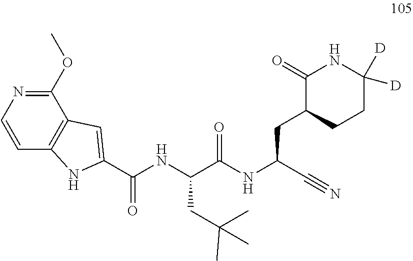 [SMILES: [2H]C1([2H])CC[C@@H](C[C@@H](C#N)NC(=O)[C@H](CC(C)(C)C)NC(=O)c2cc3c(OC)nccc3[nH]2)C(=O)N1]

Compound 105 was synthesized as in Scheme 77.

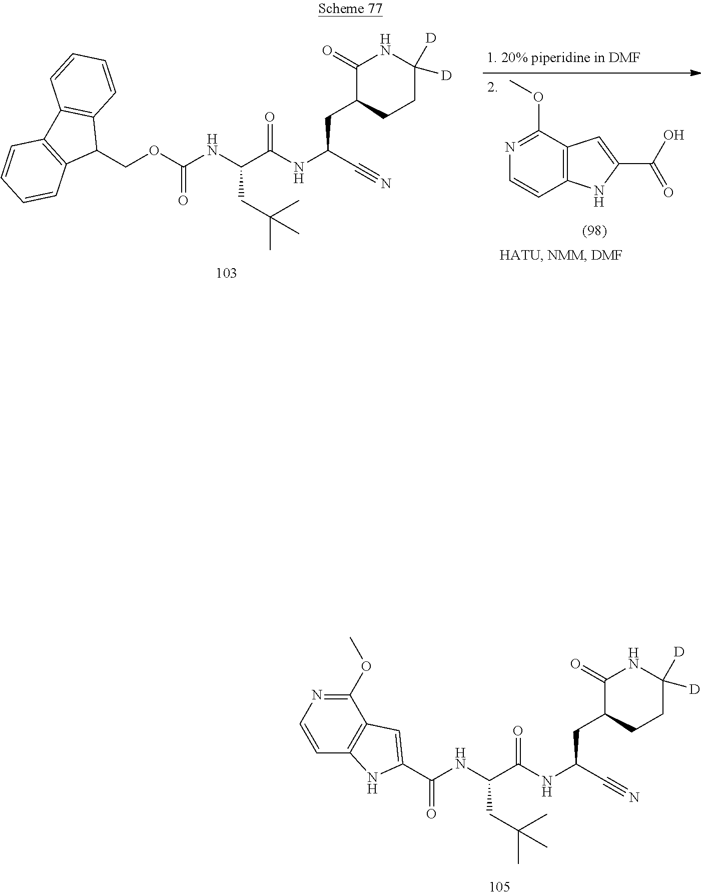 [SMILES: [2H]C1([2H])CC[C@@H](C[C@@H](C#N)NC(=O)[C@H](CC(C)(C)C)NC(=O)OCC2c3ccccc3-c3ccccc32)C(=O)N1 ; COc1nccc2[nH]c(C(=O)O)cc12 ; [2H]C1([2H])CC[C@@H](C[C@@H](C#N)NC(=O)[C@H](CC(C)(C)C)NC(=O)c2cc3c(OC)nccc3[nH]2)C(=O)N1]

Preparation of N-[(2S)-1-({(1S)-1-cyano-2-[(3S)-2-oxo(6,6-d₂)piperidin-3-yl]ethyl}amino)-4,4-dimethyl-1-oxopentan-2-yl]-4-methoxy-1H-pyrrolo[3,2-c]pyridine-2-carboxamide, 105. Removal of the Fmoc protecting group and the subsequent amide formation reaction was performed as described for the compound 88 in Scheme 61 except starting with N-{(1S)-1-cyano-2-[(3S)-2-oxo(6,6-d₂)piperidin-3-yl]ethyl}-N²-{[(9H-fluoren-9-yl)methoxy]carbonyl}-4-methyl-L-leucinamide 103 (4.1 mg, 0.0079 mmol) and using 4-methoxy-1H-pyrrolo[3,2-c]pyridine-2-carboxylic acid hydrochloride (98) (22.4 mg, 0.117 mmol). The product was purified by silica gel column chromatography (eluted with gradient of 0% to 12% methanol in 1:1 mixture ethyl acetate-chloroform), which provided 105 (2.9 mg, 78% yield) as a white powder. LC/MS: Eluent system B (retention time: 2.67 min); ESI-MS 471 [M+H]⁺, 469 [M−H]⁻.

### Compound 106

### Synthesis of N-{(1S)-1-cyano-2-[(3S)-2-oxopiperidin-3-yl]ethyl}-2-[(1R,2R)-2-phenylcyclopropane-1-carbonyl]-L-leucinamide, 106

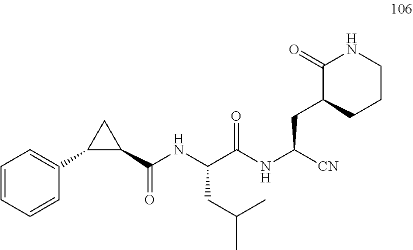 [SMILES: CC(C)C[C@H](NC(=O)[C@@H]1C[C@H]1c1ccccc1)C(=O)N[C@H](C#N)C[C@@H]1CCCNC1=O]

Compound 106 was synthesized as in Scheme 78.

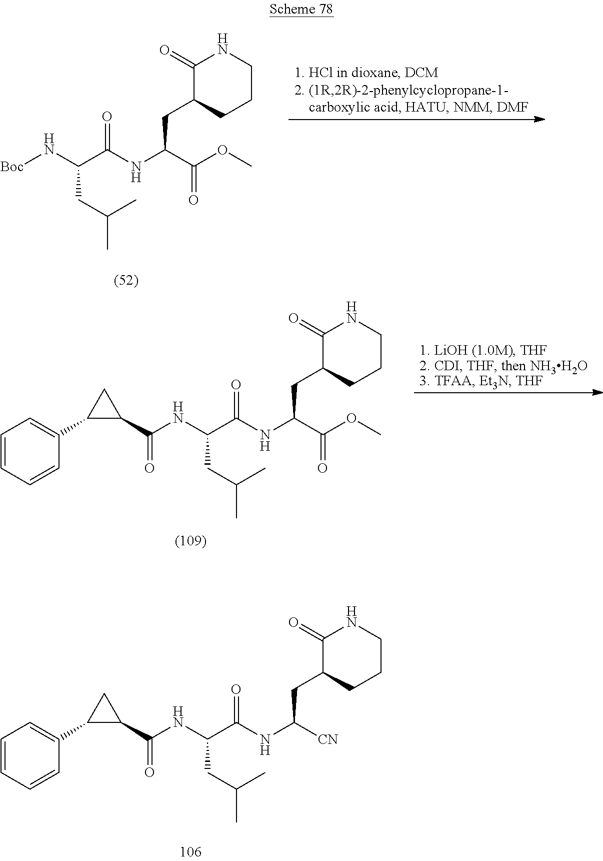

Preparation of methyl N-[(1R,2R)-2-phenylcyclopropane-1-carbonyl]-L-leucyl-3-[(3S)-2-oxopiperidin-3-yl]-L-alaninate, (109). A solution of methyl N-(tert-butoxycarbonyl)-L-leucyl-3-[(3S)-2-oxopiperidin-3-yl]-L-alaninate (52) (189 mg, 0.457 mmol) in DCM (5 mL) was cooled using an ice-water bath and then 4 M HCl in 1,4-dioxane (5 mL) was added. After 30 min, the ice bath was removed. After overnight, the mixture was concentrated under reduced pressure. The residue was co-evaporated with DCM/ether (1:1, 3×20 mL), and dried under reduced pressure for 1 h. To the residue was added anhydrous DMF (10 mL) and (1R,2R)-2-phenylcyclopropane-1-carboxylic acid (88.9 mg, 0.548 mmol) and the solution was cooled in an ice bath after which HATU (208 mg, 0.548 mmol) was added followed by dropwise addition of NMM (0.150 mL, 1.37 mmol). After 45 min, an ice/saturated aqueous NaHCO₃ mixture (1:1, 10 mL) was added and the resulting mixture was extracted with EtOAc (3×25 mL). The combined organic layer was washed with brine (10 mL), dried over anhydrous Na₂SO₄, filtered, and concentrated under reduced pressure. The product was purified by column chromatography on silica gel with a gradient of 0 to 10% MeOH in CHCl₃, which generated (109) (185 mg, 88% yield) as a white solid. LC/MS: Eluent system A (retention time: 5.24 min); ESI-MS: 458 [M+H]⁺.

Preparation of N-{(1S)-1-cyano-2-[(3S)-2-oxopiperidin-3-yl]ethyl}-N²-[(1R,2R)-2-phenylcyclopropane-1-carbonyl]-L-leucinamide, 106. To a solution of methyl N-[(1R,2R)-2-phenylcyclopropane-1-carbonyl]-L-leucyl-3-[(3S)-2-oxopiperidin-3-yl]-L-alaninate (109) (164 mg, 0.357 mmol) in THF (5 mL) cooled in an ice bath was added a 1.0 M LiOH aqueous solution (5 mL). After 2 h, the pH of the reaction mixture was adjusting to 3 by adding 1.0 M HCl aqueous solution. The two layers were separated and the aqueous layer was extracted with EtOAc (3×25 mL). The combined organic phase was washed with brine (10 mL), dried over anhydrous Na₂SO₄, filtered, concentrated under reduced pressure. To the resulting residue was added THF (10 mL) and CDI (116 mg, 0.714 mmol). After 15 min, NH₃ (aq., 28%, 0.245 mL, 1.79 mmol) was added. After 1 h, water (10 mL) was added and the mixture was extracted with EtOAc (3×25 mL). The combined organic phase was washed with brine (10 mL), dried over anhydrous Na₂SO₄, filtered, and concentrated under reduced pressure. Purification was accomplished by column chromatography on silica gel with a gradient of 0 to 10% MeOH in CHCl₃, which generated the intermediate (103 mg) as a white solid. To this intermediate (94.1 mg, 0.213 mmol) was added THF (10 mL) and the solution was cooled in an ice-bath. Then Et₃N (89.1 μL, 0.639 mmol) was added followed by slow addition of a solution of TFAA (89.5 mg, 0.426 mmol) in anhydrous THF (2 mL). After 30 min, water (10 mL) was added. The two layers were separated and the aqueous layer was extracted with EtOAc (3×25 mL). The combined organic phase was washed with brine (10 mL), dried over anhydrous Na₂SO₄, filtered, and concentrated under reduced pressure. The product was purified by column chromatography on silica gel with a gradient of 0 to 5% MeOH in CHCl₃, which generated 106 (71.9 mg, 47% yield for 3 steps) as a white solid. ¹HNMR (600 MHz, DMSO-d₆) δ 8.88 (d, J=8.0 Hz, 1H), 8.33 (d, J=7.7 Hz, 1H), 7.53 (s, 1H), 7.29-7.25 (m, 2H), 7.19-7.15 (m, 1H), 7.13-7.09 (m, 2H), 5.03-4.97 (m, 1H), 4.28-4.22 (m, 1H), 3.16-3.04 (m, 2H), 2.31-2.19 (m, 3H), 2.01-1.97 (m, 1H), 1.86-1.79 (m, 1H), 1.79-1.70 (m, 2H), 1.62-1.53 (m, 2H), 1.50-1.34 (m, 3H), 1.30-1.24 (m, 1H), 1.20-1.16 (m, 1H), 0.89 (d, J=6.6 Hz, 3H), 0.85 (d, J=6.5 Hz, 3H); LC/MS: Eluent system A (retention time: 5.07 min); ESI-MS: 425 [M+H]⁺.

### Example 2

In this example, methods and results from the development of compounds for use in treating Group IV RNA viruses are provided.

### Methods and Materials

SARS-CoV-2 3-Chymotrypsin-Like Protease (3CLP) Expression and Purification

DNA encoding 3CLP from SARS-CoV-2 was obtained from BioBasic Inc. (Ontario, Canada) and codon optimized for expression in Escherichia coli. The gene was cloned into the pET SUMO (small ubiquitin-like modifier) expression vector (Invitrogen). Clones were sequenced to ensure that the SARS-CoV-2 3CLP protein was in frame with the His-tagged SUMO fusion protein. The resulting plasmid was transformed into E. coli BL21(DE3) and the E. coli transformant was grown in Luria Broth, Miller at 37° C. with shaking (220 rpm) to an OD600 of 0.6-0.7 using kanamycin (50 μg/mL) as selective pressure. Expression of the fusion protein was induced by the addition of 0.5 mM IPTG to the cell culture and the culture was grown for an additional 4-5 h at 37° C. Cells were harvested by centrifugation (6000 g for 10 min at 4° C.) and suspended in lysis buffer (20 mM Tris-HCl pH 7.8, 150 mM NaCl). Cells were lysed by sonication on ice and the lysate was centrifuged (17000 g for 10 min at 4° C.) to remove cellular debris. The supernatant was isolated and, after adding imidazole (5 mM), mixed with Ni-NTA resin (Qiagen). The mixture was loaded on a fritted column and allowed to flow by gravity at 4° C. The resin was washed with 10 column volumes (CV) of lysis buffer containing 20 mM imidazole. The fusion protein was eluted using 2 CV of lysis buffer containing increased concentrations of imidazole (40, 60, 80, 100, 200 and 500 mM). Eluted fractions were analyzed by SDS-PAGE and those that contained the fusion protein were pooled together, dialyzed against lysis buffer containing 1 mM DTT at 4° C. and concentrated using Amicon Ultra-15 filter (Millipore) with a MWCO of 10 kDa. The fusion protein was digested by His-tagged SUMO protease (McLab, South San Francisco, Calif.) at 4° C. for 1-2 h to remove the SUMO tag. The cleavage mixture was added to Ni-NTA resin and loaded on a fritted column. The flow through containing SARS-CoV-2 3CLP was collected and analyzed by SDS-PAGE. The SARS-CoV-2 3CLP protein was further purified using size exclusion chromatography (G-100, GE Healthcare, 1 ml/min flow rate, 4° C.) in 20 mM Tris, 20 mM NaCl, 1 mM DTT, pH 7.8. Immunoglobulin G, 166 kDa; bovine serum albumin, 67 kDa; ovalbumin, 43 kDa; and lysozyme, 15 kDa were used as calibration standards. Fractions containing the SARS-CoV-2 3CLP protein were pooled and concentrated using Amicon Ultra-15 filter with a MWCO of 10 kDa.

Mass Spectrometry of SARS-CoV-2 3CLP

The mass of the free SARS-CoV-2 3CLP was confirmed by HR-MALDI on a MALDI-TOF (Bruker Ultrafelxtreme, Bruker Daltronics, USA) and LC-MS on an ESI-TOF instrument (Agilent Technologies 6220, California, USA) using electrospray ionization.

### Determination of Enzyme Inhibition in SARS-CoV-2 3-Chymotrypsin-Like Protease (3CLP) Assay

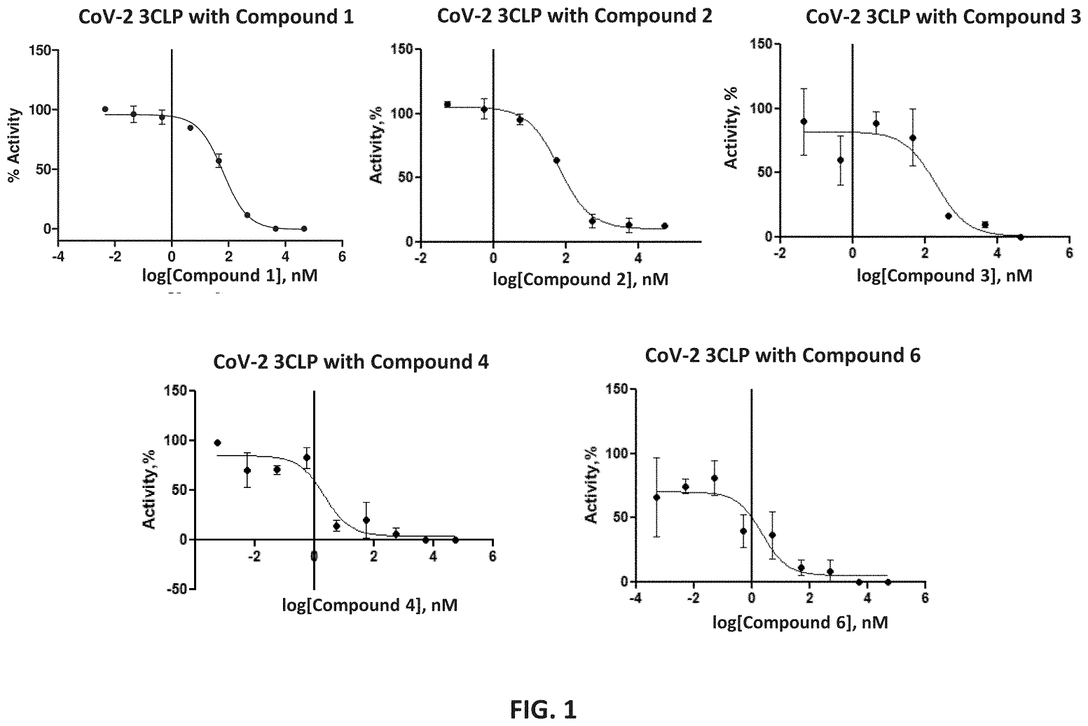

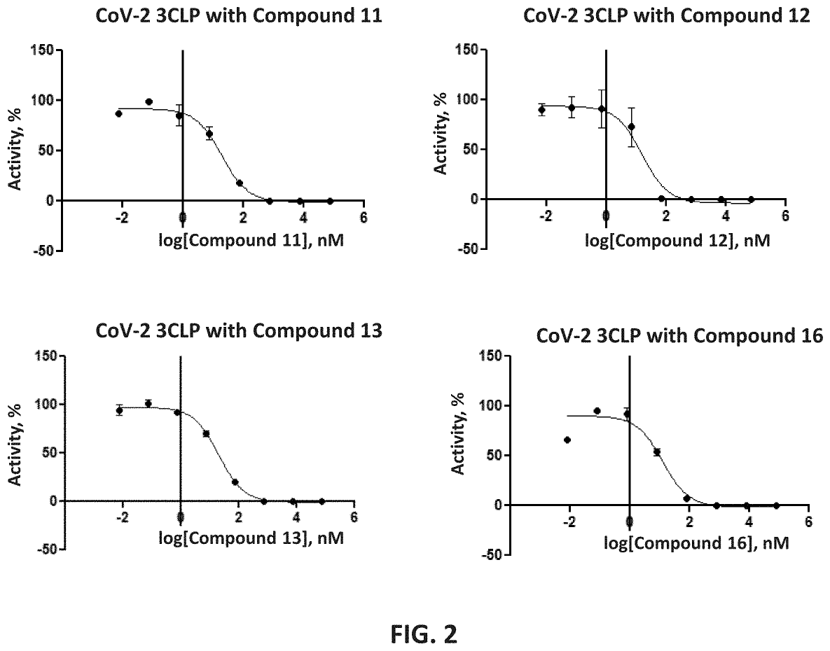

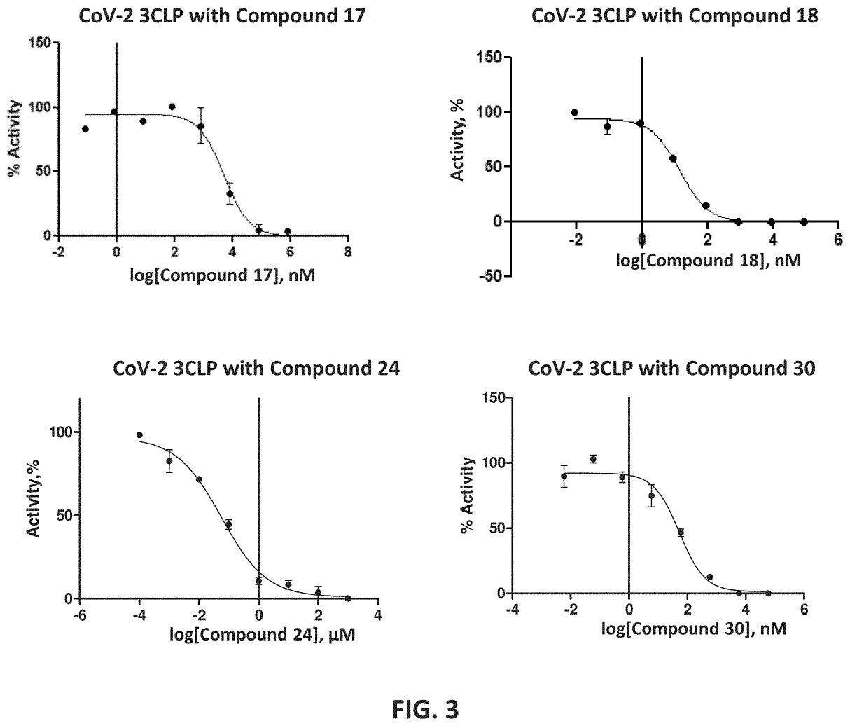

Compounds described herein were screened for SARS-CoV-2 3CLP inhibition using a Fluorescence resonance energy transfer (FRET) assay at different compound concentrations. Examples of the resulting concentration response curves are shown in FIG. 1, FIG. 2 and FIG. 3 and examples of the determined 50 percent inhibition concentration (IC₅₀) are provided in TABLE 1.

Synthesis of FRET Peptide Substrate was performed following the procedures Vuong, W. et al., Nature Communications 2020, 11, Article number: 4282.

Enzyme Kinetics of SARS-CoV-2 3CLP

A synthesized fluorescent substrate containing the cleavage site (indicated by the arrow, ↓) of SARS-CoV-2 3CLP (2-Abz-SVTLQ↓SG-Tyr(NO₂)—R—NH₂) was used for the fluorescence resonance energy transfer (FRET)-based cleavage assay 4. The protease reaction of SARS-CoV-2 3CLP towards fluorescent substrate was performed in activity buffer (20 mM Bis Tris, pH 7.8, 1 mM DTT) at 37° C. for 10 min. The final concentration of protease used in the assay was fixed at 80 nM and the concentrations of the substrate were varied from 0.1 to 500 μM. Reaction was started with the enzyme and the fluorescence signal of the Abz-SVTLQ peptide cleavage product was monitored at an emission wavelength of 420 nm with excitation at 320 nm, using an Flx800 fluorescence spectrophotometer (BioTek). Before kinetic calculations, it was verified that the proportionality between the fluorescence emitted and the amount of the substrate used in the assay was linear. The minimal concentration of the enzyme and time of reaction that gave a linear dependence of amount of generated product with time was chosen. Initial velocities in corresponding relative fluorescence units per unit of time (ARFU/s) were converted to the amount of the cleaved substrate per unit of time (M/s) by fitting to the calibration curve of free Aminobenzoyl-SVTLQ. All data are corrected for inner filter effects by an adopted literature protocol. In short, the fluorescence signal (RFU) at each substrate concentration was determined and defined as f(FRET). Then, 5 uL free Aminobenzoyl-SVTLQ at final 5 uM was added to each concentration and fluorescence was taken f(FRET+Aminobenzoyl-SVTLQ). Simultaneously, a reference reading was taken with the same free Aminobenzoyl-SVTLQ concentration and defined as f(ref). The inner-filter correction was obtained as:

corr %=(f(FRET+Aminobenzoyl-SVTLQ)−f(FRET))/f(ref)×100%

The corrected initial velocity of the reaction was calculated as

V=Vo/(corr %).

Vo represents the initial velocity of each reaction.

Kinetic constants (vmax and Km) were derived by fitting the corrected initial velocity to the Michaelis-Menten equation, v=vmax×[S]/(Km+[S]) using GraphPad Prism 6.0 software. kcat/Km was calculated according to the equation, kcat/Km=vmax/([E]×Km). Triplicate experiments were performed for each data point, and the average was determined.

Inhibition Parameters

Stock solutions of the compounds were prepared with DMSO. For the determination of the IC₅₀, 80 nM of SARS-CoV-2 3CLP was incubated with the compounds at various concentrations from 0 to 100 μM in 20 mM Bis-Tris, pH 7.8, 1 mM DTT at 37° C. for 10 min. The protease reaction was started by addition of 100 μM of the substrate. The GraphPad Prism 6.0 software (GraphPad) was used for the calculation of the IC₅₀ values.

TABLE 1
SARS-CoV-2 3CLP activity

| Compound | 3CL Protease IC₅₀ |
|---|---|
| Number | (μM) |
| 1 | 0.064 |
| 2 | 0.064 |
| 3 | 0.13 |
| 4 | 0.0040 |
| 5 | 0.0064 |
| 6 | 0.0010 |
| 9 | 0.055 |
| 10 | 0.052 |
| 11 | 0.019 |
| 12 | 0.016 |
| 13 | 0.020 |
| 14 | 0.022 |
| 15 | 0.041 |
| 16 | 0.013 |
| 17 | 4.99 |
| 18 | 0.014 |
| 19 | 0.026 |
| 20 | 0.086 |
| 21 | 0.018 |
| 22 | 0.017 |
| 23 | 0.036 |
| 24 | 0.057 |
| 25 | 0.018 |
| 26 | 0.017 |
| 27 | 0.029 |
| 30 | 0.053 |
| 34 | 0.013 |
| 35 | 0.014 |
| 40 | 0.014 |
| 66 | 0.34 |
| 67 | 0.17 |
| 68 | 0.0091 |
| 69 | 0.024 |
| 70 | 0.036 |
| 71 | 0.049 |
| 72 | 0.022 |
| 73 | 0.19 |
| 74 | 0.11 |
| 75 | 0.16 |
| 76 | 0.95 |
| 77 | 17 |
| 78 | 47 |
| 79 | 0.25 |
| 80 | 0.71 |
| 81 | 0.62 |
| 82 | 2.4 |
| 83 | 4.5 |
| 84 | 3.1 |
| 85 | 6.4 |
| 86 | 0.11 |
| 87 | 0.2 |
| 88 | 0.04 |
| 89 | 2.8 |
| 90 | 1.1 |
| 91 | 37 |
| 92 | 104 |
| 93 | 0.24 |
| 94 | 0.16 |
| 95 | 97 |
| 96 | 46 |
| 97 | >500 |
| 99 | 0.022 |
| 100 | 0.22 |
| 101 | 0.18 |
| 102 | 0.14 |
| 104 | <0.05 |
| 106 | 0.069 |

### Evaluation of In Vitro Inhibition Activity of Exemplary Compounds Against SARS-CoV-2

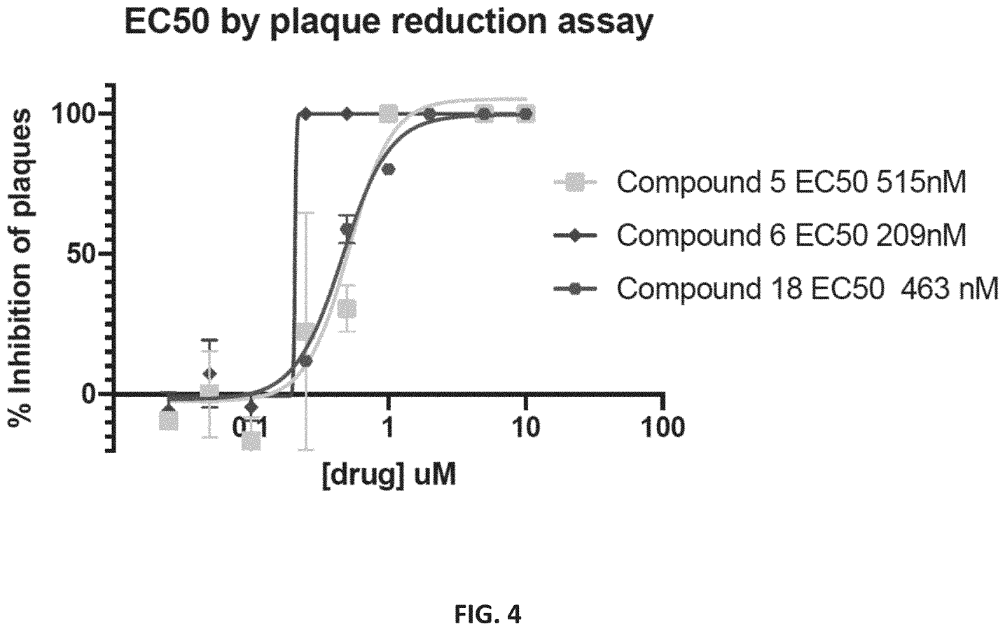

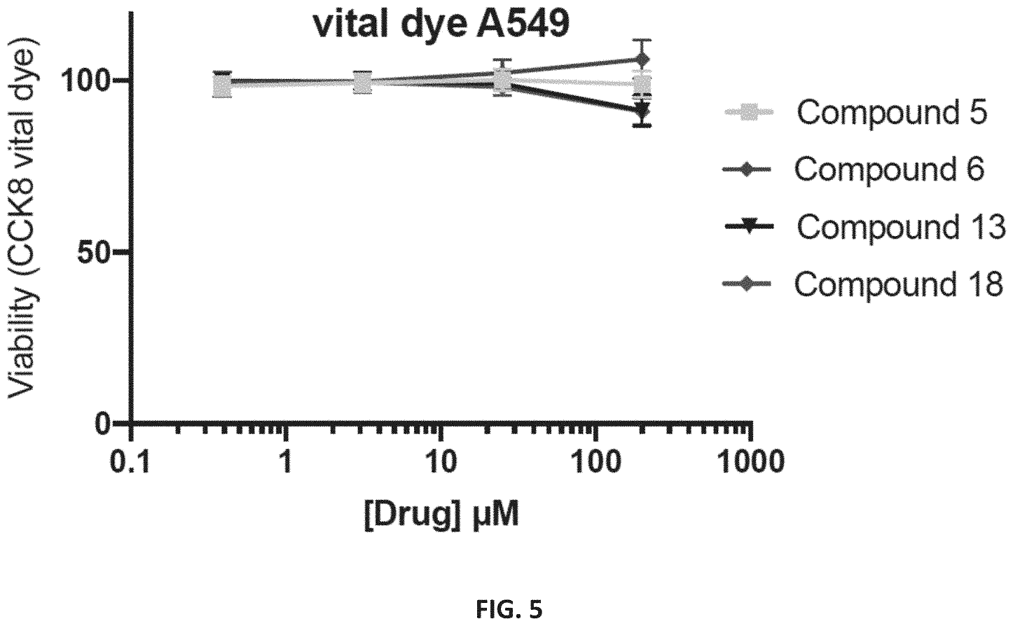

Compounds described herein were screened for inhibition of SARS-CoV-2 viral replication in an in vitro plaque reduction assay. Examples of the resulting concentration response curves are shown in FIG. 4 and examples of the determined effective concentration for 50 percent reduction (EC₅₀) of plaques are provided in TABLE 2. Compounds 5, 6, 13 and 18 were tested for cytotoxicity using a cell viability assay. Examples of the resulting concentration response curves are shown in FIG. 5. The effective concentration to reduce the viability by 50 percent (CC₅₀) are provided in TABLE 2.

Determination of Inhibition and EC₅₀ by Plaque Assay

SARS-CoV-2/CANADA/VIDO 01/2020 was a kind gift from Darryl Falzarano (University of Saskatchewan). Vero (Female green monkey kidney) E6 cells were infected with an MOI of 0.0001 pfu/cell in infection medium consisting of DMEM supplemented with 1× non-essential amino acids (Gibco), 10 mM HEPES, 2% fetal bovine serum, 50 IU/mL penicillin, 50 IU/mL streptomycin with 10 μM or different doses of antiviral drugs. After 1 h, the infecting medium was removed and monolayers were overlaid with MEM supplemented with 10 mM HEPES and 1.2% Avicel RC-591 (DuPont). After 48 h, cells were fixed in 10% formaldehyde, and stained using 0.5% (w/v) crystal violet. Plaques were counted and for screening at 10 μM and compounds that did not reduce the plaque numbers by half were assign >5 μM. The compounds that did reduce viral plaques significantly at 10 μM were tested at multiple concentrations (10, 6, 3, 1, 0.6, 0.3, 0.1, 0.06, and 0.03 μM) and the results were plotted as % inhibition vs the log 10[drug] using Prism (GraphPad). EC₅₀'s were determined using a non-linear regression analysis. Experiments were done in triplicate.

Measuring Cytotoxicity in A549 and Vero E6 cells

Cell viability was measured using the CellTiter-Glo luminescent cell viability assay (Promega). Separately A549 (male human lung epithelial) cells and VeroE6 cells were seeded at 5×103 cells/well in 96-well plates and incubated overnight before treatment. Compounds were solubilized in DMSO and added to cells in an eight-point four-fold serial dilution (200 μM to 0.0122 μM). Cells were incubated in the presence of compounds for 24 hours before addition of the luminescence substrate and measurement of ATP activity according to manufacturer's instructions. The percentage of viable cells was calculated relative to cells treated with solvent alone (0.5% DMSO).

TABLE 2
Inhibitor activity against SARS-CoV-2

| | | Vero E6 and |
|---|---|---|
| Compound | SARS-CoV-2 | A549 CC₅₀ |
| Number | Antiviral EC₅₀ (μM) | (μM) |
| 1 | >5 | |
| 2 | >5 | |
| 3 | >5 | |
| 4 | >5 | |
| 5 | 0.52 | >200 |
| 6 | 0.21 | >200 |
| 7 | >5 | |
| 8 | >5 | |
| 9 | >5 | |
| 10 | >5 | |
| 11 | >5 | |
| 12 | >5 | |
| 13 | >5 | >200 |
| 14 | >5 | |
| 15 | >5 | |
| 16 | >5 | |
| 17 | >5 | |
| 18 | 0.46 | >200 |
| 19 | >5 | |
| 20 | 0.71 | |
| 21 | >5 | |
| 22 | >5 | |
| 23 | 0.30 | |
| 24 | >5 | |
| 25 | <0.25 | |
| 26 | 0.28 | |
| 27 | >5 | |
| 30 | >5 | |
| 34 | 2.7 | >200 |
| 35 | 1.5 | >200 |
| 40 | 2.6 | >50 |
| 66 | >5 | |
| 67 | 4.1 | |
| 68 | 2.2 | >200 |
| 69 | 4.8 | >200 |
| 70 | >5 | >200 |
| 71 | >5 | >200 |
| 72 | 2.8 | |
| 73 | <1 | >200 |
| 74 | <1 | >200 |
| 75 | >5 | |
| 76 | >5 | |
| 77 | >5 | |
| 78 | >5 | |
| 79 | 4.5 | |
| 80 | >5 | |
| 81 | >5 | |
| 82 | >5 | |
| 83 | >5 | |
| 84 | >5 | |
| 85 | >5 | |
| 86 | 4.3 | |
| 87 | 1.9 | |
| 88 | >5 | |
| 89 | >5 | |
| 90 | >5 | |
| 91 | >5 | |
| 92 | >5 | |
| 93 | >5 | |
| 94 | >5 | |
| 95 | >5 | |
| 96 | >5 | |
| 97 | >5 | |
| 99 | >5 | |
| 100 | >5 | |
| 101 | >5 | |
| 102 | >5 | |
| 104 | >5 | |
| 106 | >5 | |

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A compound of formula (I): 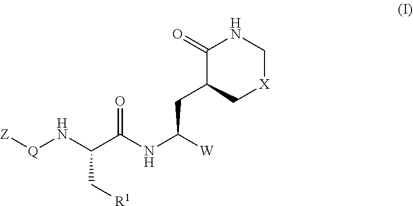
wherein:
W is selected from —CN and —C(═O)CH₂O—R²;
R¹ is selected from C₁₋₄ alkyl, substituted C₁₋₄ alkyl, and C₃₋₆ cycloalkyl;
R² is selected from aryl, substituted aryl, heterocycle, substituted heterocycle, heteroaryl, substituted heteroaryl and —C(═O)R³;
R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl;
X is selected from —CH₂— or is absent;
Q is selected from —CH₂—, —SO₂— and —C(═O)—; and
Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl;
or a pharmaceutically acceptable salt, solvate, hydrate, or isotopic variant thereof;
with the proviso that if X is absent R³ is not 2,6-dichlorophenyl, 2,6-difluorophenyl, 2,6-dimethylphenyl, 2-cyanophenyl, 4-cyano-2-fluorophenyl, 4-chloro-2-hydroxyphenyl, 2,6-dimethoxyphenyl, 4-chlorophenyl, 4-methoxyphenyl, 4-cyanophenyl, 4-fluorophenyl, 4-methylphenyl, phenyl, cyclopropyl and t-butyl.

2. The compound of claim 1, wherein W is —CN.

3. The compound of claim 1, wherein W is —C(═O)CH₂O—R².

4. The compound of claim 1, wherein R¹ is selected from —CH₂CH₃, —CH(CH₃)₂, —C(CH₃)₃, cyclopropyl, cyclobutyl, cyclopentyl and cyclohexyl.

5. The compound of claim 4, wherein R¹ is —CH(CH₃)₂.

6. The compound of claim 4, wherein R¹ is —C(CH₃)₃.

7. The compound of claim 1, wherein X is absent.

8. The compound of claim 1, wherein X is —CH₂—.

9. The compound of claim 1, wherein R² is selected from aryl, substituted aryl, heteroaryl, substituted heteroaryl and —C(═O)R³.

10. The compound of claim 9, wherein R² is selected from phenyl, 4-substituted aryl and 2-pyridyl.

11. The compound of claim 9, wherein R² is —C(═O)R³.

12. The compound of claim 1, wherein R³ is selected from substituted C₄₋₆ alkyl, substituted C₃₋₆ cycloalkyl, phenyl, substituted aryl, heterocycle, substituted heterocycle, and substituted heteroaryl.

13. The compound of claim 12, wherein R³ is halo-C₄₋₆ alkyl.

14. The compound of claim 12, wherein R³ is selected from —C(CH₃)₃, —C(CH₃)₂CF₃, and —C(CH₃)₂CN.

15. The compound of claim 12, wherein R³ is selected from:

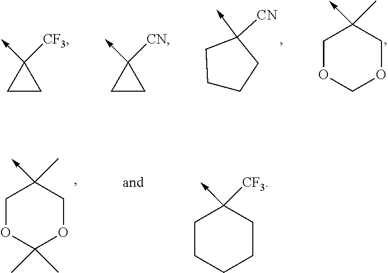

16. The compound of claim 12, wherein R³ is selected from phenyl, substituted phenyl, substituted thiazole, substituted pyrazole, and substituted pyridyl.

17. The compound of claim 12, wherein R³ is selected from

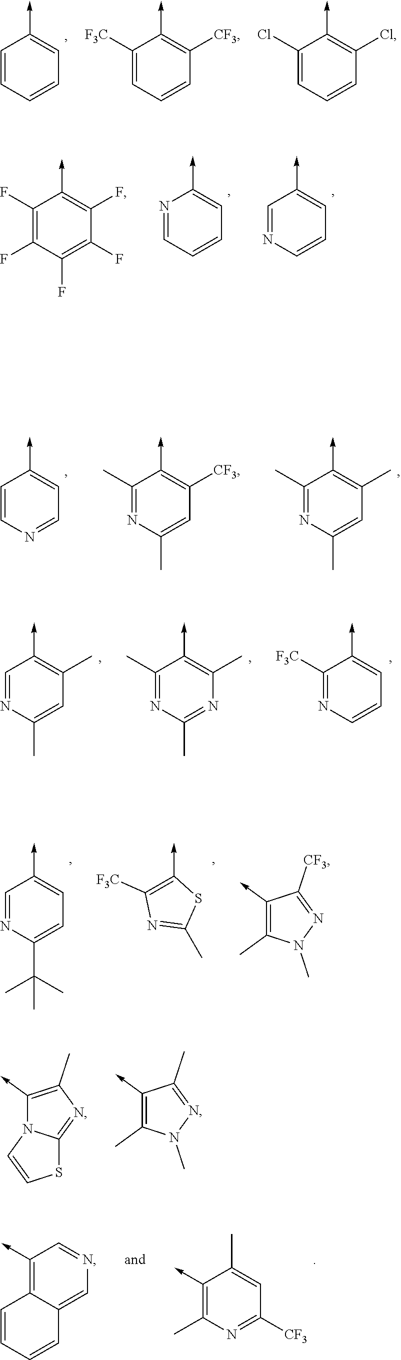

18. The compound of claim 1, Q is —C(═O)—.

19. The compound of claim 1, wherein Z is selected from cyclopropyl, substituted cyclopropyl, cyclopentyl, substituted cyclopentyl, cyclohexyl, arylalkoxy, substituted arylalkoxy, tetrahydrofuran, substituted benzothiazole, benzofuran, substituted benzofuran, indoline, substituted indoline, indole, substituted indole, imidazole, substituted imidazole, pyridinyl, substituted pyridinyl, benzodioxine, substituted benzodioxine, piperidinyl, substituted piperidinyl, pyrrolidinyl, substituted pyrrolidinyl, oxazolyl, and substituted oxazolyl.

20. The compound of claim 19, wherein Z is selected from phenylmethoxy and (3-chlorophenyl)methoxy.

21. The compound of claim 19, wherein Z is 4-methoxyindole.

22. The compound of claim 1, wherein the compound is of formula (Ia): 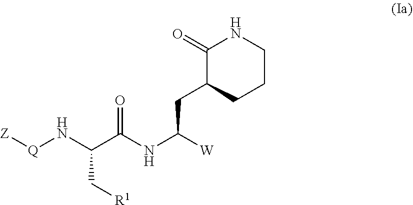
wherein:
W is selected from —CN and —C(═O)CH₂O—R²;
R¹ is selected from C₁₋₄ alkyl, substituted C₁₋₄ alkyl, and C₃₋₆ cycloalkyl;
R² is selected from aryl, substituted aryl, heterocycle, substituted heterocycle, heteroaryl, substituted heteroaryl and —C(═O)R³;
R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl;
Q is selected from —CH₂—, —SO₂— and —C(═O)—; and
Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl;
or a pharmaceutically acceptable salt, solvate, hydrate, or isotopic variant thereof.

23. The compound of claim 1, wherein the compound is of formula (Ib): 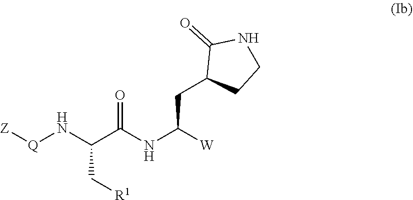
wherein:
W is selected from —CN and —C(═O)CH₂O—R²;
R¹ is selected from C₁₋₄ alkyl, substituted C₁₋₄ alkyl, and C₃₋₆ cycloalkyl;
R² is selected from aryl, substituted aryl, heterocycle, substituted heterocycle, heteroaryl, substituted heteroaryl and —C(═O)R³;
R³ is selected from C₄₋₆ alkyl, substituted C₄₋₆ alkyl, C₃₋₆ cycloalkyl, substituted C₃₋₆ cycloalkyl, aryl, substituted aryl, heterocycle, substituted heterocycle, heteroaryl and substituted heteroaryl;
Q is selected from —CH₂—, —SO₂— and —C(═O)—; and
Z is selected from C₃₋₆ cycloalkyl, substituted C₃₋₆ cycloalkyl, arylalkoxy, substituted arylalkoxy, heterocycle, substituted heterocycle, heteroaryl, and substituted heteroaryl;
or a pharmaceutically acceptable salt, solvate, hydrate, or isotopic variant thereof;
with the proviso that R³ is not 2,6-dichlorophenyl, 2,6-difluorophenyl, 2,6-dimethylphenyl, 2-cyanophenyl, 4-cyano-2-fluorophenyl, 4-chloro-2-hydroxyphenyl, 2,6-dimethoxyphenyl, 4-chlorophenyl, 4-methoxyphenyl, 4-cyanophenyl, 4-fluorophenyl, 4-methylphenyl, phenyl, cyclopropyl and t-butyl.

24. The compound of claim 1, wherein the compound is selected from the following structures:

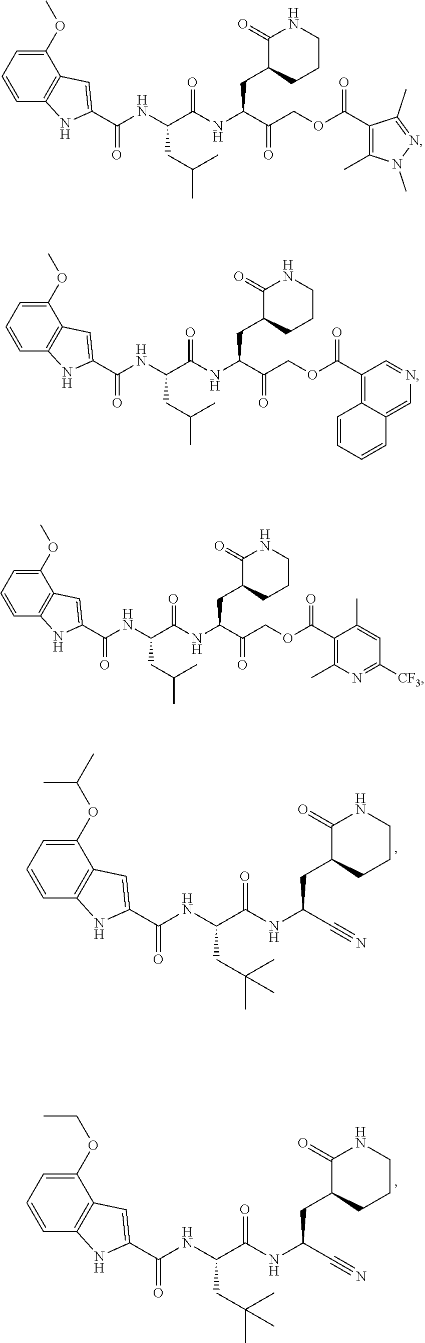 [SMILES: COc1cccc2[nH]c(C(=O)N[C@@H](CC(C)C)C(=O)N[C@@H](C[C@@H]3CCCNC3=O)C(=O)COC(=O)c3c(C)nn(C)c3C)cc12 ; COc1cccc2[nH]c(C(=O)N[C@@H](CC(C)C)C(=O)N[C@@H](C[C@@H]3CCCNC3=O)C(=O)COC(=O)c3cncc4ccccc34)cc12 ; COc1cccc2[nH]c(C(=O)N[C@@H](CC(C)C)C(=O)N[C@@H](C[C@@H]3CCCNC3=O)C(=O)COC(=O)c3c(C)cc(C(F)(F)F)nc3C)cc12 ; CC(C)Oc1cccc2[nH]c(C(=O)N[C@@H](CC(C)(C)C)C(=O)N[C@H](C#N)C[C@@H]3CCCNC3=O)cc12 ; CCOc1cccc2[nH]c(C(=O)N[C@@H](CC(C)(C)C)C(=O)N[C@H](C#N)C[C@@H]3CCCNC3=O)cc12]

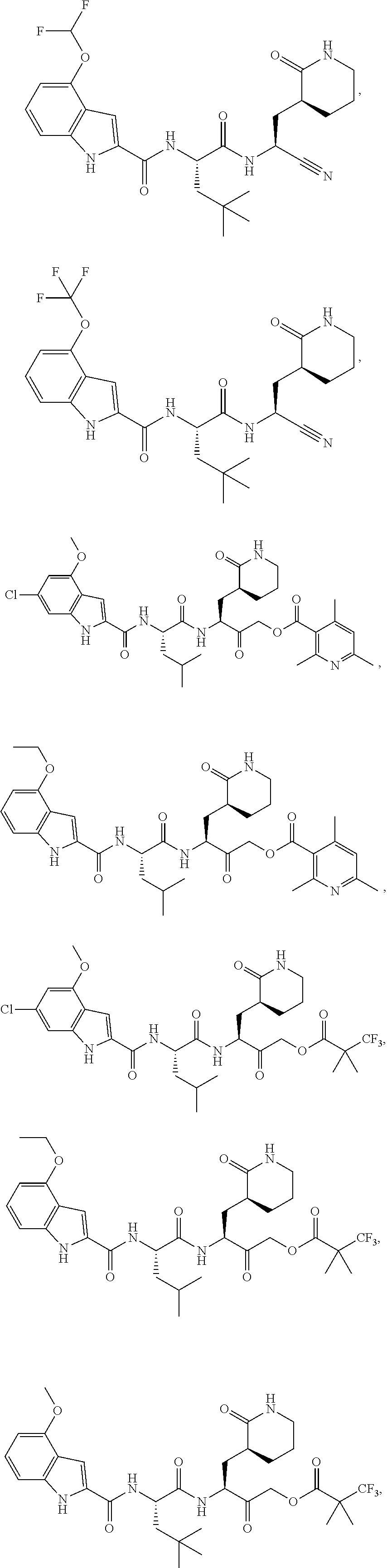

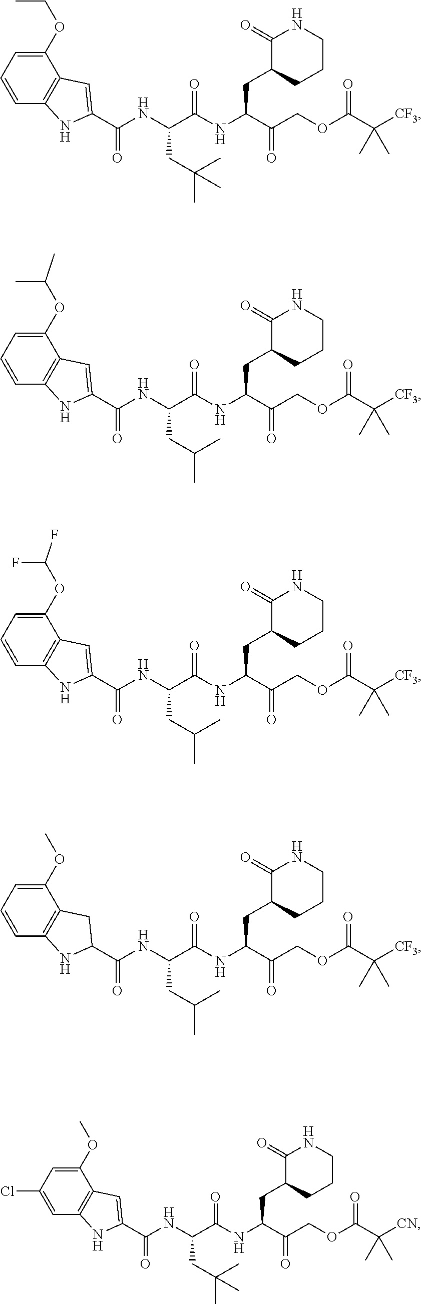

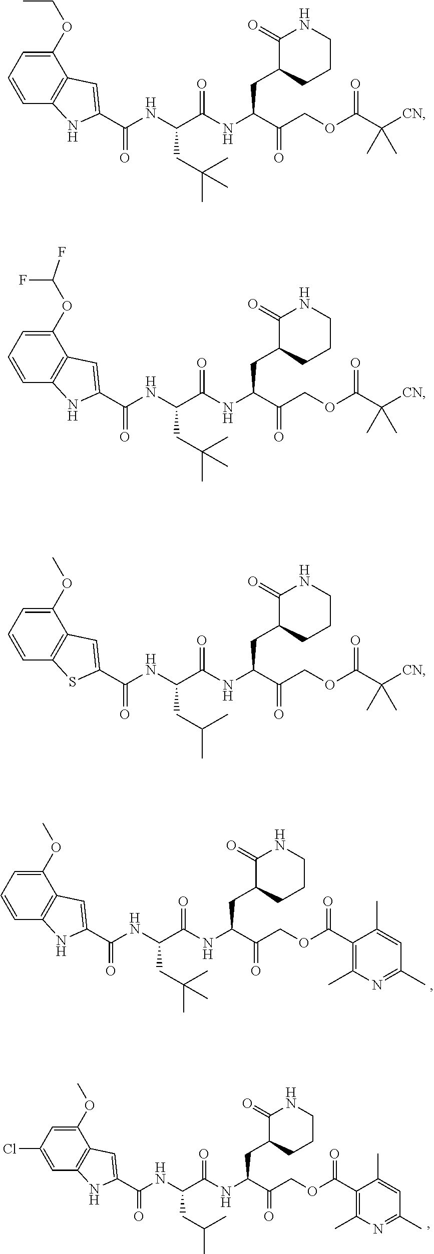 [SMILES: CCOc1cccc2[nH]c(C(=O)N[C@@H](CC(C)(C)C)C(=O)N[C@@H](C[C@@H]3CCCNC3=O)C(=O)COC(=O)C(C)(C)C#N)cc12 ; CC(C)(C)C[C@H](NC(=O)c1cc2c(OC(F)F)cccc2[nH]1)C(=O)N[C@@H](C[C@@H]1CCCNC1=O)C(=O)COC(=O)C(C)(C)C#N ; COc1cccc2sc(C(=O)N[C@@H](CC(C)C)C(=O)N[C@@H](C[C@@H]3CCCNC3=O)C(=O)COC(=O)C(C)(C)C#N)cc12 ; COc1cccc2[nH]c(C(=O)N[C@@H](CC(C)(C)C)C(=O)N[C@@H](C[C@@H]3CCCNC3=O)C(=O)COC(=O)c3c(C)cc(C)nc3C)cc12 ; COc1cc(Cl)cc2[nH]c(C(=O)N[C@@H](CC(C)C)C(=O)N[C@@H](C[C@@H]3CCCNC3=O)C(=O)COC(=O)c3c(C)cc(C)nc3C)cc12]

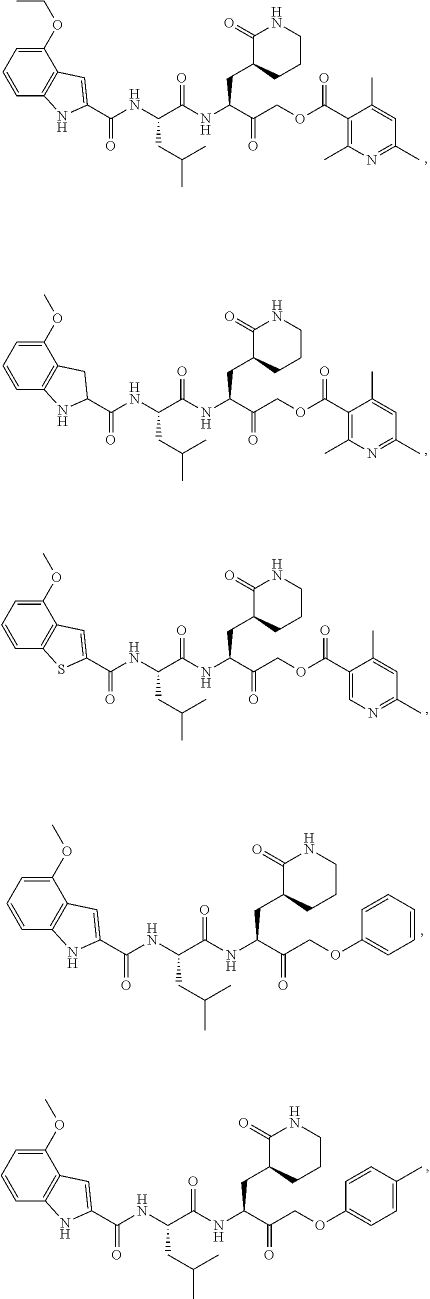 [SMILES: CCOc1cccc2[nH]c(C(=O)N[C@@H](CC(C)C)C(=O)N[C@@H](C[C@@H]3CCCNC3=O)C(=O)COC(=O)c3c(C)cc(C)nc3C)cc12 ; COc1cccc2c1CC(C(=O)N[C@@H](CC(C)C)C(=O)N[C@@H](C[C@@H]1CCCNC1=O)C(=O)COC(=O)c1c(C)cc(C)nc1C)N2 ; COc1cccc2sc(C(=O)N[C@@H](CC(C)C)C(=O)N[C@@H](C[C@@H]3CCCNC3=O)C(=O)COC(=O)c3cnc(C)cc3C)cc12 ; COc1cccc2[nH]c(C(=O)N[C@@H](CC(C)C)C(=O)N[C@@H](C[C@@H]3CCCNC3=O)C(=O)COc3ccccc3)cc12 ; COc1cccc2[nH]c(C(=O)N[C@@H](CC(C)C)C(=O)N[C@@H](C[C@@H]3CCCNC3=O)C(=O)COc3ccc(C)cc3)cc12]

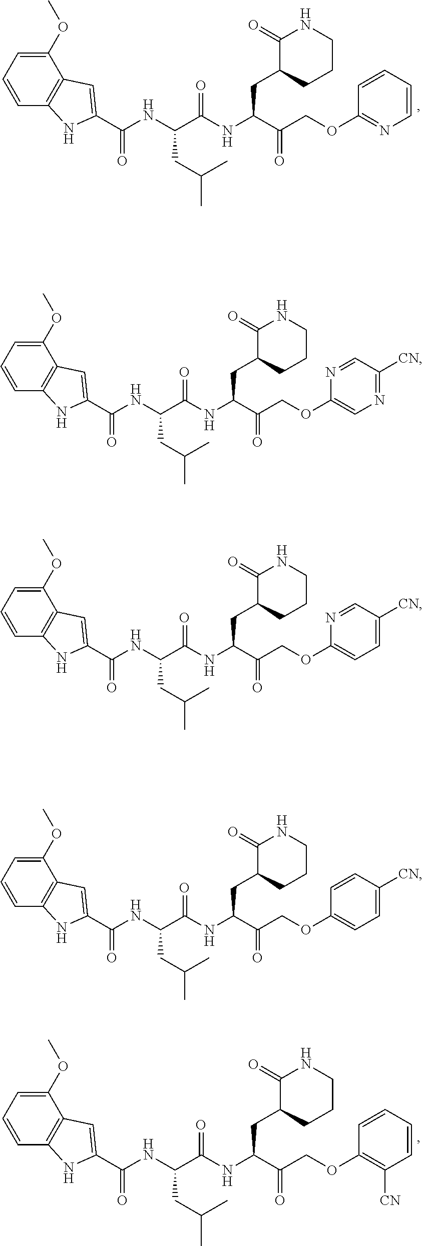 [SMILES: COc1cccc2[nH]c(C(=O)N[C@@H](CC(C)C)C(=O)N[C@@H](C[C@@H]3CCCNC3=O)C(=O)COc3ccccn3)cc12 ; COc1cccc2[nH]c(C(=O)N[C@@H](CC(C)C)C(=O)N[C@@H](C[C@@H]3CCCNC3=O)C(=O)COc3cnc(C#N)cn3)cc12 ; COc1cccc2[nH]c(C(=O)N[C@@H](CC(C)C)C(=O)N[C@@H](C[C@@H]3CCCNC3=O)C(=O)COc3ccc(C#N)cn3)cc12 ; COc1cccc2[nH]c(C(=O)N[C@@H](CC(C)C)C(=O)N[C@@H](C[C@@H]3CCCNC3=O)C(=O)COc3ccc(C#N)cc3)cc12 ; COc1cccc2[nH]c(C(=O)N[C@@H](CC(C)C)C(=O)N[C@@H](C[C@@H]3CCCNC3=O)C(=O)COc3ccccc3C#N)cc12]

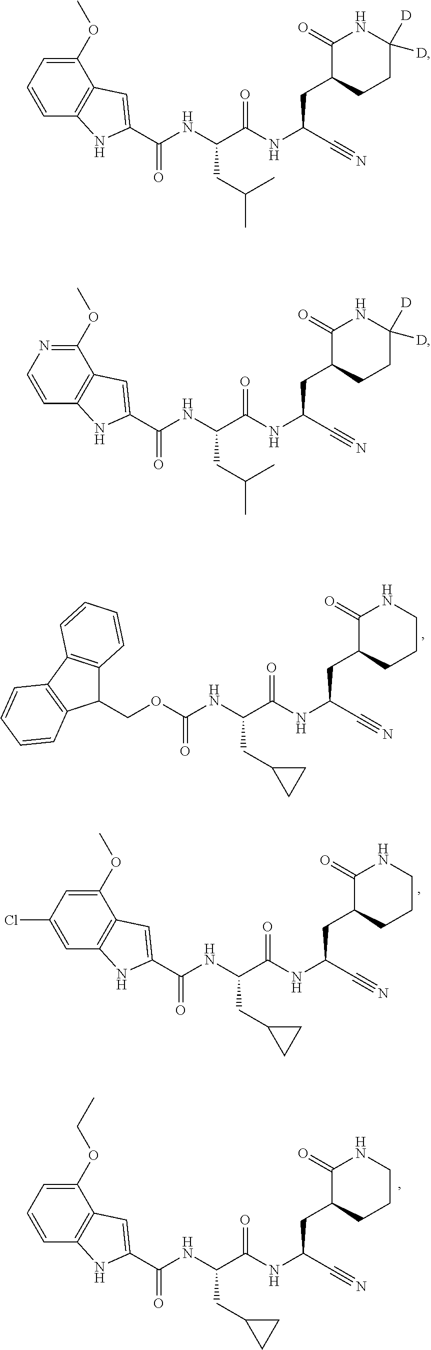

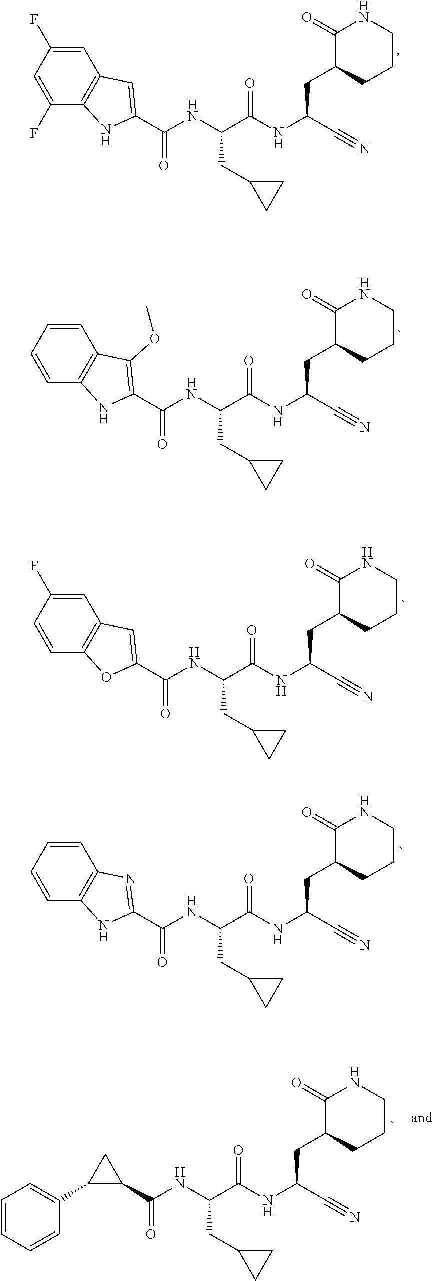 [SMILES: N#C[C@H](C[C@@H]1CCCNC1=O)NC(=O)[C@H](CC1CC1)NC(=O)c1cc2cc(F)cc(F)c2[nH]1 ; COc1c(C(=O)N[C@@H](CC2CC2)C(=O)N[C@H](C#N)C[C@@H]2CCCNC2=O)[nH]c2ccccc12 ; N#C[C@H](C[C@@H]1CCCNC1=O)NC(=O)[C@H](CC1CC1)NC(=O)c1cc2cc(F)ccc2o1 ; N#C[C@H](C[C@@H]1CCCNC1=O)NC(=O)[C@H](CC1CC1)NC(=O)c1nc2ccccc2[nH]1 ; N#C[C@H](C[C@@H]1CCCNC1=O)NC(=O)[C@H](CC1CC1)NC(=O)[C@@H]1C[C@H]1c1ccccc1]

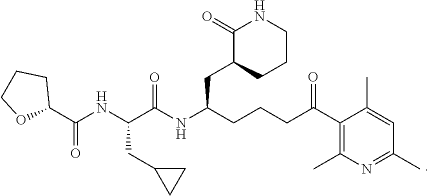 [SMILES: Cc1cc(C)c(C(=O)CCC[C@H](C[C@@H]2CCCNC2=O)NC(=O)[C@H](CC2CC2)NC(=O)[C@H]2CCCO2)c(C)n1]

25. The compound of claim 1, wherein the compound is selected from the following structures:

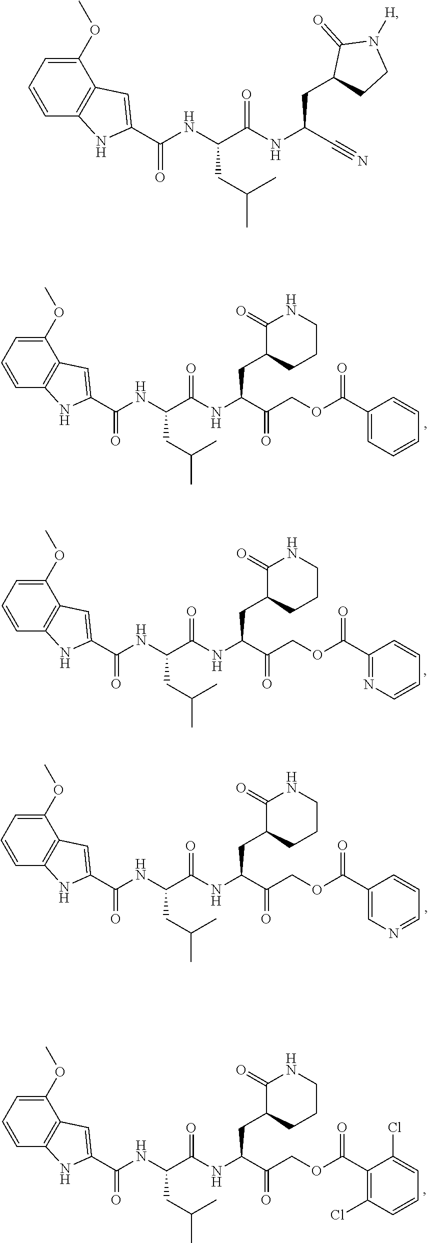

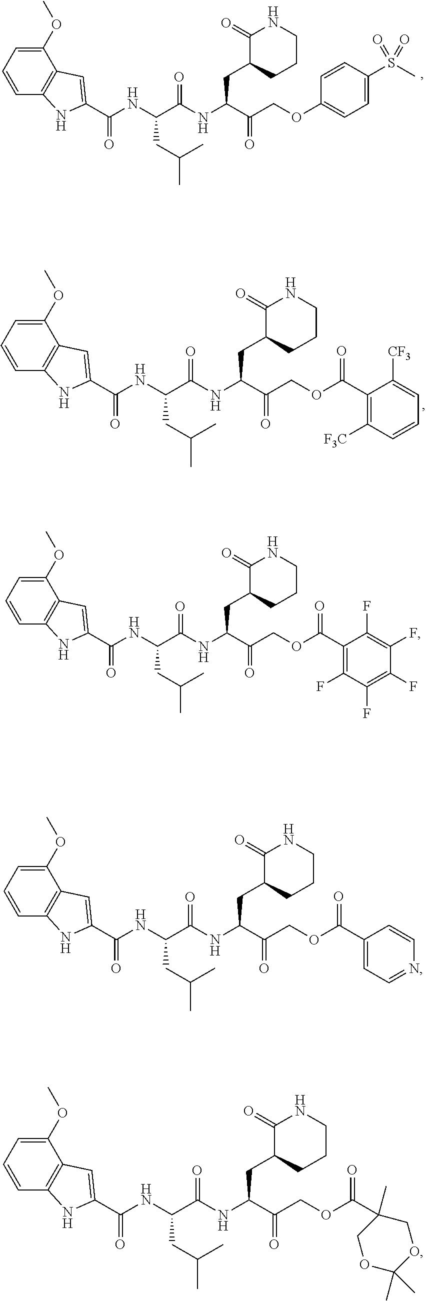

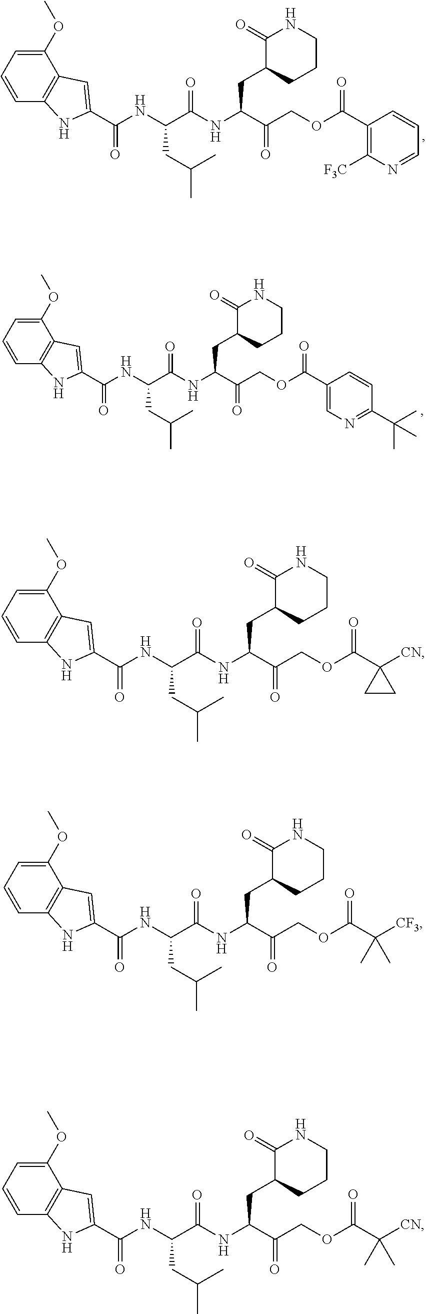 [SMILES: COc1cccc2[nH]c(C(=O)N[C@@H](CC(C)C)C(=O)N[C@@H](C[C@@H]3CCCNC3=O)C(=O)COC(=O)c3cccnc3C(F)(F)F)cc12 ; COc1cccc2[nH]c(C(=O)N[C@@H](CC(C)C)C(=O)N[C@@H](C[C@@H]3CCCNC3=O)C(=O)COC(=O)c3ccc(C(C)(C)C)nc3)cc12 ; COc1cccc2[nH]c(C(=O)N[C@@H](CC(C)C)C(=O)N[C@@H](C[C@@H]3CCCNC3=O)C(=O)COC(=O)C3(C#N)CC3)cc12 ; COc1cccc2[nH]c(C(=O)N[C@@H](CC(C)C)C(=O)N[C@@H](C[C@@H]3CCCNC3=O)C(=O)COC(=O)C(C)(C)C(F)(F)F)cc12 ; COc1cccc2[nH]c(C(=O)N[C@@H](CC(C)C)C(=O)N[C@@H](C[C@@H]3CCCNC3=O)C(=O)COC(=O)C(C)(C)C#N)cc12]

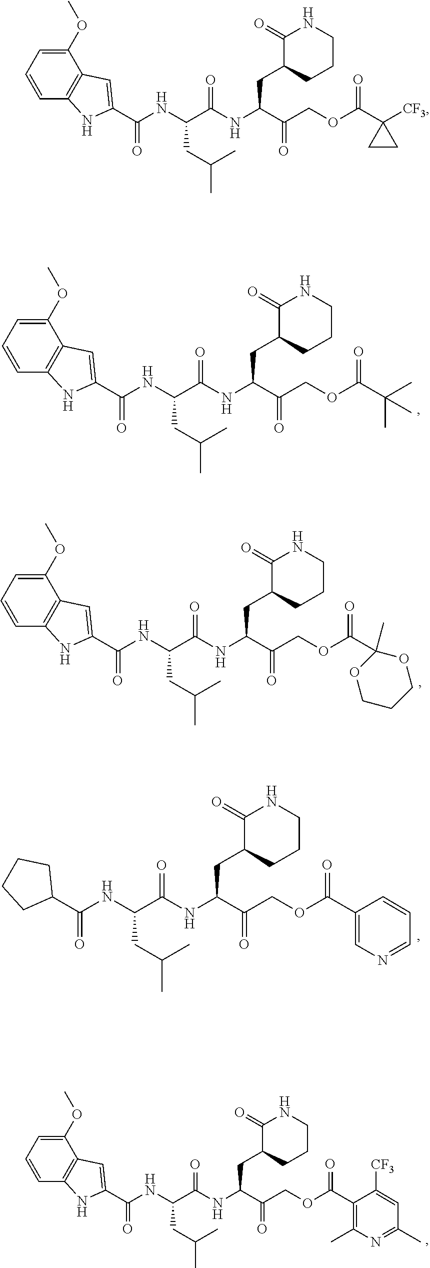 [SMILES: COc1cccc2[nH]c(C(=O)N[C@@H](CC(C)C)C(=O)N[C@@H](C[C@@H]3CCCNC3=O)C(=O)COC(=O)C3(C(F)(F)F)CC3)cc12 ; COc1cccc2[nH]c(C(=O)N[C@@H](CC(C)C)C(=O)N[C@@H](C[C@@H]3CCCNC3=O)C(=O)COC(=O)C(C)(C)C)cc12 ; COc1cccc2[nH]c(C(=O)N[C@@H](CC(C)C)C(=O)N[C@@H](C[C@@H]3CCCNC3=O)C(=O)COC(=O)C3(C)OCCCO3)cc12 ; CC(C)C[C@H](NC(=O)C1CCCC1)C(=O)N[C@@H](C[C@@H]1CCCNC1=O)C(=O)COC(=O)c1cccnc1 ; COc1cccc2[nH]c(C(=O)N[C@@H](CC(C)C)C(=O)N[C@@H](C[C@@H]3CCCNC3=O)C(=O)COC(=O)c3c(C(F)(F)F)cc(C)nc3C)cc12]

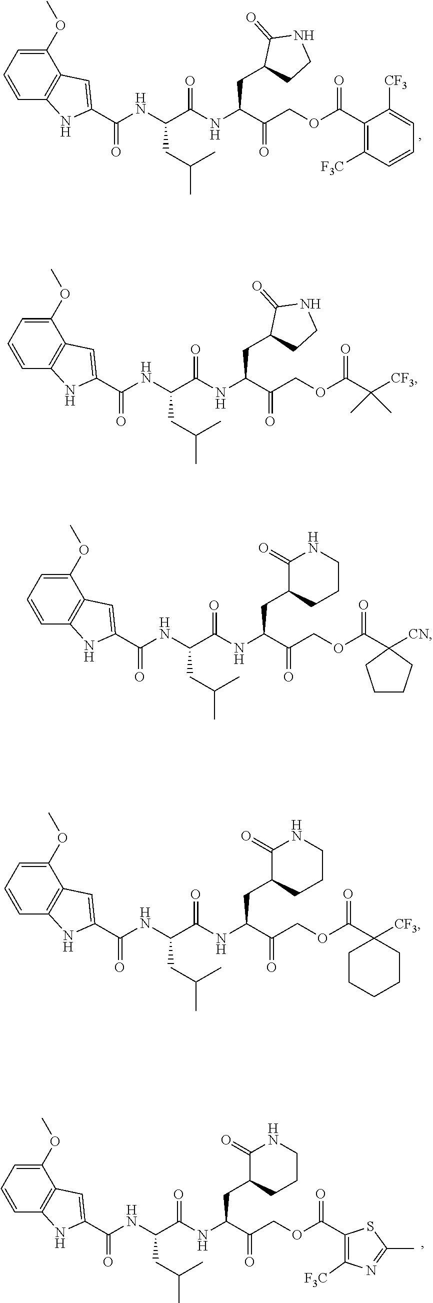

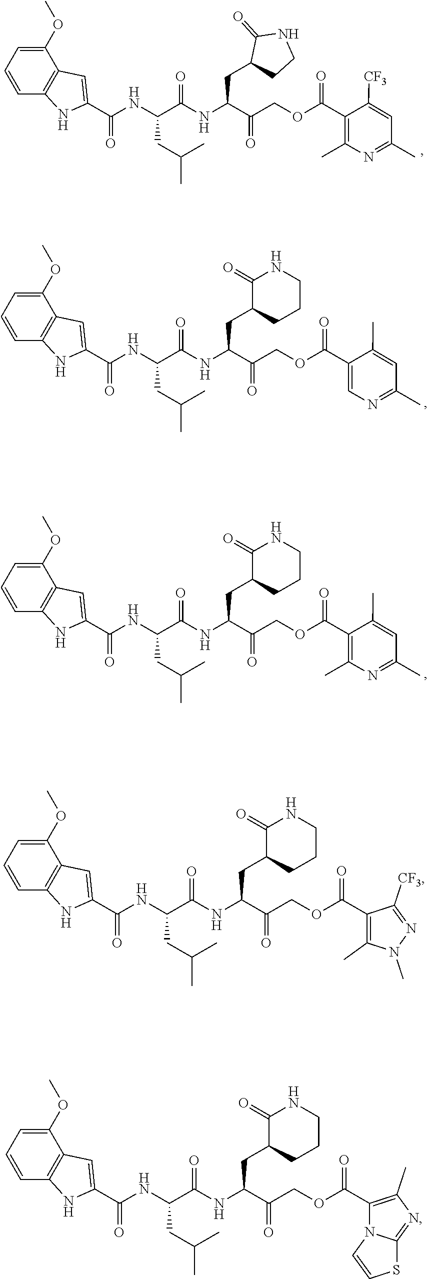

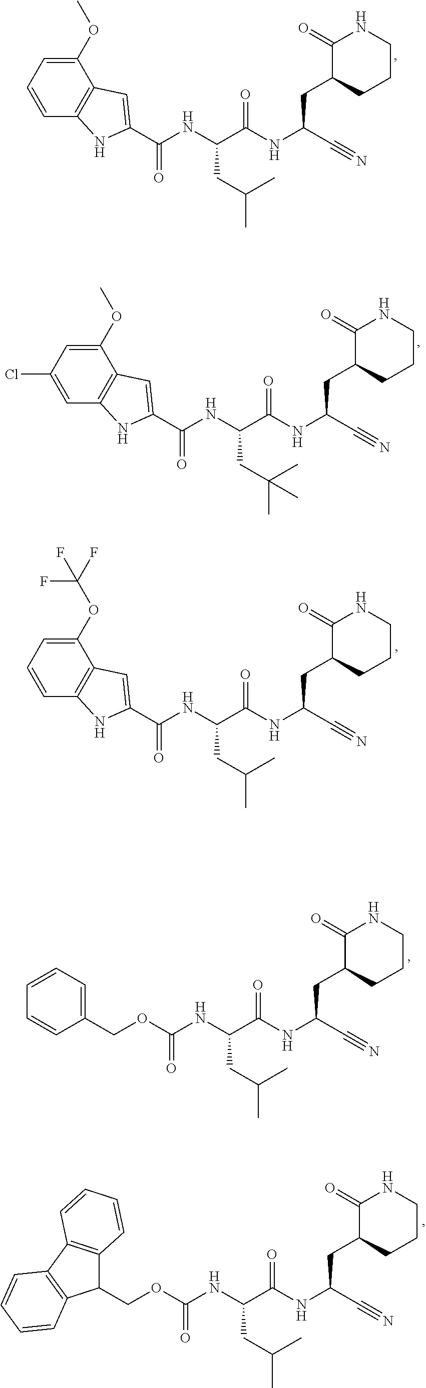 [SMILES: COc1cccc2[nH]c(C(=O)N[C@@H](CC(C)C)C(=O)N[C@H](C#N)C[C@@H]3CCCNC3=O)cc12 ; COc1cc(Cl)cc2[nH]c(C(=O)N[C@@H](CC(C)(C)C)C(=O)N[C@H](C#N)C[C@@H]3CCCNC3=O)cc12 ; CC(C)C[C@H](NC(=O)c1cc2c(OC(F)(F)F)cccc2[nH]1)C(=O)N[C@H](C#N)C[C@@H]1CCCNC1=O ; CC(C)C[C@H](NC(=O)OCc1ccccc1)C(=O)N[C@H](C#N)C[C@@H]1CCCNC1=O ; CC(C)C[C@H](NC(=O)OCC1c2ccccc2-c2ccccc21)C(=O)N[C@H](C#N)C[C@@H]1CCCNC1=O]

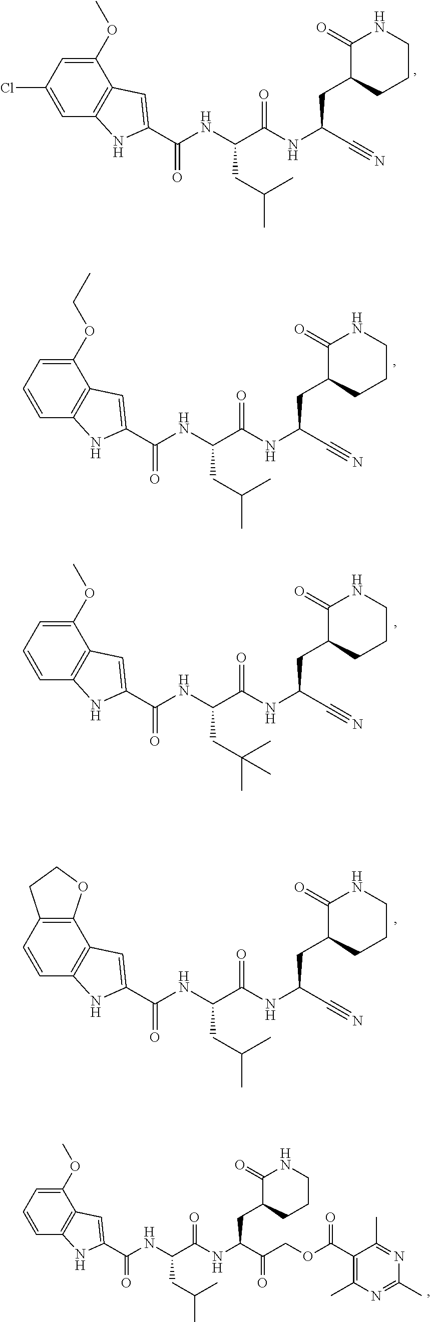 [SMILES: COc1cc(Cl)cc2[nH]c(C(=O)N[C@@H](CC(C)C)C(=O)N[C@H](C#N)C[C@@H]3CCCNC3=O)cc12 ; CCOc1cccc2[nH]c(C(=O)N[C@@H](CC(C)C)C(=O)N[C@H](C#N)C[C@@H]3CCCNC3=O)cc12 ; COc1cccc2[nH]c(C(=O)N[C@@H](CC(C)(C)C)C(=O)N[C@H](C#N)C[C@@H]3CCCNC3=O)cc12 ; CC(C)C[C@H](NC(=O)c1cc2c3c(ccc2[nH]1)CCO3)C(=O)N[C@H](C#N)C[C@@H]1CCCNC1=O ; COc1cccc2[nH]c(C(=O)N[C@@H](CC(C)C)C(=O)N[C@@H](C[C@@H]3CCCNC3=O)C(=O)COC(=O)c3c(C)nc(C)nc3C)cc12]

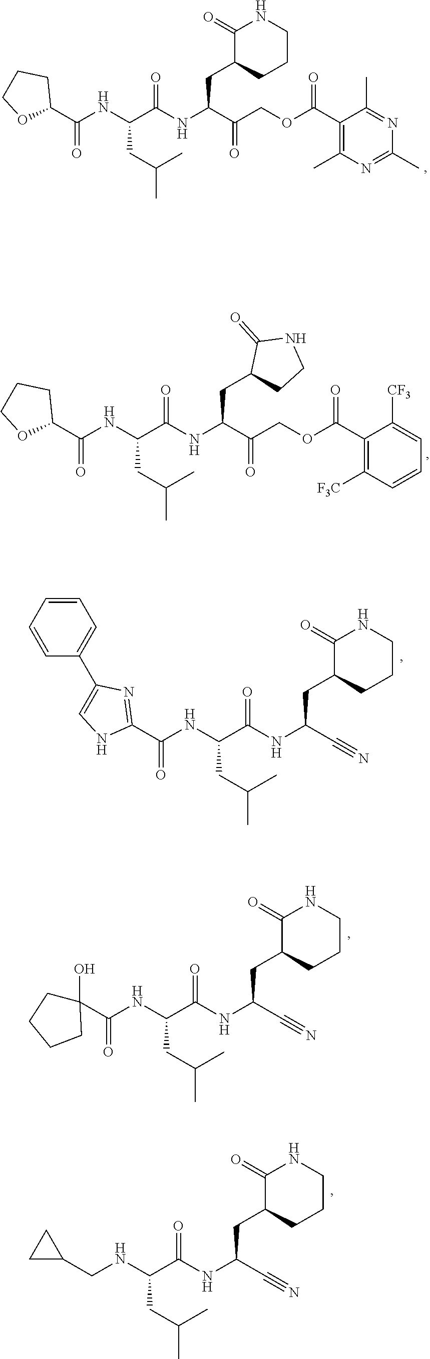 [SMILES: Cc1nc(C)c(C(=O)OCC(=O)[C@H](C[C@@H]2CCCNC2=O)NC(=O)[C@H](CC(C)C)NC(=O)[C@H]2CCCO2)c(C)n1 ; CC(C)C[C@H](NC(=O)[C@H]1CCCO1)C(=O)N[C@@H](C[C@@H]1CCNC1=O)C(=O)COC(=O)c1c(C(F)(F)F)cccc1C(F)(F)F ; CC(C)C[C@H](NC(=O)c1nc(-c2ccccc2)c[nH]1)C(=O)N[C@H](C#N)C[C@@H]1CCCNC1=O ; CC(C)C[C@H](NC(=O)C1(O)CCCC1)C(=O)N[C@H](C#N)C[C@@H]1CCCNC1=O ; CC(C)C[C@H](NCC1CC1)C(=O)N[C@H](C#N)C[C@@H]1CCCNC1=O]

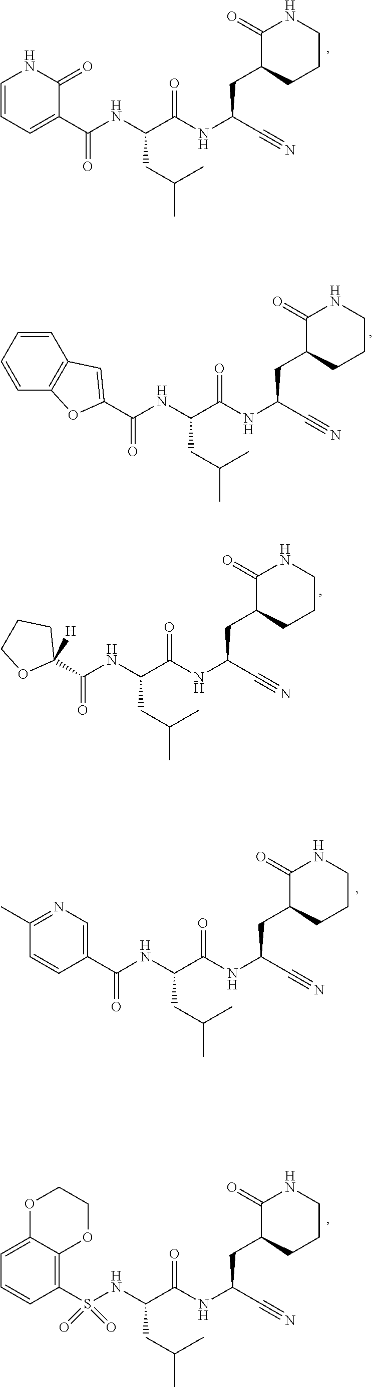

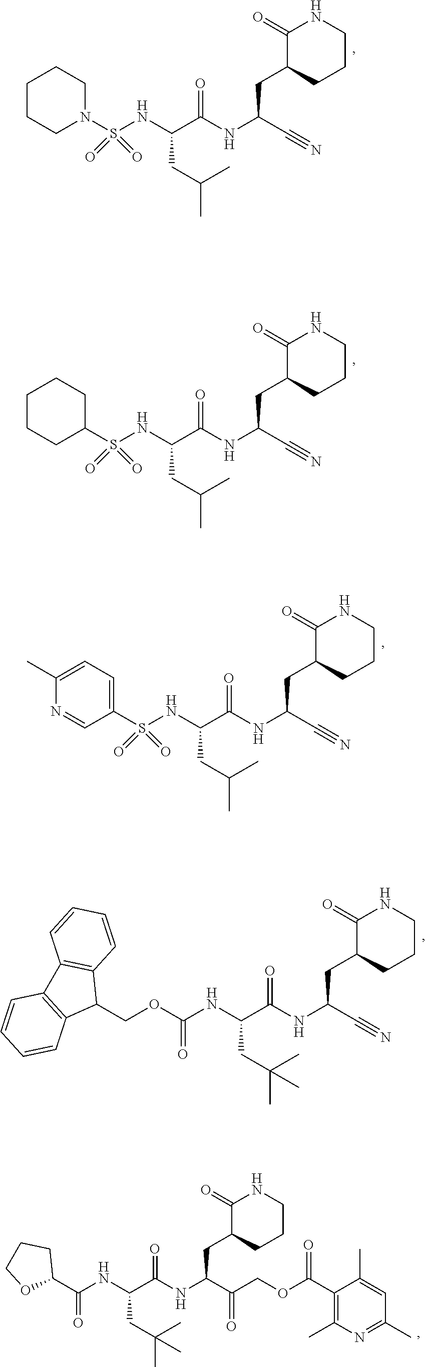 [SMILES: CC(C)C[C@H](NS(=O)(=O)N1CCCCC1)C(=O)N[C@H](C#N)C[C@@H]1CCCNC1=O ; CC(C)C[C@H](NS(=O)(=O)C1CCCCC1)C(=O)N[C@H](C#N)C[C@@H]1CCCNC1=O ; Cc1ccc(S(=O)(=O)N[C@@H](CC(C)C)C(=O)N[C@H](C#N)C[C@@H]2CCCNC2=O)cn1 ; CC(C)(C)C[C@H](NC(=O)OCC1c2ccccc2-c2ccccc21)C(=O)N[C@H](C#N)C[C@@H]1CCCNC1=O ; Cc1cc(C)c(C(=O)OCC(=O)[C@H](C[C@@H]2CCCNC2=O)NC(=O)[C@H](CC(C)(C)C)NC(=O)[C@H]2CCCO2)c(C)n1]

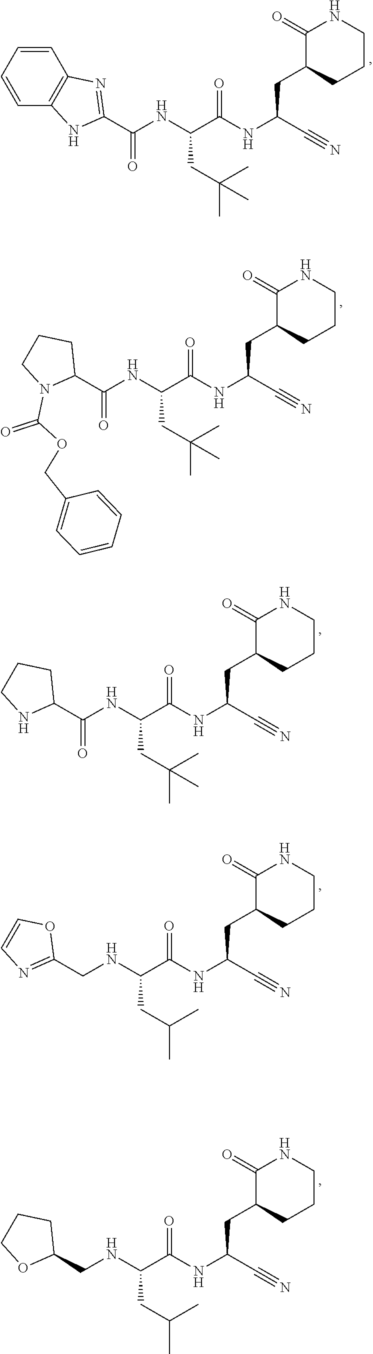 [SMILES: CC(C)(C)C[C@H](NC(=O)c1nc2ccccc2[nH]1)C(=O)N[C@H](C#N)C[C@@H]1CCCNC1=O ; CC(C)(C)C[C@H](NC(=O)C1CCCN1C(=O)OCc1ccccc1)C(=O)N[C@H](C#N)C[C@@H]1CCCNC1=O ; CC(C)(C)C[C@H](NC(=O)C1CCCN1)C(=O)N[C@H](C#N)C[C@@H]1CCCNC1=O ; CC(C)C[C@H](NCc1ncco1)C(=O)N[C@H](C#N)C[C@@H]1CCCNC1=O ; CC(C)C[C@H](NC[C@@H]1CCCO1)C(=O)N[C@H](C#N)C[C@@H]1CCCNC1=O]

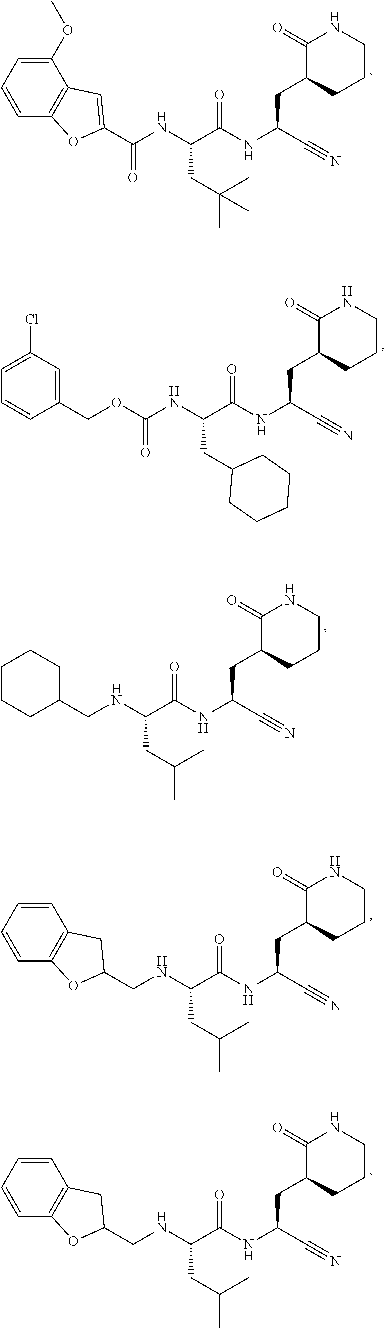 [SMILES: COc1cccc2oc(C(=O)N[C@@H](CC(C)(C)C)C(=O)N[C@H](C#N)C[C@@H]3CCCNC3=O)cc12 ; N#C[C@H](C[C@@H]1CCCNC1=O)NC(=O)[C@H](CC1CCCCC1)NC(=O)OCc1cccc(Cl)c1 ; CC(C)C[C@H](NCC1CCCCC1)C(=O)N[C@H](C#N)C[C@@H]1CCCNC1=O ; CC(C)C[C@H](NCC1Cc2ccccc2O1)C(=O)N[C@H](C#N)C[C@@H]1CCCNC1=O ; CC(C)C[C@H](NCC1Cc2ccccc2O1)C(=O)N[C@H](C#N)C[C@@H]1CCCNC1=O]

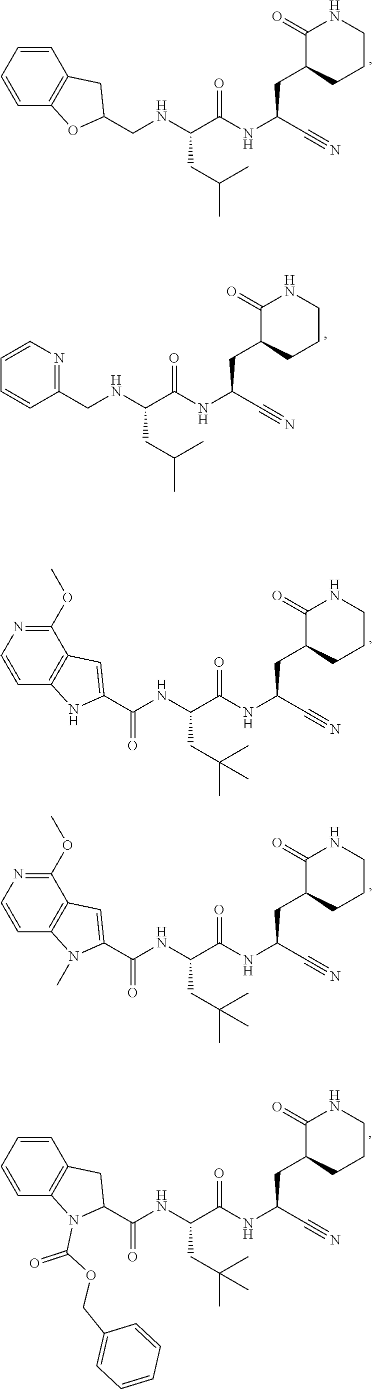 [SMILES: CC(C)C[C@H](NCC1Cc2ccccc2O1)C(=O)N[C@H](C#N)C[C@@H]1CCCNC1=O ; CC(C)C[C@H](NCc1ccccn1)C(=O)N[C@H](C#N)C[C@@H]1CCCNC1=O ; COc1nccc2[nH]c(C(=O)N[C@@H](CC(C)(C)C)C(=O)N[C@H](C#N)C[C@@H]3CCCNC3=O)cc12 ; COc1nccc2c1cc(C(=O)N[C@@H](CC(C)(C)C)C(=O)N[C@H](C#N)C[C@@H]1CCCNC1=O)n2C ; CC(C)(C)C[C@H](NC(=O)C1Cc2ccccc2N1C(=O)OCc1ccccc1)C(=O)N[C@H](C#N)C[C@@H]1CCCNC1=O]

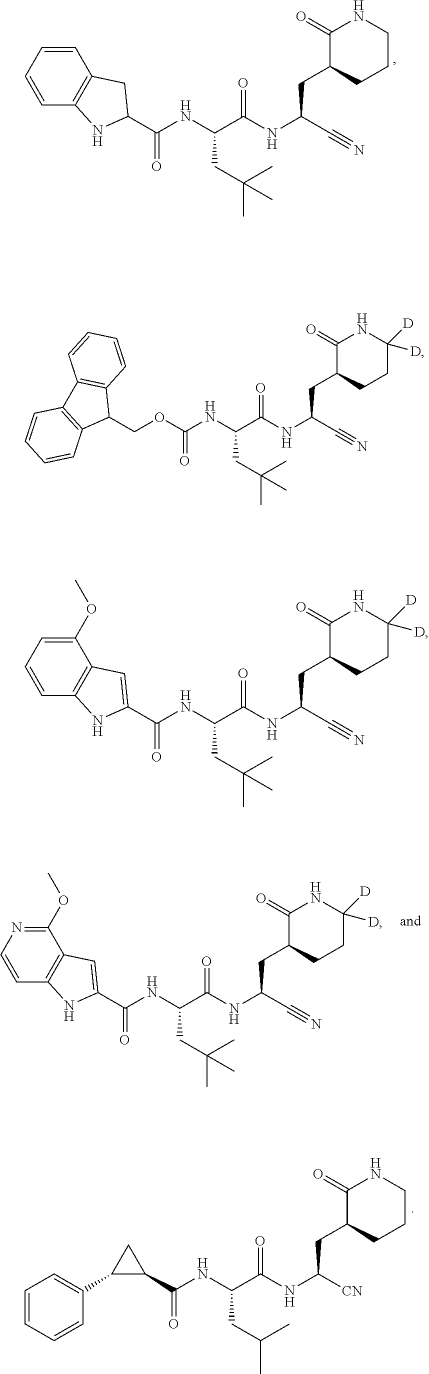

26. A method of inhibiting a Baltimore Group IV RNA virus in a cell infected with a Baltimore Group IV RNA virus, the method comprising contacting the cell with a compound of claim 1.

27.-35. (canceled)

36. A method of treating a Baltimore Group IV RNA virus infection in a mammal, the method comprising administering to the mammal an effective amount of a compound according to claim 1.

37.-47. (canceled)
